(19) United States
(12) Patent Application Publication
(10) Pub. No.: US 2018/0133220 A1
(43) Pub. Date: May 17, 2018

(54) COMPOUNDS FOR USE IN TREATING ACUTE CORONARY SYNDROME AND RELATED CONDITIONS

(71) Applicant: Vitae Pharmaceuticals, Inc., Parsippany, NJ (US)

(72) Inventor: Richard E. Gregg, Pennington, NJ (US)

(21) Appl. No.: 15/869,300

(22) Filed: Jan. 12, 2018

Related U.S. Application Data
(63) Continuation of application No. 15/319,999, filed on Dec. 19, 2016, now abandoned.
(60) Provisional application No. 62/014,266, filed on Jun. 19, 2014.

(51) Int. Cl. A61K 31/506

(52) U.S. Cl. CPC A61K 31/506

(57) ABSTRACT

Provided herein are compounds and pharmaceutically acceptable salts thereof that are useful therapeutics for acute coronary syndrome and related disorders.

### RELATED APPLICATIONS

This application claims the benefit of U.S. Provisional Application No. 62/014,266, filed Jun. 19, 2014, the entire contents of which are incorporated by reference herein.

### FIELD OF THE INVENTION

The present invention relates to compounds that are useful as therapeutic agents for the treatment of acute coronary syndrome and related conditions.

### BACKGROUND OF THE INVENTION

Acute coronary syndrome (ACS) refers to a spectrum of clinical presentations ranging from those for ST-segment elevation myocardial infarction (STEMI) to presentations found in nonST-segment elevation myocardial infarction (NSTEMI) or in unstable angina. It is almost always associated with rupture of an atherosclerotic plaque and partial or complete thrombosis of the infarct-related artery. The pathogenesis of ACS involves an intricate interplay among the endothelium, the inflammatory cells, and the thrombogenicity of the blood. ACS has a very high incidence (10-20%) of a completed myocardial infarction within six months of an ACS event. Autopsy studies have shown that plaque rupture causes approximately 75% of fatal myocardial infarctions, whereas superficial endothelial erosion accounts for the remaining 25%. The hallmarks of an ACS atherosclerotic plaque are lipid rich foam cells, inflammatory cell infiltrates, thin cap of the atherosclerotic plaque prone to rupture, and a surface to the plaque that is prone to form blood clots (thrombogenic).

Because ACS, a common complication of coronary heart disease, is associated with more than 2.5 million hospitalizations each year (Grech E D, Ramsdale D R. Acute coronary syndrome: unstable angina and non-ST segment elevation myocardial infarction. BMJ 2003; 326:1259-1261), there is an ongoing need for new therapeutic agents for the treatment of acute coronary syndrome and related conditions.

### SUMMARY OF THE INVENTION

Disclosed are compounds that are useful as therapeutic agents for the treatment of acute coronary syndrome and related conditions. Such related conditions include e.g., heart attack, myocardial infarction, acute myocardial infarction, non-ST-segment elevation myocardial infarction, ST-segment elevation myocardial infarction, unstable angina, stable angina, angina pectoris, exercise induced angina, coronary artery disease, coronary heart disease, acute myocardial ischemia, ischaemic heart disease, ischemia, recurrent ischemia, congestive heart disease, congestive heart failure, cardiomyopathy, hypertensive heart disease, heart failure, diastolic heart failure, systolic heart failure, cor pulmonale, cardiac dysrhythmias, abnormalities of heart rhythm, inflammatory heart disease, endocarditis, inflammatory cardiomegaly, myocarditis, cerebrovascular disease, peripheral arterial disease, reperfusion injury, restenosis, atherosclerotic lesions, or chronic atherosclerotic inflammation.

In one aspect, the present disclosure provides a method of treating treating a condition selected from acute coronary syndrome, heart attack, myocardial infarction, acute myocardial infarction, non-ST-segment elevation myocardial infarction, ST-segment elevation myocardial infarction, unstable angina, stable angina, angina pectoris, exercise induced angina, coronary artery disease, coronary heart disease, acute myocardial ischemia, ischaemic heart disease, ischemia, recurrent ischemia, congestive heart disease, congestive heart failure, cardiomyopathy, hypertensive heart disease, heart failure, diastolic heart failure, systolic heart failure, cor pulmonale, cardiac dysrhythmias, abnormalities of heart rhythm, inflammatory heart disease, endocarditis, inflammatory cardiomegaly, myocarditis, cerebrovascular disease, peripheral arterial disease, reperfusion injury, restenosis, atherosclerotic lesions, and chronic atherosclerotic inflammation in a subject, comprising administering to the subject an effective amount of a compound represented by structural Formula I:

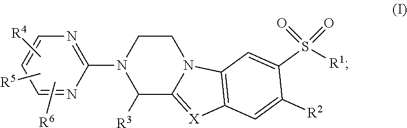

or a pharmaceutically acceptable salt thereof, wherein:

X is N or CR^{c};

R¹ is alkyl or —NR^{a}R^{b};

R² is H, halogen, —CN, —NRC(O)R, —C(O)OR, —C(O)NR^{a}R^{b}, monocyclic heteroaromatic optionally substituted with one or more groups selected from alkyl, —CN, —NRC(O)R, —C(O)OR, —C(O)NR^{a}R^{b} and halogen, monocyclic non-aromatic heterocycle optionally substituted with one or more groups selected from alkyl, halogen, —CN and ═O, or alkyl optionally substituted by one or more groups selected from halogen, hydroxy, alkoxy, —NR^{a}R^{b}, —NRC(O)R, —NRC(O)O(alkyl), —NRC(O)N(R)₂, —C(O)OR, thiol, alkylthiol, nitro, —CN, ═O, —OC(O)H, —OC(O)(alkyl), —OC(O)O(alkyl), —OC(O)N(R)₂ and —C(O)NR^{a}R^{b};

R³ is alkyl, haloalkyl, hydroxyalkyl, alkoxyalkyl, cycloalkyl, monocyclic non-aromatic heterocycle, monocyclic heteroaromatic or phenyl, wherein the phenyl, monocyclic non-aromatic heterocycle and monocyclic heteroaromatic group represented by R³ are optionally substituted with one or more groups selected from alkyl, halogen, haloalkyl, alkoxy, haloalkoxy, nitro and CN;

R⁴ is halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic or alkyl, wherein the alkyl, monocyclic non-aromatic heterocycle and monocyclic heteroaromatic group represented by R⁴ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;

R⁵ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic or alkyl, wherein the alkyl, monocyclic non-aromatic heterocycle and monocyclic heteroaromatic group represented by R⁵ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;

R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂; or R⁵ and R⁶, taken together with the carbon atoms to which they are bonded, form a moncyclic non-aromatic heterocycle opotionally substituted with one or more groups selected from alkyl, halogen, hydroxyalkyl, alkoxyalkyl, haloalkyl and =0;

each R independently is H or alkyl;

R^{a} and R^{b} are independently H or alkyl, or R^{a} and R^{b} can be taken together with the nitrogen to which they are attached to form a monocyclic non-aromatic heterocycle; and

R^{c} is H, alkyl, or halogen;

In another aspect the subject is human and the condition is acute coronary syndrome (ACS).

### BRIEF DESCRIPTION OF THE DRAWINGS

FIG. 1A is a bar graph showing average carotid artery cholesterol ester content (X-axis) versus the total daily dose of administered compound (Y-axis). Dosages are total amount per day based on twice per day dosing. “Vehicle” indicates no compound was administered.

FIG. 1B is a bar graph showing the average carotid artery FDG-6 phosphate content (Y-axis) versus the total daily dose of administered compound (Y-axis). The numbers in the bars indicates the number of mice in the cohort.

### DETAILED DESCRIPTION OF THE INVENTION

Compounds

The compound(s) described in the methods herein include both the neutral form and a pharmaceutically acceptable salt thereof.

In a first alternative embodiment of the methods described herein, R³ is alkyl, haloalkyl, hydroxyalkyl, alkoxyalkyl, cycloalkyl, or phenyl, wherein the phenyl group represented by R³ is optionally substituted with one or more groups selected from alkyl, halogen, haloalkyl, alkoxy, haloalkoxy, nitro and —CN;

R⁴ is halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl represented by R⁴ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;

R⁵ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl represented by R⁵ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂; and

R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂, wherein the values for the variables are as defined for Formula I above.

In a second alternative embodiment of the methods described herein, the compound is represented by structural Formula II, III, IV, V, VI, or VII:

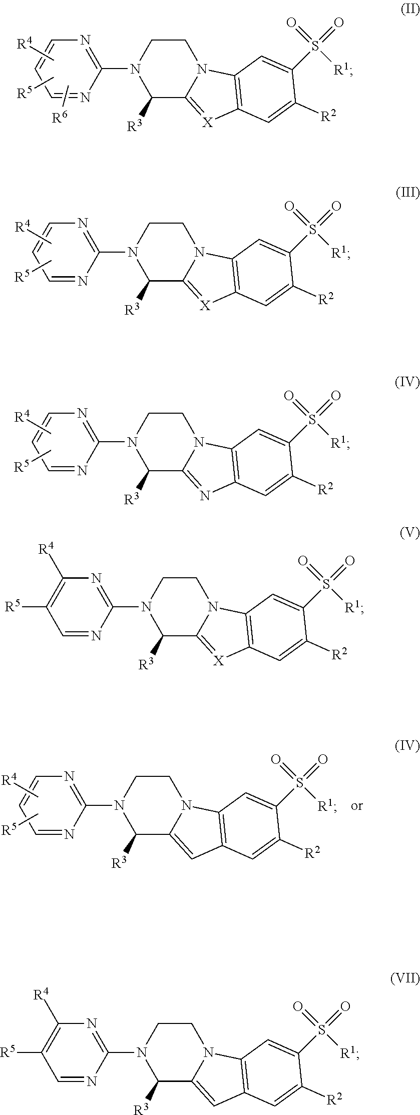

or a pharmaceutically acceptable salt thereof, wherein the values for the variables are as defined for Formula I above or in the first alternative embodiment.

In a third alternative embodiment of the methods described herein, in any compound of formulas I through VII,

R¹ is methyl or —NH₂;

R² is H or methyl, wherein the methyl group represented by R² is optionally substituted with one or more groups selected from halogen, hydroxy, alkoxy, —NR^{a}R^{b}, —NRC(O)R, —NRC(O)O(alkyl), —NRC(O)N(R)₂, —C(O)OR, thiol, alkylthiol, nitro, —CN, ═O, —OC(O)H, —OC(O)(alkyl), —OC(O)O(alkyl), —C(O)NR^{a}R^{b} and —OC(O)N(R)₂;

R³ is methyl, ethyl, propyl, isopropyl, tert-butyl, sec-butyl, iso-butyl, —CH₂CF₃, —CH(CH₂F)₂, —CH(CHF₂)₂, —CH(CF₃)₂, —CF(CH₃)₂, —CF₃, cyclopropyl, cyclobutyl, cyclopentyl, cyclohexyl, —C(OH)(CH₃)₂, —CH(OH)(CH₃), or phenyl, wherein the phenyl group represented by R³ is optionally substituted with one or more groups selected from alkyl, halogen, haloalkyl, alkoxy, haloalkoxy, nitro and —CN; and

R^{c}, where present, is H, wherein the values for the remaining variables are as defined for Formula I or for the first or second alternative embodiment. Alternatively, R² is H or —CH₂OH, wherein the values for the remaining variables are as defined for Formula I or for the first or second alternative embodiments.

In a fourth alternative embodiment of the methods described herein, in any compound of formulas I through VII, R is methyl; R² is —CH₂OH; and R³ is isopropyl, wherein the values for the remaining variables are as defined for Formula I or for the first, second, or third alternative embodiments.

In a fifth alternative embodiment of the methods described herein, in any compound of formulas I through VII, R⁴ is halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl or dihydroxyalkyl; and R⁵ is H, halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl or dihydroxyalkyl, wherein the values for the remaining variables are as defined for Formula I or for the first, second, third, or fourth alternative embodiments.

In a sixth alternative embodiment of the methods described herein, in any compound of formulas I through VII, R⁴ is methyl, ethyl, hydroxy, CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂ or —OCH₃; and

R⁵ is H, methyl, ethyl, hydroxy, CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂ or —OCH₃, wherein the values for the remaining variables are as defined for Formula I or for the first, second, third, fourth, or fifth alternative embodiments. Alternatively, R⁴ is as just described and R⁵ is H or C(OH)(CH₃)₂, wherein the values for the remaining variables are as defined for Formula I or for the first, second, third, fourth, or fifth alternative embodiments.

In a seventh alternative embodiment of the methods described herein, in any compound of formulas I through VII, R⁴ is alkyl, haloalkyl, cycloalkyl, alkoxy, or haloalkoxy, wherein the values for the remaining variables are as defined for Formula I or for the first, second, third, fourth, fifth, or sixth alternative embodiments. Alternatively, R⁴ is methyl, halogenated methyl, cyclopropyl, —OCHF₂ or —OCH₃, wherein the values for the remaining variables are as defined for Formula I or for the first, second, third, fourth, fifth, or sixth alternative embodiments. In another alternative, R⁴ is CF₃, wherein the values for the remaining variables are as defined for Formula I or for the first, second, third, fourth, fifth, or sixth alternative embodiments. In another alternative, R⁴ is as just described and R⁵ is H or C(OH)(CH₃)₂, wherein the values for the remaining variables are as defined for Formula I or for the first, second, third, fourth, fifth, or sixth alternative embodiments.

In a eighth alternative embodiment of the methods described herein, in any compound of formulas I through VII, R⁵ is H; and the remaining variables are as defined for Formula I or for the first, second, third, fourth, fifth, sixth, or seventh alternative embodiments.

In a ninth alternative embodiment of the methods described herein, in any compound of formulas I through VII, R⁵ is not H; and the remaining variables are as defined for Formula I or for the first, second, third, fourth, fifth, sixth, seventh, or eighth alternative embodiments.

Another embodiment is a method of a treating acute coronary syndrome or a related condition in a subject comprising administering to the subject an effective amount of a compound represented by formulas I, II, III, IV, V, VI or VII or a pharmaceutically acceptable salt thereof, wherein the variables are as defined for formula I or in the first, second, third, fourth, fifth, sixth, seventh, eighth, or ninth alternative embodiments, provided that the compound comprises at least one group represented by —C(O)OR.

Another embodiment is a method of a treating acute coronary syndrome or a related condition in a subject comprising administering to the subject an effective amount of a compound represented by formulas I, II, III, IV, V, VI or VII or a pharmaceutically acceptable salt thereof, wherein the variables are as defined for formula I or in the first, second, third, fourth, fifth, sixth, seventh, eighth, or ninth alternative embodiments, provided that the compound comprises no groups represented by —C(O)OR.

The compounds of the methods described herein contain at least one chiral center and, therefore, exist as enantiomers. When compounds of the methods described herein are depicted or named without indicating the stereochemistry, it is to be understood that enantiomerically pure forms and mixtures of enantiomers, including racemic mixtures, are encompassed.

When a compound is designated by a name or structure that indicates a single enantiomer, unless indicated otherwise, the compound is at least 50%, 60%, 70%, 80%, 90%, 95%, 99%, 99.5% or 99.9% optically pure (also referred to as “enantiomerically pure”). Optical purity is the weight in the mixture of the named or depicted enantiomer divided by the total weight in the mixture of both enantiomers.

When a compound is designated by a name or structure that indicates a specific stereochemistry at a chiral center, unless indicated otherwise, the compound is at least 50%, 60%, 70%, 80%, 90%, 95%, 99%, 99.5% or 99.9% stereoisomerically pure. Stereoisomeric purity is the weight in the mixture of the named or depicted stereoisomer(s) divided by the total weight in the mixture of all stereoisomers.

In a tenth alternative embodiment, the present disclosure provides a method of treating acute coronary syndrome or a related condition (e.g., those described above) in a subject comprising administering to the subject an effective amount of a compound depicted by a structural formula in Table 1, or a pharmaceutically acceptable salt thereof.

TABLE 1

| Com- | | |
|---|---|---|
| pound | | |
| No. | Example No. | Structure |
| E1 | Example 01 | 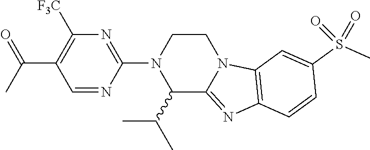 [SMILES: CC(=O)c1cnc(N2CCn3c(nc4ccc(S(C)(=O)=O)cc43)C2C(C)C)nc1C(F)(F)F] |
| E2 | Example 02 | 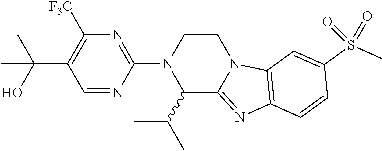 [SMILES: CC(C)C1c2nc3ccc(S(C)(=O)=O)cc3n2CCN1c1ncc(C(C)(C)O)c(C(F)(F)F)n1] |
| E3 | Example 03 | 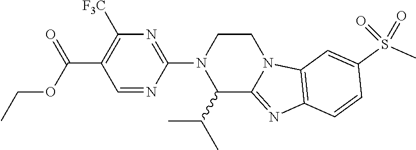 [SMILES: CCOC(=O)c1cnc(N2CCn3c(nc4ccc(S(C)(=O)=O)cc43)C2C(C)C)nc1C(F)(F)F] |
| E4 | Example 04 | 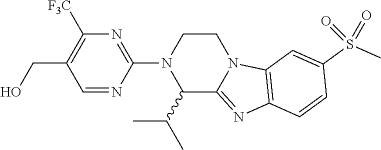 [SMILES: CC(C)C1c2nc3ccc(S(C)(=O)=O)cc3n2CCN1c1ncc(CO)c(C(F)(F)F)n1] |
| E5 | Example 05 | 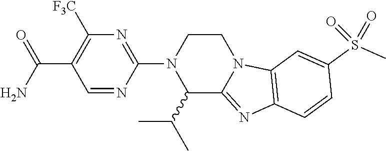 [SMILES: CC(C)C1c2nc3ccc(S(C)(=O)=O)cc3n2CCN1c1ncc(C(N)=O)c(C(F)(F)F)n1] |
| E6a | Example 06, isomer 1 | 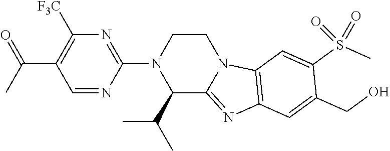 [SMILES: CC(=O)c1cnc(N2CCn3c(nc4cc(CO)c(S(C)(=O)=O)cc43)[C@H]2C(C)C)nc1C(F)(F)F] |
| E6b | Example 06, isomer 2 | 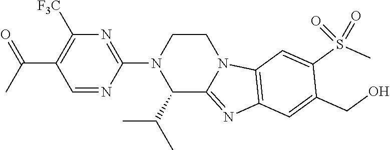 [SMILES: CC(=O)c1cnc(N2CCn3c(nc4cc(CO)c(S(C)(=O)=O)cc43)[C@@H]2C(C)C)nc1C(F)(F)F] |
| E7a | Example 07, isomer 1 | 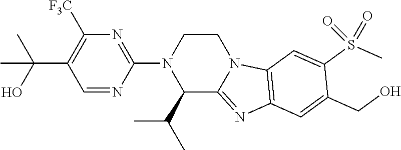 [SMILES: CC(C)[C@@H]1c2nc3cc(CO)c(S(C)(=O)=O)cc3n2CCN1c1ncc(C(C)(C)O)c(C(F)(F)F)n1] |
| E7b | Example 07, isomer 2 | 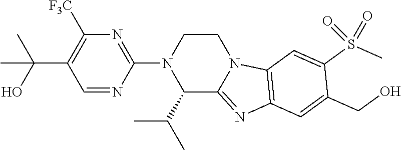 [SMILES: CC(C)[C@H]1c2nc3cc(CO)c(S(C)(=O)=O)cc3n2CCN1c1ncc(C(C)(C)O)c(C(F)(F)F)n1] |
| E8a | Example 08, isomer 1 | 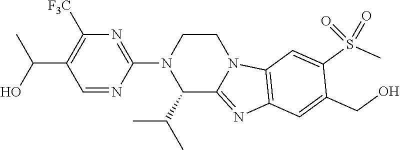 [SMILES: CC(O)c1cnc(N2CCn3c(nc4cc(CO)c(S(C)(=O)=O)cc43)[C@@H]2C(C)C)nc1C(F)(F)F] |
| E8b | Example 08, isomer 2 | 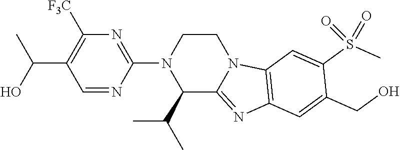 [SMILES: CC(O)c1cnc(N2CCn3c(nc4cc(CO)c(S(C)(=O)=O)cc43)[C@H]2C(C)C)nc1C(F)(F)F] |
| E8c | Example 08, isomer 3 | 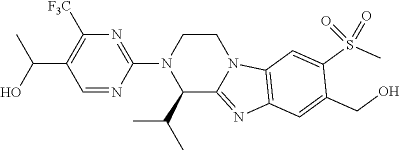 [SMILES: CC(O)c1cnc(N2CCn3c(nc4cc(CO)c(S(C)(=O)=O)cc43)[C@H]2C(C)C)nc1C(F)(F)F] |
| E8d | Example 08, isomer 4 | 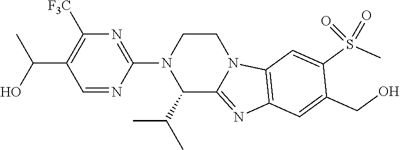 [SMILES: CC(O)c1cnc(N2CCn3c(nc4cc(CO)c(S(C)(=O)=O)cc43)[C@@H]2C(C)C)nc1C(F)(F)F] |
| E9a | Example 09, isomer 1 | 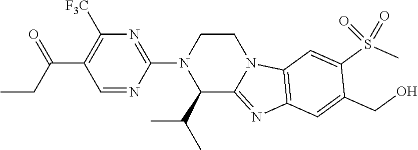 [SMILES: CCC(=O)c1cnc(N2CCn3c(nc4cc(CO)c(S(C)(=O)=O)cc43)[C@H]2C(C)C)nc1C(F)(F)F] |
| E9b | Example 09, isomer 2 | 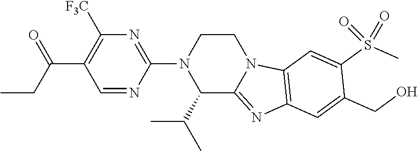 [SMILES: CCC(=O)c1cnc(N2CCn3c(nc4cc(CO)c(S(C)(=O)=O)cc43)[C@@H]2C(C)C)nc1C(F)(F)F] |
| E10a | Example 10, isomer 1 | 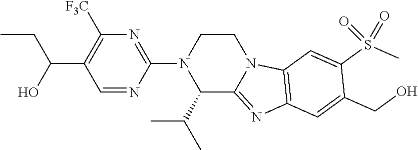 [SMILES: CCC(O)c1cnc(N2CCn3c(nc4cc(CO)c(S(C)(=O)=O)cc43)[C@@H]2C(C)C)nc1C(F)(F)F] |
| E10b | Example 10, isomer 2 | 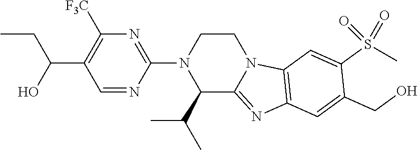 [SMILES: CCC(O)c1cnc(N2CCn3c(nc4cc(CO)c(S(C)(=O)=O)cc43)[C@H]2C(C)C)nc1C(F)(F)F] |
| E10c | Example 10, isomer 3 | 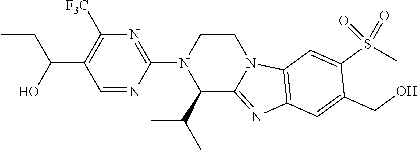 [SMILES: CCC(O)c1cnc(N2CCn3c(nc4cc(CO)c(S(C)(=O)=O)cc43)[C@H]2C(C)C)nc1C(F)(F)F] |
| E10d | Example 10, isomer 4 | 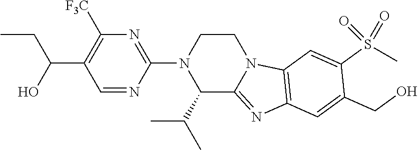 [SMILES: CCC(O)c1cnc(N2CCn3c(nc4cc(CO)c(S(C)(=O)=O)cc43)[C@@H]2C(C)C)nc1C(F)(F)F] |
| E11a | Example 11, isomer 1 | 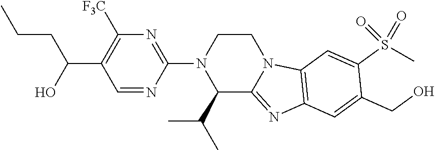 [SMILES: CCCC(O)c1cnc(N2CCn3c(nc4cc(CO)c(S(C)(=O)=O)cc43)[C@H]2C(C)C)nc1C(F)(F)F] |
| E11b | Example 11, isomer 2 | 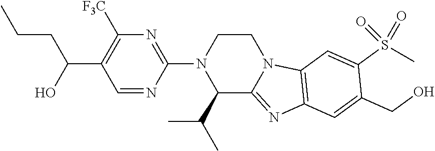 [SMILES: CCCC(O)c1cnc(N2CCn3c(nc4cc(CO)c(S(C)(=O)=O)cc43)[C@H]2C(C)C)nc1C(F)(F)F] |
| E12 | Example 12 | 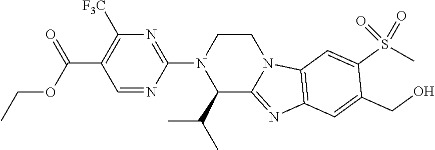 [SMILES: CCOC(=O)c1cnc(N2CCn3c(nc4cc(CO)c(S(C)(=O)=O)cc43)[C@H]2C(C)C)nc1C(F)(F)F] |
| E13a | Example 13, isomer 1 | 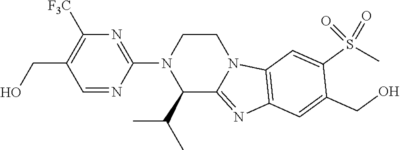 [SMILES: CC(C)[C@@H]1c2nc3cc(CO)c(S(C)(=O)=O)cc3n2CCN1c1ncc(CO)c(C(F)(F)F)n1] |
| E13b | Example 13, isomer 2 | 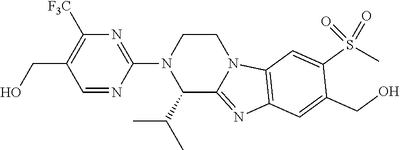 [SMILES: CC(C)[C@H]1c2nc3cc(CO)c(S(C)(=O)=O)cc3n2CCN1c1ncc(CO)c(C(F)(F)F)n1] |
| E14a | Example 14, isomer 1 | 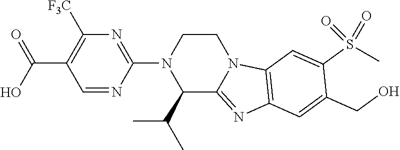 [SMILES: CC(C)[C@@H]1c2nc3cc(CO)c(S(C)(=O)=O)cc3n2CCN1c1ncc(C(=O)O)c(C(F)(F)F)n1] |
| E14b | Example 14, isomer 2 | 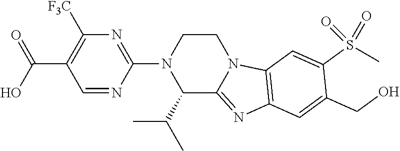 [SMILES: CC(C)[C@H]1c2nc3cc(CO)c(S(C)(=O)=O)cc3n2CCN1c1ncc(C(=O)O)c(C(F)(F)F)n1] |
| E15a | Example 15, isomer 1 | 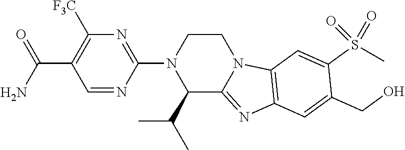 [SMILES: CC(C)[C@@H]1c2nc3cc(CO)c(S(C)(=O)=O)cc3n2CCN1c1ncc(C(N)=O)c(C(F)(F)F)n1] |
| E15b | Example 15, isomer 2 | 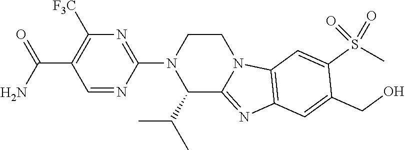 [SMILES: CC(C)[C@H]1c2nc3cc(CO)c(S(C)(=O)=O)cc3n2CCN1c1ncc(C(N)=O)c(C(F)(F)F)n1] |
| E16a | Example 16, isomer 1 | 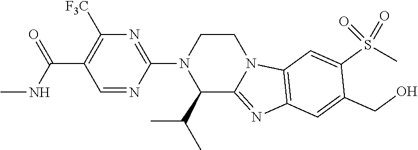 [SMILES: CNC(=O)c1cnc(N2CCn3c(nc4cc(CO)c(S(C)(=O)=O)cc43)[C@H]2C(C)C)nc1C(F)(F)F] |
| E16b | Example 16, isomer 2 | 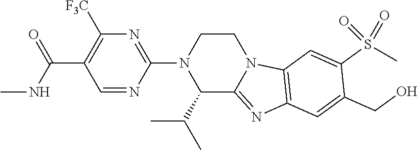 [SMILES: CNC(=O)c1cnc(N2CCn3c(nc4cc(CO)c(S(C)(=O)=O)cc43)[C@@H]2C(C)C)nc1C(F)(F)F] |
| E17a | Example 17, isomer 1 | 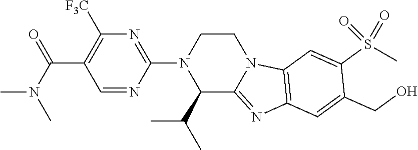 [SMILES: CC(C)[C@@H]1c2nc3cc(CO)c(S(C)(=O)=O)cc3n2CCN1c1ncc(C(=O)N(C)C)c(C(F)(F)F)n1] |
| E17b | Example 17, isomer 2 | 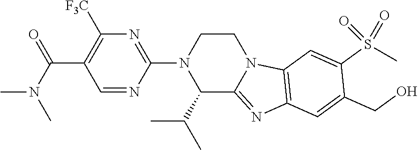 [SMILES: CC(C)[C@H]1c2nc3cc(CO)c(S(C)(=O)=O)cc3n2CCN1c1ncc(C(=O)N(C)C)c(C(F)(F)F)n1] |
| E18 | Example 18 | 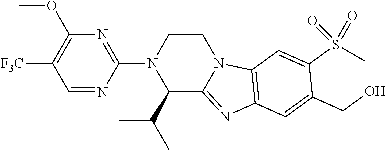 [SMILES: COc1nc(N2CCn3c(nc4cc(CO)c(S(C)(=O)=O)cc43)[C@H]2C(C)C)ncc1C(F)(F)F] |
| E19 | Example 19 | 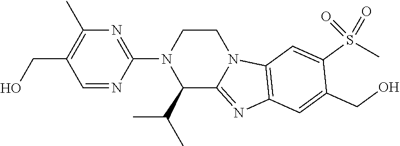 [SMILES: Cc1nc(N2CCn3c(nc4cc(CO)c(S(C)(=O)=O)cc43)[C@H]2C(C)C)ncc1CO] |
| E20a | Example 20, isomer 1 | 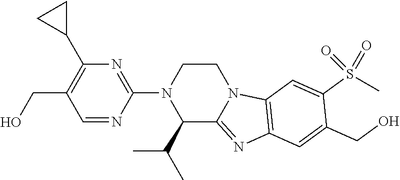 [SMILES: CC(C)[C@@H]1c2nc3cc(CO)c(S(C)(=O)=O)cc3n2CCN1c1ncc(CO)c(C2CC2)n1] |
| E20b | Example 20, isomer 2 | 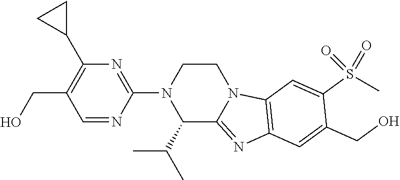 [SMILES: CC(C)[C@H]1c2nc3cc(CO)c(S(C)(=O)=O)cc3n2CCN1c1ncc(CO)c(C2CC2)n1] |
| E21 | Example 21 | 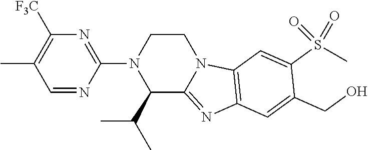 [SMILES: Cc1cnc(N2CCn3c(nc4cc(CO)c(S(C)(=O)=O)cc43)[C@H]2C(C)C)nc1C(F)(F)F] |
| E22a | Example 22, isomer 1 | 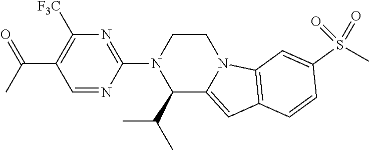 [SMILES: CC(=O)c1cnc(N2CCn3c(cc4ccc(S(C)(=O)=O)cc43)[C@H]2C(C)C)nc1C(F)(F)F] |
| E22b | Example 22, isomer 2 | 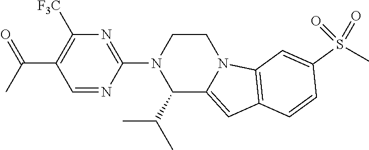 [SMILES: CC(=O)c1cnc(N2CCn3c(cc4ccc(S(C)(=O)=O)cc43)[C@@H]2C(C)C)nc1C(F)(F)F] |
| E23a | Example 23, isomer 1 | 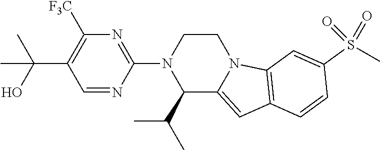 [SMILES: CC(C)[C@@H]1c2cc3ccc(S(C)(=O)=O)cc3n2CCN1c1ncc(C(C)(C)O)c(C(F)(F)F)n1] |
| E23b | Example 23, isomer 2 | 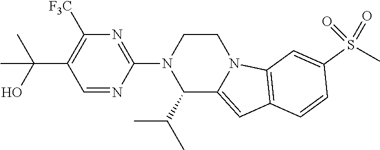 [SMILES: CC(C)[C@H]1c2cc3ccc(S(C)(=O)=O)cc3n2CCN1c1ncc(C(C)(C)O)c(C(F)(F)F)n1] |
| E24a | Example 24, isomer 1 | 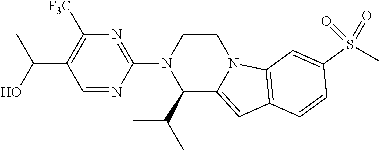 [SMILES: CC(O)c1cnc(N2CCn3c(cc4ccc(S(C)(=O)=O)cc43)[C@H]2C(C)C)nc1C(F)(F)F] |
| E24b | Example 24, isomer 2 | 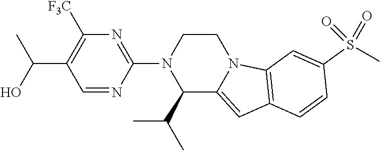 [SMILES: CC(O)c1cnc(N2CCn3c(cc4ccc(S(C)(=O)=O)cc43)[C@H]2C(C)C)nc1C(F)(F)F] |
| E24c | Example 24, isomer 3 | 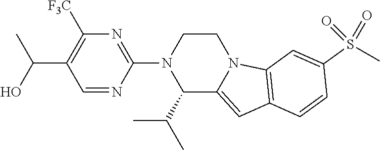 [SMILES: CC(O)c1cnc(N2CCn3c(cc4ccc(S(C)(=O)=O)cc43)[C@@H]2C(C)C)nc1C(F)(F)F] |
| E24d | Example 24, isomer 4 | 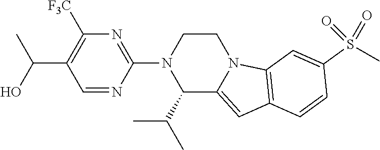 [SMILES: CC(O)c1cnc(N2CCn3c(cc4ccc(S(C)(=O)=O)cc43)[C@@H]2C(C)C)nc1C(F)(F)F] |
| E25a | Example 25, isomer 1 | 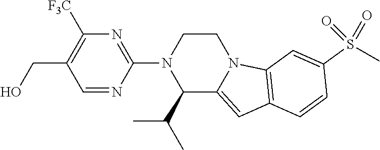 [SMILES: CC(C)[C@@H]1c2cc3ccc(S(C)(=O)=O)cc3n2CCN1c1ncc(CO)c(C(F)(F)F)n1] |
| E25b | Example 25, isomer 2 | 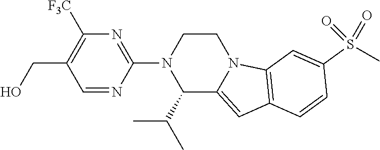 [SMILES: CC(C)[C@H]1c2cc3ccc(S(C)(=O)=O)cc3n2CCN1c1ncc(CO)c(C(F)(F)F)n1] |
| E26 | Example 26 | 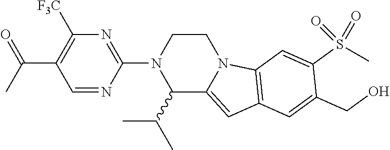 [SMILES: CC(=O)c1cnc(N2CCn3c(cc4cc(CO)c(S(C)(=O)=O)cc43)C2C(C)C)nc1C(F)(F)F] |
| E27a | Example 27, isomer 1 | 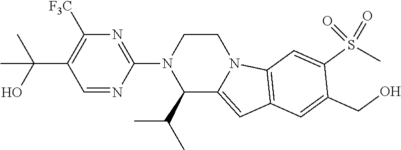 [SMILES: CC(C)[C@@H]1c2cc3cc(CO)c(S(C)(=O)=O)cc3n2CCN1c1ncc(C(C)(C)O)c(C(F)(F)F)n1] |
| E27b | Example 27, isomer 2 | 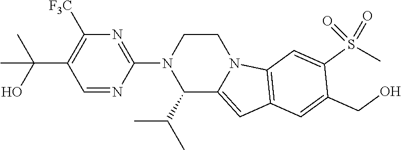 [SMILES: CC(C)[C@H]1c2cc3cc(CO)c(S(C)(=O)=O)cc3n2CCN1c1ncc(C(C)(C)O)c(C(F)(F)F)n1] |
| E28 | Example 28 | 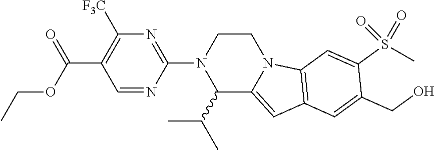 [SMILES: CCOC(=O)c1cnc(N2CCn3c(cc4cc(CO)c(S(C)(=O)=O)cc43)C2C(C)C)nc1C(F)(F)F] |
| E29a | Example 29, isomer 1 | 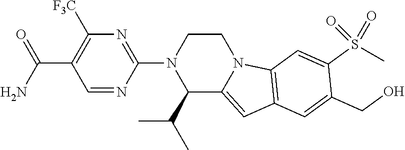 [SMILES: CC(C)[C@@H]1c2cc3cc(CO)c(S(C)(=O)=O)cc3n2CCN1c1ncc(C(N)=O)c(C(F)(F)F)n1] |
| E29b | Example 29, isomer 2 | 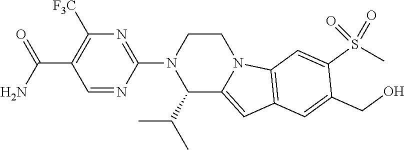 [SMILES: CC(C)[C@H]1c2cc3cc(CO)c(S(C)(=O)=O)cc3n2CCN1c1ncc(C(N)=O)c(C(F)(F)F)n1] |
| E30 | Example 30 | 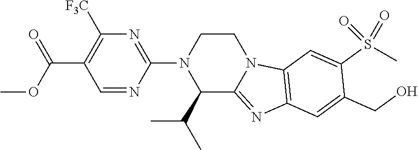 [SMILES: COC(=O)c1cnc(N2CCn3c(nc4cc(CO)c(S(C)(=O)=O)cc43)[C@H]2C(C)C)nc1C(F)(F)F] |
| E31 | Example 31 | 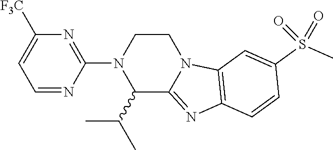 [SMILES: CC(C)C1c2nc3ccc(S(C)(=O)=O)cc3n2CCN1c1nccc(C(F)(F)F)n1] |
| E32a | Example 32, isomer 1 | 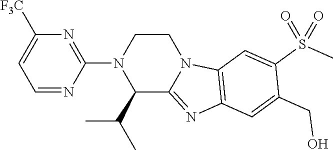 [SMILES: CC(C)[C@@H]1c2nc3cc(CO)c(S(C)(=O)=O)cc3n2CCN1c1nccc(C(F)(F)F)n1] |
| E32b | Example 32, isomer 2 | 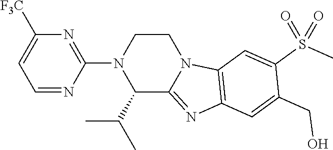 [SMILES: CC(C)[C@H]1c2nc3cc(CO)c(S(C)(=O)=O)cc3n2CCN1c1nccc(C(F)(F)F)n1] |
| E33a | Example 33, isomer 1 | 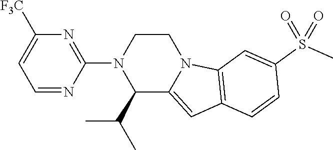 [SMILES: CC(C)[C@@H]1c2cc3ccc(S(C)(=O)=O)cc3n2CCN1c1nccc(C(F)(F)F)n1] |
| E33b | Example 33, isomer 2 | 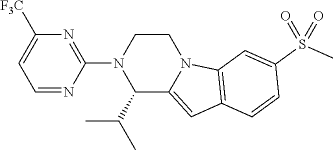 [SMILES: CC(C)[C@H]1c2cc3ccc(S(C)(=O)=O)cc3n2CCN1c1nccc(C(F)(F)F)n1] |
| E34a | Example 34, isomer 1 | 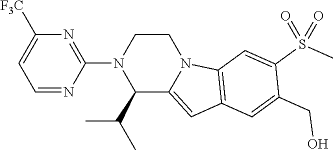 [SMILES: CC(C)[C@@H]1c2cc3cc(CO)c(S(C)(=O)=O)cc3n2CCN1c1nccc(C(F)(F)F)n1] |
| E34b | Example 34, isomer 2 | 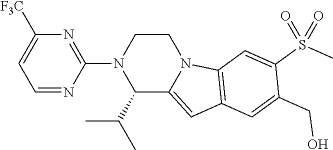 [SMILES: CC(C)[C@H]1c2cc3cc(CO)c(S(C)(=O)=O)cc3n2CCN1c1nccc(C(F)(F)F)n1] |

### Definitions

Unless otherwise specified, the below terms used herein are defined as follows.

“Subject”, “patient” and “mammal” are used interchangeably herein. In one embodiment, the subject is a non-human animal such as a non-human primate (e.g., a monkey, chimpanzee), a farm animal (e.g., a horse, cow, pig, chicken, or sheep), a laboratory animal (e.g., a rat or mouse), or a companion animal (e.g., dog, cat, guinea pig or rabbit). In a preferred embodiment, the subject is a human.

“Compound(s) of the methods described herein” refers to compounds represented by Structural Formula I, II, III, IV, V, VI, VII; a compound depicted in Table 1; a compound named or depicted in the examples herein as the final compound(s) of the example; or a pharmaceutically acceptable salt thereof. “Compound(s) of the methods described herein” also includes the neutral form of the compounds as depicted herein.

“Pharmaceutically acceptable” refers to a component that is, within the scope of sound medical judgment, suitable for use in contact with the tissues of the subject, such as humans and other mammals, without undue toxicity, irritation, allergic response and the like, and are commensurate with a reasonable benefit/risk ratio.

Included in the invention are pharmaceutically acceptable salts of the compounds of the methods described herein. The disclosed compounds have basic amine groups and therefore can form pharmaceutically acceptable salts with pharmaceutically acceptable acid(s). Suitable pharmaceutically acceptable acid addition salts of the compounds of the methods described herein include salts of inorganic acids (such as hydrochloric acid, hydrobromic, phosphoric, metaphosphoric, nitric, and sulfuric acids) and of organic acids (such as, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic, and tartaric acids). Compounds of the methods described herein with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts). Lists of suitable salts are found in Remington's Pharmaceutical Sciences, 18th ed., Mack Publishing Company, Easton, Pa., 1990, p 1445, the disclosure of which is hereby incorporated by reference.

“Treat” or “treating” includes therapeutic treatments and means to decrease, suppress, diminish, arrest, or stabilize the development or progression of acute coronary syndrome or related disorders.

“Effective amount” is the quantity of the compound of the methods described herein which is sufficient to treat (therapeutically or prophylactically) the target disorder or in which a beneficial clinical outcome is achieved when the compound is administered to a subject in a proper dosing regimen. Effective doses will also vary, as recognized by one of ordinary skill in the art, depending on the disease being treated, the severity of the disease, the route of administration, the sex, age and general health condition of the patient, excipient usage, the possibility of co-usage with other therapeutic treatments such as use of other agents and the judgment of the treating physician or other medical provider.

“Halo” or “halogen” means chloro, bromo, fluoro, or iodo. In one embodiment, halo is fluoro.

“Alkyl” means a straight or branched hydrocarbon group having 1 to 15 carbon atoms in the chain. In one embodiment, alkyl groups have 1 to 12 carbon atoms in the chain. In another embodiment, alkyl groups have 1 to 6 carbon atoms. Exemplary alkyl groups include, but are not limited to, methyl, ethyl, n-propyl, isopropyl, n-butyl, tert-butyl, sec-butyl, n-pentyl, 3-pentyl, heptyl, octyl, nonyl, decyl, and dodecyl.

“Alkoxy” is an alkyl group which is attached to another moiety via an oxygen linker (O(alkyl)). Non-limiting examples include methoxy, ethoxy, propoxy, and butoxy.

“Haloalkyl” or “halogenated alkyl” means an alkyl group in which one or more, including all, of the hydrogen radicals are replaced by a halo group, wherein each halo group is independently selected from —F, —Cl, —Br, and —I. For example, the term “halomethyl” or “halogenated methyl” means a methyl in which one to three hydrogen radical(s) have been replaced by a halo group. Representative haloalkyl groups include fluoromethyl, difluoromethyl, trifluoromethyl, bromomethyl, 1,2-dichloroethyl, 4-iodobutyl, 2-fluoropentyl, and the like. Other examples include groups such as but are not limited to —CH₂CF₃, —CH(CH₂F)₂, —CH(CHF₂)₂, —CH(CF₃)₂, —CF(CH₃)₂, —CF₃.

“Haloalkoxy” is a haloalkyl group which is attached to another moiety via an oxygen linker such as but are not limited to —OCHCF₂ or —OCF₃.

“Alkoxyalkyl” is an alkoxy group which is attached to another moiety via an alkyl linker. “Hydroxyalkyl” or “dihydroxyalkyl” is one or two hydroxy groups, respectively, which are attached to another moiety via an alkyl linker. Representative “hydroxyalkyl” or “dihydroxyalkyl” include —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(CH₃)₂(OH), and the like.

“Cycloalkyl” means a non-aromatic monocyclic ring system of 3 to 10 carbon atoms. In one embodiment, the cycloalkyl group has 3 to 6 carbon atoms. Exemplary cycloalkyl rings include cyclopropyl, cyclobutyl, cyclopentyl, and cyclohexyl.

“Cycloalkoxy” means a cycloalkyl group which is attached to another moiety via an oxygen linker (O(cycloalkyl)).

“Monocyclic non-aromatic heterocycle” means a single saturated heterocyclic ring, typically having 3- to 10-members and more typically 3 to 7-members in the ring, wherein at least one atom in the ring is a heteroatom such as, for example, nitrogen, oxygen, sulfur, including sulfoxide and sulfone. A 3- to 4-membered monocyclic non-aromatic heterocycle can contain up to 2 heteroatoms; a 5-6 membered monocyclic heterocycle can contain up to 3 heteroatoms and a 7- to 10-membered monocyclic non-aromatic heterocycle can contain up to 4 heteroatoms. The monocyclic non-aromatic heterocycle may be attached to another group via any heteroatom or carbon atom of the monocyclic non-aromatic heterocycle. Representative monocyclic non-aromatic heterocycles include morpholinyl, thiomorpholinyl, pyrrolidinonyl, pyrrolidinyl, piperidinyl, piperazinyl, imidazolidinyl, pyrazolidinyl, oxazolidinyl, isothiazolidinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. In one embodiment, a monocyclic non-aromatic heterocycle is a heterocyclic ring of 4, 5, 6, or 7 members.

“Monocyclic heteroaromatic” comprises carbon atom ring members and one or more heteroatom ring members. Each heteroatom is independently selected from nitrogen, oxygen, and sulfur, including sulfoxide and sulfone. The point of attachment of a monocyclic heteroaromatic ring to another group may be at either a carbon atom or a heteroatom of the heteroaromatic. In one embodiment, the monocyclic heteroaromatic ring is selected from 5 to 8 membered monocyclic heteroaromatic rings. Representative monocyclic heteroaromatic groups include pyridyl, 1-oxo-pyridyl, furanyl, thienyl, pyrrolyl, oxazolyl, imidazolyl, thiazolyl, isoxazolyl, pyrazolyl, isothiazolyl, pyridazinyl, pyrimidinyl, pyrazinyl, a triazinyl, triazolyl, thiadiazolyl, and tetrazolyl.

### Pharmaceutical Compositions, Formulations and Dosages

The compounds of the methods described herein can be formulated as pharmaceutical compositions and administered to a subject, such as a human, in a variety of forms adapted to the chosen route of administration. Typical routes of administering such pharmaceutical compositions include, without limitation, oral, topical, buccal, transdermal, inhalation, parenteral, sublingual, rectal, vaginal, and intranasal. The term parenteral as used herein includes subcutaneous injections, intravenous, intramuscular, intrathecal, intrasternal injection or infusion techniques. Methods of formulating pharmaceutical compositions are well known in the art, for example, as disclosed in “Remington: The Science and Practice of Pharmacy,” University of the Sciences in Philadelphia, ed., 21st edition, 2005, Lippincott, Williams & Wilkins, Philadelphia, Pa. Each of the compounds described for the disclosed methods may be used alone or in combination as a part of a pharmaceutical composition of the invention.

Pharmaceutical compositions of the invention can be prepared by combining a compound of the methods described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Thus, the present compounds of the methods described herein may be systemically administered, e.g., orally, in combination with a pharmaceutically acceptable excipient such as an inert diluent or an assimilable edible carrier. They may be enclosed in hard or soft shell gelatin capsules, may be compressed into tablets or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like.

Suitable tablets may be obtained, for example, by mixing one or more compounds of the methods described herein with known excipients, for example inert diluents, carriers, disintegrants, adjuvants, surfactants, binders and/or lubricants. The tablets may also consist of several layers.

The compounds described herein can be suitably formulated into pharmaceutical compositions for administration to a subject. The pharmaceutical compositions of the invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5th Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

Typically, for oral therapeutic administration, a compound of the methods described herein may be incorporated with excipient and used in the form of ingestible tablets, buccal tablets, troches, capsules, elixirs, suspensions, syrups, wafers, and the like.

Typically for parenteral administration, solutions of a compound of the methods described herein can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the methods described hereinfor the extemporaneous preparation of sterile injectable solutions or dispersions.

Topical and/or local administration of the compounds of the methods described herein can be achieved in a variety of ways including but not limited to ointments, lotions, pastes, creams, gels, powders, drops, sprays, solutions, inhalants, patches, suppositories, retention enemas, chewable or suckable tablets or pellets and aerosols. Topical and/or local administration may also involve the use of transdermal administration such as transdermal patches or iontophoresis devices. For topical and/or local administration, the compounds of the methods described herein can be formulated as ointments, creams, milks, salves, powders, impregnated pads, syndets, solutions, gels, sprays, foams, suspensions, lotions, sticks, shampoos or washing bases. Compounds of the methods described herein may also be administered in the form of suspensions of lipid or polymer vesicles or nanospheres or microspheres or polymer patches and hydrogels for controlled release.

### Methods of Treatment and Use

Compounds used in the disclosed methods are useful for the treatment of acute coronary syndrome and related conditions. Such related conditions include e.g., heart attack, myocardial infarction, acute myocardial infarction, non-ST-segment elevation myocardial infarction, ST-segment elevation myocardial infarction, unstable angina, stable angina, angina pectoris, exercise induced angina, coronary artery disease, coronary heart disease, acute myocardial ischemia, ischaemic heart disease, ischemia, recurrent ischemia, congestive heart disease, congestive heart failure, cardiomyopathy, hypertensive heart disease, heart failure, diastolic heart failure, systolic heart failure, cor pulmonale, cardiac dysrhythmias, abnormalities of heart rhythm, inflammatory heart disease, endocarditis, inflammatory cardiomegaly, myocarditis, cerebrovascular disease, peripheral arterial disease, reperfusion injury, restenosis, atherosclerotic lesions, or chronic atherosclerotic inflammation.

The compounds of the methods described herein can be used alone (i.e., as a monotherapy) or in combination with one or more other therapeutic agent effective for treating any of the above indications. That is, in some embodiments, the present disclosure provides for combination therapies, where e.g., the combination therapy comprises administering at least one compound represented by Structural Formula I, II, III, IV, V, VI, or VII in combination with one or more agents for treating or ameliorating a disease or a disorder described herein. The pharmaceutical compositions can comprise the disclosed compounds alone as the only pharmaceutically active agent or can comprise one or more additional pharmaceutically active agents.

In some embodiments, the combination therapy comprises administering at least one compound represented by Structural Formula I, II, III, IV, V, VI, or VII in combination with one or more agents for treating or ameliorating other diseases, including hyperlipidemia, hypercholesterolemia, hyperlipoproteinemia, hypertriglyceridemia, lipodystrophy, hepatic steatosis, NASH, NAFLD, hyperglycemia, insulin resistance, diabetes mellitus, dyslipidemia, atherosclerosis, gallstone disease, acne vulgaris, dermatitis (including but not limited to, psoriasis, contact dermatitis, atopic dermatitis, and eczema), skin wounds, skin aging, photoaging, wrinkling, diabetes, Niemann-Pick disease type C, Parkinson's disease, Alzheimer's disease, inflammation, xanthoma, obesity, metabolic syndrome, syndrome X, stroke, peripheral occlusive disease, memory loss, diabetic neuropathies, proteinuria, glomerulopathies (including but not limited to, diabetic nephropathy, hypertensive nephropathy, IGA nephropathy, focal segmental glomerulosclerosis), hyperphosphatemia, associated cardiovascular complications of hyperphosphatemia, cancer, multiple sclerosis or osteoporosis.

In some embodiments, the compounds of the methods described herein are used in combination with one or more additional therapies, including therapies to alleviate pain and anxiety, prevent recurrences of ischaemia and prevent or limit progression to acute myocardial infarction. Such additional therapies include antithrombotic treatment, as well as coronary angiography followed by revascularization. Further additional therapies include smoking cessation, exercise, and management of hypertension and blood glucose.

In some embodiments, the compounds of the methods described herein are used in combination with one or more agents for the treatment of diabetes, dyslipidemia, cardiovascular disease, hypertension, or obesity. Agents for the treatment of diabetes include insulins, such as Humulin® (Eli Lilly), Lantus® (Sanofi Aventis), Novolin® (Novo Nordisk), and Exubera® (Pfizer); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® and (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophage/Glucophage XR® (metformin HCl, Bristol Myers Squibb) and Glumetza® (metformin HCl extended release tablets, Depomed); thiazolidinediones; amylin analogs, GLP-1 analogs or agonists (including Byetta® (exenatide, Amylin/Eli Lilly) and Victoza® (recombinant liraglutide, Novo Nordisk)); DPP-IV inhibitors including Tradjenta™ (Eli Lilly/Boehringer Ingelheim), Januvia® (Merck), Galvus® (Novartis), and Onglyza® (Bristol-Myers Squibb/AstraZeneca); PTB-1 B inhibitors; protein kinase inhibitors (including AMP-activated protein kinase inhibitors); glucagon antagonists, glycogen synthase kinase-3 beta inhibitors; glucose-6-phoshatase inhibitors; glycogen phosphorylase inhibitors; sodium glucose co-transporter inhibitors, and alpha-glucosidase inhibitors, such as Precose®/Glucobay®/Prandase®/Glucor® (acarbose, Bayer) and Glyset® (miglitol, Pfizer). Agents for the treatment of dyslipidemia and cardiovascular disease include statins, fibrates, and ezetimibe. Agents for the treatment of hypertension include alpha-blockers, beta-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, or endothelin receptor antagonists. Agents for the treatment of obesity include orlistat, phentermine, sibutramine and rimonabant.

An embodiment of the invention includes administering a compound of the methods described or composition thereof in a combination therapy with combination products, such as Avandamet® (metformin HCl and rosiglitazone maleate, GSK); Avandaryl® (glimepiride and rosiglitazone maleate, GSK); Metaglip® (glipizide and metformin HCl, Bristol Myers Squibb); and Glucovance® (glyburide and metformin HCl, Bristol Myers Squibb).

In some embodiments, the combination therapy comprises administering at least one compound of the methods described herein in combination with one or more compound selected from the group of, for example, beta secretase (BACE1) inhibitors; gamma-secretase inhibitors; amyloid aggregation inhibitors (e.g., ELND-005); directly or indirectly acting neuroprotective and/or disease-modifying substances; anti-oxidants (e.g., vitamin E or ginkolide); anti-inflammatory substances (e.g., Cox inhibitors, NSAIDs); HMG-CoA reductase inhibitors (statins); acetylcholinesterase inhibitors (e.g., donepezil, rivastigmine, tacrine, galantamine, memantine; tacrine); NMDA receptor antagonists (e.g., memantine); AMPA receptor agonists; AMPA receptor positive modulators, AMPAkines, monoamine receptor reuptake inhibitors, substances modulating the concentration or release of neurotransmitters; substances inducing the secretion of growth hormone (e.g., ibutamoren mesylate and capromorelin); CB-1 receptor antagonists or inverse agonists; antibiotics (e.g., minocyclin or rifampicin); PDE2, PDE4, PDE5, PDE9, PDE10 inhibitors, GABAA receptor inverse agonists, GABAA receptor antagonists, nicotinic receptor agonists or partial agonists or positive modulators, alpha4beta2 nicotinic receptor agonists or partial agonists or positive modulators, alpha7 nicotinic receptor agonists or partial agonists or positive modulators; histamine H3 antagonists, 5 HT-4 agonists or partial agonists, 5HT-6 antagonists, alpha2-adrenoreceptor antagonists, calcium antagonists, muscarinic receptor M1 agonists or partial agonists or positive modulators, muscarinic receptor M2 antagonists, muscarinic receptor M4 antagonists, metabotropic glutamate-receptor 5 positive modulators, antidepressants, such as citalopram, fluoxetine, paroxetine, sertraline and trazodone; anxiolytics, such as lorazepam and oxazepam; antiphychotics, such as aripiprazole, clozapine, haloperidol, olanzapine, quetiapine, risperidone and ziprasidone, and other substances that modulate receptors or enzymes in a manner such that the efficacy and/or safety of the compounds according to the invention is increased and/or unwanted side effects are reduced.

In some embodiments, the compounds of the methods described herein are used in combination with one or more agents for antiplatelet or anticoagulant therapy, including aspirin, clopidogrel, prasugrel, ticagrelor, and glycoprotein IIb/IIIa inhibitors including eptifibatide, tirofiban and abciximab.

In some embodiments, the compounds of the methods described herein are used in combination with one or more agents for antithrombin therapy, including fondaparinux, heparin, and bivalirudin.

In some embodiments, the compounds of the methods described herein are used in combination with one or more lipid lowering agents, including a statin, nicotinic acid, bile acid binding resin, and ezetimibe.

In some embodiments, the compounds of the methods described herein are used in combination with one or more treatments for revascularization, including coronary angiography and bypass surgery.

In some embodiments, the compounds of the methods described herein are used in combination with one or more agents, including nitrates (sublingual, oral or intravenous), beta-blockers, calcium antagonists (e.g., diltiazem, verapamil), and angiotensin-converting enzyme (ACE) inhibitors.

In some embodiments, the compounds of the methods described herein are used in combination with one or more agents selected from anti-platelets, nitrates, beta blockers, glycoprotein IIB/IIIA inhibitors, anticoagulants, low molecular weight heparins, direct thrombin inhibitors, and adenosine diphosphate receptor antagonists.

The compounds described in the disclosed methods may also be used in combination with immunotherapies for the treatment of a disease or disorder disclosed herein.

Combination therapy includes co-administration of a compound of the methods described herein and one or more other agent, sequential administration of a compound of the methods described herein and one or more other agent, administration of a composition containing a compound of the described methods and one or more other agent, or simultaneous administration of separate compositions containing a compound of the described methods and one or more other agent.

### Exemplary Synthesis

### General Description of Synthetic Methods

The compounds of the methods described herein can be readily prepared according to the following reaction schemes and examples, or modifications thereof, using readily available starting materials, reagents and conventional synthesis procedures. Many of the reactions can also be carried out under microwave conditions or using conventional heating or utilizing other technologies such as solid phase reagents/scavengers or flow chemistry. In these reactions, it is also possible to make use of variants which are themselves known to those of ordinary skill in this art, but are not mentioned in greater detail. Furthermore, other methods for preparing compounds of the disclosed methods will be readily apparent to a person of ordinary skill in the art in light of the following reaction schemes and examples. In cases where synthetic intermediates and final products contain potentially reactive functional groups, for example amino, hydroxy, thiol and carboxylic acid groups, that may interfere with the desired reaction, it may be advantageous to employ protected forms of the intermediate. Methods for the selection, introduction and subsequent removal of protecting groups are well known to those skilled in the art. In the discussion below X, R¹, R², R³, R⁴, R⁵ and R⁶ have the meanings indicated above unless otherwise indicated. The abbreviations used in these experimental details are listed below and additional ones should be known to a person skilled in the art of synthesis. In addition one can refer to the following references for suitable methods of synthesis as described in March, Advanced Organic Chemistry, 3rd edition, John Wiley & Sons, 1985, Greene and Wuts, Protective Groups in Organic Synthesis, 2nd edition, John Wiley & Sons, 1991, and Richard Larock, Comprehensive Organic Transformations, 4th edition, VCH publishers Inc., 1989.

Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

Where NMR data are presented, spectra were obtained on a Varian 400 (400 MHz) or 300 (300 MHz) and are reported as ppm downfield from tetramethylsilane with number of proton, multiplicities and coupling constants indicated parenthetically along with reference to deuterated solvent.

LC-MS data were obtained by utilizing the following chromatographic conditions:

Method 1 (10-80, 2 min)

| Column | Xtimate ™ C18 2.1 * 30 mm, 3 μm | | |
|---|---|---|---|
| | A: water (4 L) + TFA (1.5 mL) | | |
| | B: acetonitrile (4 L) + TFA (0.75 mL) | | |
| | TIME (min) | A % | B % |
| Mobile Phase | 0 | 90 | 10 |
| | 0.9 | 20 | 80 |
| | 1.5 | 20 | 80 |
| | 1.51 | 90 | 10 |
| | 2 | 90 | 10 |

| | |
|---|---|
| Flow Rate | 1.2 mL/min |
| wavelength | UV 220 nm |
| Oven Temp | 50° C. |
| MS ionization | ESI |

Method 2 (30-90, 2 min)

| Column | Xtimate ™ C18 2.1 * 30 mm, 3 μm | | |
|---|---|---|---|
| | A: water (4 L) + TFA (1.5 mL) | | |
| | B: acetonitrile (4 L) + TFA (0.75 mL) | | |
| | TIME (min) | A % | B % |
| Mobile Phase | 0 | 70 | 30 |
| | 0.9 | 10 | 90 |
| | 1.5 | 10 | 90 |
| | 1.51 | 70 | 30 |
| | 2 | 70 | 30 |

| | |
|---|---|
| Flow Rate | 1.2 mL/min |
| wavelength | UV 220 nm |
| Oven Temp | 50° C. |
| MS ionization | ESI |

Method 3 (0-60, 2 min)

| Column | Xtimate ™ C18 2.1 * 30 mm, 3 μm | | |
|---|---|---|---|
| | A: water (4 L) + TFA (1.5 mL) | | |
| | B: acetonitrile (4 L) + TFA (0.75 mL) | | |
| | TIME (min) | A % | B % |
| Mobile Phase | 0 | 100 | 0 |
| | 0.9 | 40 | 60 |
| | 1.5 | 40 | 60 |
| | 1.51 | 100 | 0 |
| | 2 | 100 | 0 |

| | |
|---|---|
| Flow Rate | 1.2 mL/min |
| wavelength | UV 220 nm |
| Oven Temp | 50° C. |
| MS ionization | ESI |

### Method 4:

HPLC System: Waters ACQUITY; Column: Waters ACQUITY CSH™ C18 1.7 μM
Guard column: Waters Assy. Frit, 0.2 μM, 2.1 mm; Column tem: 40° C. Mobile Phase: A: TFA: Water (1:1000, v:v) Mobile phase B: TFA: ACN (1:1000, v:v); Flow Rate: 0.65 mL/min; Injection Volume: 2 μL; Acquisition time: approximately 1.5 minute.

Gradient Program:

| Time (min) | B % |
|---|---|
| 0 | 10 |
| 0.8 | 90 |
| 1.20 | 90 |
| 1.21 | 10 |

### Mass Spectrometer Parameters

Mass Spectrometer: Waters SQD; Ionization: Positive Electrospray Ionization (ESI); Mode Scan (100-1400 m/z in every 0.2 second); ES Capillary Voltage: 3.5 kv; ES Cone Voltage: 25 v Source Temperature: 120° C.; Disolvation Temperature: 500° C.; Desolvation Gas Flow: Nitrogen Setting 650 (L/hr); Cone Gas Flow: Nitrogen Setting 50 (L/hr)

SFC separation of compounds of the methods described herein were carried out under the following methods.

Method A:
Instrument: Thar SFC 80; Column: AD 250 mm*30 mm, 5 μm; Mobile phase: A: Supercritical CO₂, B: IPA (0.05% DEA), A:B=80:20 at 60 ml/min; Column Temp: 38° C.; Nozzle Pressure: 100 Bar; Nozzle Temp: 60° C.; Evaporator Temp: 20° C.; Trimmer Temp: 25° C.; Wavelength: 220 nm.

Method B:
Instrument: SFC MG2; Column: OJ 250 mm*30 mm, 5 μm; Mobile phase: A: Supercritical CO₂, B: MeOH(0.05% DEA), A:B=90:10 at 70 ml/min; Column Temp: 38° C.; Nozzle Pressure: 100 Bar Nozzle Temp: 60° C.; Evaporator Temp: 20° C.; Trimmer Temp: 25° C.; Wavelength: 220 nm

### Analytical Chiral HPLC

The chiral purity of compounds of the described methods was determined by analytical chiral HPLC, which was carried out using Chiralcel® or Chiralpak® columns, using CO₂, together with from 5% to 40% methanol, ethanol or isopropanol, containing 0.05% DEA, as eluents.

| Method | Detailed information |
|---|---|
| OJ-H_3_5_40_2.35 ML | Column: Chiralcel ® OJ-H 250 × 4.6 mm I.D., 5 μm Mobile phase: |
| | methanol (0.05% DEA) in CO₂ from 5% to 40% Flow rate: |
| | 2.35 mL/min Wavelength: 220 nm |
| OJ-H_3_5_40_2.5 ML | Column: Chiralcel ® OJ-H 250 × 4.6 mm I.D., 5 μm Mobile phase: |
| | methanol (0.05% DEA) in CO₂ from 5% to 40% Flow rate: |
| | 2.5 mL/min Wavelength: 220 nm |
| AS-H_3_5_40_2.35 ML | Column: Chiralpak ® AS-H 250 × 4.6 mm I.D., 5 μm Mobile |
| | phase: methanol (0.05% DEA) in CO₂ from 5% to 40% Flow |
| | rate: 2.35 mL/min Wavelength: 220 nm |
| AS-H_4_5_40_2.5 ML | Column: Chiralpak ® AS-H 250 × 4.6 mm I.D., 5 μm Mobile |
| | phase: |
| | iso-propanol (0.05% DEA) in CO2 from5% to 40% Flow rate: |
| | 2.5 mL/min Wavelength: 220 nm |
| AS-H_5_5_40_2.35 ML | Column: Chiralpak ® AS-H 250 × 4.6 mm I.D., 5 μm Mobile |
| | phase: ethanol (0.05% DEA) in CO₂ from 5% to 40% Flow rate: |
| | 2.35 mL/min Wavelength: 220 nm |
| AS-H_3_5_40_2.5 ML | Column: Chiralpak ® AS-H 250 × 4.6 mm I.D., 5 μm Mobile |
| | phase: methanol (0.05% DEA) in CO2 from 5% to 40% Flow |
| | rate: 2.5 mL/min Wavelength: 220 nm |
| AD-H_3_5_40_2.35 ML | Column: Chiralpak ® AD-H 250 × 4.6 mm I.D., 5 μm Mobile |
| | phase: methanol (0.05% DEA) in CO₂ from 5% to 40% Flow |
| | rate: 2.35 mL/min Wavelength: 220 nm |
| AD-H_5_5_40_2.35 ML | Column: Chiralpak ® AD-H 250 × 4.6 mm I.D., 5 μm Mobile |
| | phase: ethanol (0.05% DEA) in CO₂ from 5% to 40% Flow rate: |
| | 2.35 mL/min Wavelength: 220 nm |
| OD-3_3_5_40_2.5 ML | Column: Chiralcel ® OD-3 150 × 4.6 mm I.D., 3 μm Mobile phase: |
| | methanol (0.05% DEA) in CO₂ from 5% to 40% Flow rate: |
| | 2.5 mL/min Wavelength: 220 nm |
| OD-3_4_5_40_2.5 ML | Column: Chiralcel ® OD-3 150 × 4.6 mm I.D., 3 μm Mobile phase: |
| | iso-propanol (0.05% DEA) in CO₂ from 5% to 40% Flow rate: |
| | 2.5 mL/min Wavelength: 220 nm |
| OD-3_5_5_40_2.5 ML | Column: Chiralcel ® OD-3 150 × 4.6 mm I.D., 3 μm Mobile phase: |
| | ethanol (0.05% DEA) in CO₂ from 5% to 40% Flow rate: |
| | 2.5 mL/min Wavelength: 220 nm |
| AD-3_3_5_40_2.5 ML | Column: Chiralpak ® AD-3 150 × 4.6 mm I.D., 3 μm Mobile |
| | phase: methanol (0.05% DEA) in CO₂ from 5% to 40% Flow |
| | rate: 2.5 mL/min Wavelength: 220 nm |
| AD-3_4_5_40_2.5 ML | Column: Chiralpak ® AD-3 150 × 4.6 mm I.D., 3 μm Mobile |
| | phase: iso-propanol (0.05% DEA) in CO₂ from 5% to 40% Flow |
| | rate: 2.5 mL/min Wavelength: 220 nm |
| AD-3_5_5_40_2.5 ML | Column: Chiralpak ® AD-3 150 × 4.6 mm I.D., 3 μm Mobile phase: |
| | ethanol (0.05% DEA) in CO₂ from 5% to 40% Flow rate: |
| | 2.5 mL/min Wavelength: 220 nm |
| OD-H_3_5_40_2.35 ML | Column: Chiralcel ® OD-H 250 × 4.6 mm I.D., 5 μm Mobile phase: |
| | methanol (0.05% DEA) in CO₂ from5% to 40% Flow rate: |
| | 2.35 mL/min Wavelength: 220 nm |
| OD-H_5_5_40_2.35 ML | Column: Chiralcel ® OD-H 250 × 4.6 mm I.D., 5 μm Mobile phase: |
| | ethanol (0.05% DEA) in CO₂ from5% to 40% Flow rate: |
| | 2.35 mL/min Wavelength: 220 nm |

The invention is illustrated by way of the following examples, in which the following abbreviations may be employed:

| Abbreviation | Meaning |
|---|---|
| ACN, MeCN, CH₃CN | acetonitrile |
| aq. | aqueous |
| Boc | tert-butoxy carbonyl or t-butoxy carbonyl |
| brine | saturated aqueous NaCl |
| Cbz | benzyloxy carbonyl |
| CeCl₃ | ceric chloride |
| Cs₂CO₃ | cesium carbonate |
| CuI | cuprous iodide |
| DCM or CH₂Cl₂ | methylene chloride |
| DIEA | diisopropyl ethyl amine |
| DMF | dimethyl formamide |
| DMS/Me2S | dimethyl sulfide |
| DMSO | dimethyl sulfoxide |
| EDCI | 1-(3-dimethylaminopropyl)-3-ethylcarbodiiimide |
| | hydrochloride |
| EtI | ethyl iodide |
| Et | ethyl |
| Et₂O | ethyl ether |
| Et₃SiH | triethylsilane |
| Et₃N | triethylamine |
| EtOAc, EA, AcOEt | ethyl acetate |
| EtOH | ethanol |
| FeCl₃ | ferric chloride |
| h, hr | hour(s) |
| HATU | O-(7-azabenzotriazol-1-yl)-N,N,N′,N′- |
| | tetramethyluronium-hexafluorophosphate |
| HBTU | O-Benzotriazole-1-yl-N,N,N′,N′- |
| | tetramethyluronium-hexafluorophosphate |
| HCl | hydrochloric acid |
| H₂O | water |
| H₂O₂ | hydrogen peroxide |
| HPLC | high performance liquid chromatography |
| i-BuOCOCl | iso-butoxycarbonyl chloride |
| ICl | iodochloride |
| K₂CO₃ | potassium carbonate |
| K₃PO₄ | tripotassium phosphate |
| LC-MS | liquid chromatography-mass spectrometry |
| LDA | lithium diiisopropylamide |
| LiCl | lithium chloride |
| LiOH | lithium hydroxide |
| MCPBA, m-CPBA | meta-chloroperoxybenzoic acid |
| MeOH | methanol |
| MeI | methyl iodide |
| Me | methyl |
| mg | milligram |
| Mg₂SO₄ | magnesium sulfate (anhydrous) |
| min | minute(s) |
| mL | milliliters |
| mmol | millimoles |
| mp, m.p. | melting point |
| MS | mass spectrometry |
| MW | microwave |
| NaBH₄ | sodium borohydride |
| NaBH₃CN | sodium cyanoborohydride |
| NaH | sodium hydride |
| NaHCO₃ | sodium bicarbonate |
| NaOH | sodium hydroxide |
| NaOMe | sodium methoxide |
| Na₂S₂O₃ | sodium thiosulfate |
| Na₂S₂O₅ | sodium dithionate |
| Na₂SO₄ | sodium sulfate |
| NH₄OH | ammonium hydroxide |
| (NH₄)₂CO₃ | ammonium carbonate |
| NH₄Cl | ammonium chloride |
| Na₂CO₃ | sodium carbonate |
| NaHCO₃ | sodium bicarbonate |
| NaH | sodium hydride |
| n-BuLi | n-butyllithium |
| NMM | N-methyl-morpholine |
| NMP | N-methyl-pyrrolidin-2-one |
| OTf | trifluoromethanesulfonate |
| OTs | tosylate |
| PdCl₂dppf | [1,1- |
| | bis(diphenylphosphino)ferrocene] |
| | dichloropalladium(ii) |
| Pd₂(dba)3 | tris(dibenzylideneacetone)dipalladium(0) |
| PE | petroleum ether |
| rt | room temperature |
| sat. | saturated |
| SFC | supercritical fluid chromatography |
| t-BuOK | potassium tert butoxide |
| t-BuLi | tert butyl lithium |
| t-BuOOH | tert butyl peroxide |
| TBAF | tetrabutylammonium fluoride |
| TFA | trifluoroacetic acid |
| THF | tetrahydrofuran |
| TLC | thin layer chromatography |
| Ti(OEt)₄ | titanium tetra ethoxide |
| Zn | zinc |
| Zn(CN)₂ | zinc cyanide |

In the first process, a compound of Formula I can be prepared by S_{N}Ar or palladium catalyzed reactions of reagents 1, where G¹ is Cl, Br, I, OTf or OTs, with intermediates of Formula 2. Reagents 1 are either commercially available or can be prepared readily from commercially available precursors based on literature precedents.

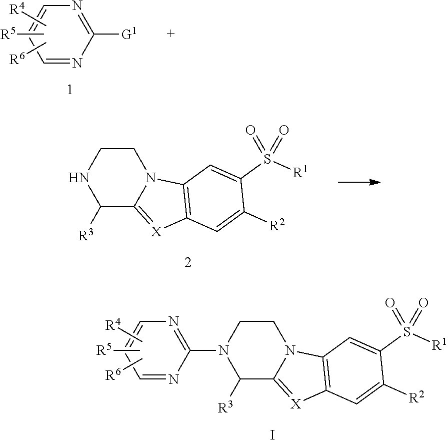

Intermediates 2 can be prepared by one of the several different methods depicted below.

When X═N, intermediates of Formula 2 can be prepared by cyclization of intermediates of Formula 3a followed by removal of G² when G² is not hydrogen. G² is an amine protecting group, such as Boc, Cbz and trifluoroacetamide, etc.

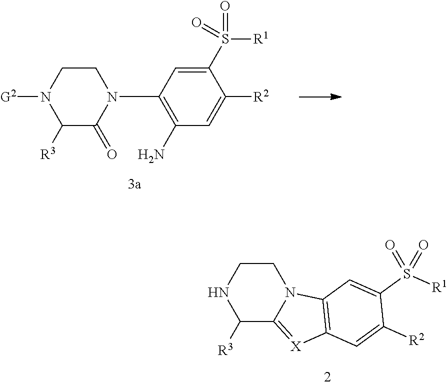

Intermediates of Formula 3a can be prepared by one of the two methods: 1) copper mediated coupling of piperazinone 4a and aniline 5a, where G³ is Br, I, Cl or OTf; 2) S_{N}Ar reaction between 4a and fluorinated nitrobenzene 6a to give intermediate of Formula 7a followed by reduction of the nitro group. The intermediate 7a can also be prepared from an intermediate of Formula 8a by displacement of fluorine with either sodium alkanesulfinate (R¹SO₂Na) or sodium alkylsulfide (R′SNa) followed by oxidation of the resulting thioether. The intermediate 8a in turn can be prepared from piperazinone 4a and difluoro nitrobenzene 9a, which are either commercially available or can be readily prepared from commercial precursors based on literature procedures, well known to those of ordinary skill in the art.

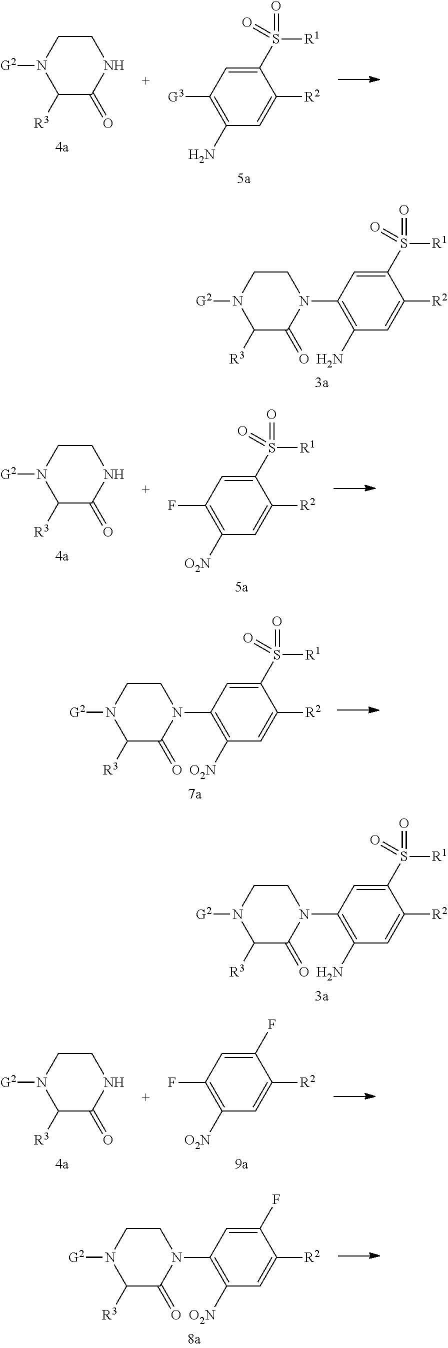

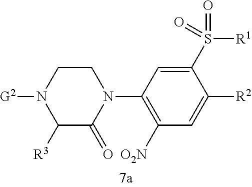

For example, when R³=isopropyl, piperazinone 4a can be prepared by one of the methods presented below.

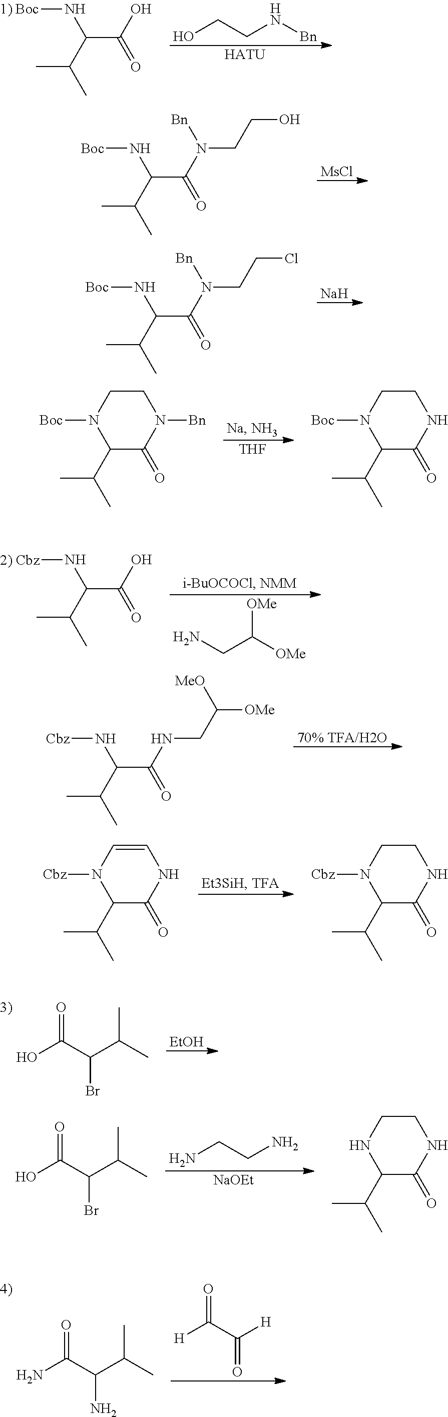

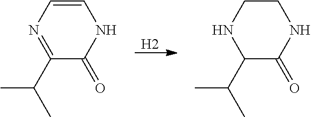

When X═CH, intermediates of Formula 2 can be prepared from intermediates of Formula 3b by deprotection of G² followed by reductive amination. G² are amine protecting groups, such as Boc, Cbz and trifluoroacetamide etc.

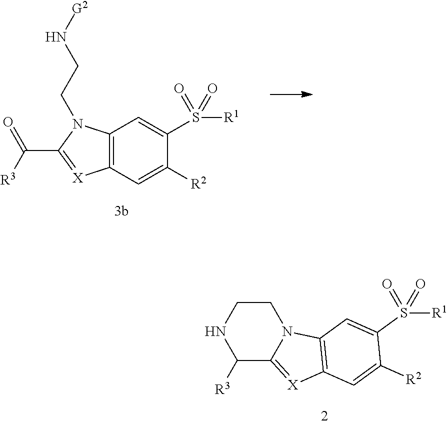

Intermediates of Formula 3b can be prepared by N-alkylation of indole 4b with commercially available alkyl halide 5b, where G³ is Br or I. Intermediates of Formula 4b can be prepared by removal of G⁴ from intermediates of Formula 6b, where G⁴ is methanesulfonate or phenylsulfonate.

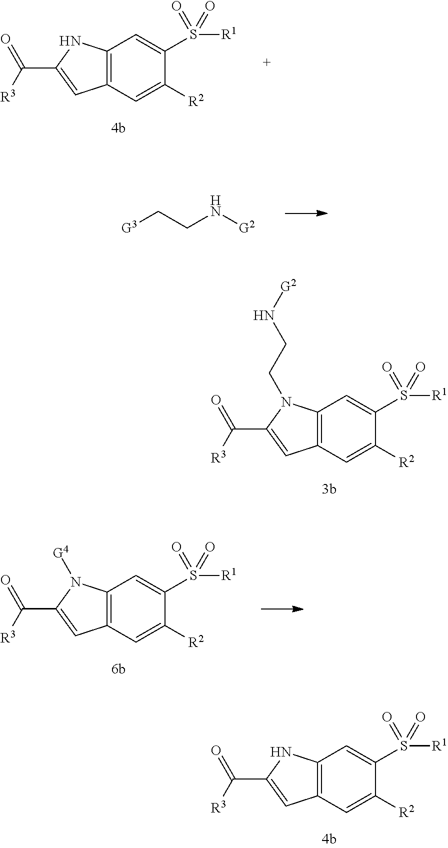

Intermediates of Formula 6b can be prepared by sequential Sonogashira coupling reaction between aryl halides 7b (where G⁵ is Br or I) and propargyl alcohols 8b, followed by cyclization, to give intermediates of Formula 9b, followed by oxidation of the alcohol.

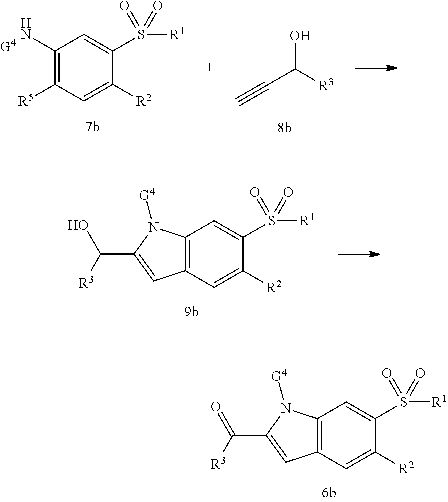

Intermediates of Formula 7b can be prepared from commercially available aniline 10b via the following transformations: 1) Displacement of fluorine with sodium alkyl sulfide R¹SNa (yielding 11b); 2) Halogenation (yielding 12b); 3) Protection of the aniline (yielding 13b); 4) Oxidation of the sulfide (yielding 7b).

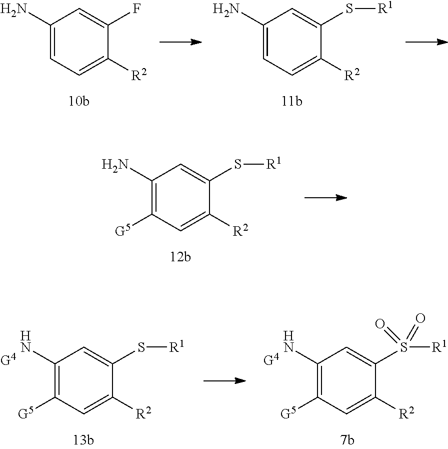

In the second process, a compound of Formula I, where R¹=alkyl, R²═H and X═CH, can be prepared by oxidation of the thioether group in intermediates of Formula 1c. Intermediate 1c in turn can be prepared from coupling of reagents 1 and intermediates of Formula 2c via S_{N}Ar or palladium catalyzed reactions.

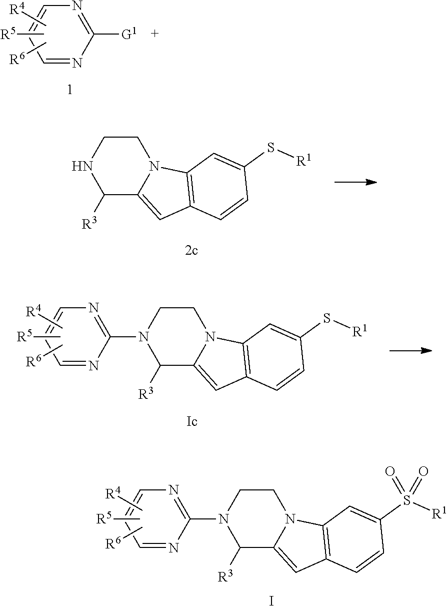

Intermediates 2c can be prepared according to following scheme.

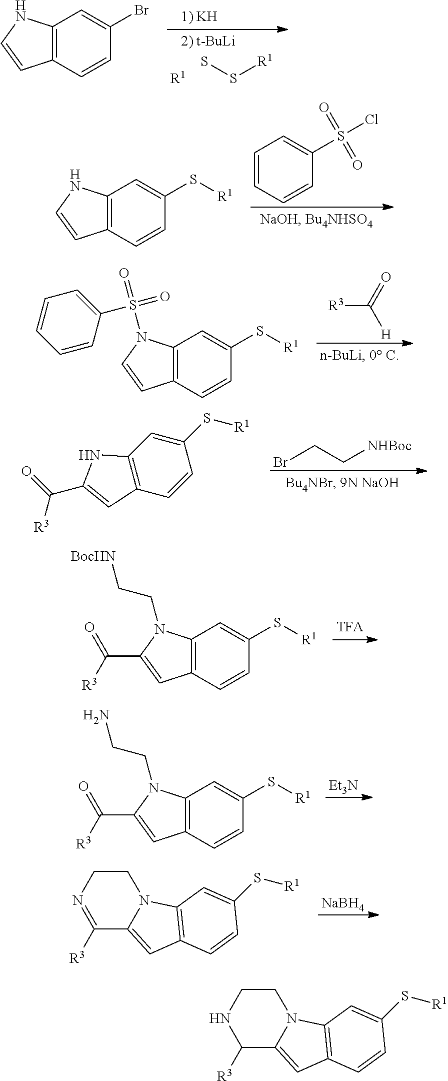

All patents, patent applications, books and literature cited in the specification are hereby incorporated by reference in their entirety. In the case of any inconsistencies, the present disclosure, including any definitions therein will prevail.

The invention will be further described by reference to the following detailed examples, which are given for illustration of the invention, and are not intended to be limiting thereof.

### Preparation 1

### Tert-butyl 1-isopropyl-7-(methylsulfonyl)-3,4-dihydrobenzo[4,5]imidazo[1,2-a]pyrazine-2(1H)-carboxylate

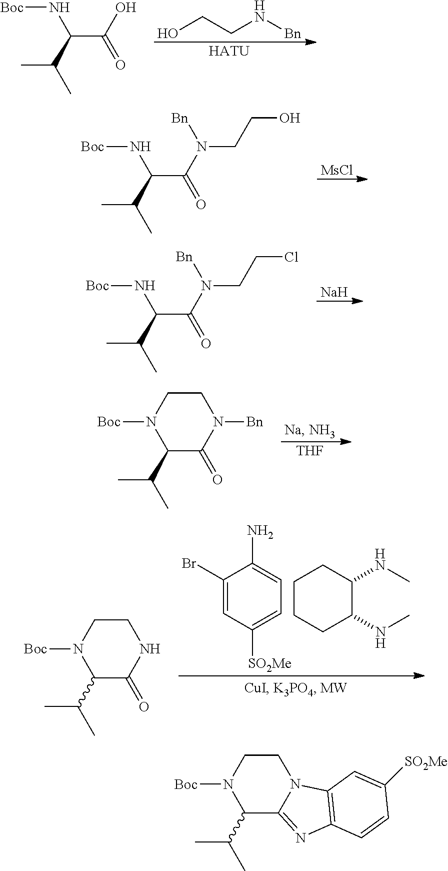

Racemization occurred during the course of synthesis

### Step 1:

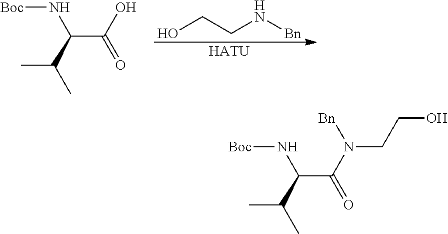

To a solution of (R)-2-((tert-butoxycarbonyl)amino)-3-methylbutanoic acid (2.0 g, 9.20 mmol) in CH₂Cl₂ (40 mL) were added 2-(benzylamino)ethanol (1.3 g, 8.80 mmol), HATU (5.30 g, 13.8 mmol) and Et₃N (2.80 g, 27.6 mmol) under N₂. The mixture was stirred at rt overnight. The mixture was diluted with water (20 mL) and extracted with EtOAc (3×30 mL). The combined organic layers were washed with brine (20 mL), dried over anhydrous Na₂SO₄, filtered, concentrated and purified by column chromatography on silica gel to afford (R)-tert-butyl (1-(benzyl(2-hydroxyethyl)amino)-3-methyl-1-oxobutan-2-yl)carbamate (2.80 g, 88% yield) as a white solid. LC-MS m/z 351.2 [M+H]⁺.

### Step 2:

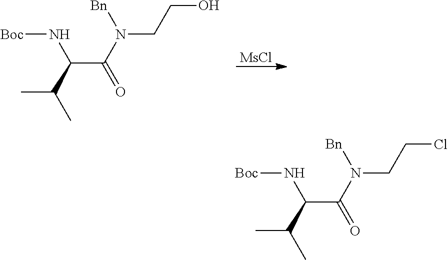 [SMILES: "MsCl" ; CC(C)[C@@H](NC(=O)OC(C)(C)C)C(=O)N(CCO)Cc1ccccc1 ; CC(C)[C@@H](NC(=O)OC(C)(C)C)C(=O)N(CCCl)Cc1ccccc1]

To a solution of (R)-tert-butyl (1-(benzyl(2-hydroxyethyl)amino)-3-methyl-1-oxobutan-2-yl)carbamate (2.80 g, 8.0 mmol) in CH₂Cl₂ (20 mL) were added Et₃N (1.60 g, 16 mmol) and MsCl (1.40 g, 12.0 mmol) dropwise at −10° C. under N₂. The mixture was stirred at rt overnight. The mixture was quenched with water (20 mL) and extracted with CH₂Cl₂ (3×20 mL). The combined organic layers were washed with brine (20 mL) and dried over anhydrous Na₂SO₄, filtered, concentrated to afford (R)-tert-butyl (1-(benzyl(2-chloroethyl)amino)-3-methyl-1-oxobutan-2-yl)carbamate (3.0 g, 100% yield) as a yellow solid, which was used for the next step without further purification. LC-MS m/z 369.2 [M+H]⁺. ¹H NMR (CDCl₃ 400 MHz): δ 7.37-7.28 (m, 3H), 7.22-7.20 (m, 2H), 5.27-5.18 (m, 1H), 4.93-4.86 (m, 1H), 4.64-4.39 (m, 2H), 3.85-3.66 (m, 2H), 3.61-3.39 (m, 2H), 2.03-1.97 (m, 1H), 1.45 (s, 9H), 0.98 (d, J=6.8 Hz, 3H), 0.93 (d, J=6.8 Hz, 3H).

### Step 3:

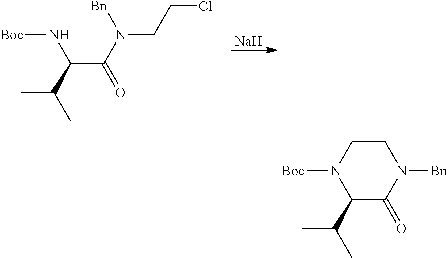

To a solution of (R)-tert-butyl (1-(benzyl(2-chloroethyl)amino)-3-methyl-1-oxobutan-2-yl)carbamate (2.0 g, 5.40 mmol) in DMF (30 mL) was added NaH (1.0 g, 27.0 mmol, 60% in oil mineral) at 0° C. under N₂. The mixture was stirred at rt for 2 h. The mixture was quenched with water (20 mL) and extracted with EtOAc (3×20 mL). The combined organic layers were washed with brine (20 mL) and dried over anhydrous Na₂SO₄, filtered, concentrated and purified by column chromatography to afford (R)-tert-butyl 4-benzyl-2-isopropyl-3-oxopiperazine-1-carboxylate (1.13 g, 63% yield) as a white solid. LC-MS m/z 277.1 [M−56+H]⁺. ¹H NMR (CDCl₃ 400 MHz): δ 7.38-7.29 (m, 3H), 7.29-7.22 (m, 2H), 5.02-4.86 (m, 1H), 4.49-4.39 (m, 1H), 4.31-4.06 (m, 2H), 3.41-3.18 (m, 3H), 2.42-2.31 (m, 1H), 1.46 (s, 9H), 1.12 (d, J=6.8 Hz, 3H), 1.00 (d, J=6.8 Hz, 3H).

### Step 4:

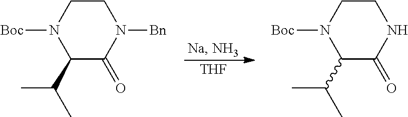

To a three-necked bottle containing THF (10 mL) was bubbled with NH₃ (gas) at −78° C. for 5 mins. Na (300 mg, 13.0 mmol) was added to the mixture slowly at −78° C. After stirring for 30 min, (R)-tert-butyl 4-benzyl-2-isopropyl-3-oxopiperazine-1-carboxylate (700 mg, 2.11 mmol) was added dropwise at −78° C. The mixture was stirred at −78° C. for 30 min. The mixture was quenched with sat. aq NH₄Cl(10 mL) and extracted with EtOAc (3×10 mL). The combined organic layers were washed with brine (10 mL), dried over anhydrous Na₂SO₄, filtered, concentrated and purified by preparative TLC with PE/EtOAc 1/1 to afford tert-butyl 2-isopropyl-3-oxopiperazine-1-carboxylate (300 mg, 59% yield) as a white solid. The product was found to be a racemic mixture. The cause of racemization was not investigated. LC-MS m/z 187.1 [M−56+H]⁺, 265.1 [M+Na]⁺. ¹H NMR (CDCl₃ 400 MHz): δ 6.29 (s, 1H), 4.55-3.99 (m, 2H), 3.51-3.36 (m, 1H), 3.32-3.12 (m, 2H), 2.34-2.29 (m, 1H), 1.46 (s, 9H), 1.09 (d, J=6.8 Hz, 3H), 0.99 (d, J=7.2 Hz, 3H).

### Step 5:

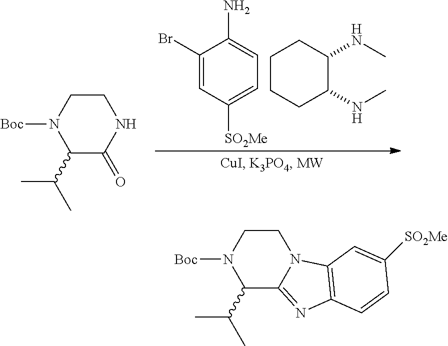

To a solution of tert-butyl 2-isopropyl-3-oxopiperazine-1-carboxylate (200 mg, 0.83 mmol) in NMP (3 mL) was added 2-bromo-4-(methylsulfonyl)aniline (207 mg, 0.83 mmol), (1R,2S)—N1,N2-dimethylcyclohexane-1,2-diamine (12.0 mg, 0.08 mmol), K₃PO₄.3H₂O (660 mg, 2.48 mmol), Cul (16 mg, 0.08 mmol). The mixture was stirred at 150° C. for 1 h under microwave. The mixture was diluted with water (10 mL) and extracted with EtOAc (3×10 mL). The combined organic layers were washed with brine (10 mL), dried over anhydrous Na₂SO₄, filtered, concentrated and purified by preparative TLC with CH₂Cl₂/MeOH 35/1 to afford tert-butyl 1-isopropyl-7-(methylsulfonyl)-3,4-dihydrobenzo[4,5]imidazo[1,2-a]pyrazine-2(1H)-carboxylate (110 mg, 34% yield) as a white solid.

LC-MS m/z 394.1 [M+H]⁺. ¹H NMR (CDCl₃ 400 MHz): δ 7.94 (s, 1H), 7.83-7.76 (m, 2H), 5.35-5.17 (m, 1H), 4.73-4.42 (m, 1H), 4.22-4.12 (m, 1H), 4.11-3.99 (m, 1H), 3.53-3.37 (m, 1H), 3.03 (s, 3H), 2.38-2.27 (m, 1H), 1.42 (s, 9H), 1.19 (d, J=6.8 Hz, 3H), 0.97 (d, J=6.8 Hz, 3H).

### Preparation 2

### (R)-2-tert-butyl 8-methyl 1-isopropyl-7-(methylsulfonyl)-3,4-dihydrobenzo[4,5]imidazo[1,2-a]pyrazine-2,8(1H)-dicarboxylate

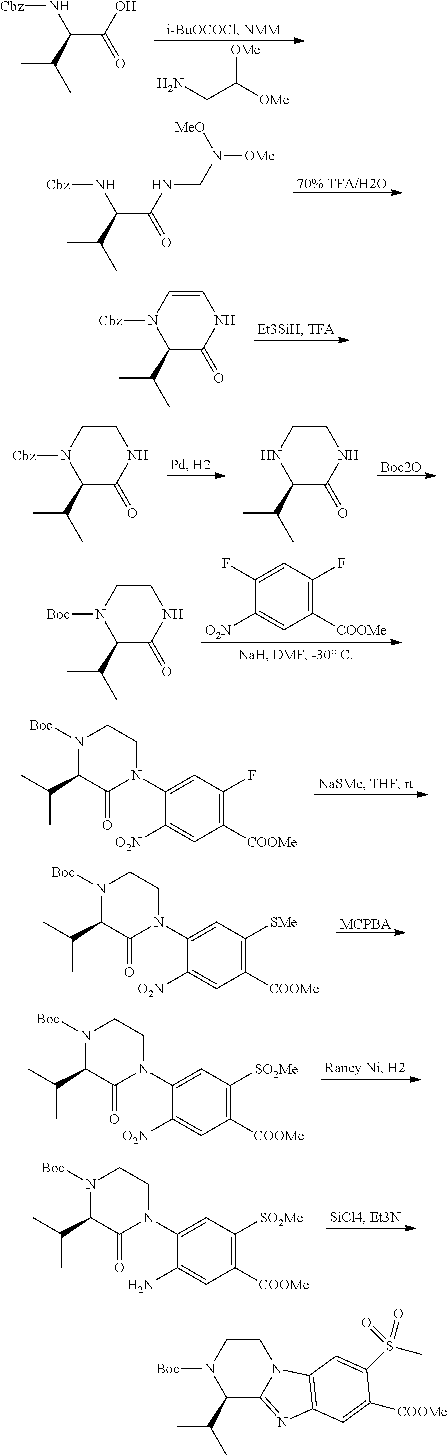

### Step 1:

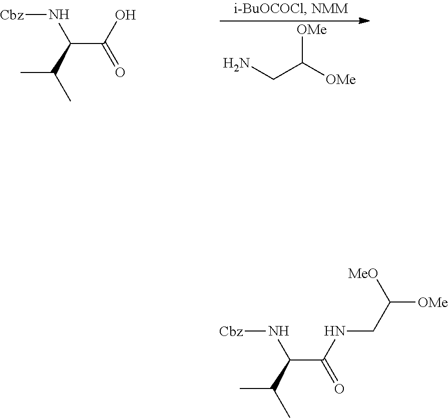 [SMILES: CC(C)[C@@H](NC(=O)OCc1ccccc1)C(=O)O ; COC(CN)OC ; COC(CNC(=O)[C@H](NC(=O)OCc1ccccc1)C(C)C)OC]

A solution of Cbz-D-Valine (500 g, 1.99 mol) and N-methylmorpholine (201.8 g, 1.99 mol) in anhydrous THF (8 L) was cooled to −15° C., i-butylchlorofomate (299 g, 2.19 mol) was added dropwise under stirring. After 30 min, a solution of 1-amino-2,2-dimethyoxypropane (209.5 g, 1.99 mol) in THF (1 L) was added slowly and the temperature was maintained at −15° C. for 2 h. The reaction mixture was washed with brine (2 L) and the organic phase was concentrated to remove the THF. The residue was diluted with EtOAc (4 L), washed with 1N aqueous HCl (2×2 L), washed with sat. NaHCO₃ (2 L) and Na₂CO₃ (2 L), and washed with brine (1.5 L). After drying over Na₂SO₄, the organic solvent was removed under reduce pressure to afford (R)-benzyl (1-((2,2-dimethoxyethyl)amino)-3-methyl-1-oxobutan-2-yl)carbamate as a white solid (670 g, yield 99.5%), which was used for next step without further purification. LC-MS m/z 360.9 [M+Na]⁺. ¹H NMR (CD₃OD 300 MHz): δ 7.35-7.30 (m, 5H), 5.08 (s, 2H), 4.45-4.35 (m, 1H), 3.95-3.85 (m, 1H), 3.34-3.25 (m, 8H), 2.10-1.90 (m, 1H), 0.94-0.91 (m, 6H).

### Step 2:

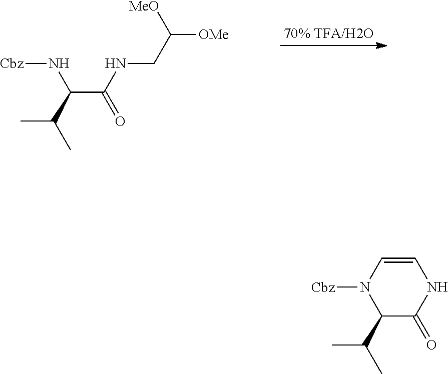 [SMILES: COC(CNC(=O)[C@H](NC(=O)OCc1ccccc1)C(C)C)OC ; CC(C)[C@@H]1C(=O)NC=CN1C(=O)OCc1ccccc1]

(R)-benzyl (1-((2,2-dimethoxyethyl)amino)-3-methyl-1-oxobutan-2-yl)carbamate (335 g, 0.99 mol) was added in portions to a cooled TFA-H₂O (temperature <5° C., V_{TFA}/V_{H2O}=7/3, 2 L), and the solution was stirred at rt for 12 h. The solution was added slowly into a stirring cooled sat. aq. Na₂CO₃ (2.5 L) to keep the pH>8. Then the mixture was extracted with EtOAc (5×2 L). The combined organic layers were washed with brine (2 L), dried over anhydrous Na₂SO₄, filtered and evaporated in vacuo to give (R)-benzyl 2-isopropyl-3-oxo-3,4-dihydropyrazine-1(2H)-carboxylate as a white solid (259 g, 95.4%), which was used for next step without further purification. LC-MS m/z 274.9 [M+H]⁺. ¹H NMR (CD₃OD 300 MHz): δ7.36-7.34 (m, 5H), 6.33-6.30 (m, 1H), 5.79-5.68 (m, 1H), 5.26-5.13 (m, 2H), 4.38-4.29 (m, 1H), 2.01-1.96 (m, 1H), 1.00-0.84 (m, 6H).

### Step 3:

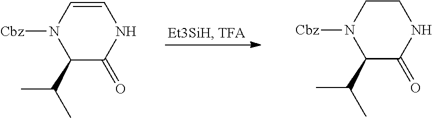 [SMILES: CC(C)[C@@H]1C(=O)NC=CN1C(=O)OCc1ccccc1 ; CC(C)[C@@H]1C(=O)NCCN1C(=O)OCc1ccccc1]

To a stirring solution of (R)-benzyl 2-isopropyl-3-oxo-3,4-dihydropyrazine-1(2H)-carboxylate (400 g, 1.46 mol) in DCE (2 L) was added Et₃SiH (424 g, 3.65 mol) and TFA (665 g, 5.8 mol) at rt. The reaction was stirred under reflux for 36 h. After cooled to rt, the solution was concentrated to remove the solvent. The residue was diluted with EtOAc (2 L), and it was added slowly into a stirring cooled sat. aq. NaHCO₃ (2 L) to make sure that the pH>8. The mixture was extracted with EtOAc (2×2.5 L). The combined organic layers were washed with brine, dried over anhydrous Na₂SO₄, filter and concentrated to give (R)-benzyl 2-isopropyl-3-oxopiperazine-1-carboxylate (402 g, yield 99.75%), which was used for next step without further purification. LC-MS m/z 276.9 [M+H]⁺. ¹H NMR (DMSO-d₆ 400 MHz): δ 7.93 (s, 1H), 7.39-7.31 (m, 5H), 5.09 (s, 2H), 4.06-4.01 (m, 1H), 3.99-3.92 (m, 1H), 3.23-3.14 (m, 3H), 2.20-2.12 (m, 1H), 0.96-0.94 (m, 3H), 0.85 (d, J=6.0 Hz, 3H).

### Step 4:

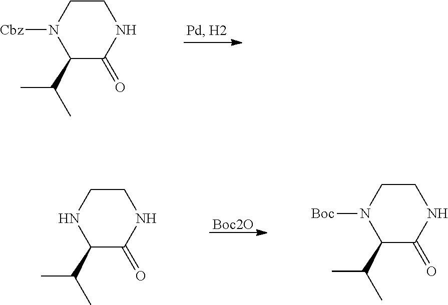

To a 1 L round-bottom flask containing (R)-benzyl 2-isopropyl-3-oxopiperazine-1-carboxylate (50 g, 0.181 mol) in MeOH (800 mL) was added Pd/C (dry, w/w 15%, 5 g). The mixture was stirred at rt under H₂ (1 atm) overnight. When TLC and LCMS showed that the starting material was consumed, (Boc)₂O (76.74 g, 0.352 mol) was added to the reaction mixture, and the mixture was stirred at rt overnight until the intermediate (R)-3-isopropylpiperazin-2-one was consumed. The mixture was filtered and concentrated under vacuum to give a residue. The residue was purified by column chromatography on silica gel (eluting with PE:EtOAc=3:1) to give (R)-tert-butyl 2-isopropyl-3-oxopiperazine-1-carboxylate as a white solid (26 g, yield 61%). For (R)-3-isopropyl-piperazin-2-one: LC-MS m/z 143.2 [M+H]⁺. ¹H NMR (HCl salt, CD₃OD 400 MHz): δ 3.95 (d, J=3.6 Hz, 1H), 3.65-3.39 (m, 4H), 2.63-2.54 (m, 1H), 1.15 (d, J=6.8 Hz, 3H), 1.09 (d, J=7.2 Hz, 3H). For (R)-tert-butyl 2-isopropyl-3-oxopiperazine-1-carboxylate: LC-MS m/z 186.9 [M−56+H]⁺. ¹H NMR (DMSO-d₆ 400 MHz): δ 7.93 (s, 1H), 4.02-3.82 (m, 2H), 3.17-3.15 (m, 3H), 2.16 (s, 1H), 1.41 (s, 9H), 0.98 (d, J=6.8 Hz, 3H), 0.89 (d, J=6.4 Hz, 3H).

### Step 5:

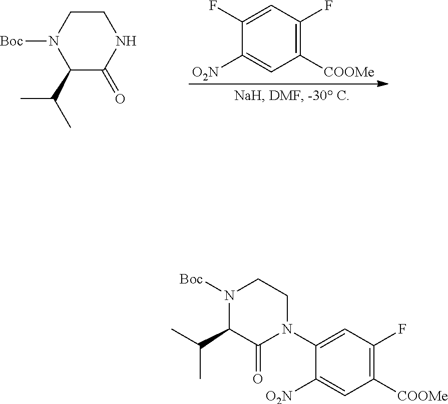 [SMILES: CC(C)[C@@H]1C(=O)NCCN1C(=O)OC(C)(C)C ; COC(=O)c1cc([N+](=O)[O-])c(F)cc1F ; COC(=O)c1cc([N+](=O)[O-])c(N2CCN(C(=O)OC(C)(C)C)[C@H](C(C)C)C2=O)cc1F]

Under N₂ atmosphere, NaH (8.8 g, 0.22 mol, 60% in mineral oil, 1.1 eq.) was added in portions at −10° C. to a 1 L three-neck flask containing (R)-tert-butyl 2-isopropyl-3-oxopiperazine-1-carboxylate (26.7 g, 0.11 mol) in DMF (300 mL). The mixture was stirred at −10° C. for 30 min. The mixture was added dropwise to a 1 L three-neck flask containing methyl 2,4-difluoro-5-nitrobenzoate (26.3 g, 0.121 mol, 1.1 eq.) in DMF (200 mL) at −20° C. over 10 min. After addition, the resulting mixture was stirred between −20° C. and −30° C. for another 10 min. The reaction was quenched with sat. aq ammonium chloride (200 mL) and then water (800 mL). The aqueous layer was extracted with EtOAc (3×1 L). The combined organic layers were washed with water (3×1 L) and brine, and then dried over anhydrous Na₂SO₄. After the mixture was filtered and the filter was evaporated under vacuum, the residue was purified by column chromatography on silica gel eluting with PE: EtOAc 8:1˜4:1 to give (R)-tert-butyl 4-(5-fluoro-4-(methoxycarbonyl)-2-nitrophenyl)-2-isopropyl-3-oxopiperazine-1-carboxylate (32 g, 66.3% yield) as a yellow solid. LC-MS MS (ESI) m/z 384.1 [M−56+H]⁺, 462.1[M+Na]⁺. ¹H NMR (CDCl₃ 300 MHz): δ 8.63 (d, J=6.9 Hz, 1H), 7.16 (d, J=10.2 Hz, 1H), 4.61-4.30 (m, 2H), 3.97-3.89 (m, 4H), 3.62-3.48 (m, 2H), 2.40-2.34 (m, 1H), 1.49 (s, 9H), 1.08 (d, J=6.9 Hz, 3H), 1.01 (d, J=6.9 Hz, 3H).

### Step 6:

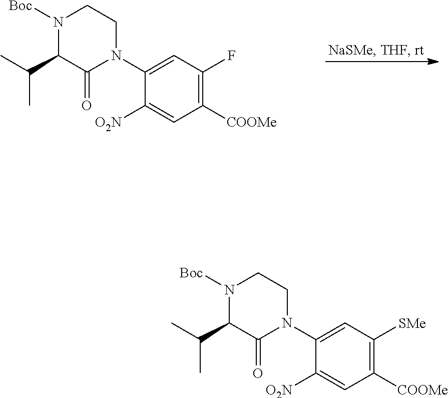 [SMILES: COC(=O)c1cc([N+](=O)[O-])c(N2CCN(C(=O)OC(C)(C)C)[C@H](C(C)C)C2=O)cc1F ; COC(=O)c1cc([N+](=O)[O-])c(N2CCN(C(=O)OC(C)(C)C)[C@H](C(C)C)C2=O)cc1SC]

To a 1 L round-bottom flask containing (R)-tert-butyl 4-(5-fluoro-4-(methoxycarbonyl)-2-added NaSMe (14.3 g, 0.204 mmol, 3 eq.). The mixture was stirred at rt for 1 h. Water (500 mL) was added and the mixture was concentrated under vacuum to remove THF. The aqueous layer was extracted with EtOAc (3×800 mL). The combined organic layers were washed with brine, dried over anhydrous Na₂SO₄, filtered and concentrated under vacuum to give (R)-tert-butyl 2-isopropyl-4-(4-(methoxycarbonyl)-5-(methylthio)-2-nitrophenyl)-3-oxopiperazine-1-carboxylate (31.9 g, 100% yield) as a yellow solid. The residue was used directly for the next step without further purification. LC-MS MS (ESI) m/z 412.1 [M−56+H]⁺, 490.2[M+Na]⁺.

### Step 7:

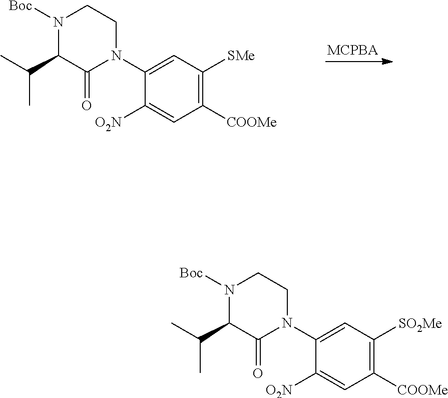 [SMILES: COC(=O)c1cc([N+](=O)[O-])c(N2CCN(C(=O)OC(C)(C)C)[C@H](C(C)C)C2=O)cc1SC ; COC(=O)c1cc([N+](=O)[O-])c(N2CCN(C(=O)OC(C)(C)C)[C@H](C(C)C)C2=O)cc1S(C)(=O)=O]

To a 2 L round-bottom flask containing (R)-tert-butyl 2-isopropyl-4-(4-(methoxycarbonyl)-5-(methylthio)-2-nitrophenyl)-3-oxopiperazine-1-carboxylate (crude 91.7 g, 0.196 mol) in CH₂Cl₂ (1 L) was added m-CPBA (84.6 g, 0.49 mmol, 2.5 eq). The mixture was stirred at rt overnight. Sat. Na₂S₂O₃ was added slowly to quench the reaction. The mixture was extracted with CH₂Cl₂ (4×3 L). The combined organic layers were washed successively with Na₂S₂O₃ solution (500 mL), NaHCO₃ solution (500 mL) and brine, dried over anhydrous Na₂SO₄, filtered and concentrated under vacuum. The residue was purified by column chromatography on silica gel eluting with dichloromethane to give (R)-tert-butyl 2-isopropyl-4-(4-(methoxycarbonyl)-5-(methylsulfonyl)-2-nitrophenyl)-3-oxopiperazine-1-carboxylate (83.7 g, 85.4% yield) as a yellow solid. LC-MS MS (ESI) m/z 444.0 [M−56+H]⁺, 522.1 [M+Na]⁺. ¹H NMR (CDCl₃ 300 MHz): δ 8.29 (s, 1H), 8.12 (s, 1H), 4.61-4.17 (m, 2H), 4.00-3.94 (m, 4H), 3.70-3.60 (m, 1H), 3.51-3.43 (m, 4H), 2.39-2.32 (m, 1H), 1.50 (s, 9H), 1.07 (d, J=6.9 Hz, 3H), 1.01 (d, J=6.9 Hz, 3H).

### Step 8:

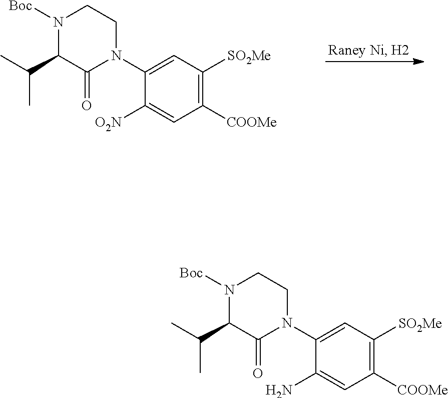 [SMILES: COC(=O)c1cc([N+](=O)[O-])c(N2CCN(C(=O)OC(C)(C)C)[C@H](C(C)C)C2=O)cc1S(C)(=O)=O ; COC(=O)c1cc(N)c(N2CCN(C(=O)OC(C)(C)C)[C@H](C(C)C)C2=O)cc1S(C)(=O)=O]

To a 1 L round-bottom flask containing (R)-tert-butyl 2-isopropyl-4-(4-(methoxycarbonyl)-5-(methylsulfonyl)-2-nitrophenyl)-3-oxopiperazine-1-carboxylate (26.3 g, 0.0526 mol) in THF (200 mL) and methanol (200 mL) was added Raney Nickel (in H₂O, 4 g). The mixture was stirred under H₂ (30 psi) at rt overnight. The mixture was filtered and concentrated under vacuum to give (R)-tert-butyl 4-(2-amino-4-(methoxycarbonyl)-5-(methylsulfonyephenyl)-2-isopropyl-3-oxopiperazine-1-carboxylate (24.7 g, 100% yield) as a yellow solid. The residue was used directly for the next step without further purification. LC-MS MS (ESI) m/z 414.0 [M−56+H]⁺, 492.0 [M+Na]⁺. ¹H NMR (CDCl₃ 300 MHz): δ 7.77 (brs, 1H), 7.04 (s, 1H), 4.68-4.45 (m, 1H), 4.45-4.38 (m, 2H), 3.92 (s, 3H), 3.70-3.58 (m, 1H), 3.58-3.41 (m, 1H), 3.30 (s, 3H), 2.49-2.25 (m, 1H), 1.50 (s, 9H), 1.12 (d, J=6.9 Hz, 3H), 1.05 (d, J=6.9 Hz, 3H).

### Step 9:

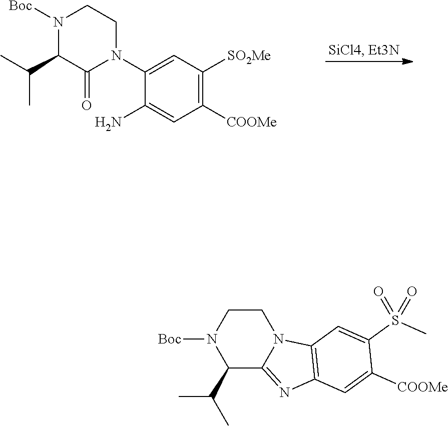 [SMILES: COC(=O)c1cc(N)c(N2CCN(C(=O)OC(C)(C)C)[C@H](C(C)C)C2=O)cc1S(C)(=O)=O ; COC(=O)c1cc2nc3n(c2cc1S(C)(=O)=O)CCN(C(=O)OC(C)(C)C)[C@@H]3C(C)C]

To a 1 L round-bottom flask containing (R)-tert-butyl 4-(2-amino-4-(methoxycarbonyl)-5-(methylsulfonyl)phenyl)-2-isopropyl-3-oxopiperazine-1-carboxylate (25 g, 0.0532 mol) in dichloromethane (500 mL) was added Et₃N (64.5 g, 0.638 mol, 12 eq.) and SiCl₄ (27.1 g, 0.160 mol, 3 eq.). The mixture was stirred at rt overnight. The mixture was added dropwise to aq. NaHCO₃ solution (54.1 g in 1 L of water, 0.644 mol, 12.1 eq.) at 0° C. slowly and adjusted to pH=8. The mixture was filtered and the aqueous layer was extracted with dichloromethane (3×600 mL). The combined organic layers were washed with brine, and then dried over anhydrous Na₂SO₄. The mixture was filtered and concentrated under vacuum to give the residue. The residue was purified by column chromatography on silica gel eluting with PE:EtOAc 2:1 to give (R)-2-tert-butyl 8-methyl 1-isopropyl-7-(methylsulfonyl)-3,4-dihydrobenzo[4,5]imidazo[1,2-a]pyrazine-2,8(1H)-dicarboxylate (13.2 g, 55% yield) as a pale yellow solid. Analytical chiral HPLC: t_{R}=9.03 min in 15 min chromatography (Method: OD-3_3_5_40_2.5 ML). LC-MS MS (ESI) m/z 452.2 [M+H]⁺. ¹H NMR (CD₃OD 400 MHz): δ 8.31 (s, 1H), 8.01 (s, 1H), 5.30-5.18 (m, 1H), 4.70-4.52 (m, 1H), 4.47 (dd, J=3.2 and 12.4 Hz, 1H), 4.18 (dt, J=5.2 and 11.6 Hz, 1H), 3.98 (s, 3H), 3.70-3.52 (m, 1H), 3.44 (s, 3H), 2.50-2.38 (m, 1H), 1.53 (s, 9H), 1.25 (d, J=6.8 Hz, 3H), 1.06 (d, J=6.8 Hz, 3H).

### Preparation 3

### 1-isopropyl-7-(methylthio)-1,2,3,4-tetrahydropyrazino[1,2-a]indole

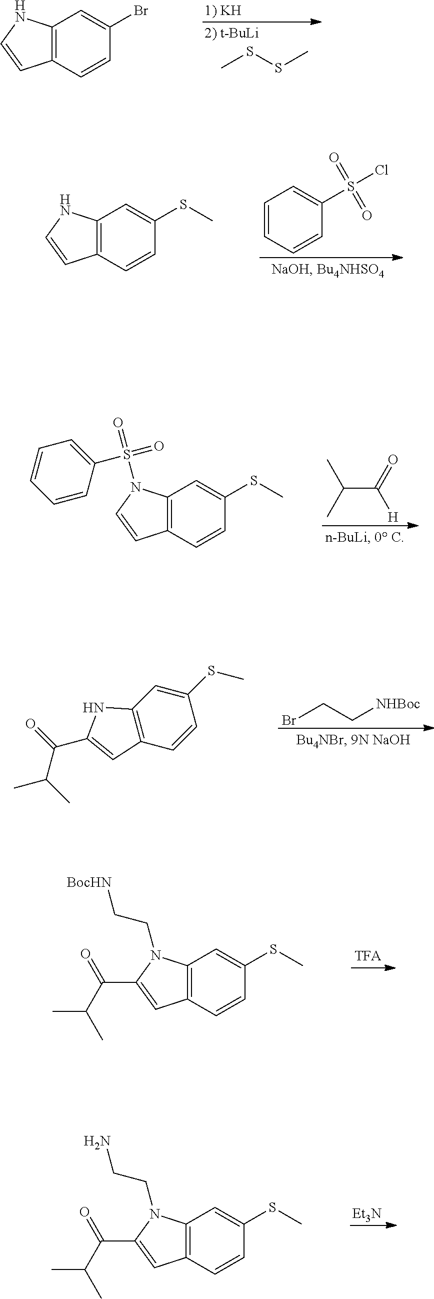

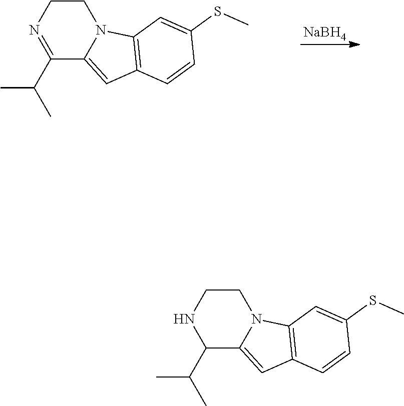

### Step 1:

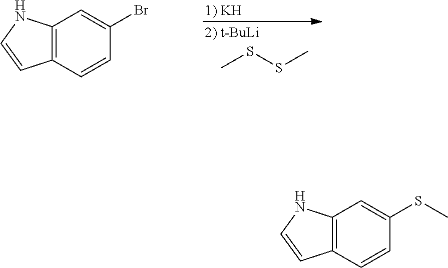

To a solution of 6-bromo-1H-indole (5 g, 25.50 mmol) in anhydrous THF (60 mL) at 0° C. was added KH (6.80 g, 51.00 mmol, 30% wt in mineral oil). After stirring for 30 min and cooling to −78° C., t-BuLi (39.23 mL, 51.0 mmol, 1.3 M) was added to the formed mixture under nitrogen.

After 30 min, 1,2-dimethyldisulfane (4.80 g, 51.0 mmol) was added to the formed mixture. The reaction mixture was stirred at −78° C. for 1 h. The mixture was quenched with sat. NH₄Cl (30 mL) at −78° C. slowly (Caution: flame), adjusted pH=7 with 1 N aqueous phosphoric acid and extracted with EtOAc (50 mL×3). The combined organic layers were dried over anhydrous sodium sulfate, filtered, concentrated and purified by column chromatography on silica gel eluted with PE/EtOAc 10:1 to give 6-(methylthio)-1H-indole (3.9 g, 93.67% yield) as a grey solid. LC-MS MS (ESI) m/z 164.1 [M+H]⁺. ¹H NMR (CDCl₃ 400 MHz): δ 8.14 (brs, 1H), 7.56 (d, J=8.0 Hz, 1H), 7.37 (s, 1H), 7.18-7.11 (m, 1H), 6.56-6.51 (m, 1H), 2.52 (s, 3H).

### Step 2:

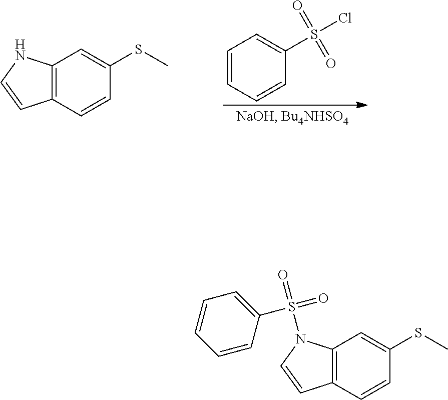 [SMILES: CSc1ccc2cc[nH]c2c1 ; O=S(=O)(Cl)c1ccccc1 ; CSc1ccc2ccn(S(=O)(=O)c3ccccc3)c2c1]

To a solution of 6-(methylthio)-1H-indole (1 g, 6.13 mmol), NaOH (4.90 g, 122.6 mmol) and Bu₄NHSO₄ (207.8 mg, 0.613 mmol) in dichloromethane (20 mL) was added benzenesulfonyl chloride (1.29 g, 7.36 mmol). The reaction mixture was stirred at rt overnight. The mixture was quenched with water (30 mL) and extracted with dichloromethane (30 mL×3). The combined organic layers were dried over anhydrous sodium sulfate, filtered, concentrated and purified by column chromatography on silica gel eluted with PE/EtOAc 10:1 to afford 6-(methylthio)-1-(phenylsulfonyl)-1H-indole (1.1 g, 59.18% yield) as a white solid.

LC-MS MS (ESI) m/z 304.0 [M+H]⁺. ¹H NMR (CDCl₃ 400 MHz): δ 7.93-7.75 (m, 3H), 7.58-7.41 (m, 5H), 7.17 (dd, J₁=8.0 Hz, J₂=1.6 Hz, 1H), 6.63-6.60 (m, 1H), 2.53 (s, 3H).

### Step 3:

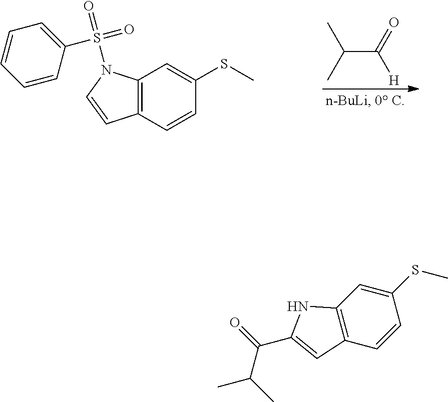

To a solution of 6-(methylthio)-1-(phenylsulfonyl)-1H-indole (890 mg, 2.93 mmol) in anhydrous THF (10 mL) at 0° C. under nitrogen was added n-BuLi (5.86 mL, 14.65 mmol, 2.5 M). After stirring for 30 min, isobutyraldehyde (1.05 g, 14.65 mmol) was added to the formed mixture. The reaction mixture was stirred at 0° C. for 1 h. The mixture was quenched with sat. NH₄Cl (10 mL) at 0° C. and extracted with EtOAc (20 mL×3). The combined organic layers were dried over anhydrous sodium sulfate, filtered, concentrated and purified by column chromatography on silica gel eluted with PE/EtOAc 20:1 to give 2-methyl-1-(6-(methylthio)-1H-indol-2-yl)propan-1-one (440 mg, 64.28% yield) as a colorless oil. LC-MS MS (ESI) m/z 234.1 [M+H]⁺. ¹H NMR (CDCl₃ 400 MHz): δ 8.86 (brs, 1H), 7.52 (d, J=8.4 Hz, 1H), 7.19 (s, 1H), 7.14-7.11 (m, 1H), 7.01 (dd, J₁=8.4 Hz, J₂=1.6, 1H), 3.42-3.38 (m, 1H), 2.47 (s, 3H), 1.20 (d, J=6.8 Hz, 6H).

### Step 4:

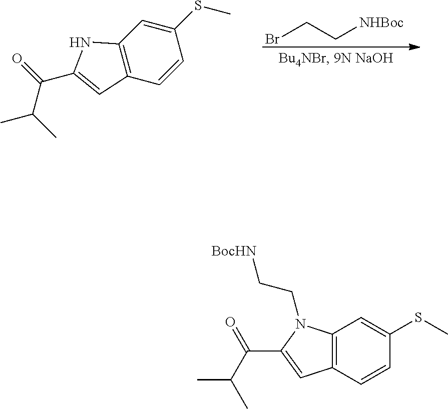 [SMILES: CSc1ccc2cc(C(=O)C(C)C)[nH]c2c1 ; CC(C)(C)OC(=O)NCCBr ; CSc1ccc2cc(C(=O)C(C)C)n(CCNC(=O)OC(C)(C)C)c2c1]

To a solution of 2-methyl-1-(6-(methylthio)-1H-indol-2-yl)propan-1-one (600 mg, 2.57 mmol) and Bu₄NBr (4.12 g, 12.85 mmol) in 9 N NaOH (10 mL, cooled) was added tert-butyl (2-bromoethyl)carbamate (2.87 g, 12.85 mmol). The reaction mixture was stirred at rt for 72 h. The mixture was diluted with water (20 mL) at 0° C., extracted with EtOAc (20 mL×3). The combined organic layers were dried over anhydrous sodium sulfate, filtered, concentrated and purified by column chromatography on silica gel eluting with PE/EtOAc 10:1 to afford tert-butyl (2-(2-isobutyryl-6-(methylthio)-1H-indol-1-yl)ethyl)carbamate (200 mg, 20.66% yield) as a colorless oil. LC-MS MS (ESI) m/z 321.1 [M−56+H]⁺, 277.1 [M−100+H]⁺. ¹H NMR (CDCl₃ 400 MHz): δ 7.57 (d, J=8.4 Hz, 1H), 7.38 (s, 1H), 7.29 (s, 1H), 7.10 (d, J=8.4 Hz, 1H), 4.80 (brs, 1H), 4.62 (t, J=6.4 Hz, 2H), 3.58-3.42 (m, 3H), 2.58 (s, 3H), 1.38 (s, 9H), 1.24 (d, J=6.8 Hz, 6H).

### Step 5:

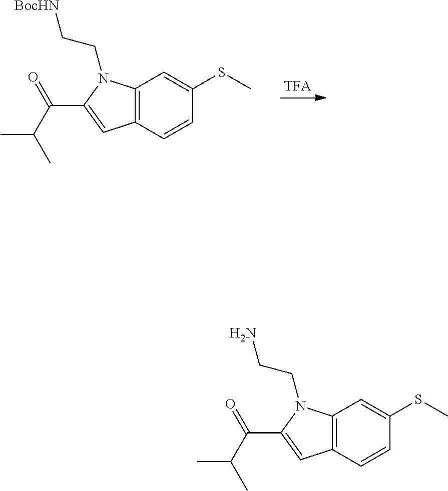 [SMILES: CSc1ccc2cc(C(=O)C(C)C)n(CCNC(=O)OC(C)(C)C)c2c1 ; CSc1ccc2cc(C(=O)C(C)C)n(CCN)c2c1]

To a solution afford tert-butyl (2-(2-isobutyryl-6-(methylthio)-1H-indol-1-yl)ethyl)carbamate (200 mg, 0.53 mmol) in dichloromethane (9 mL) at 0° C. was added TFA (1 mL). The reaction mixture was stirred at rt for 1 h. The mixture was concentrated (T<25° C.), treated with water (5 mL), adjusted pH=11 with sat. NaHCO₃ and extracted with EtOAc (20 mL×3). The combined organic layers were dried over anhydrous sodium sulfate, filtered, concentrated to afford 1-(1-(2-aminoethyl)-6-(methylthio)-1H-indol-2-yl)-2-methylpropan-1-one (210 mg, 100% yield) as a colorless oil. LC-MS MS (ESI) m/z 258.8 [M−18+H]⁺.

### Step 6:

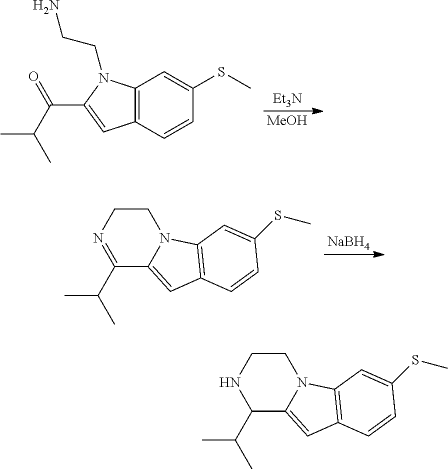

To a solution of 1-(1-(2-aminoethyl)-6-(methylthio)-1H-indol-2-yl)-2-methylpropan-1-one (200 mg, 0.724 mmol) in MeOH (5 mL) was added Et₃N (219.3 mg, 2.172 mmol). The reaction mixture was stirred at 60° C. for 1 h. Then NaBH₄ (82.53 mg, 2.172 mmol) was added to the formed mixture. The mixture was stirred at 60° C. for another 1 h. The mixture was concentrated, treated with water (10 mL) and extracted with EtOAc (20 mL×3). The combined organic layers were dried over anhydrous sodium sulfate, filtered, concentrated and purified by preparative TLC on silica gel eluted with PE/EtOAc 1:1 to afford 1-isopropyl-7-(methylthio)-1,2,3,4-tetrahydropyrazino[1,2-a]indole (80 mg, 42.46% yield, store at 0° C.) as a colorless oil. LC-MS of 1-Isopropyl-7-methylsulfanyl-3,4-dihydro-pyrazino[1,2-a]indole MS (ESI) m/z 259.1[M+H]⁺. LC-MS of 1-isopropyl-7-(methylthio)-1,2,3,4-tetrahydropyrazino[1,2-a]indole MS (ESI) m/z 261.2[M+H]⁺. ¹H NMR (CDCl₃ 400 MHz): δ 7.41 (d, J=8.4 Hz, 1H), 7.20 (s, 1H), 7.05 (dd, J₁=8.4 Hz, J₂=1.6 Hz, 1H), 6.12 (s, 1H), 4.02-3.97 (m, 2H), 3.86-3.80 (m, 1H), 3.46-3.42 (m, 1H), 3.16-3.10 (m, 1H), 2.48 (s, 3H), 2.32-2.27 (m, 1H), 1.09 (d, J=6.8 Hz, 3H), 0.86 (d, J=6.8 Hz, 3H).

### Preparation 4

### 8-(((tert-butyldiphenylsilyl)oxy)methyl)-1-isopropyl-7-(methylsulfonyl)-1,2,3,4-tetrahydropyrazino[1,2-a]indole

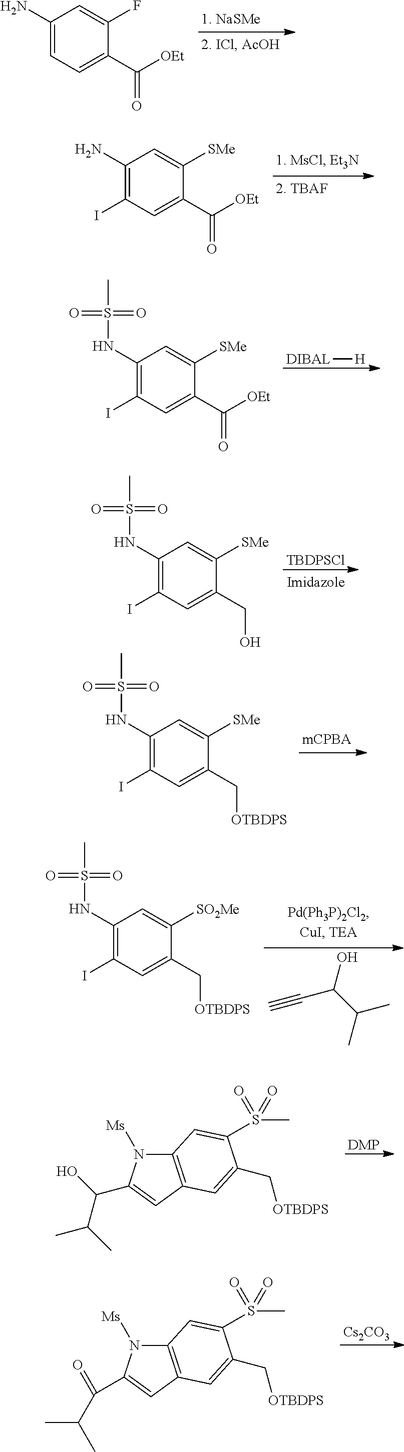

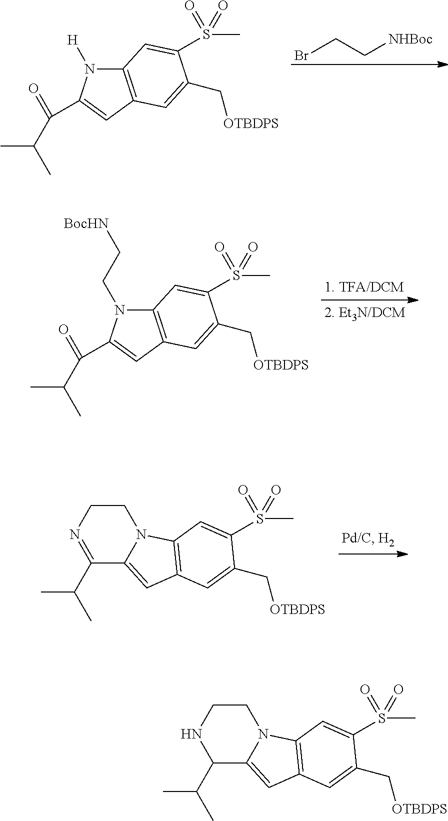

### Step 1:

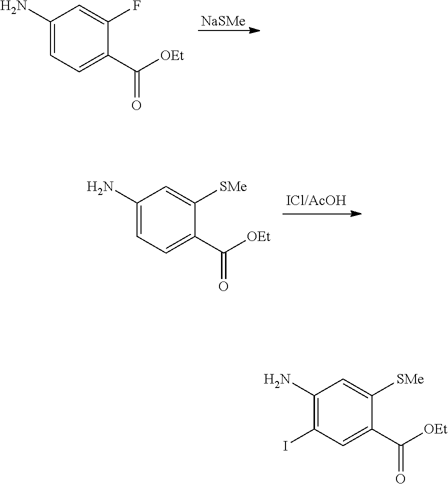

To a solution of ethyl 4-amino-2-fluorobenzoate (12 g, 65.5 mmol) in DMF (100 mL) was added NaSMe (9.17 g, 131 mmol) and the mixture was stirred at 60° C. for 20 h. After cooling to rt, the reaction was diluted with H₂O and extracted with EtOAc (3×100 mL). The combined organic phases were washed with brine, dried over anhydrous sodium sulfate, filtered and concentrated in vacuo to afford ethyl 4-amino-2-(methylthio)benzoate. To a pre-heated 60° C. solution of ethyl 4-amino-2-(methylthio)benzoate (65 mmol) in acetic acid (150 mL) was added ICl/AcOH solution (1M, 72 mL, 72 mmol) dropwise during 40 min and. the temperature was maintained at 60° C. for 3 h. After cooling to rt the reaction was diluted with EtOAc (500 mL) and washed with 5% sodium thiosulfate solution (3×100 mL) and brine (200 mL), dried over anhydrous sodium sulfate, filtered and concentrated in vacuo. The crude product was purified by silica gel chromatography (0-20% EtOAc/Hexanes) to afford ethyl 4-amino-5-iodo-2-(methylthio)benzoate (13.67 g, 53% yield). For ethyl 4-amino-2-(methylthio)benzoate: LC-MS m/z 212 [M+H]⁺. For ethyl 4-amino-5-iodo-2-(methylthio)benzoate: LC-MS m/z 338 [M+H]⁺. ¹H NMR (400 MHz, CDCl₃): δ 8.29 (s, 1H), 6.47 (s, 1H), 4.49 (br s, 2H), 4.31 (q, J=7.2 Hz, 2H), 2.38 (s, 3H), 1.37 (t, J=7.2 Hz, 3H).

### Step 2:

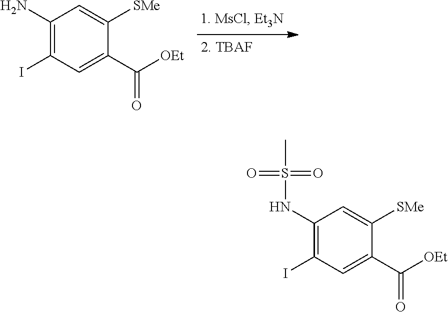 [SMILES: CCOC(=O)c1cc(I)c(N)cc1SC ; CCOC(=O)c1cc(I)c(NS(C)(=O)=O)cc1SC]

To a solution of ethyl 4-amino-5-iodo-2-(methylthio)benzoate (13.6 g, 40 mmol) in DCM (100 mL) was added Et₃N (13.8 mL, 100 mmol), followed by MsCl (7.7 mL, 100 mmol) at 0° C. After addition the mixture was stirred at rt for 2 h. 1N HCl solution (50 mL) was added to the mixture and the aqueous phase was extracted with DCM (1×100 mL). The organic solution was washed with brine, dried over anhydrous sodium sulfate, filtered and concentrated in vacuo to give ethyl 5-iodo-4-(N-(methylsulfonyl)methylsulfonamido)-2-(methylthio)benzoate.

The crude reaction mixture above was dissolved into 100 mL THF. To this solution was added TBAF THF solution (1 M, 100 mL) and the mixture was stirred at rt for 2 h. H₂O was added to the mixture and the aqueous phase was extracted with EtOAc (3×100 mL). The combined organic solution was washed with brine, dried over anhydrous sodium sulfate, filtered and concentrated in vacuo to afford ethyl 5-iodo-4-(methylsulfonamido)-2-(methylthio)benzoate. It was used for next step without further purification. For ethyl 5-iodo-4-(N-(methylsulfonyl)methylsulfonamido)-2-(methylthio)benzoate: LC-MS m/z 494 [M+H]⁺. For ethyl 5-iodo-4-(methylsulfonamido)-2-(methylthio)benzoate: LC-MS m/z 415 [M+H]⁺.

### Step 3:

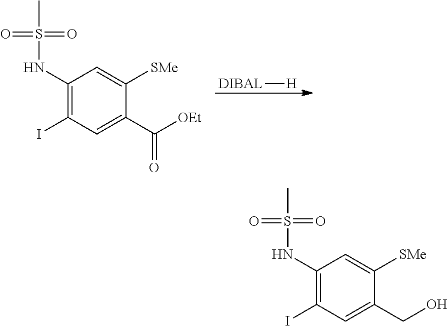

To a solution of ethyl 5-iodo-4-(methylsulfonamido)-2-(methylthio)benzoate (crude, from step 2) in dry toluene (200 mL) at 0° C. was added diisobutylaluminium hydride (1.0 M in toluene, 100 mL, 100 mmol) slowly. After addition, the mixture was stirred at 0° C. for 3 h and quenched with methanol/H₂O (1/1). The reaction mixture was poured into a vigorously stirred solution of potassium sodium tartrate (1M, 300 mL) and stirred vigorously for 2 h, after which time it settled to two clear phases. The organic layer was separated, and the aqueous layer was extracted with EtOAc (3×200 mL). The combined organic solution was washed with brine, dried over anhydrous sodium sulfate, filtered and concentrated in vacuo. The crude product was purified by silica gel chromatography (0-40% EtOAc/Hexanes) to afford N-(4-(hydroxymethyl)-2-iodo-5-(methylthio)phenyl)methanesulfonamide (11.9 g, 80% yield for two steps). LC-MS m/z 356 [M+H]⁺. ¹H NMR (400 MHz, CDCl₃): δ 7.82 (s, 1H), 7.49 (s, 1H), 4.67 (s, 2H), 2.99 (s, 3H), 2.50 (s, 3H).

### Step 4:

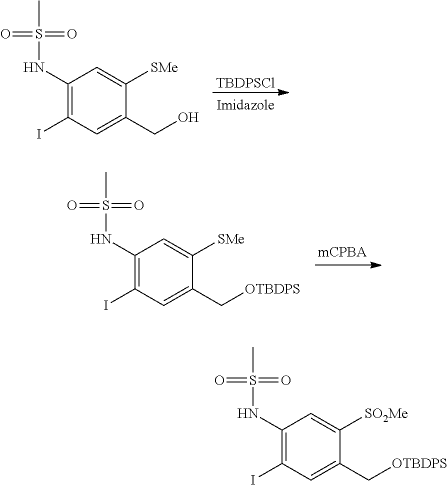

To a stirred solution of N-(4-(hydroxymethyl)-2-iodo-5-(methylthio)phenyl)methanesulfonamide (6.4 g, 17.2 mmol) and imidazole (1.76 g, 25.8 mmol) in CH₂Cl₂ (100 mL) and DMF (50 mL) at 0° C. was added tert-butyldiphenylsilyl chloride (5.8 mL, 22.4 mmol). The mixture was allowed to stir at rt overnight. The mixture was diluted with CH₂Cl₂ (100 mL), washed with 1N HCl solution, sat. aq. NaHCO₃ and brine, dried over anhydrous Na₂SO₄, filtered and concentrated in vacuo to afford N-(4-(((tert-butyldiphenylsilyl)oxy)methyl)-2-iodo-5-(methylthio)phenyl)methanesulfonamide. It was used for next step without further purification.

A suspension of crude N-(4-(((tert-butyldiphenylsilyl)oxy)methyl)-2-iodo-5-(methylthio)phenyl)methanesulfonamide, mCPBA (8.9 g, 51.6 mmol) in CH₂Cl₂ (100 mL) was stirred for 2 h at rt. Sat. aq. NaHCO₃ (50 mL) and Na₂S₂O₃ (50 mL) were added and the layers separated. The aqueous layer was extracted with CH₂Cl₂ (2×100 mL). Combined organic layers were dried over anhydrous Na₂SO₄, filtered and concentrated under reduced pressure. The residue was purified by flash chromatography over silica gel eluting with EtOAc/hexanes (3/7) to provide N-(4-(((tert-butyldiphenylsilyl)oxy)methyl)-2-iodo-5-(methylsulfonyl)phenyl)methanesulfonamide (8.8 g, 80% yield for two steps). For N-(4-(((tert-butyldiphenylsilyl)oxy)methyl)-2-iodo-5-(methylthio)phenyl)methanesulfonamide LC-MS m/z 612 [M+H]⁺. For N-(4-(((tert-butyldiphenylsilyl)oxy)methyl)-2-iodo-5-(methylsulfonyl)phenyl)methanesulfonamide: LC-MS m/z 644 [M+H]⁺. ¹H NMR (400 MHz, CDCl₃): δ 8.25 (s, 1H), 8.08 (s, 1H), 7.67-7.65 (m, 4H), 7.46-7.37 (m, 6H), 6.77 (s, 1H), 5.05 (s, 2H), 3.11 (s, 3H), 2.83 (s, 3H), 1.12 (s, 9H).

### Step 5:

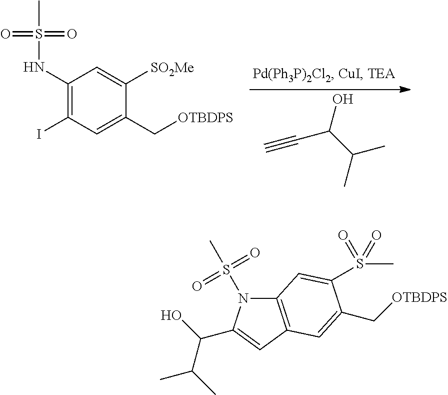

PdCl₂(PPh₃)₂ (277 mg, 0.38 mmol) and CuI (73 mg, 0.38 mmol) were added to a solution of N-(4-(((tert-butyldiphenylsilyl)oxy)methyl)-2-iodo-5-(methylsulfonyl)phenyl)methanesulfonamide (2.45 g, 3.8 mmol) in THF (20 mL) and Et₃N (10 mL). The mixture was purged with nitrogen for 10 mins followed by addition of 4-methylpent-1-yn-3-ol (745 mg, 7.6 mmol) and stirred at 65° C. for 8 h. The reaction mixture was diluted with EtOAc (50 mL) and washed with 1N HCl (50 mL). The organic layer was separated, and the aqueous layer was extracted with EtOAc (3×50 mL). The combined organic solution was washed with brine, dried over anhydrous Na₂SO₄, filtered and concentrated under reduced pressure. The residue was purified by flash chromatography over silica gel eluting with EtOAc/hexanes (3/7) to provide 1-(5-(((tert-butyldiphenylsilyl)oxy)methyl)-1,6-bis(methylsulfonyl)-1H-indol-2-yl)-2-methylpropan-1-ol (2.1 g, 90% yield). LC-MS m/z 614 [M+H]⁺. ¹H NMR (400 MHz, CDCl₃): δ 8.68 (s, 1H), 7.90 (s, 1H), 7.71-7.67 (s, 4H), 7.46-7.35 (m, 6H), 6.77 (s, 1H), 5.21 (d, J=3.2 Hz, 2H), 6.94 (t, J=6.8 Hz, 1H), 3.22 (s, 3H), 2.90 (s, 3H), 2.61 (d, J=6.8 Hz, 1H), 2.37-2.32 (m, 1H), 1.12 (s, 9H), 1.05 (d, J=6.8 Hz, 3H), 1.03 (d, J=6.8 Hz, 3H). ¹³C NMR (100 MHz, CDCl₃): δ 147.25, 135.54, 135.28, 135.00, 133.66, 133.00, 132.89, 129.96, 127.85, 121.68, 115.96, 108.69, 72.30, 62.98, 44.33, 41.59, 32.88, 26.89, 20.23, 19.30, 17.61.

### Step 6:

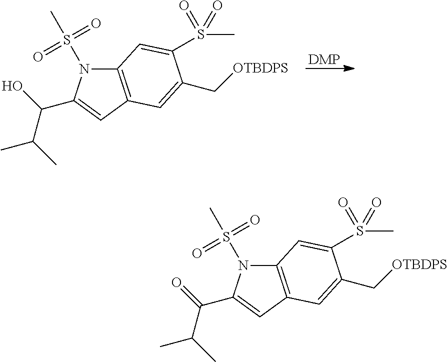

To a stirred solution of 1-(5-(((tert-butyldiphenylsilyl)oxy)methyl)-1,6-bis(methylsulfonyl)-1H-indol-2-yl)-2-methylpropan-1-ol (2.3 g, 3.8 mmol) in dry CH₂Cl₂ (25 mL) was added Dess-Martin periodiane (1.94 g, 4.56 mmol) in one portion. The mixture was allowed to stir at rt for 2 h. The reaction was quenched with a solution of Na₂S₂O₃ (5 g in 30 mL H₂O) and sat. NaHCO₃ solution (40 mL). The mixture was extracted with EtOAc (3×80 mL). The combined organic solution was washed with brine, dried over anhydrous Na₂SO₄, filtered and concentrated under reduced pressure. The residue was purified by flash chromatography over silica gel eluting with EtOAc/hexanes (2/8) to provide 1-(5-(((tert-butyldiphenylsilyl)oxy)methyl)-1,6-bis(methylsulfonyl)-1H-indol-2-yl)-2-methylpropan-1-one (2.0 g, 86% yield). LC-MS m/z 612 [M+H]⁺. ¹H NMR (400 MHz, CDCl₃): δ 8.69 (s, 1H), 8.03 (s, 1H), 7.70-7.68 (m, 4H), 7.46-7.36 (m, 6H), 7.22 (s, 1H), 5.20 (s, 2H), 3.80 (s, 3H), 3.36 (m, 1H), 2.89 (s, 3H), 1.29 (d, J=6.8 Hz, 6H), 1.13 (s, 9H). ¹³C NMR (100 MHz, CDCl₃): δ 197.83, 141.53, 136.69, 136.05, 135.50, 135.04, 132.81, 131.14, 129.99, 127.88, 123.17, 117.12, 114.21, 62.87, 44.19, 44.03, 39.09, 26.88, 19.30, 18.41.

### Step 7:

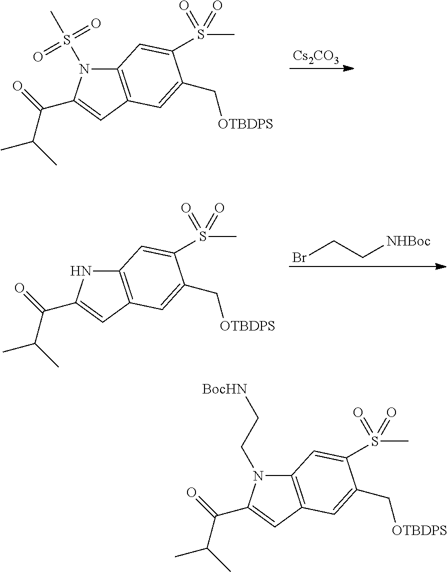

To a stirred solution of 1-(5-(((tert-butyldiphenylsilyl)oxy)methyl)-1,6-bis(methylsulfonyl)-1H-indol-2-yl)-2-methylpropan-1-one (780 mg, 1.27 mmol) in THF/methanol (15 mL/15 mL) was added Cs₂CO₃ (1.25 g, 3.83 mmol) in one portion. The mixture was allowed to stir at rt for 4 h and concentrated in vacuo to afford the crude product 1-(5-(((tert-butyldiphenylsilyl)oxy)methyl)-6-(methylsulfonyl)-1H-indol-2-yl)-2-methylpropan-1-one. It was used for the next step reaction without further purification. To a solution of crude 1-(5-(((tert-butyldiphenylsilyl)oxy)methyl)-6-(methylsulfonyl)-1H-indol-2-yl)-2-methylpropan-1-one, 2-(Boc-amino)ethyl bromide (2.8 g, 12 mmol) and tetrabutylammonium iodide (235 mg, 0.63 mmol) in CH₂Cl₂/toluene (2 mL/4 mL) was added 40% NaOH aq. solution (20 mL). The mixture was allowed to stir at rt for 20 h. The reaction mixture was diluted with CH₂Cl₂ (40 mL) and washed with H₂O (50 mL). The organic layer was separated, and the aqueous layer was extracted with CH₂Cl₂ (4×50 mL). The combined organic solution was washed with brine, dried over anhydrous Na₂SO₄, filtered and concentrated under reduced pressure. The residue was purified by flash chromatography over silica gel eluting with CH₂Cl₂/methanol (95/5) to provide tert-butyl (2-(5-(((tert-butyldiphenylsilyl)oxy)methyl)-2-isobutyryl-6-(methylsulfonyl)-1H-indol-1-yl)ethyl)carbamate (300 mg, 35% yield for two steps). For 1-(5-(((tert-butyldiphenylsilyl)oxy)methyl)-6-(methylsulfonyl)-1H-indol-2-yl)-2-methylpropan-1-one: LC-MS m/z 556 [M+Na]⁺. For tert-butyl (2-(5-(((tert-butyldiphenylsilyl)oxy)methyl)-2-isobutyryl-6-(methylsulfonyl)-1H-indol-1-yl)ethyl)carbamate: LC-MS m/z 699 [M+Na]⁺. ¹H NMR (400 MHz, CDCl₃): δ 8.20 (s, 1H), 7.93 (s, 1H), 7.72 (dd, J₁=8.0 Hz, J₂=1.6 Hz, 4H), 7.47-7.35 (m, 7H), 5.21 (s, 2H), 4.72 (d, J=6.8 Hz, 2H), 3.55 (d, J=6.8 Hz, 2H), 3.33-3.26 (m, 1H), 3.00 (s, 3H), 1.46 (s, 9H), 1.30 (d, J=6.4 Hz, 3H), 1.28 (d, J=6.4 Hz, 3H), 1.11 (s, 9H).

### Step 8:

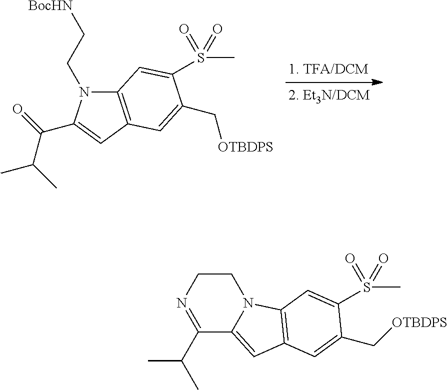

To a solution of tert-butyl (2-(5-4(tert-butyldiphenylsilyl)oxy)methyl)-2-isobutyryl-6-(methylsulfonyl)-1H-indol-1-yl)ethyl)carbamate (250 mg, 0.37 mmol) in CH₂Cl₂ (5.0 mL) was added trifluoroacetic acid (1.0 mL) and the mixture was allowed to stir at rt for 1 h. The excess amount of TFA was removed by azeotropic evaporation with toluene under reduced pressure. The residue was redissolved in CH₂Cl₂ (5 mL) and Et₃N (0.5 mL) was added. The reaction mixture was stirred at rt for 45 min and concentrated in vacuo. The residue was purified by flash chromatography over silica gel eluting with CH₂Cl₂/methanol (98/2) to provide 8-(((tert-butyldiphenylsilyl)oxy)methyl)-1-isopropyl-7-(methylsulfonyl)-3,4-dihydropyrazino[1,2-a]indole (135 mg, 65% yield). LC-MS m/z 559 [M+H]⁺.

### Step 9:

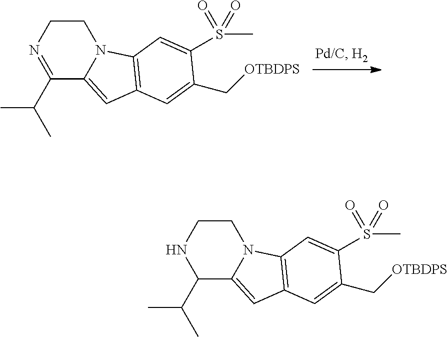

A solution of 8-(((tert-butyldiphenylsilyl)oxy)methyl)-1-isopropyl-7-(methylsulfonyl)-3,4-dihydropyrazino[1,2-a]indole (140 mg, 0.25 mmol), 10% Palladium on charcoal (37 mg, 0.025 mmol) and methanol (5 mL) was stirred at rt under 1 atmosphere of hydrogen for 3 h. The mixture was filtered through Celite® and the Celite® was washed thoroughly with methanol. Combined solvent was removed under reduced pressure to afford 8-(((tert-butyldiphenylsilyl)oxy)methyl)-1-isopropyl-7-(methylsulfonyl)-1,2,3,4-tetrahydropyrazino[1,2-a]indole. It was used directly without further purification. A small portion of product was purified by chromatography for characterization. LC-MS m/z 561 [M+H]⁺. ¹H NMR (400 MHz, CD₃OD): δ 8.00 (s, 1H), 7.73-7.70 (m, 5H), 7.47-7.40 (m, 6H), 6.36 (s, 1H), 5.20 (d, J=2.0 Hz, 2H), 4.24-4.19 (m, 1H), 4.11-4.00 (m, 2H), 3.52-3.47 (m, 1H), 3.20-3.13 (m, 1H), 3.03 (s, 3H), 2.47-2.39 (m, 1H), 1.18 (d, J=6.8 Hz, 3H), 1.09 (s, 9H), 0.96 (d, J=6.8 Hz, 3H). ¹³C NMR (100 MHz, CDCl₃): δ 143.06, 135.68, 134.04, 133.31, 131.52, 130.26, 129.79, 129.51, 127.77, 121.39, 111.22, 97.14, 63.76, 59.28, 45.00, 42.94, 42.47, 31.55, 26.94, 19.72, 19.31, 16.49.

### Preparation 5

### 1-(2-chloro-4-(trifluoromethyl)pyrimidin-5-yl)ethanone

### Method 1:

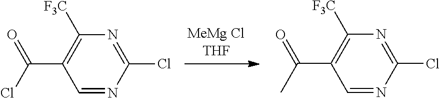

At −78° C., to a solution of 2-chloro-4-(trifluoromethyl)pyrimidine-5-carbonyl chloride (2.45 g, 10 mmol) in dry THF (50 mL) was added MeMgCl THF solution (3.0 M, 4 mL, 12 mmol) slowly and the reaction mixture was allowed to stir at −78° C. for 45 min. Sat. aq. NH₄Cl (2 mL) and water (4 mL) were added. The aqueous layer was extracted with EtOAc (2×10 mL), and the combined extracts were dried with Na₂SO₄ and concentrated under reduced pressure. The crude residue was purified by silica chromatography eluting with EtOAc/hexanes (1/9) to give to give 1-(2-chloro-4-(trifluoromethyl)pyrimidin-5-yl)ethanone (675 mg, 30% yield). LC-MS m/z 225 [M+H]⁺. ¹H NMR (400 MHz, CDCl₃): δ 8.86 (s, 1H), 2.65 (s, 3H).

### Method 2:

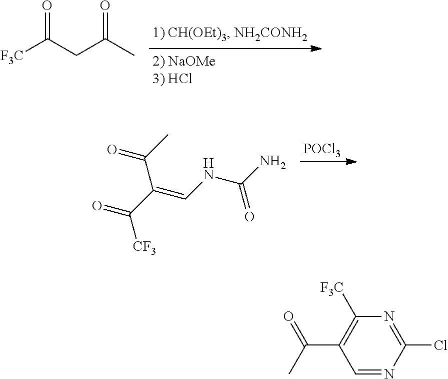

To a solution of 1,1,1-trifluoropentane-2,4-dione (200 g, 1.30 mol) in ethanol (200 mL) were added urea (78 g, 1.30 mol) and CH(OEt)₃ (211.5 g, 1.43 mol). The mixture was stirred at 80° C. for 4 h. The resulting slurry was filtered. The filter cake was suspended in methanol (300 mL) and MeONa (77.2 g, 1.43 mol) was added. The mixture was stirred at reflux for 5 h, followed by slow addition of HCl (4N) to pH 3 at rt. The resulting slurry was filtered and the filter cake was dried under vacuum to give compound (E)-1-(2-acetyl-4,4,4-trifluoro-3-oxobut-1-en-1-yl)urea (196 g, 67.3% yield) as a white solid. ¹H NMR (DMSO-d₆ 300 MHz): (Z/E) δ 10.15-10.13 (m, 1H), 8.64 (s, 1H), 7.69-7.66 (m, 2H), 2.25 (s, 3H). LC-MS MS (ESI) m/z 206.8 [M−18+H]⁺.

A mixture of compound (E)-1-(2-acetyl-4,4,4-trifluoro-3-oxobut-1-en-1-yl)urea (55 g, 0.25 mol) and POCl₃ (240.7 g, 1.57 mol) was stirred at 100° C. for 3 h. The mixture was added dropwise to water (1.5 L) at rt and extracted with EtOAc (3×500 mL). The combined organic layers were washed with brine (500 mL), dried over anhydrous Na₂SO₄, filtered and concentrated. The residue was purified by column chromatography eluting with PE/EtOAc 3/1 to give compound 1-(2-chloro-4-(trifluoromethyl)pyrimidin-5-yl)ethanone (23.5 g, 42.7% yield) as a pale-yellow oil. ¹H NMR (CDCl₃ 300 MHz): δ 8.80 (s, 1H), 2.58 (s, 3H).

¹⁹F NMR (920-083-1A CDCl₃ 400 MHz): δ−65.5 ppm. ¹³C NMR (903-158-1A CDCl₃ 400 MHz): δ 195.9, 162.3, 160.1, 153.8 (dd, J=50 Hz), 130.9, 119.5 (dd, J=366 Hz), 30.7.

### 1-(2-chloro-4-(trifluoromethyl)pyrimidin-5-yl)propan-1-one and 1-(2-chloro-4-(trifluoromethyl)pyrimidin-5-yl)butan-1-one

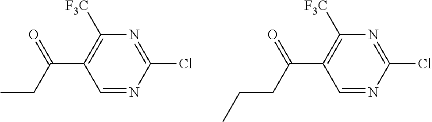 [SMILES: CCC(=O)c1cnc(Cl)nc1C(F)(F)F ; CCCC(=O)c1cnc(Cl)nc1C(F)(F)F]

The title compounds were prepared by method 1 using appropriate Grignard reagents.

### Preparation 6

### 5-bromo-2-chloro-4-(trifluoromethyl)pyrimidine

The title compound was prepared using a modified procedure based on Ondi, L. et al., Eur. J. Org. Chem. 2004, 3714.

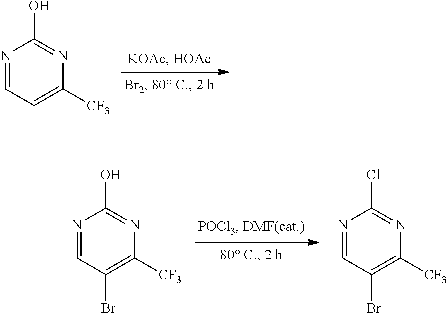 [SMILES: Oc1nccc(C(F)(F)F)n1 ; Oc1ncc(Br)c(C(F)(F)F)n1 ; FC(F)(F)c1nc(Cl)ncc1Br]

A mixture of 4-(trifluoromethyl)pyrimidin-2-ol (6.05 g, 36.9 mmol), KOAc (10.85 g, 3eq.), acetic acid (80 mL), and bromine (5.9 g, 1 eq.) was heated for 2 h at 80° C. After being cooled to rt, the mixture was concentrated. The residue was partitioned between EtOAc and water. The aqueous layer was extracted with EtOAc (2×). The combined organic layers were washed with brine, dried over Na₂SO₄. After filtration and concentration, the crude white solid product (9.38 g, quant. yield) was used for next steps without further purification.

A mixture of 5-bromo-4-(trifluoromethyl)pyrimidin-2-ol (1.35 g, 5.56 mmol), POCl₃ (15 mL), and DMF(2 drops, cat. Amount) was heated for 2 h at 80° C. The mixture was cooled to 0° C. by ice/water bath. Some ice pellets were added to the stirred mixture (exotherm). After stirring for 20 min. (the ice added should have melted), some sat. aq. NaHCO₃ (c.a. 15 mL) was added carefully to neutralize some acid. The mixture was extracted with hexanes (3×). The combined organic layers were washed with brine, dried over Na₂SO₄. After filtration and concentration (by rotavapor only! The product is volatile), 5-bromo-2-chloro-4-(trifluoromethyl)pyrimidine, as a clear oil (1.32 g, 91% yield) was used as crude for next steps without further purification.

### Example 1

### 1-(2-(isopropyl-7-(methyl sulfonyl)-3,4-dihydrobenzo[4,5]imidazo[1,2-a]pyrazin-2 (1H)— yl)-4-(trifluoromethyl)pyrimidin-5-yl)ethanone

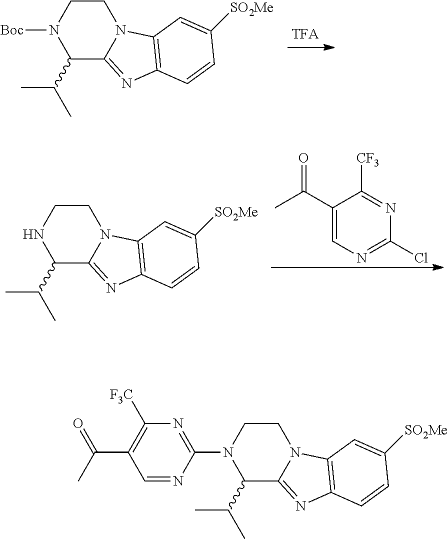 [SMILES: CC(C)C1c2nc3ccc(S(C)(=O)=O)cc3n2CCN1C(=O)OC(C)(C)C ; CC(C)C1NCCn2c1nc1ccc(S(C)(=O)=O)cc12 ; CC(=O)c1cnc(Cl)nc1C(F)(F)F ; CC(=O)c1cnc(N2CCn3c(nc4ccc(S(C)(=O)=O)cc43)C2C(C)C)nc1C(F)(F)F]

To a solution of tert-butyl 1-isopropyl-7-(methylsulfonyl)-3,4-dihydrobenzo[4,5]imidazo[1,2-a]pyrazine-2(1H)-carboxylate (30. mg, 0.08 mmol, from preparation 1) in CH₂Cl₂ (1 mL) was added TFA (0.2 mL) under N₂. The mixture was stirred at rt for 1 h. The mixture was concentrated to afford 1-isopropyl-7-(methylsulfonyl)-1,2,3,4-tetrahydrobenzo[4,5]imidazo[1,2-a]pyrazine (30 mg, TFA salt) as a yellow solid, which was used for the next step without further purification.

To a solution of 1-isopropyl-7-(methylsulfonyl)-1,2,3,4-tetrahydrobenzo[4,5]imidazo[1,2-a]pyrazine (7.50 mg, 0.03 mmol) in DMSO (1 mL) were added 1-(2-chloro-4-(trifluoromethyl)pyrimidin-5-yl)ethanone (11.5 mg, 0.05 mmol) and DIEA (9.90 mg, 0.08 mmol) under N₂. The mixture was stirred at 100° C. for 2 h. The mixture was diluted with water (10 mL) and extracted with EtOAc (3×10 mL). The combined organic layers were washed with brine (10 mL), dried over anhydrous Na₂SO₄, filtered, concentrated and then purified by preparative HPLC to afford a racemic mixture of 1-(2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone (2.40 mg, 20% yield) as a white solid. LC-MS m/z 482.1 [M+H]⁺. ¹H NMR (CD₃OD 400 MHz): δ 8.76 (s, 1H), 8.02 (s, 1H), 7.91 (d, J=8.4 Hz, 1H), 7.84-7.83 (m, 1H), 6.15 (d, J=8.0 Hz, 1H), 5.45 (d, J=13.6 Hz, 1H), 4.42-4.36 (m, 1H), 4.20 (brs, 1H), 3.91-3.80 (m, 1H), 3.09 (s, 3H), 2.60-2.46 (m, 4H), 1.34 (d, J=6.8 Hz, 3H), 1.09 (d, J=6.0 Hz, 3H).

### Example 2

### 2-(2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol

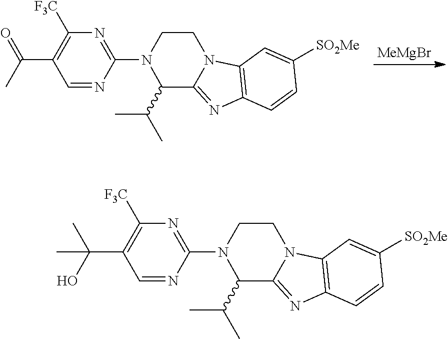 [SMILES: "MeMgBr" ; CC(=O)c1cnc(N2CCn3c(nc4ccc(S(C)(=O)=O)cc43)C2C(C)C)nc1C(F)(F)F ; CC(C)C1c2nc3ccc(S(C)(=O)=O)cc3n2CCN1c1ncc(C(C)(C)O)c(C(F)(F)F)n1]

To a solution of 1-(2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone (20 mg, 0.04 mmol, prepared according to example 1) in THF (5 mL) was added MeMgBr (0.6 mL, 0.20 mmol) dropwise at 0° C. under N₂. The mixture was stirred at 0° C. for 2 h. The mixture was quenched with sat. aq. NH₄Cl(10 mL) and extracted with CH₂Cl₂ (3×10 mL). The combined organic layers were washed with brine (10 mL), dried over anhydrous Na₂SO₄, filtered, concentrated and then purified by preparative TLC to afford a racemic mixture of 2-(2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol (6.90 mg, 33% yield) as a white solid. LC-MS m/z 498.2 [M+H]⁺. ¹H NMR (CDCl₃ 400 MHz): δ 8.79 (s, 1H), 7.99 (d, J=1.2 Hz, 1H), 7.90-7.81 (m, 2H), 6.08 (d, J=8.0 Hz, 1H), 5.35 (dd, J=4.4 and 14.0 Hz, 1H), 4.33-4.27 (m, 1H), 4.25-4.12 (m, 1H), 3.88-3.73 (m, 1H), 3.09 (s, 3H), 2.57-2.48 (m, 1H), 1.99 (s, 1H), 1.66 (s, 6H), 1.32 (d, J=6.8 Hz, 3H), 1.09 (d, J=6.8 Hz, 3H).

### Example 3

### Ethyl 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate

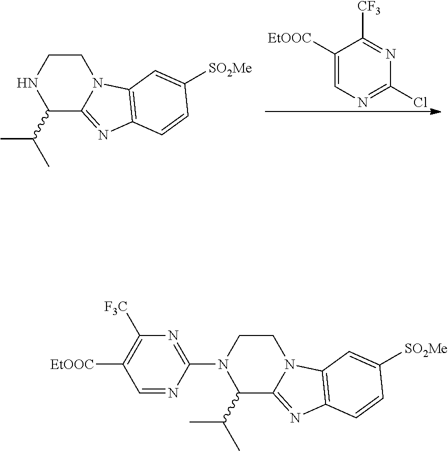 [SMILES: CC(C)C1NCCn2c1nc1ccc(S(C)(=O)=O)cc12 ; CCOC(=O)c1cnc(Cl)nc1C(F)(F)F ; CCOC(=O)c1cnc(N2CCn3c(nc4ccc(S(C)(=O)=O)cc43)C2C(C)C)nc1C(F)(F)F]

To a solution of 1-isopropyl-7-(methylsulfonyl)-1,2,3,4-tetrahydrobenzo[4,5]imidazo[1,2-a]pyrazine (5.0 mg, 0.02 mmol, prepared according to example 1) in DMSO (1 mL) was added ethyl 2-chloro-4-(trifluoromethyl)pyrimidine-5-carboxylate (8.7 mg, 0.03 mmol), DIEA (6.6 mg, 0.05 mmol) under N₂. The mixture was stirred at 100° C. for 2 h. The mixture was diluted with water (5 mL) and extracted with EtOAc (3×5 mL). The combined organic layers were washed with brine (5 mL), dried over anhydrous Na₂SO₄, filtered, concentrated and then purified by preparative TLC to afford a racemic mixture of ethyl 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (5.6 mg, 64% yield) as a white solid. LC-m/z 512.2 [M+H]⁺. ¹H NMR (CDCl₃ 400 MHz): δ 8.98 (s, 1H), 8.07-7.98 (m, 1H), 7.93-7.83 (m, 2H), 6.16 (d, J=6.4 Hz, 1H), 5.53-5.41 (m, 1H), 4.43-4.32 (m, 3H), 4.28-4.16 (m, 1H), 3.92-3.79 (m, 1H), 3.09 (s, 3H), 2.61-2.46 (m, 1H), 1.40 (s, 3H), 1.38-1.32 (m, 3H), 1.08 (d, J=6.8 Hz, 3H).

### Example 4

### (2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol

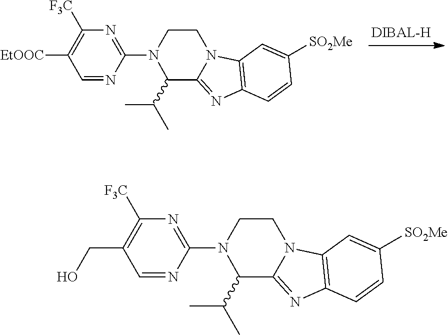 [SMILES: CCOC(=O)c1cnc(N2CCn3c(nc4ccc(S(C)(=O)=O)cc43)C2C(C)C)nc1C(F)(F)F ; CC(C)C1c2nc3ccc(S(C)(=O)=O)cc3n2CCN1c1ncc(CO)c(C(F)(F)F)n1]

To a solution of ethyl 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (10 mg, 0.02 mmol, prepared according to example 3) in anhydrous toluene (0.5 mL) was added DIBAL-H (0.2 mL, 0.20 mmol, 1M in THF) dropwise at −78° C. under N₂. The mixture was stirred at −78° C. for 2 h. Sat. NH₄Cl (5 mL) at −78° C. was added to the mixture, which was then extracted with EtOAc (3×5 mL). The combined organic layers were washed with brine (5 mL), dried over anhydrous Na₂SO₄, filtered, concentrated and then purified by basic preparative HPLC to afford a racemic mixture of (2-(1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol (5.80 mg, 63% yield) as a white solid. LC-MS m/z 470.1 [M+H]⁺. ¹H NMR (CDCl₃ 400 MHz): δ 8.68 (s, 1H), 8.01 (s, 1H), 7.93-7.81 (m, 2H), 6.11 (d, J=8.0 Hz, 1H), 5.38 (dd, J=5.2 Hz, J=14.4 Hz, 1H), 4.74 (s, 2H), 4.38-4.30 (m, 1H), 4.25-4.15 (m, 1H), 3.86-3.76 (m, 1H), 3.10 (s, 3H), 2.56-2.47 (m, 1H), 1.32 (d, J=6.8 Hz, 3H), 1.08 (d, J=6.8 Hz, 3H).

### Example 5

### 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide

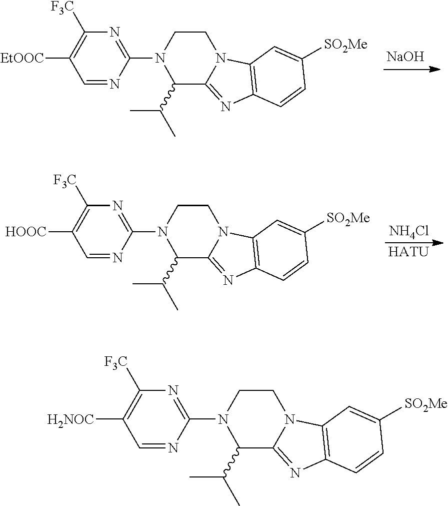 [SMILES: CCOC(=O)c1cnc(N2CCn3c(nc4ccc(S(C)(=O)=O)cc43)C2C(C)C)nc1C(F)(F)F ; "NaOH" ; CC(C)C1c2nc3ccc(S(C)(=O)=O)cc3n2CCN1c1ncc(C(=O)O)c(C(F)(F)F)n1 ; "HATU" ; "NH4Cl" ; CC(C)C1c2nc3ccc(S(C)(=O)=O)cc3n2CCN1c1ncc(C(N)=O)c(C(F)(F)F)n1]

To a solution of ethyl 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (20 mg, 0.04 mmol, prepared according to example 3) in MeOH (3 mL), H₂O (1 mL) was added NaOH (4.7 mg, 0.12 mmol). The mixture was stirred at rt overnight. The mixture was diluted with water (10 mL), acidified with 1N HCl to pH=3-4 and extracted with EtOAc (3×10 mL). The combined organic layers were washed with brine (10 mL), dried over anhydrous Na₂SO₄, filtered, and concentrated to afford 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid (20 mg, 100% yield) as a yellow solid, which was used for the next step without further purification.

To a solution of 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid (50 mg, 0.10 mmol) in DMF (10 mL) was added HATU (59 mg, 0.16 mmol), NH₄Cl (100 mg, 1.97 mmol), Et₃N (30 mg, 0.31 mmol) under N₂. The mixture was stirred at rt for 2 h. The mixture was diluted with water (15 mL) and extracted with EtOAc (3×15 mL). The combined organic layers were washed with brine (15 mL), dried over anhydrous Na₂SO₄, filtered, concentrated and purified by basic preparative HPLC to afford a racemic mixture of 2-(1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide (13.4 mg, 27% yield) as a white solid. LC-MS m/z 483.1 [M+H]⁺. ¹H NMR (CDCl₃ 400 MHz): δ 8.77 (s, 1H), 8.02 (s, 1H), 7.92-7.83 (m, 2H), 6.12 (d, J=8.4 Hz, 1H), 8.78 (brs, 2H), 5.48-5.38 (m, 1H), 4.41-4.34 (m, 1H), 4.25-4.12 (m, 1H), 3.87-3.77 (m, 1H), 3.10 (s, 3H), 2.52 (brs, 1H), 1.34 (d, J=6.8 Hz, 3H), 1.10 (d, J=6.8 Hz, 3H).

### Example 6

### (R)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone and

### (S)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone

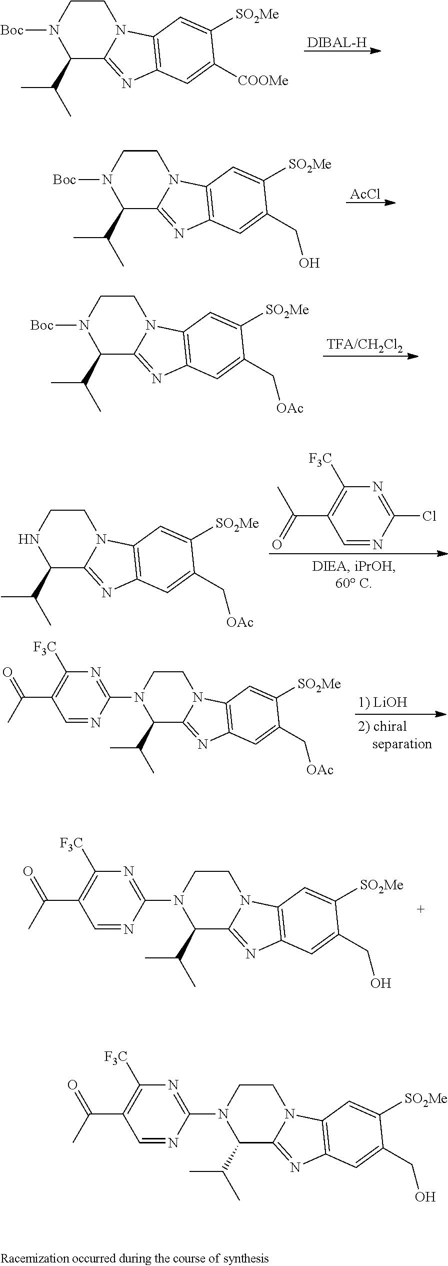

### Step 1

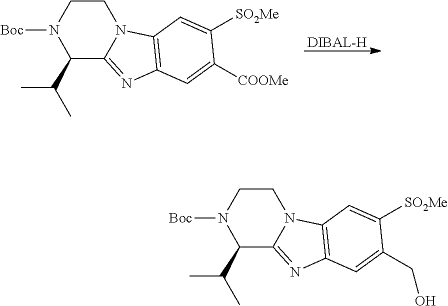 [SMILES: COC(=O)c1cc2nc3n(c2cc1S(C)(=O)=O)CCN(C(=O)OC(C)(C)C)[C@@H]3C(C)C ; "DIBAL-H" ; CC(C)[C@@H]1c2nc3cc(CO)c(S(C)(=O)=O)cc3n2CCN1C(=O)OC(C)(C)C]

To a 50 mL three-necked flask containing (R)-2-tert-butyl 8-methyl 1-isopropyl-7-(methylsulfonyl)-3,4-dihydrobenzo[4,5]imidazo[1,2-a]pyrazine-2,8(1H)-dicarboxylate (250 mg, 0.554 mmol, from preparation 2) in DCM (5 mL) was added DIBAL-H (1.70 mL, 1.67 mmol, 1.0 M in toluene) dropwise at −78° C. under N₂. The mixture was stirred at −78° C. for 3 h. The reaction was quenched with sat. aq. ammonium chloride (10 mL) at −78° C. The aqueous layer was extracted with EtOAc (3×20 mL). The combined organic layers were washed with water (15 mL) and brine, and then dried over anhydrous Na₂SO₄. The mixture was filtered and the filtrate was evaporated under reduced pressure. The residue was purified by column chromatography on silica gel eluting with PE:EtOAc 8:1-2:1 to give (R)-tert-butyl 8-(hydroxymethyl)-1-isopropyl-7-(methylsulfonyl)-3,4-dihydrobenzo[4,5]imidazo[1,2-a]pyrazine-2(1H)-carboxylate (175 mg, 74.1% yield) as a white solid.

### Step 2

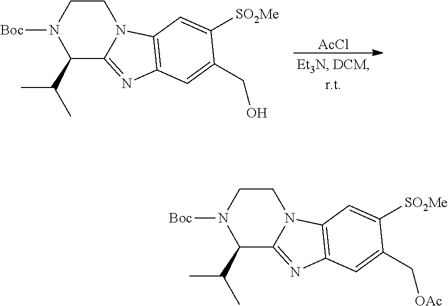 [SMILES: "AcCl" ; CC(C)[C@@H]1c2nc3cc(CO)c(S(C)(=O)=O)cc3n2CCN1C(=O)OC(C)(C)C ; CC(=O)OCc1cc2nc3n(c2cc1S(C)(=O)=O)CCN(C(=O)OC(C)(C)C)[C@@H]3C(C)C]

To a 50 mL round-bottomed flask containing (R)-tert-butyl 8-(hydroxymethyl)-1-isopropyl-7-(methylsulfonyl)-3,4-dihydrobenzo[4,5]imidazo[1,2-a]pyrazine-2(1H)-carboxylate (236 mg, 0.558 mmol) in CH₂Cl₂ (5 mL) was added Et₃N (169 mg, 1.67 mmol) and AcCl (87 mg, 1.12 mmol) under N₂. The mixture was stirred at rt for 10 min. The reaction was quenched with water (20 mL). The aqueous layer was extracted with CH₂Cl₂ (3×20 mL). The combined organic layers were washed with water (25 mL) and brine, and then dried over anhydrous Na₂SO₄. The mixture was filtered and the filtrate was evaporated under reduced pressure. The residue was purified by column chromatography on silica gel eluting with PE:EtOAc 8:1-4:1 to give (R)-tert-butyl 8-(acetoxymethyl)-1-isopropyl-7-(methylsulfonyl)-3,4-dihydrobenzo[4,5]imidazo[1,2-a]pyrazine-2(1H)-carboxylate (210 mg, 81.1% yield) as a yellow solid.

### Step 3

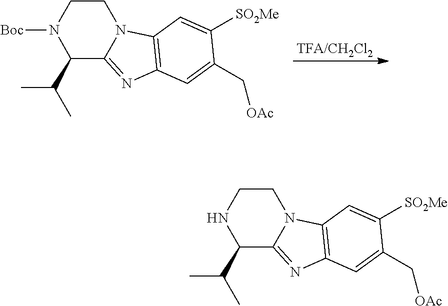 [SMILES: CC(=O)OCc1cc2nc3n(c2cc1S(C)(=O)=O)CCN(C(=O)OC(C)(C)C)[C@@H]3C(C)C ; "TFA/CH2Cl2" ; CC(=O)OCc1cc2nc3n(c2cc1S(C)(=O)=O)CCN[C@@H]3C(C)C]

TFA (1 mL) was added dropwise to a solution containing (R)-tert-butyl 8-(acetoxymethyl)-1-isopropyl-7-(methylsulfonyl)-3,4-dihydrobenzo[4,5]imidazo[1,2-a]pyrazine-2(1H)-carboxylate (210 mg, 0.452 mmol) in DCM (5 mL) at rt. The mixture was stirred at rt for 2 h. TLC showed compound 3 was consumed completely. The solvents were removed under reduced pressure at 30° C. and then DCM (10 mL) was added. The mixture was neutralized by sat. NaHCO₃ solution to pH=8. The mixture was extracted with DCM (3×20 mL) and the combined organic layers were dried over anhydrous Na₂SO₄, filtered and concentrated under vacuum to afford (R)-(1-isopropyl-7-(methylsulfonyl)-1,2,3,4-tetrahydrobenzo[4,5]imidazo[1,2-a]pyrazin-8-yl)methyl acetate (160 mg, 97.1% yield) as a white solid, which was used directly for the next step without further purification.

### Step 4

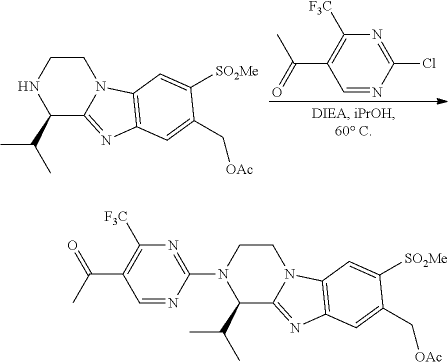 [SMILES: CC(=O)OCc1cc2nc3n(c2cc1S(C)(=O)=O)CCN[C@@H]3C(C)C ; CC(=O)c1cnc(Cl)nc1C(F)(F)F ; CC(=O)OCc1cc2nc3n(c2cc1S(C)(=O)=O)CCN(c1ncc(C(C)=O)c(C(F)(F)F)n1)[C@@H]3C(C)C]

To a solution of (R)-(1-isopropyl-7-(methylsulfonyl)-1,2,3,4-tetrahydrobenzo[4,5]imidazo[1,2-a]pyrazin-8-yl)methyl acetate (160 mg, 0.456 mmol) in ⁱPrOH (4 mL) and DCM (2 mL) was added 1-(2-chloro-4-(trifluoromethyl)pyrimidin-5-yl)ethanone (306 mg, 1.37 mmol) and DIEA (353 mg, 2.74 mmol). The mixture was stirred at 60° C. overnight. Water (5 mL) was added to the mixture and the aqueous layer was extracted with EtOAc (2×10 mL). The combined organic layers were washed with water (2×10 mL) and brine, dried over anhydrous Na₂SO₄, filtered and concentrated under reduced pressure. The residue was purified by preparative TLC to afford (R)-(2-(5-acetyl-4-(trifluoromethyl)pyrimidin-2-yl)-1-isopropyl-7-(methylsulfonyl)-1,2,3,4-tetrahydrobenzo[4,5]imidazo[1,2-a]pyrazin-8-yl)methyl acetate (150 mg, 44.4% yield) as a yellow oil.

### Step 5

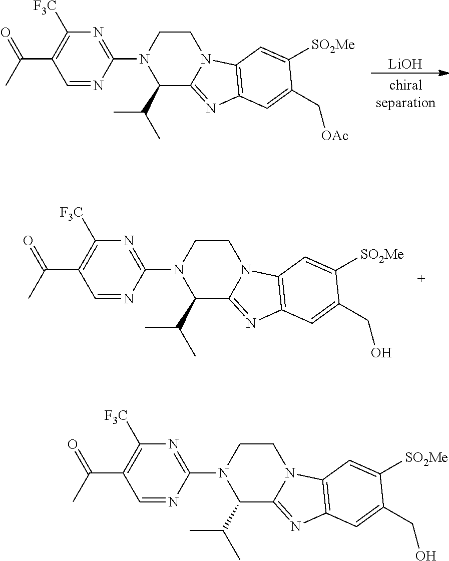 [SMILES: CC(=O)OCc1cc2nc3n(c2cc1S(C)(=O)=O)CCN(c1ncc(C(C)=O)c(C(F)(F)F)n1)[C@@H]3C(C)C ; "LiOH" ; CC(=O)c1cnc(N2CCn3c(nc4cc(CO)c(S(C)(=O)=O)cc43)[C@H]2C(C)C)nc1C(F)(F)F ; CC(=O)c1cnc(N2CCn3c(nc4cc(CO)c(S(C)(=O)=O)cc43)[C@@H]2C(C)C)nc1C(F)(F)F]

To a solution of (R)-(2-(5-acetyl-4-(trifluoromethyl)pyrimidin-2-yl)-1-isopropyl-7-(methylsulfonyl)-1,2,3,4-tetrahydrobenzo[4,5]imidazo[1,2-a]pyrazin-8-yl)methyl acetate (100 mg, 0.181 mmol) in THF (2 mL) and H₂O (2 mL) was added LiOH (38 mg, 0.905 mmol). The mixture was stirred at rt for 10 min. The mixture was extracted with EtOAc (3×10 mL). The combined organic layers were washed with brine, dried over anhydrous Na₂SO₄, filtered and concentrated under vacuum. The residue was purified by basic preparative HPLC to afford the crude product (53.1 mg, 55.9% yield). The crude product was separated by SFC to afford (R)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone (32.90 mg, isomer 1) as a white solid and (S)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone (16.90 mg, isomer 2) as a white solid. Isomer 1: (R)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone. Analytical chiral HPLC: t_{R}=7.280 min in 15 min chromatography (Method: AS-H_5_5_40_2.35 ML). LC-MS m/z 512.1 [M+H]⁺. ¹H NMR (CD₃OD 400 MHz): 9.00 (s, 1H), 8.23 (s, 1H), 7.95 (s, 1H), 6.11-6.07 (m, 1H), 5.49-5.32 (m, 1H), 5.10 (s, 2H), 4.54 (dd, J=3.6 Hz and 12.4 Hz, 1H), 4.24 (dt, J=4.8 and 12.0 Hz, 1H), 3.98-3.90 (m, 1H), 3.25 (s, 3H), 2.61-2.57 (m, 1H), 2.56 (s, 3H), 1.29 (d, J=6.8 Hz, 3H), 1.07 (d, J=6.8 Hz, 3H). Isomer 2: (S)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanone

Analytical chiral HPLC: t_{R}=8.485 min in 15 min chromatography (Method: AS-H_5_5_40_2.35 ML). LC-MS m/z 512.1 [M+H]⁺. ¹H NMR (CD₃OD 400 MHz): δ 9.00 (s, 1H), 8.24 (s, 1H), 7.96 (s, 1H), 6.12-6.08 (m, 1H), 5.48-5.31 (m, 1H), 5.11 (s, 2H), 4.56 (dd, J=3.6 Hz and 12.4 Hz, 1H), 4.24 (dt, J=4.8 and 12.0 Hz, 1H), 3.98-3.91 (m, 1H), 3.26 (s, 3H), 2.61-2.58 (m, 1H), 2.56 (s, 3H), 1.29 (d, J=6.0 Hz, 3H), 1.07 (d, J=6.8 Hz, 3H).

### Example 7

### (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol and

### (S)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol

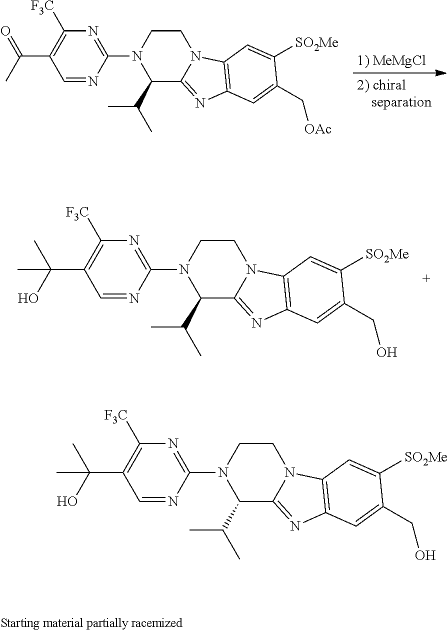 [SMILES: CC(=O)OCc1cc2nc3n(c2cc1S(C)(=O)=O)CCN(c1ncc(C(C)=O)c(C(F)(F)F)n1)[C@@H]3C(C)C ; CC(C)[C@@H]1c2nc3cc(CO)c(S(C)(=O)=O)cc3n2CCN1c1ncc(C(C)(C)O)c(C(F)(F)F)n1 ; CC(C)[C@H]1c2nc3cc(CO)c(S(C)(=O)=O)cc3n2CCN1c1ncc(C(C)(C)O)c(C(F)(F)F)n1]

(R)-(2-(5-acetyl-4-(trifluoromethyl)pyrimidin-2-yl)-1-isopropyl-7-(methylsulfonyl)-1,2,3,4-tetrahydrobenzo[4,5]imidazo[1,2-a]pyrazin-8-yl)methyl acetate (150 mg, 0.271 mmol, partially racemized) was added MeMgCl (3.0 M in toluene, 0.50 mL, 1.36 mmol) at −10° C. The mixture was stirred at −10° C. for 3 h. Sat. NH₄Cl solution (10 mL) was added at −10° C. and the mixture was filtered. The aqueous layer was extracted with DCM (3×20 mL). The combined organic layers were washed with brine, dried over anhydrous Na₂SO₄, filtered and concentrated under reduced pressure. The residue was purified by preparative TLC to give the racemic mixture (65.0 mg, 45.5% yield) as a white solid. The racemic mixture was purified by SFC separation to give (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol (16.20 mg, isomer 1) and (S)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol (9.10 mg, isomer 2) as white solids.

Isomer 1: (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol. Analytical chiral HPLC: t_{R}=8.600 min in 15 min chromatography (Method: OD-H_3_5_40_2.35 ML). LC-MS MS (ESI) m/z 528.2 [M+H]⁺. ¹H NMR (CD₃OD 400 MHz): δ 8.87 (s, 1H), 8.20 (s, 1H), 7.93 (s, 1H), 6.02 (d, J=8.0 Hz, 1H), 5.31 (dd, J=5.2 and 14.4 Hz, 1H), 5.09 (s, 2H), 4.50-4.46 (m, 1H), 4.23-4.16 (m, 1H), 3.90-3.83 (m, 1H), 3.24 (s, 3H), 2.59-2.51 (m, 1H), 1.59 (s, 6H), 1.26 (d, J=6.8 Hz, 3H), 1.05 (d, J=6.8 Hz, 3H).

Isomer 1 can be recrystallized as a hydrochloric acid salt according to following procedure:

To a solution of (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol (52 mg, 0.1 mmol) in methanol (2 mL) was added acetyl chloride (7 μL, 0.1 mmol) and the mixture was stirred at rt for 4 h. Methanol was removed under reduced pressure. The crude resultant was dissolved into mixture of acetone and. EtOAc (2.5/2.5 mL) followed by filtration. To the filtrate, hexanes (0.4 mL) were slowly added with intermittent heating. Leave the solution stay at rt until crystals form. The crystal was collected by filtration. m.p. 176-179° C.

LC-MS m/z 528 [M+H]⁺. ¹H NMR (400 MHz, CD₃OD): δ 8.95 (s, 1H), 8.48 (s, 1H), 8.17 (s, 1H), 6.28 (d, J=8.0 Hz, 1H), 5.43 (dd, J, =14.4 Hz, J₂=4.8 Hz, 1H), 5.15 (s, 2H), 4.70 (dd, J₁=12.8 Hz, J₂=3.6 Hz, 1H), 4.34 (td, J, =12.0 Hz, J₂=4.8 Hz, 1H), 3.92 (dddd, J₁=14.4 Hz, J₂=12.8 Hz, J₃=4.8 Hz, 1H), 3.26 (s, 3H), 2.70-2.62 (m, 1H), 1.60 (s, 6H), 1.31 (d, J=6.8 Hz, 3H), 1.13 (d, J=6.8 Hz, 3H).

Isomer 2: (S)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol. Analytical chiral HPLC: t_{R}=6.680 min in 15 min chromatography (Method: OD-H_3_5_40_2.35 ML). LC-MS MS (ESI) m/z 528.2 [M+H]⁺. ¹H NMR (CD₃OD 400 MHz): δ 8.87 (s, 1H), 8.22 (s, 1H), 7.94 (s, 1H), 6.03 (d, J=8.0 Hz, 1H), 5.31 (dd, J=4.4 and 14.0 Hz, 1H), 5.09 (s, 2H), 4.51-4.47 (m, 1H), 4.23-4.16 (m, 1H), 3.91-3.83 (m, 1H), 3.25 (s, 3H), 2.60-2.51 (m, 1H), 1.59 (s, 6H), 1.26 (d, J=6.8 Hz, 3H), 1.05 (d, J=6.8 Hz, 3H).

Alternatively, a racemic mixture of 2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol were prepared by following method.

### (rac)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol

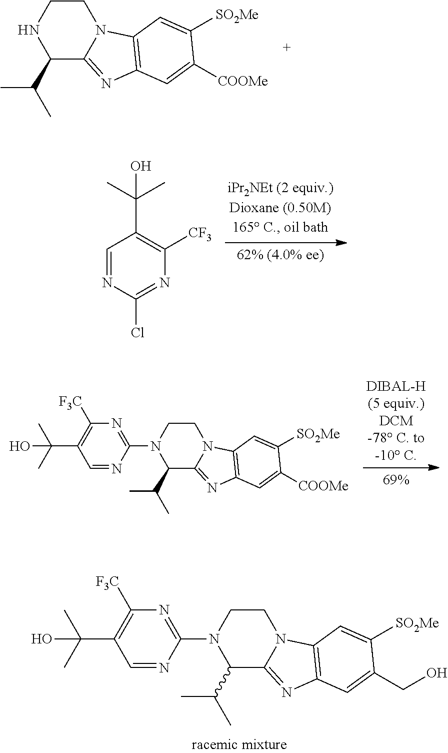 [SMILES: COC(=O)c1cc2nc3n(c2cc1S(C)(=O)=O)CCN[C@@H]3C(C)C ; CC(C)(O)c1cnc(Cl)nc1C(F)(F)F ; COC(=O)c1cc2nc3n(c2cc1S(C)(=O)=O)CCN(c1ncc(C(C)(C)O)c(C(F)(F)F)n1)[C@@H]3C(C)C ; CC(C)C1c2nc3cc(CO)c(S(C)(=O)=O)cc3n2CCN1c1ncc(C(C)(C)O)c(C(F)(F)F)n1]

(R)-methyl-1-isopropyl-7-(methylsulfonyl)-1,2,3,4-tetrahydrobenzo[4,5]imidazo[1,2-a]pyrazine-8-carboxylate (224 mg, 0.639 mmol) and 2-(2-chloro-4-(trifluoromethyl)pyrimidin-5-yl)propan-2-ol (192 mg, 0.799 mmol) were combined in a small vial and azeotroped with benzene to remove any residual water. A mixture of dioxane (1 mL) and iPr₂NEt (0.22 mL, 1.28 mmol) was degassed with N₂ for 10 minutes. This mixture was then added to the reaction vial and sealed with a Teflon® coated cap which was then wrapped with Teflon® tape. The resulting suspension was then placed in a 165° C. silicone oil bath at which point the mixture became homogeneous. The resulting solution stirred at 165° C. for 21 h. After the solvents were removed by rotovap, the mixture was purified using ISCO FCC, eluting with 50% EtOAc in Hexanes to obtain 219 mg of (R)-methyl-2-(5-(2-hydroxypropan-2-yl)-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate as a white solid (62% yield, 40% ee).

LC-MS MS (ESI) m/z 556.0 [M+H]⁺. ¹H NMR (CD₃OD, 400 MHz): δ 8.88 (s, 1H), 8.28 (s, 1H), 7.98 (s, 1H), 6.05 (d, J=8.0 Hz, 1H), 5.32 (dd, J=4.8 and 14.0 Hz, 1H), 4.53 (dd, J=3.2 and 12 Hz, 1H), 4.27-4.20 (m, 1H), 3.95 (s, 3H), 3.90-3.83 (m, 1H), 3.41 (s, 3H), 2.61-2.52 (m, 1H), 1.59 (s, 6H), 1.27 (d, J=6.8 Hz, 3H), 1.06 (d, J=6.8 Hz, 3H).

To a solution of (R)-methyl-2-(5-(2-hydroxypropan-2-yl)-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (109 mg, 0.196 mmol) in DCM (4 mL) was added a solution of DIBAL-H in DCM (0.98 mL of a 1.0 M solution, 0.98 mmol) dropwise at −78° C. The reaction slowly warmed to −10° C. over approximately 1 h, at which point 1 mL of MeOH was added to quench the excess DIBAL-H. Sat. aq. Rochelle salt (potassium sodium tartrate (KNaC₄H₄O₆)) solution (5 mL) and DCM (5 mL) were added and the mixture stirred vigorously for 15 min. The DCM layer was separated and the aqueous layer was extracted with DCM (2×5 mL). The DCM layers were combined, dried using Na₂SO₄ and evaporated to give the crude product. Purification using ISCO FCC eluting with 70% EtOAc in Hexanes gave 71 mg of 2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol as a racemic mixture (69%). LC-MS MS (ESI) m/z 528.25 [M+H]⁺. ¹H NMR (CDCl₃, 400 MHz): δ 8.72 (s, 1H), 8.05 (s, 1H), 7.80 (s, 1H), 6.01 (d, J=7.6 Hz, 1H), 5.27 (d, J=4.4 and 14.0 Hz, 1H), 5.00-4.86 (m, 2H), 4.26-4.08 (m, 2H), 3.74-3.67 (m, 1H), 3.15 (s, 3H), 3.15-3.10 (m, 1H), 2.47-2.40 (m, 1H), 1.96 (b, 1H), 1.59 (s, 6H), 1.23 (d, J=6.8 Hz, 3H), 1.10 (d, J=6.8 Hz, 3H).

2-(2-chloro-4-(trifluoromethyl)pyrimidin-5-yl)propan-2-ol was prepared as following:

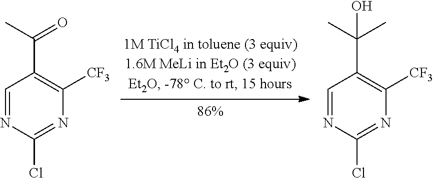

To a 1.0 M solution of TiCl₄ in toluene (6.67 mL, 6.67 mmol) was added a 1.6 M solution of MeLi in Et₂O (4.18 mL, 6.69 mmol) dropwise a −78° C. (dry ice/acetone bath). The resulting dark solution stirred at −78° C. for 30 minutes. A solution of 1-(2-chloro-4-(trifluoromethyl)pyrimidin-5-yl)ethanone (500 mg, 2.23 mmol) in Et₂O (3 mL) was added dropwise at −78° C. The reaction was slowly allowed to warm to rt in the dewar over a 15 h period. TLC analysis showed complete conversion to the more polar tertiary alcohol product. The mixture was then cooled to 0° C. and quenched with sat. aq. NH₄Cl (10 mL) followed by EtOAc (10 mL) for the workup. The EtOAc layer was separated and the aqueous layer was extracted with EtOAc (2×10 mL). The EtOAc layers were combined, dried using Na₂SO₄ and evaporated to give the crude product. Purification using ISCO FCC eluting with 20% EtOAc in Hexanes gave 459 mg of 2-(2-chloro-4-(trifluoromethyl)pyrimidin-5-yl)propan-2-ol (86% yield) as a colorless oil. LC-MS MS (ESI) m/z 241.12 [M+H]⁺. ¹H NMR (CDCl₃, 400 MHz): δ 9.05 (s, 1H), 1.99 (s, 1H), 1.67 (s, 6H).

### Example 8

### 1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)ethanol (4 isomers)

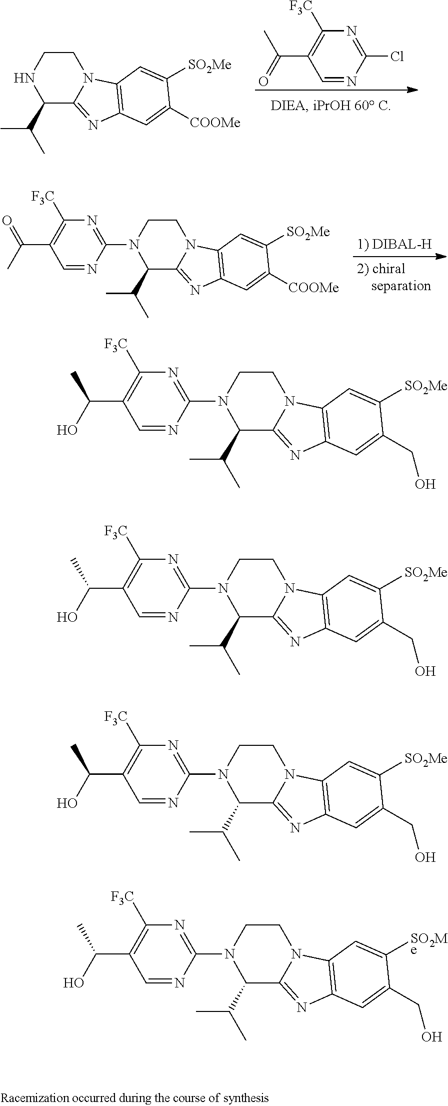

To a solution of (R)-methyl 1-isopropyl-7-(methylsulfonyl)-1,2,3,4-tetrahydrobenzo[4,5]imidazo[1,2-a]pyrazine-8-carboxylate (220 mg, 0.627 mmol) in ⁱPrOH (4 mL) and DCM (2 mL) was added 1-(2-chloro-4-(trifluoromethyl)pyrimidin-5-yl)ethanone (421 mg, 1.88 mmol) and DIEA (485 mg, 3.76 mmol). The mixture was stirred at 60° C. overnight. Water (5 mL) was added to the mixture and the aqueous layer was extracted with EtOAc (2×10 mL). The combined organic layers were washed with water (2×10 mL) and brine, dried over anhydrous Na₂SO₄, filtered and concentrated under reduced pressure. The residue was purified by preparative TLC to afford (R)-methyl 2-(5-acetyl-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (150 mg, 44.4% yield) as a yellow solid.

To a 50 mL three-necked flask containing (R)-methyl 2-(5-acetyl-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (150 mg, 0.278 mmol) in DCM (3 mL) was added DIBAL-H (1.10 mL, 1.11 mmol, 1.0 M in toluene) dropwise at −78° C. under N₂. The mixture was stirred at −78° C. for 3 h. Sat. NH₄Cl solution (10 mL) was added at 78° C. and the mixture was filtered. The aqueous layer was extracted with DCM (3×20 mL). The combined organic layers were washed with brine, dried over anhydrous Na₂SO₄, filtered and concentrated under reduced pressure. The residue was purified by preparative TLC to give the racemic mixture (81.0 mg, 56.7% yield) as a white solid. The racemic mixture was purified by SFC separation to give isomer 1 (10.60 mg, 47.1% yield) as a white solid, isomer 2 (7.10 mg, 31.6% yield) as a white solid, isomer 3 (4.70 mg, 26.1% yield) as a white solid and isomer 4 (6.00 mg, 33.3% yield) as a white solid.

### Isomer 1

Analytical chiral HPLC: t_{R}=8.397 min in 15 min chromatography (Method: OD-H_5_5_40_2.35 ML). LC-MS MS (ESI) m/z 514.1 [M+H]⁺. ¹H NMR (CD₃OD 400 MHz): δ 8.89 (s, 1H), 8.20 (s, 1H), 7.93 (s, 1H), 6.03 (d, J=8.0 Hz, 1H), 5.32 (dd, J=4.4 and 14.4 Hz, 1H), 5.11-5.08 (m, 3H), 4.50-4.46 (m, 1H), 4.28-4.16 (m, 1H), 3.90-3.83 (m, 1H), 3.24 (s, 3H), 2.72-2.39 (m, 1H), 1.42 (d, J=6.4 Hz, 3H), 1.26 (d, J=6.8 Hz, 3H), 1.04 (d, J=6.8 Hz, 3H).

### Isomer 2

Analytical chiral HPLC: t_{R}=6.700 min in 15 min chromatography (Method: AS-H_5_5_40_2.35 ML). LC-MS MS (ESI) m/z 514.1 [M+H]⁺. ¹H NMR (CD₃OD 400 MHz): δ 8.89 (s, 1H), 8.21 (s, 1H), 7.93 (s, 1H), 6.03 (d, J=8.0 Hz, 1H), 5.32 (dd, J=4.4 and 14.4 Hz, 1H), 5.11-5.08 (m, 3H), 4.50-4.46 (m, 1H), 4.23-4.16 (m, 1H), 3.95-3.79 (m, 1H), 3.25 (s, 3H), 2.59-2.51 (m, 1H), 1.42 (d, J=6.0 Hz, 3H), 1.26 (d, J=6.4 Hz, 3H), 1.04 (d, J=6.4 Hz, 3H).

### Isomer 3

Analytical chiral HPLC: t_{R}=7.666 min in 15 min chromatography (Method: AS-H_5_5_40_2.35 ML). LC-MS MS (ESI) m/z 514.2 [M+H]⁺. ¹H NMR (CD₃OD 400 MHz): δ 8.89 (s, 1H), 8.22 (s, 1H), 7.94 (s, 1H), 6.03 (d, J=8.0 Hz, 1H), 5.32 (dd, J=4.0 and 14.0 Hz, 1H), 5.11-5.08 (m, 3H), 4.51-4.47 (m, 1H), 4.24-4.15 (m, 1H), 3.91-3.84 (m, 1H), 3.25 (s, 3H), 2.61-2.43 (m, 1H), 1.42 (d, J=6.0 Hz, 3H), 1.26 (d, J=6.8 Hz, 3H), 1.05 (d, J=6.8 Hz, 3H).

### Isomer 4

Analytical chiral HPLC: t_{R}=9.621 min in 15 min chromatography (Method: OD-H_5_5_40_2.35 ML). LC-MS MS (ESI) m/z 514.1 [M+H]⁺. ¹H NMR (CD₃OD 400 MHz): δ 8.89 (s, 1H), 8.21 (s, 1H), 7.93 (s, 1H), 6.03 (d, J=8.0 Hz, 1H), 5.32 (dd, J=4.0 and 14.0 Hz, 1H), 5.11-5.08 (m, 3H), 4.50-4.23 (m, 1H), 4.23-4.15 (m, 1H), 3.91-3.80 (m, 1H), 3.25 (s, 3H), 2.59-2.51 (m, 1H), 1.42 (d, J=6.4 Hz, 3H), 1.26 (d, J=6.8 Hz, 3H), 1.05 (d, J=6.8 Hz, 3H).

### Example 9

### (R)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-1-one and

### (S)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-1-one

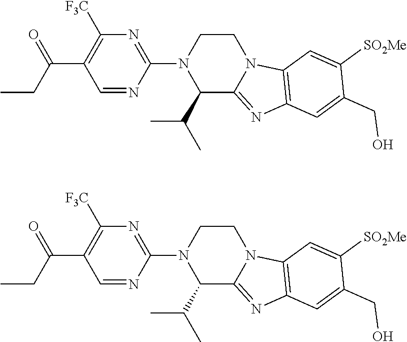 [SMILES: CCC(=O)c1cnc(N2CCn3c(nc4cc(CO)c(S(C)(=O)=O)cc43)[C@H]2C(C)C)nc1C(F)(F)F ; CCC(=O)c1cnc(N2CCn3c(nc4cc(CO)c(S(C)(=O)=O)cc43)[C@@H]2C(C)C)nc1C(F)(F)F]

The title compounds were prepared following procedure analogous to those described in Example 6 by using 1-(2-chloro-4-(trifluoromethyl)pyrimidin-5-yl)propan-1-one in stead of 1-(2-chloro-4-(trifluoromethyl)pyrimidin-5-yl)ethanone.

Isomer 1: (R)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-1-one. Analytical chiral HPLC: t_{R}=6.87 min in 15 min chromatography (Method: OJ-H_3_5_40_2.5 ML). LC-MS m/z 526.1 [M+H]⁺. ¹H NMR (CD₃OD 400 MHz): δ 8.94 (s, 1H), 8.24 (s, 1H), 7.96 (s, 1H), 6.17-6.02 (m, 1H), 5.43-5.32 (m, 1H), 5.11 (s, 2H), 4.54 (dd, J=4.0 and 12.0 Hz, 1H), 4.24 (dt, J=4.8 and 12.0 Hz, 1H), 3.98-3.90 (m, 1H), 3.27 (s, 3H), 2.93 (q, J=6.8 Hz, 2H), 2.61-2.55 (m, 1H), 1.29 (d, J=6.8 Hz, 3H), 1.15 (t, J=7.2 Hz, 3H), 1.07 (d, J=6.8 Hz, 3H).

Isomer 2: (S)-1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-1-one. Analytical chiral HPLC: t_{R}=8.40 min in 15 min chromatography (Method: OJ-H_3_5_40_2.5 ML). LC-MS m/z 526.1 [M+H]⁺. ¹H NMR (CD₃OD 400 MHz): δ 8.93 (s, 1H), 8.21 (s, 1H), 7.93 (s, 1H), 6.15-6.01 (m, 1H), 5.47-5.31 (m, 1H), 5.09 (s, 2H), 4.53 (dd, J=4.0 and 12.0 Hz, 1H), 4.24 (dt, J=4.8 and 12.0 Hz, 1H), 3.97-3.90 (m, 1H), 3.24 (s, 3H), 2.92 (q, J=6.8 Hz, 2H), 2.60-2.55 (m, 1H), 1.28 (d, J=6.8 Hz, 3H), 1.14 (t, J=7.2 Hz, 3H), 1.07 (d, J=6.8 Hz, 3H).

### Example 10

### 1-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-1-ol (4 isomers)

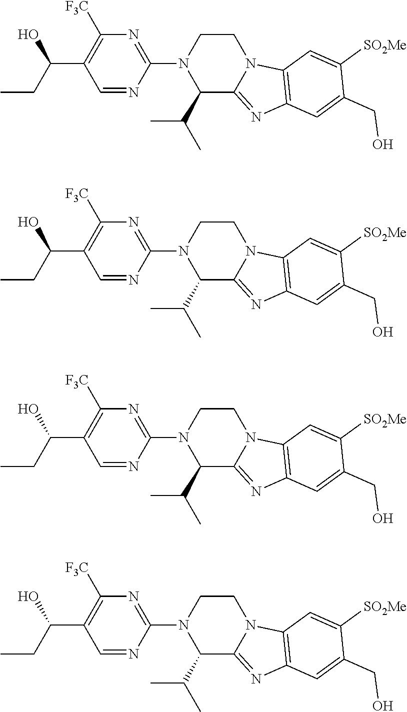 [SMILES: CC[C@@H](O)c1cnc(N2CCn3c(nc4cc(CO)c(S(C)(=O)=O)cc43)[C@H]2C(C)C)nc1C(F)(F)F ; CC[C@@H](O)c1cnc(N2CCn3c(nc4cc(CO)c(S(C)(=O)=O)cc43)[C@@H]2C(C)C)nc1C(F)(F)F ; CC[C@H](O)c1cnc(N2CCn3c(nc4cc(CO)c(S(C)(=O)=O)cc43)[C@H]2C(C)C)nc1C(F)(F)F ; CC[C@H](O)c1cnc(N2CCn3c(nc4cc(CO)c(S(C)(=O)=O)cc43)[C@@H]2C(C)C)nc1C(F)(F)F]

The title compounds were prepared following procedure analogous to those described in Example 8 by using 1-(2-chloro-4-(trifluoromethyl)pyrimidin-5-yl)propan-1-one in stead of 1-(2-chloro-4-(trifluoromethyl)pyrimidin-5-yl)ethanone.

Isomer 1: a white solid. Analytical chiral HPLC: t_{R}=6.529 min in 15 min chromatography (Method: OD-H_3_5_40_2.35 ML). LC-MS m/z 528.2 [M+H]⁺. ¹H NMR (CDCl₃ 400 MHz): δ 8.81 (s, 1H), 8.13 (s, 1H), 7.88 (s, 1H), 6.10 (d, J=7.6 Hz, 1H), 5.38-5.34 (m, 1H), 5.05-5.02 (m, 2H), 4.98-4.90 (m, 1H), 4.34-4.30 (m, 1H), 4.21-4.17 (m, 1H), 3.82-3.74 (m, 1H), 3.23 (s, 3H), 3.06 (t, J=7.2 Hz, 1H), 2.52-2.50 (m, 1H), 1.94-1.93 (m, 1H), 1.80-1.76 (m, 2H), 1.31 (d, J=6.8 Hz, 3H), 1.08 (d, J=6.8 Hz, 3H), 0.99 (t, J=7.2 Hz, 3H).

Isomer 2: a white solid. Analytical chiral HPLC: t_{R}=7.502 min in 15 min chromatography (Method: OD-H_3_5_40_2.35 ML). LC-MS m/z 528.2 [M+H]⁺. ¹H NMR (CDCl₃ 400 MHz): δ 8.82 (s, 1H), 8.13 (s, 1H), 7.88 (s, 1H), 6.10 (d, J=7.6 Hz, 1H), 5.39-5.34 (m, 1H), 5.05-5.02 (m, 2H), 4.98-4.90 (m, 1H), 4.34-4.30 (m, 1H), 4.21-4.16 (m, 1H), 3.82-3.74 (m, 1H), 3.22 (s, 3H), 3.10 (t, J=6.8 Hz, 1H), 2.52-2.49 (m, 1H), 2.01-2.00 (m, 1H), 1.80-1.75 (m, 2H), 1.30 (d, J=6.8 Hz, 3H), 1.08 (d, J=6.8 Hz, 3H), 0.99 (t, J=7.2 Hz, 3H).

Isomer 3: a white solid. Analytical chiral HPLC: t_{R}=5.173 min in 15 min chromatography (Method: OJ-H_3_5_40_2.35 ML). LC-MS m/z 528.2 [M+H]⁺. ¹H NMR (CDCl₃ 400 MHz): δ 8.81 (s, 1H), 8.13 (s, 1H), 7.88 (s, 1H), 6.10 (d, J=7.6 Hz, 1H), 5.38-5.34 (m, 1H), 5.05-5.02 (m, 2H), 4.98-4.90 (m, 1H), 4.34-4.30 (m, 1H), 4.21-4.17 (m, 1H), 3.82-3.76 (m, 1H), 3.23 (s, 3H), 3.07 (t, J=6.8 Hz, 1H), 2.52-2.50 (m, 1H), 1.94-1.93 (m, 1H), 1.80-1.75 (m, 2H), 1.31 (d, J=6.8 Hz, 3H), 1.08 (d, J=6.8 Hz, 3H), 0.99 (t, J=7.2 Hz, 3H).

Isomer 4: a white solid. Analytical chiral HPLC: t_{R}=5.817 min in 15 min chromatography (Method: OJ-H_3_5_40_2.35 ML). LC-MS m/z 528.2 [M+H]⁺. ¹H NMR (CDCl₃ 400 MHz): δ 8.81 (s, 1H), 8.13 (s, 1H), 7.88 (s, 1H), 6.10 (d, J=8.0 Hz, 1H), 5.38-5.35 (m, 1H), 5.05-5.04 (m, 2H), 4.98-4.90 (m, 1H), 4.34-4.30 (m, 1H), 4.21-4.18 (m, 1H), 3.81-3.76 (m, 1H), 3.23 (s, 3H), 3.06 (t, J=6.4 Hz, 1H), 2.52-2.50 (m, 1H), 1.94-1.93 (m, 1H), 1.81-1.75 (m, 2H), 1.31 (d, J=6.8 Hz, 3H), 1.08 (d, J=6.8 Hz, 3H), 0.99 (t, J=7.2 Hz, 3H).

### Example 11

### (R)-1-(2-((R)-8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)butan-1-ol and

### (S)-1-(2-((R)-8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)butan-1-ol

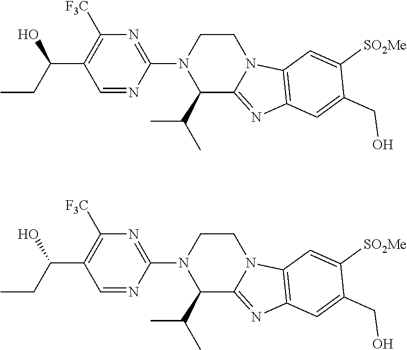 [SMILES: CC[C@@H](O)c1cnc(N2CCn3c(nc4cc(CO)c(S(C)(=O)=O)cc43)[C@H]2C(C)C)nc1C(F)(F)F ; CC[C@H](O)c1cnc(N2CCn3c(nc4cc(CO)c(S(C)(=O)=O)cc43)[C@H]2C(C)C)nc1C(F)(F)F]

The title compounds were prepared following procedure analogous to those described in Example 8 by using 1-(2-chloro-4-(trifluoromethyl)pyrimidin-5-yl)butan-1-one in stead of 1-(2-chloro-4-(trifluoromethyl)pyrimidin-5-yl)ethanone.

Isomer 1: Analytical chiral HPLC: t_{R}=8.450 min in 15 min chromatography (Method: OD-H_3_5_40_2.35 ML). LC-MS MS (ESI) m/z 542.1 [M+H]⁺. ¹H NMR (CD₃OD 400 MHz): δ 8.87 (s, 1H), 8.26 (s, 1H), 7.98 (s, 1H), 6.06 (d, J=8.0 Hz, 1H), 5.36-5.33 (m, 1H), 5.14 (s, 2H), 4.54-4.51 (m, 1H), 4.27-4.20 (m, 1H), 3.94-3.86 (m, 1H), 3.29 (s, 3H), 2.60-2.52 (m, 1H), 1.78-1.71 (m, 1H), 1.60-1.49 (m, 2H), 1.43-1.26 (m, 5H), 1.08 (d, J=6.8 Hz, 3H), 0.96 (t, J=6.8 Hz, 3H).

Isomer 2: Analytical chiral HPLC: t_{R}=10.264 min in 15 min chromatography (Method: OD-H_3_5_40_2.35 ML). LC-MS MS (ESI) m/z 542.1 [M+H]⁺. ¹H NMR (CD₃OD 400 MHz): δ 8.87 (s, 1H), 8.26 (s, 1H), 7.98 (s, 1H), 6.06 (d, J=8.0 Hz, 1H), 5.36-5.33 (m, 1H), 5.14 (s, 2H), 4.54-4.51 (m, 1H), 4.27-4.20 (m, 1H), 3.94-3.86 (m, 1H), 3.29 (s, 3H), 2.60-2.52 (m, 1H), 1.78-1.71 (m, 1H), 1.60-1.49 (m, 2H), 1.43-1.26 (m, 5H), 1.08 (d, J=6.8 Hz, 3H), 0.96 (t, J=6.8 Hz, 3H).

### Example 12

### (R)-ethyl 2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate

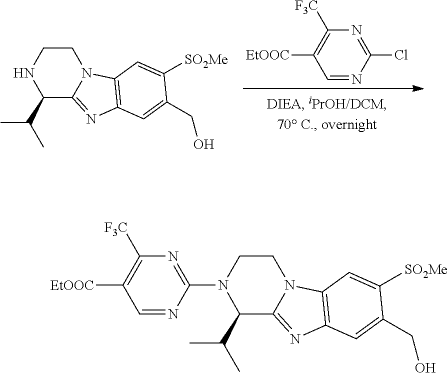 [SMILES: CC(C)[C@H]1NCCn2c1nc1cc(CO)c(S(C)(=O)=O)cc12 ; CCOC(=O)c1cnc(Cl)nc1C(F)(F)F ; CCOC(=O)c1cnc(N2CCn3c(nc4cc(CO)c(S(C)(=O)=O)cc43)[C@H]2C(C)C)nc1C(F)(F)F]

To a solution of (R)-(1-isopropyl-7-(methylsulfonyl)-1,2,3,4-tetrahydrobenzo[4,5]imidazo[1,2-a]pyrazin-8-yl)methanol (30 mg, 0.093 mmol) in CH₂Cl₂ (0.5 mL) and ⁱPrOH (0.5 mL) was added ethyl 2-chloro-4-(trifluoromethyl)pyrimidine-5-carboxylate (36 mg, 0.14 mmol) and DIEA (36 mg, 0.28 mmol). The mixture was stirred at 70° C. overnight. The mixture was concentrated under reduced pressure. Water (5 mL) was added to the mixture and the aqueous layer was extracted with EtOAc (3×10 mL). The combined organic layers were washed with water and brine, dried over anhydrous Na₂SO₄, filtered and concentrated under vacuum. The residue was purified by preparative TLC to afford (R)-ethyl 2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (42.0 mg, 83.7% yield) as a white solid. Analytical chiral HPLC: t_{R}=9.927 min in 15 min chromatography, 96.74% ee (Method: OD-H_5_5_40_2.35 ML). LC-MS MS (ESI) m/z 542.1+H⁺. ¹H NMR (CD₃OD 300 MHz): δ 8.96 (s, 1H), 8.12 (s, 1H), 7.85 (s, 1H), 6.12-6.00 (m, 1H), 5.45-5.29 (m, 1H), 5.01 (s, 2H), 4.51-4.47 (m, 1H), 4.33-4.18 (m, 3H), 3.91 (t, J=10.8 Hz, 1H), 3.18 (s, 3H), 2.63-2.47 (m, 1H), 1.35-1.26 (m, 6H), 1.05 (d, J=6.6 Hz, 3H).

### Example 13

### (R)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol and

### (S)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol

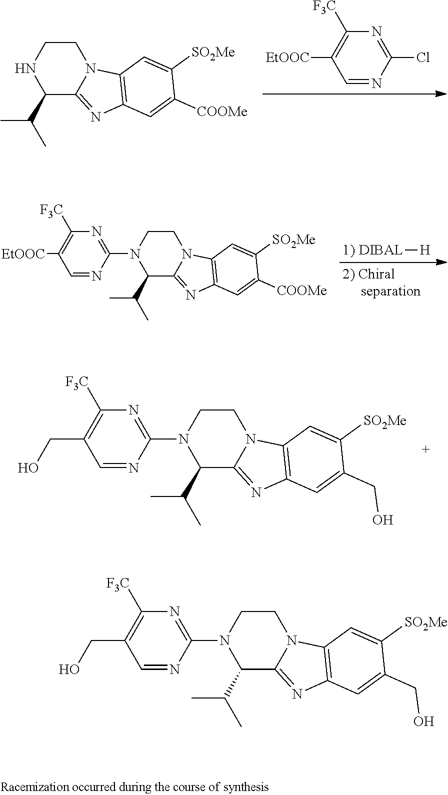

To a solution of (R)-methyl 1-isopropyl-7-(methylsulfonyl)-1,2,3,4-tetrahydrobenzo[4,5]imidazo[1,2-a]pyrazine-8-carboxylate (80 mg, 0.228 mmol) in ⁱPrOH (2 mL) and DCM (1 mL) was added ethyl 2-chloro-4-(trifluoromethyl)pyrimidine-5-carboxylate (174 mg, 0.684 mmol) and DIEA (177 mg, 1.37 mmol). The mixture was stirred at 60° C. overnight. Water (5 mL) was added to the mixture and the aqueous layer was extracted with EtOAc (2×10 mL). The combined organic layers were washed with water (2×10 mL) and brine, dried over anhydrous Na₂SO₄, filtered and concentrated under vacuum. The residue was purified by preparative TLC to afford (R)-methyl 2-(5-(ethoxycarbonyl)-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (75 mg, 57.8% yield) as a yellow solid.

To a solution of (R)-methyl 2-(5-(ethoxycarbonyl)-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (75 mg, 0.132 mmol) in DCM (3 mL) was added DIBAL-H (1M in toluene, 0.50 mL, 0.528 mmol) at −78° C. The mixture was stirred at −78° C. for 3 h. Sat. NH₄Cl solution (10 mL) was added at −78° C. and the mixture was filtered. The aqueous layer was extracted with DCM (3×20 mL). The combined organic layers were washed with brine, dried over anhydrous Na₂SO₄, filtered and concentrated under reduced pressure. The residue was purified by preparative TLC to give the racemic mixture (56.0 mg, 85.1% yield) as a white solid. The racemic mixture was separated by SFC separation to give (R)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol (38.10 mg, isomer 1) and (S)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol (11.90 mg, isomer 2) as white solids.

Isomer 1: (R)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol. Analytical chiral HPLC: t_{R}=10.281 min in 15 min chromatography (Method: AD-H_5_5_40_2.35 ML). LC-MS MS (ESI) m/z 500.1 [M+H]⁺. ¹H NMR (CD₃OD 400 MHz): δ 8.73 (s, 1H), 8.20 (s, 1H), 7.93 (s, 1H), 6.04 (d, J=8.0 Hz, 1H), 5.33 (dd, J=4.4 and 14.0 Hz, 1H), 5.09 (s, 2H), 4.63 (s, 2H), 4.50-4.46 (m, 1H), 4.28-4.06 (m, 1H), 3.91-3.83 (m, 1H), 3.24 (s, 3H), 2.59-2.47 (m, 1H), 1.26 (d, J=6.8 Hz, 3H), 1.04 (d, J=6.8 Hz, 3H).

Isomer 2: (S)-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)methanol. Analytical chiral HPLC: t_{R}=8.340 min in 15 min chromatography (Method: AD-H_5_5_40_2.35 ML). LC-MS MS (ESI) m/z 500.1 [M+H]⁺. ¹H NMR (CD₃OD 400 MHz): δ 8.73 (s, 1H), 8.22 (s, 1H), 7.94 (s, 1H), 6.04 (d, J=8.4 Hz, 1H), 5.33 (dd, J=4.8 and 14.4 Hz, 1H), 5.09 (s, 2H), 4.63 (s, 2H), 4.52-4.45 (m, 1H), 4.24-4.17 (m, 1H), 3.91-3.84 (m, 1H), 3.25 (s, 3H), 2.68-2.46 (m, 1H), 1.26 (d, J=6.8 Hz, 3H), 1.05 (d, J=6.8 Hz, 3H).

### Example 14

### (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid and

### (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid

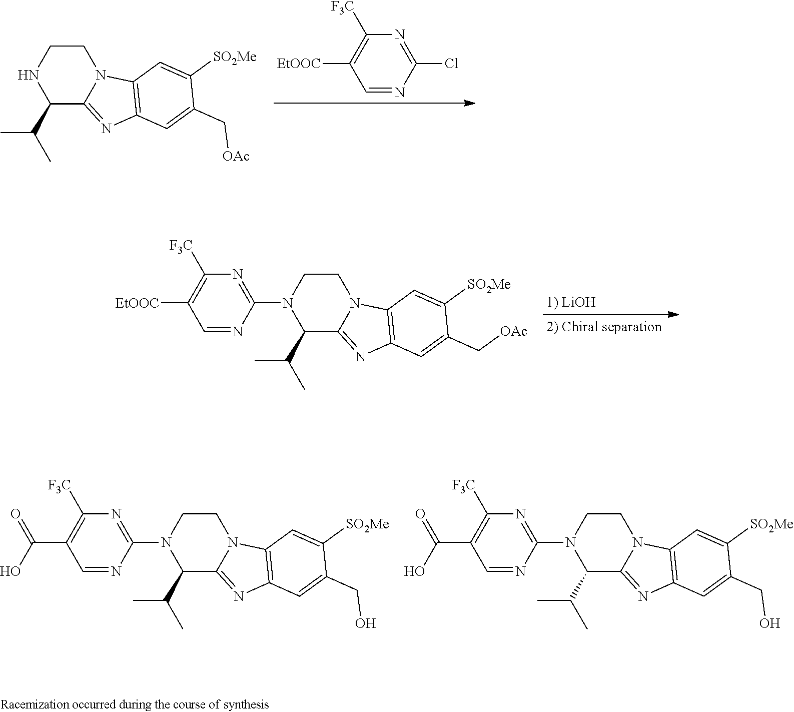 [SMILES: CC(=O)OCc1cc2nc3n(c2cc1S(C)(=O)=O)CCN[C@@H]3C(C)C ; CCOC(=O)c1cnc(Cl)nc1C(F)(F)F ; CCOC(=O)c1cnc(N2CCn3c(nc4cc(COC(C)=O)c(S(C)(=O)=O)cc43)[C@H]2C(C)C)nc1C(F)(F)F ; CC(C)[C@@H]1c2nc3cc(CO)c(S(C)(=O)=O)cc3n2CCN1c1ncc(C(=O)O)c(C(F)(F)F)n1 ; CC(C)[C@H]1c2nc3cc(CO)c(S(C)(=O)=O)cc3n2CCN1c1ncc(C(=O)O)c(C(F)(F)F)n1]

The mixture of (R)-(1-isopropyl-7-(methylsulfonyl)-1,2,3,4-tetrahydrobenzo[4,5]imidazo[1,2-a]pyrazin-8-yl)methyl acetate (284 mg, 0.8 mmol), ethyl 2-chloro-4-(trifluoromethyl)pyrimidine-5-carboxylate (296 mg, 1.2 mmol, 1.5 eq.) and DIEA (310 mg, 2.4 mmol, 3 eq.) in CH₂Cl₂/i-PrOH (3 mL/3 mL) was stirred at 80° C. for 16 h. TLC showed compound was consumed completely PE:EtOAc=1:1. The solvents were removed under vacuum and the residue was dissolved in EtOAc (10 mL). Water (10 mL) was added to the mixture. The mixture was extracted with EtOAc (10 mL×3). The organic layers were dried over anhydrous Na₂SO₄, filtered and concentrated under vacuum. The residue was purified by column chromatography on silica gel eluting with PE:EtOAc 5:1 to give (R)-ethyl 2-(8-(acetoxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (0.41 g, 87% yield) as a pale yellow solid.

(R)-ethyl 2-(8-(acetoxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate (0.1 g, 0.17 mmol) in MeOH/H₂O (5 mL/1 mL) was added LiOH H₂O (86 mg, 2 mmol). The mixture was stirred at rt for 16 h. The excess methanol was removed by vacuum at 40° C. Water (5 ml) was added and the mixture was neutralized by 1N HCl at 0° C. slowly to pH=6. The aqueous layer was extracted with CH₂Cl₂ (4×10 mL). The combined organic layers were dried over anhydrous Na₂SO₄, filtered and concentrated under vacuum to give crude product. The crude product was purified by SFC to afford (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid (34.50 mg, 40% yield, isomer 1) as a white solid and (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid (6.40 mg, 7% yield, isomer 2) as white solid.

Isomer 1: (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid. Analytical chiral HPLC: t_{R}=6.474 min in 15 min chromatography (Method: OJ-H_3_5_40_2.35 ML). LC-MS MS (ESI) m/z 514.0[M+H]⁺. ¹H NMR (CD₃OD 400 MHz): δ 9.05 (s, 1H), 8.20 (s, 1H), 7.91 (s, 1H), 6.20-6.15 (m, 1H), 5.50-5.35 (m, 1H), 5.08 (s, 2H), 4.60-4.50 (m, 1H), 4.35-4.20 (m, 1H), 4.03-3.89 (m, 1H), 3.25 (s, 3H), 2.70-2.53 (m, 1H), 1.30 (d, J=6.8 Hz, 3H), 1.08 (d, J=6.8 Hz, 3H).

Isomer 2: (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid. Analytical chiral HPLC: t_{R}=8.468 min in 15 min chromatography (Method: OJ-H_3_5_40_2.35 ML). LC-MS MS (ESI) m/z 514.0[M+H]⁺. ¹H NMR (CD₃OD 400 MHz): δ 9.02 (s, 1H), 8.25 (s, 1H), 8.00 (s, 1H), 6.20-6.15 (m, 1H), 5.50-5.35 (m, 1H), 5.12 (s, 2H), 4.60-4.50 (m, 1H), 4.35-4.20 (m, 1H), 4.03-3.89 (m, 1H), 3.28 (s, 3H), 2.70-2.50 (m, 1H), 1.30 (d, J=6.8 Hz, 3H), 1.08 (d, J=6.8 Hz, 3H).

### Example 15

### (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide and

### (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide

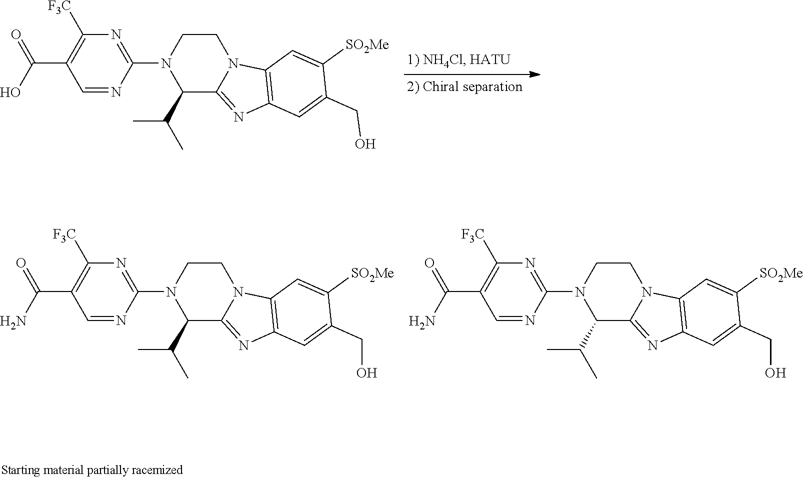 [SMILES: CC(C)[C@@H]1c2nc3cc(CO)c(S(C)(=O)=O)cc3n2CCN1c1ncc(C(=O)O)c(C(F)(F)F)n1 ; CC(C)[C@@H]1c2nc3cc(CO)c(S(C)(=O)=O)cc3n2CCN1c1ncc(C(N)=O)c(C(F)(F)F)n1 ; CC(C)[C@H]1c2nc3cc(CO)c(S(C)(=O)=O)cc3n2CCN1c1ncc(C(N)=O)c(C(F)(F)F)n1]

To a solution of (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylic acid (100 mg, 0.19 mmol, partially racemized) in DMF (2 mL) was added HATU (97 mg, 0.25 mmol) and Et₃N (52 mg, 0.51 mmol). The mixture was stirred at rt for 1 h. NH₄Cl (19 mg, 0.34 mmol) was added in one portion. The mixture was stirred at rt for 16 h. Water (10 mL) was added and the aqueous layer was extracted with EtOAc (4×10 mL). The combined organic layers were washed with water (3×10 mL) and brine, dried over anhydrous Na₂SO₄, filtered and concentrated under vacuum. The residue was purified by preparative TLC and SFC separation to afford (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide (55.2 mg, 56.7% yield, isomer 1) as a white solid and (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide (10.5 mg, 10.8% yield, isomer 2) as white solid.

Isomer 1: (R)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide Analytical chiral HPLC: t_{R}=6.084 min in 15 min chromatography (Method: OJ-H_3_5_40_2.35 ML). LC-MS MS (ESI) m/z 513.0[M+H]⁺. ¹H NMR (CD₃OD 400 MHz): δ 8.71 (s, 1H), 8.25 (s, 1H), 7.97 (s, 1H), 6.13-6.01 (m, 1H), 5.45-5.30 (m, 1H), 5.13 (s, 2H), 4.59-4.50 (m, 1H), 4.31-4.18 (m, 1H), 4.00-3.86 (m, 1H), 3.27 (s, 3H), 2.65-2.50 (m, 1H), 1.28 (d, J=6.8 Hz, 3H), 1.06 (d, J=6.8 Hz, 3H).

Isomer 2: (S)-2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxamide Analytical chiral HPLC: t_{R}=7.218 min in 15 min chromatography (Method: OJ-H_3_5_40_2.35 ML). LC-MS MS (ESI) m/z 513.1 [M+H]⁺. ¹H NMR (CD₃OD 400 MHz): 8.71 (s, 1H), 8.25 (s, 1H), 7.97 (s, 1H), 6.13-6.02 (m, 1H), 5.45-5.30 (m, 1H), 5.12 (s, 2H), 4.60-4.50 (m, 1H), 4.32-4.18 (m, 1H), 4.00-3.86 (m, 1H), 3.28 (s, 3H), 2.65-2.50 (m, 1H), 1.28 (d, J=6.8 Hz, 3H), 1.06 (d, J=6.8 Hz, 3H).

### Example 16

### (R)-2-(8-(hydroxymethyl)-1-isopropyl-7-(methylsulfonyl)-3,4-dihydrobenzo[4,5]imidazo[1,2-a]pyrazin-2(1H)-yl)-N-methyl-4-(trifluoromethyl)pyrimidine-5-carboxamide and

### (S)-2-(8-(hydroxymethyl)-1-isopropyl-7-(methylsulfonyl)-3,4-dihydrobenzo[4,5]imidazo[1,2-a]pyrazin-2(1H)-yl)-N-methyl-4-(trifluoromethyl)pyrimidine-5-carboxamide

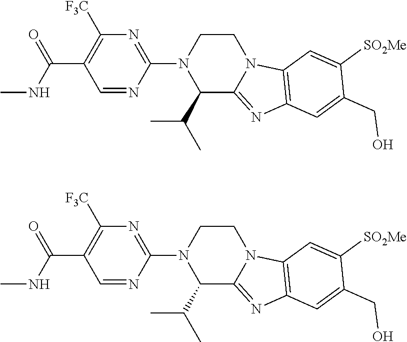 [SMILES: CNC(=O)c1cnc(N2CCn3c(nc4cc(CO)c(S(C)(=O)=O)cc43)[C@H]2C(C)C)nc1C(F)(F)F ; CNC(=O)c1cnc(N2CCn3c(nc4cc(CO)c(S(C)(=O)=O)cc43)[C@@H]2C(C)C)nc1C(F)(F)F]

The title compounds were prepared by a procedure analogous to those described in Example 15 by using methylamine hydrochloric acid salt instead of ammonium chloride as a reagent.

Isomer 1: (R)-2-(8-(hydroxymethyl)-1-isopropyl-7-(methylsulfonyl)-3,4-dihydrobenzo[4,5]imidazo[1,2-a]pyrazin-2(1H)-yl)-N-methyl-4-(trifluoromethyl)pyrimidine-5-carboxamide. Analytical chiral HPLC: t_{R}=6.355 min in 15 min chromatography (Method: AS-H_3_5_40_2.35 ML). LC-MS MS (ESI) m/z 548.9[M+Na]⁺. ¹H NMR (CD₃OD 400 MHz): δ 8.68 (s, 1H), 8.27 (s, 1H), 7.99 (s, 1H), 6.14-6.00 (m, 1H), 5.45-5.30 (m, 1H), 5.13 (s, 2H), 4.60-4.50 (m, 1H), 4.30-4.18 (m, 1H), 4.00-3.86 (m, 1H), 3.30 (s, 3H), 2.90 (s, 3H), 2.66-2.51 (m, 1H), 1.31 (d, J=6.8 Hz, 3H), 1.15 (d, J=6.8 Hz, 3H).

Isomer 2: (S)-2-(8-(hydroxymethyl)-1-isopropyl-7-(methylsulfonyl)-3,4-dihydrobenzo[4,5]imidazo[1,2-a]pyrazin-2(1H)-yl)-N-methyl-4-(trifluoromethyl)pyrimidine-5-carboxamide. Analytical chiral HPLC: t_{R}=6.486 min in 15 min chromatography (Method: AS-H_3_5_40_2.35 ML). LC-MS MS (ESI) m/z 549.0[M+Na]⁺. ¹H NMR (CD₃OD 400 MHz): δ 8.68 (s, 1H), 8.29 (s, 1H), 8.00 (s, 1H), 6.14-6.00 (m, 1H), 5.45-5.30 (m, 1H), 5.13 (s, 2H), 4.62-4.52 (m, 1H), 4.32-4.20 (m, 1H), 4.02-3.87 (m, 1H), 3.30 (s, 3H), 2.91 (s, 3H), 2.67-2.51 (m, 1H), 1.31 (d, J=6.8 Hz, 3H), 1.11 (d, J=6.8 Hz, 3H).

### Example 17

### (R)-2-(8-(hydroxymethyl)-1-isopropyl-7-(methylsulfonyl)-3,4-dihydrobenzo[4,5]imidazo[1,2-a]pyrazin-2(1H)-yl)-N,N-dimethyl-4-(trifluoromethyl)pyrimidine-5-carboxamide and

### (S)-2-(8-(hydroxymethyl)-1-isopropyl-7-(methylsulfonyl)-3,4-dihydrobenzo[4,5]imidazo[1,2-a]pyrazin-2(1H)-yl)-N,N-dimethyl-4-(trifluoromethyl)pyrimidine-5-carboxamide

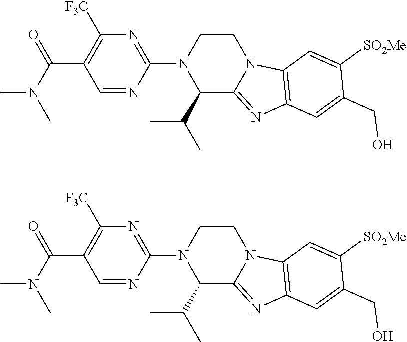 [SMILES: CC(C)[C@@H]1c2nc3cc(CO)c(S(C)(=O)=O)cc3n2CCN1c1ncc(C(=O)N(C)C)c(C(F)(F)F)n1 ; CC(C)[C@H]1c2nc3cc(CO)c(S(C)(=O)=O)cc3n2CCN1c1ncc(C(=O)N(C)C)c(C(F)(F)F)n1]

The title compounds were prepared by a procedure analogous to those described in Example 15 by using dimethylamine hydrochloric acid salt instead of ammonium chloride as a reagent.

Isomer 1: (R)-2-(8-(hydroxymethyl)-1-isopropyl-7-(methylsulfonyl)-3,4-dihydrobenzo[4,5]imidazo[1,2-a]pyrazin-2(1H)-yl)-N,N-dimethyl-4-(trifluoromethyl)pyrimidine-5-carboxamide. Analytical chiral HPLC: t_{R}=3.649 min in 8 min chromatography (Method: AS-H_S_3_5_40_3 ML). LC-MS MS (ESI) m/z 563.1 [M+Na]⁺. ¹H NMR (CD₃OD 400 MHz): δ 8.62 (s, 1H), 8.23 (d, J=6.4 Hz, 1H), 7.95 (d, J=5.2 Hz, 1H), 6.09-5.98 (m, 1H), 5.50-5.26 (m, 1H), 5.10 (d, J=4.4 Hz, 2H), 4.59-4.50 (m, 1H), 4.32-4.20 (m, 1H), 4.03-3.87 (m, 1H), 3.30-3.25 (m, 3H), 3.11 (s, 3H), 2.95 (s, 3H), 2.67-2.51 (m, 1H), 1.31 (d, J=6.8 Hz, 3H), 1.09 (d, J=6.8 Hz, 3H).

Isomer 2: (S)-2-(8-(hydroxymethyl)-1-isopropyl-7-(methylsulfonyl)-3,4-dihydrobenzo[4,5]imidazo[1,2-a]pyrazin-2(1H)-yl)-N,N-dimethyl-4-(trifluoromethyl)pyrimidine-5-carboxamide. Analytical chiral HPLC: t_{R}=4.502 min in 8 min chromatography (Method: AS-H_S_3_5_40_3 ML). LC-MS MS (ESI) m/z 563.1 [M+Na]⁺.

¹H NMR (CD₃OD 400 MHz): δ 8.62 (s, 1H), 8.27 (s, 1H), 7.98 (s, 1H), 6.09-5.98 (m, 1H), 5.49-5.28 (m, 1H), 5.13 (s, 2H), 4.59-4.50 (m, 1H), 4.33-4.20 (m, 1H), 4.05-3.90 (m, 1H), 3.30 (s, 3H), 3.12 (s, 3H), 2.95 (s, 3H), 2.68-2.52 (m, 1H), 1.32 (d, J=6.8 Hz, 3H), 1.10 (d, J=6.8 Hz, 3H).

### Example 18

### (R)-(1-isopropyl-2-(4-methoxy-5-(trifluoromethyl)pyrimidin-2-yl)-7-(methylsulfonyl)-1,2,3,4-tetrahydrobenzo[4,5]imidazo[1,2-a]pyrazin-8-yl)methanol

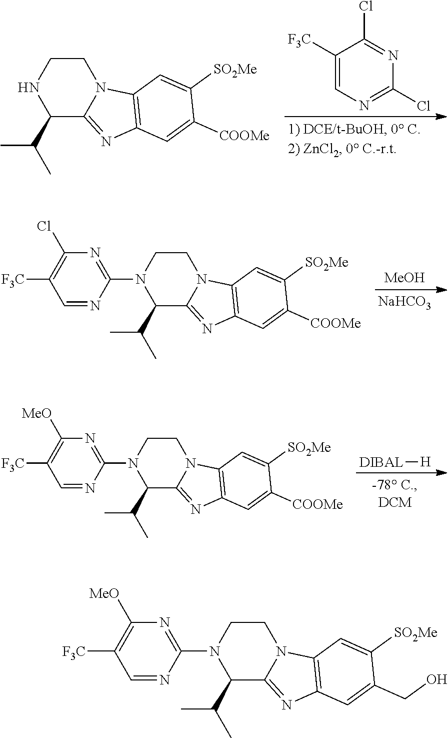

To a solution of 2,4-dichloro-5-(trifluoromethyl)pyrimidine (20.0 mg, 0.092 mmol) in DCE (1 mL) and t-BuOH (1 mL) was added ZnCl₂ (1M in diethyl ether, 0.2 mL, 0.2 mmol) at 0° C. The mixture was stirred at 0° C. for 1 h. Then a solution of (R)-methyl 1-isopropyl-7-(methylsulfonyl)-1,2,3,4-tetrahydrobenzo[4,5]imidazo[1,2-a]pyrazine-8-carboxylate (31 mg, 0.09 mmol) in DCE (1 mL) and t-BuOH (1 mL) was added to the reaction mixture via syringe over 1 min at 0° C. After addition, the resulting mixture was stirred at rt overnight. The reaction mixture was concentrated under vacuum. Water (5 mL) and EtOAc (5 mL) were added to the mixture. The aqueous phase was extracted with EtOAc (3×10 mL). The combined organic layers were washed with brine, dried over anhydrous Na₂SO₄, filtered and concentrated under vacuum. The residue was purified by preparative TLC to afford (R)-methyl 2-(4-chloro-5-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (20 mg, 42.6% yield) as a white solid.

To a solution of (R)-methyl 2-(4-chloro-5-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (15 mg, 0.028 mmol) in methanol (4 mL) was added NaHCO₃ (25 mg, 0.29 mmol). The mixture was stirred at 60° C. for 20 h. The mixture was concentrated under vacuum. Water (5 mL) and EtOAc (5 mL) were added to the mixture. The aqueous phase was extracted with EtOAc (3×10 mL). The combined organic layers were washed with brine, dried over anhydrous Na₂SO₄, filtered and concentrated under vacuum. The residue was purified by preparative TLC to afford (R)-methyl 1-isopropyl-2-(4-methoxy-5-(trifluoromethyl)pyrimidin-2-yl)-7-(methylsulfonyl)-1,2,3,4-tetrahydrobenzo[4,5]imidazo[1,2-a]pyrazine-8-carboxylate (13.0 mg, 87.2% yield) as a white solid.

To a solution of (R)-methyl 1-isopropyl-2-(4-methoxy-5-(trifluoromethyl)pyrimidin-2-yl)-7-(methylsulfonyl)-1,2,3,4-tetrahydrobenzo[4,5]imidazo[1,2-a]pyrazine-8-carboxylate (15 mg, 0.028 mmol) in CH₂Cl₂ (2 mL) was added DIBAL H (1M in toluene, 0.1 mL, 0.1 mmol) at −78° C. The mixture was stirred at −78° C. for 2 h and warmed to rt for 6 h. Sat. NH₄Cl solution (1 mL) was added and the mixture was filtered. The filtrate was concentrated under vacuum. Water (5 mL) and EtOAc (5 mL) were added to the mixture. The aqueous layer was extracted with EtOAc (3×5 mL). The combined organic layers were washed with brine, dried over anhydrous Na₂SO₄, filtered and concentrated under vacuum. The residue was purified by preparative TLC to afford (R)-(1-isopropyl-2-(4-methoxy-5-(trifluoromethyl)pyrimidin-2-yl)-7-(methylsulfonyl)-1,2,3,4-tetrahydrobenzo[4,5]imidazo[1,2-a]pyrazin-8-yl)methanol (3.50 mg, 24.6% yield) as a white solid. Analytical chiral HPLC: t_{R}=6.598 min in 15 min chromatography, 94.60% ee (Method: OD-H_3_5_40_2.35 ML). LC-MS MS (ESI) m/z 500.1 [M+H]⁺. ¹H NMR (CDCl₃ 300 MHz): δ 8.31 (s, 1H), 8.12 (s, 1H), 7.86 (s, 1H), 6.02-5.98 (m, 1H), 5.43-5.38 (m, 1H), 5.04-4.98 (m, 2H), 4.32-4.28 (m, 1H), 4.16 (dt, J=5.1 and 12.0 Hz, 1H), 4.01 (s, 3H), 3.77-3.68 (m, 1H), 3.21 (s, 3H), 3.11-3.06 (m, 1H), 2.50-2.47 (m, 1H), 1.30 (d, J=6.6 Hz, 3H), 1.07 (d, J=6.6 Hz, 3H).

### Example 19

### (R)-(2-(8-(hydroxymethyl)-1-isopropyl-7-(methylsulfonyl)-3,4-dihydrobenzo[4,5]imidazo[1,2-a]pyrazin-2(1H)-yl)-4-methylpyrimidin-5-yl)methanol

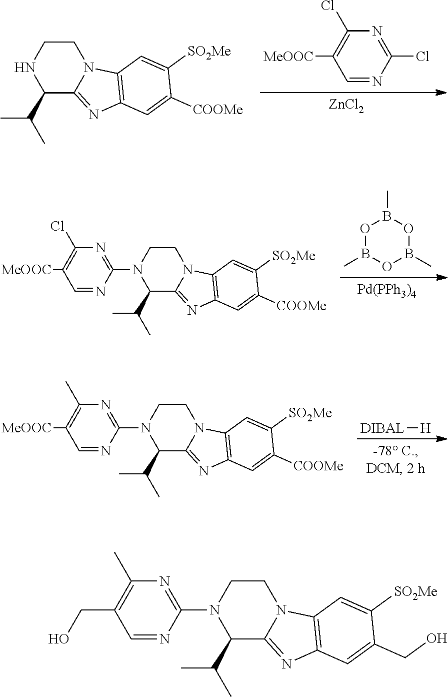

A solution of methyl 2,4-dichloropyrimidine-5-carboxylate (27 mg, 0.13 mmol) in dichloroethane/t-butanol (1:1, 2 mL) was cooled to 0° C. ZnCl₂ solution (1.0 M in ether, 0.29 mL, 0.29 mmol, 2.2 eq.) was added. After stirring for 1 h, a solution of (R)-methyl 1-isopropyl-7-(methylsulfonyl)-1,2,3,4-tetrahydrobenzo[4,5]imidazo[1,2-a]pyrazine-8-carboxylate (30 mg, 0.09 mmol) in dichloroethane/t-butanol (1:1, 2 mL) was added slowly at 0° C. The mixture was stirred at rt overnight. Water (10 mL) was added and the mixture was extracted with EtOAc (10 mL×3). The combined organic layers were dried over anhydrous Na₂SO₄, filtered and concentrated under vacuum. The residue was purified by preparative TLC to afford (R)-methyl 2-(4-chloro-5-(methoxycarbonyl)pyrimidin-2-yl)-1-isopropyl-7-(methylsulfonyl)-1,2,3,4-tetrahydrobenzo[4,5]imidazo[1,2-a]pyrazine-8-carboxylate (34 mg, 77.3% yield) as a solid.

To a solution of (R)-methyl 2-(4-chloro-5-(methoxycarbonyl)pyrimidin-2-yl)-1-isopropyl-7-(methylsulfonyl)-1,2,3,4-tetrahydrobenzo[4,5]imidazo[1,2-a]pyrazine-8-carboxylate (20 mg, 0.04 mmol) and 2,4,6-trimethyl-1,3,5,2,4,6-trioxatriborinane (50 mg, 0.4 mmol) in 5 mL of dioxane was added K₂CO₃ (54 mg, 0.4 mmol) followed by Pd(PPh₃)₄ (5 mg, 0.004 mmol) under N₂ with stirring. The mixture was refluxed for 2 h until the material was disappeared. The reaction mixture was cooled to rt. The dioxane was removed under vacuum. Water (10 mL) was added and the mixture was extracted with EtOAc (10 mL×3). The organic layers were dried over anhydrous Na₂SO₄, filtered and concentrated under vacuum. The residue was purified by preparative TLC to afford (R)-methyl 1-isopropyl-2-(5-(methoxycarbonyl)-4-methylpyrimidin-2-yl)-7-(methylsulfonyl)-1,2,3,4-tetrahydrobenzo[4,5]imidazo[1,2-a]pyrazine-8-carboxylate (15 mg, 75% yield) as a colorless oil. LC-MS MS (ESI) m/z 502.1 [M+H]⁺.

To a solution of (R)-methyl 1-isopropyl-2-(5-(methoxycarbonyl)-4-methylpyrimidin-2-yl)-7-(methylsulfonyl)-1,2,3,4-tetrahydrobenzo[4,5]imidazo[1,2-a]pyrazine-8-carboxylate (15 mg, 0.03 mmol) in toluene (2 mL) was added DIBAL H (1M in toluene, 0.3 mL, 0.3 mmol) at −78° C. The mixture was stirred at −78° C. for 2 h and then rt for 30 mins. Sat. NH₄Cl solution (5 mL) was added slowly at 0° C. and the mixture was filtered. The aqueous layer was extracted with EtOAc (3×10 mL). The combined organic layers were washed with brine, dried over anhydrous Na₂SO₄, filtered and concentrated under vacuum. The residue was purified by preparative TLC to afford (R)-(2-(8-(hydroxymethyl)-1-isopropyl-7-(methylsulfonyl)-3,4-dihydrobenzo[4,5]imidazo[1,2-a]pyrazin-2(1H)-yl)-4-methylpyrimidin-5-yl)methanol (1.6 mg, 12.3% yield) as a colorless oil. LC-MS MS (ESI) m/z 446.2 [M+H]⁺. ¹H NMR (CD₃OD 400 MHz): δ 8.26 (s, 1H), 8.23 (s, 1H), 7.99 (s, 1H), 6.00 (d, J=8.8 Hz, 1H), 5.17 (s, 2H), 4.90-4.79 (m, 1H), 4.67 (s, 2H), 4.45-4.44 (m, 1H), 4.25-4.18 (m, 1H), 4.15-4.04 (m, 1H), 3.31 (s, 3H), 2.58-2.49 (m, 1H), 2.45 (s, 3H), 1.25 (d, J=6.8 Hz, 3H), 1.12 (d, J=6.8 Hz, 3H).

### Example 20

### (R)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol and

### (S)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol

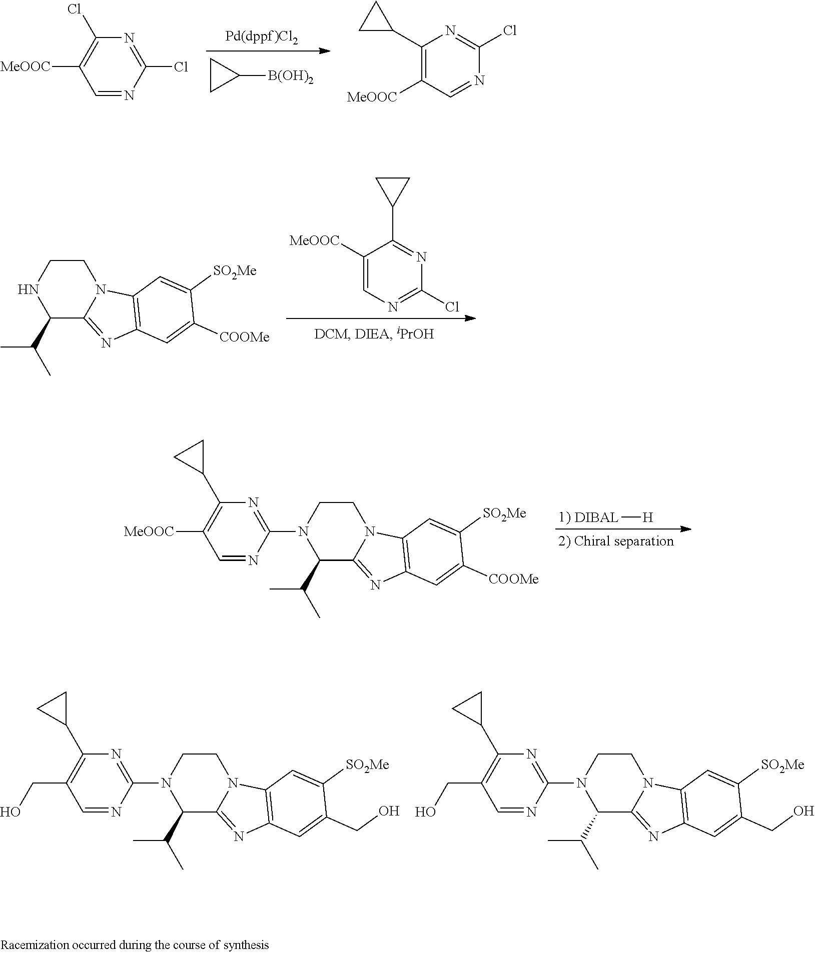

To a solution of methyl 2,4-dichloropyrimidine-5-carboxylate (852 mg, 4 mmol) and cyclopropylboronic acid (344 mg, 4 mmol) in THF (10 mL) was added K₃PO₄ (3.1 g, 12 mmol) followed by Pd(dppf) Cl₂ (292 mg, 0.4 mmol) under N₂. The mixture was refluxed for 4 h until the material was disappeared. The reaction mixture was cooled to rt. THF was removed under vacuum. Water (20 mL) was added and the mixture was extracted with EtOAc (20 mL×3). The organic layers were dried over anhydrous Na₂SO₄, filtered and concentrated under vacuum. The residue was purified by preparative TLC to afford methyl 2-chloro-4-cyclopropylpyrimidine-5-carboxylate (220 mg, 26% yield) as a white solid.

The mixture of (R)-methyl 1-isopropyl-7-(methylsulfonyl)-1,2,3,4-tetrahydrobenzo[4,5]imidazo[1,2-a]pyrazine-8-carboxylate (77 mg, 0.22 mmol), methyl 2-chloro-4-cyclopropylpyrimidine-5-carboxylate (57 mg, 0.26 mmol, 1.2 eq.) and DIEA (172 mg, 1.3 mmol, 6 eq.) in CH₂Cl₂/i-PrOH (1 mL/1 mL) was stirred at 120° C. for 16 h. TLC showed (R)-methyl 1-isopropyl-7-(methylsulfonyl)-1,2,3,4-tetrahydrobenzo[4,5]imidazo[1,2-a]pyrazine-8-carboxylate was consumed completely (PE:EtOAc=1:1). Water (10 mL) was added and the mixture was extracted with EtOAc (10 mL×3). The organic layers were dried over anhydrous Na₂SO₄, filtered and concentrated under vacuum. The residue was purified by preparative TLC to afford (R)-methyl 2-(4-cyclopropyl-5-(methoxycarbonyl)pyrimidin-2-yl)-1-isopropyl-7-(methylsulfonyl)-1,2,3,4-tetrahydrobenzo[4,5]imidazo[1,2-a]pyrazine-8-carboxylate (50 mg, 43% yield) as a colorless oil.

To a solution of (R)-methyl 2-(4-cyclopropyl-5-(methoxycarbonyl)pyrimidin-2-yl)-1-isopropyl-7-(methylsulfonyl)-1,2,3,4-tetrahydrobenzo[4,5]imidazo[1,2-a]pyrazine-8-carboxylate (50 mg, 0.1 mmol) in CH₂Cl₂ (2 mL) was added DIBAL H (1M in toluene, 1 mL, 1 mmol) at −78° C. The mixture was stirred at −78° C. for 1 h and then rt for 1 h. Sat. NH₄Cl solution (5 mL) was added slowly at 0° C. and the mixture was filtered. The aqueous layer was extracted with EtOAc (3×5 mL). The combined organic layers were washed with brine, dried over anhydrous Na₂SO₄, filtered and concentrated under vacuum. The residue was purified by preparative TLC and then SFC separation to afford (R)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol (11.50 mg, 24.5% yield, isomer 1) as a colorless oil and (S)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol (7.10 mg, 15.1% yield, isomer 2) as a colorless oil.

Isomer 1: (R)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol. Analytical chiral HPLC: t_{R}=9.898 min in 15 min chromatography (Method: OD-H_3_5_40_2.35 ML). LC-MS MS (ESI) m/z 472.2 [M+H]⁺. ¹H NMR (CD₃OD 400 MHz): δ 8.23 (s, 1H), 8.18 (s, 1H), 8.00 (s, 1H), 6.00 (d, J=8.0 Hz, 1H), 5.27-5.18 (m, 1H), 5.13 (s, 2H), 4.63 (s, 2H), 4.48-4.39 (m, 1H), 4.23-4.11 (m, 1H), 3.85-3.73 (m, 1H), 3.28 (s, 3H), 2.58-2.42 (m, 1H), 2.35-2.22 (m, 1H), 1.28 (d, J=6.8 Hz, 3H), 1.21-1.09 (m, 4H), 1.05 (d, J=6.8 Hz, 3H).

Isomer 2: (S)-(4-cyclopropyl-2-(8-(hydroxymethyl)-1-isopropyl-7-(methylsulfonyl)-3,4-dihydrobenzo[4,5]imidazo[1,2-a]pyrazin-2(1H)-yl)pyrimidin-5-yl)methanol. Analytical chiral HPLC: t_{R}=10.770 min in 15 min chromatography (Method: OD-H_3_5_40_2.35 ML). LC-MS MS (ESI) m/z 472.2 [M+H]⁺. ¹H NMR (G000237343 901-086-P1 CD₃OD 400 MHz): δ 8.23 (s, 1H), 8.18 (s, 1H), 8.00 (s, 1H), 6.02 (d, J=8.0 Hz, 1H), 5.27-5.22 (m, 1H), 5.13 (s, 2H), 4.64 (s, 2H), 4.47-4.40 (m, 1H), 4.23-4.11 (m, 1H), 3.85-3.74 (m, 1H), 3.28 (s, 3H), 2.57-2.44 (m, 1H), 2.32-2.20 (m, 1H), 1.27 (d, J=6.8 Hz, 3H), 1.21-1.09 (m, 4H), 1.05 (d, J=6.8 Hz, 3H).

### Example 21

### (R)-(1-isopropyl-2-(5-methyl-4-(trifluoromethyl)pyrimidin-2-yl)-7-(methylsulfonyl)-1,2,3,4-tetrahydrobenzo[4,5]imidazo[1,2-a]pyrazin-8-yl)methanol

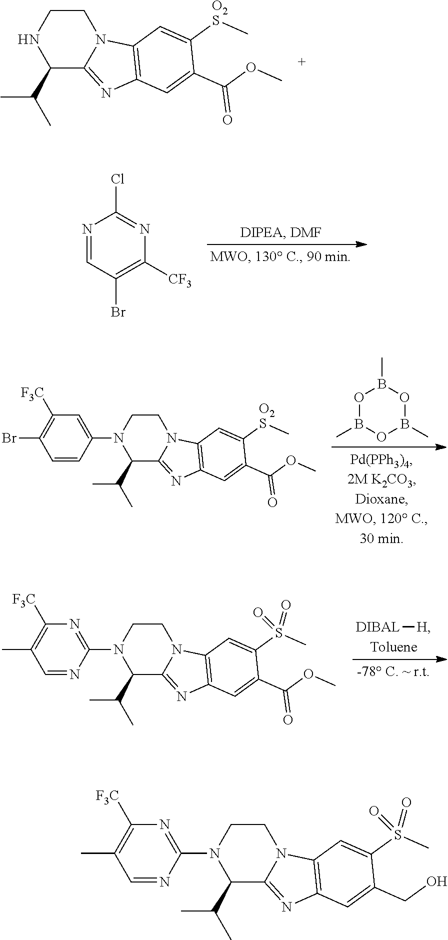

A mixture of (R)-methyl 1-isopropyl-7-(methylsulfonyl)-1,2,3,4-tetrahydrobenzo[4,5]imidazo[1,2-a]pyrazine-8-carboxylate (45 mg, 0.128 mmol), 5-bromo-2-chloro-4-(trifluoromethyl)pyrimidine (40 mg, 1.2eq.), DIPEA (90 μL, 4eq.) and DMF(1.5 mL) was put in Microwave Oven and heated 90 min. at 130° C. The mixture was participated between EtOAc and water. The aqueous layer was extracted twice by EtOAc. The combined organic layers were washed by brine, dried over Na₂SO₄. After filtration and concentration, the residue was purified by ISCO (12 g column, 10-40% EtOAc in Hexanes) to afford 34.4 mg (47% yield) of (R)-methyl 2-(4-bromo-3-(trifluoromethyl)phenyl)-1-isopropyl-7-(methylsulfonyl)-1,2,3,4-tetrahydrobenzo[4,5]imidazo[1,2-a]pyrazine-8-carboxylate.

A mixture of (R)-methyl 2-(5-bromo-4-(trifluoromethyl)pyrimidin-2-yl)-1-isopropyl-7-(methylsulfonyl)-1,2,3,4-tetrahydrobenzo[4,5]imidazo[1,2-a]pyrazine-8-carboxylate (7 mg, 0.012 mmol), Pd(PPh₃)₄ (1 mg, cat. Amount), 2M aq. K₂CO₃ solution (100 μL, excess), and dry 1,4-Dioxane (700 μL) was degassed and refilled with nitrogen gas for 3 times. A solution of 2,4,6-trimethyl-1,3,5,2,4,6-trioxatriborinane (5 mg, excess) in dry 1,4-Dioxane (100 μL) was added. The mixture was heated in a microwave oven for 30 minutes at 120° C. After concentration, the residue was filtered and purified by Gilson to afford 3.5 mg (R)-methyl 1-isopropyl-2-(5-methyl-4-(trifluoromethyl)pyrimidin-2-yl)-7-(methylsulfonyl)-1,2,3,4-tetrahydrobenzo[4,5]imidazo[1,2-a]pyrazine-8-carboxylate (56% yield). LC-MS: m/z 512.3 [M+H]⁺.

A solution of (R)-methyl 1-isopropyl-2-(5-methyl-4-(trifluoromethyl)pyrimidin-2-yl)-7-(methylsulfonyl)-1,2,3,4-tetrahydrobenzo[4,5]imidazo[1,2-a]pyrazine-8-carboxylate (3.5 mg, 0.007 mmol) in dry toluene (3 mL) was cooled to −78° C. A solution of DIBAL-H in toluene (1M, 35 μL, 5equiv.) was added. The mixture was stirred for 3 h. LC-MS indicated the reaction was complete. The mixture was quenched by sat. NH₄Cl solution (200 μL) and methanol (200 μL), before being warmed to r.t. After concentration, the residue was filtered and purified by Gilson to afford 1.53 mg (R)-(1-isopropyl-2-(5-methyl-4-(trifluoromethyl)pyrimidin-2-yl)-7-(methylsulfonyl)-1,2,3,4-tetrahydrobenzo[4,5]imidazo[1,2-a]pyrazin-8-yl)methanol (46% yield). LC-MS: m/z 484.3 [M+H]⁺. ¹H NMR (CD₃OD, 400 MHz): δ 8.52 (s, 1H), 8.27 (s, 1H), 7.99 (s, 1H), 6.05 (d, J=8.0 Hz, 1H), 5.30 (dd, J=14.4 Hz, 4.8 Hz, 1H), 5.12 (s, 2H), 4.51 (dd, J=12.0 Hz, 3.2 Hz, 1H), 4.22 (td, 12.0 Hz, 5.2 Hz, 1H), 3.88 (m, 1H), 3.26 (s, 1H), 2.57 (m, 1H), 2.29 (s, 3H), 1.27 (d, 6.4 Hz, 3H), 1.06 (d, 6.4 Hz, 3H).

### Example 22

### (R)-1-(2-(1-isopropyl-7-(methylsulfonyl)-3,4-dihydropyrazino[1,2-a]indol-2 (1H)— yl)-4-(trifluoromethyl)pyrimidin-5-yl)ethanone and

### (S)-1-(2-(1-isopropyl-7-(methylsulfonyl)-3,4-dihydropyrazino[1,2-a]indol-2 (1H)— yl)-4-(trifluoromethyl)pyrimidin-5-yl)ethanone

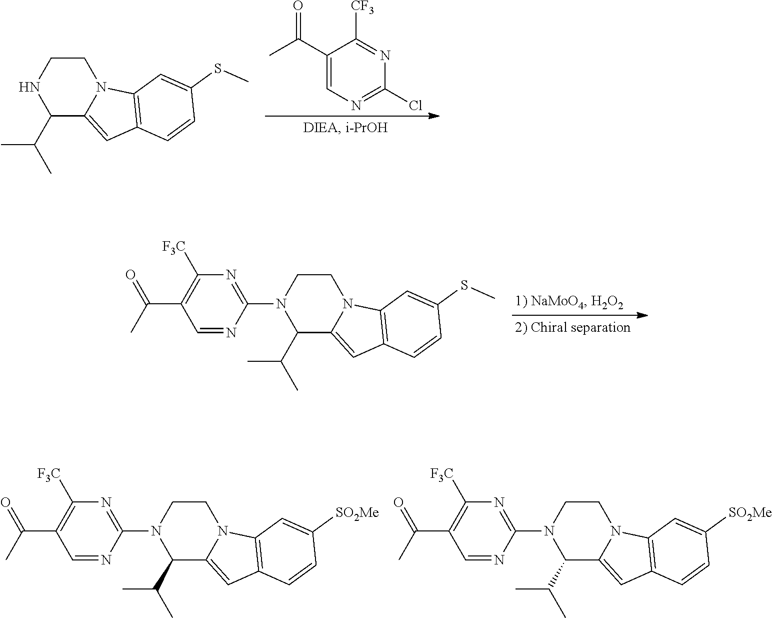

To a solution of 1-isopropyl-7-(methylthio)-1,2,3,4-tetrahydropyrazino[1,2-a]indole (100 mg, 0.38 mmol) and DIPEA (246.72 mg, 1.91 mmol) in i-PrOH (2 mL) was added 1-(2-chloro-4-(trifluoromethyl)pyrimidin-5-yl)ethanone (172.50 mg, 0.76 mmol). The reaction mixture was stirred at rt overnight. The mixture was concentrated and purified by preparative TLC on silica gel eluting with PE/EtOAc 1:1 to afford 1-(2-(1-isopropyl-7-(methylthio)-3,4-dihydropyrazino[1,2-a]indol-2(1H)-yl)-4-(trifluoromethyl)pyrimidin-5-yl)ethanone (120 mg, 69.67% yield) as a colorless oil. LC-MS MS (ESI) m/z 449.2 [M+H]⁺. ¹H NMR (CDCl₃ 400 MHz): δ 8.66 (s, 1H), 7.44-7.40 (m, 1H), 7.06 (d, J=8.4 Hz, 1H), 6.27-6.25 (m, 1H), 5.79-577 (m, 1H), 5.13-5.02 (m, 1H), 4.21-4.16 (m, 1H), 3.97-3.91 (m, 1H), 3.82-3.71 (m, 1H), 2.48 (s, 3H), 2.46 (s, 3H), 2.21-2.15 (m, 1H), 1.12-1.08 (m, 3H), 0.96-0.93 (m, 3H).

To a solution of 1-(2-(1-isopropyl-7-(methylthio)-3,4-dihydropyrazino[1,2-a]indol-2(1H)-yl)-4-(trifluoromethyl)pyrimidin-5-yl)ethanone (110 mg, 0.23 mmol) in methanol (3 mL) at 0° C. was added NaMoO₄-2H₂O (107.61 mg, 0.49 mmol). The reaction mixture was stirred at 0° C. for 10 min. Then H₂O₂ (5 mL, 30% wt) was added to the formed mixture. The mixture was stirred at rt for 1 h. The mixture was extracted with a mixture solvent of dichloromethane (30 mL)/i-PrOH (10 mL) three times. The combined organic layers were concentrated, purified by preparative TLC on silica gel eluting with PE/EtOAc 1:1 and purified by SFC separation to afford (R)-1-(2-(1-isopropyl-7-(methylsulfonyl)-3,4-dihydropyrazino[1,2-a]indol-2(1H)-yl)-4-(trifluoromethyl)pyrimidin-5-yl)ethanone (26.90 mg, 22.38% yield, isomer 1) as a colorless oil and (S)-1-(2-(1-isopropyl-7-(methylsulfonyl)-3,4-dihydropyrazino[1,2-a]indol-2(1H)-yl)-4-(trifluoromethyl)pyrimidin-5-yl)ethanone (23.80 mg, 20.20% yield, isomer 2) as a colorless oil.

Isomer 1: (R)-1-(2-(1-isopropyl-7-(methylsulfonyl)-3,4-dihydropyrazino[1,2-a]indol-2(1H)-yl)-4-(trifluoromethyl)pyrimidin-5-yl)ethanone. Analytical chiral HPLC: t_{R}=7.972 min in 15 min chromatography (Method: AD-H_5_5_40_2.35 ML). LC-MS (ESI) m/z 481.2[M+H]⁺, 503.1 [M+Na]⁺. ¹H NMR (CDCl₃ 400 MHz): δ 8.75-8.73 (m, 1H), 7.95 (s, 1H), 7.72 (d, J=8.4 Hz, 1H), 7.65 (d, J₁=8.4 Hz, J₂=1.6 Hz, 1H), 6.51-6.48 (m, 1H), 5.94-5.92 (m, 1H), 5.25-5.22 (m, 1H), 4.41-4.36 (m, 1H), 4.15-4.10 (m, 1H), 3.88-3.84 (m, 1H), 3.08 (s, 3H), 2.54 (s, 3H), 2.34-2.27 (m, 1H), 1.21-1.18 (m, 3H), 1.06-1.05 (m, 3H).

Isomer 2: (R)-1-(2-(1-isopropyl-7-(methylsulfonyl)-3,4-dihydropyrazino[1,2-a]indol-2(1H)-yl)-4-(trifluoromethyl)pyrimidin-5-yl)ethanone. Analytical chiral HPLC: t_{R}=11.077 min in 15 min chromatography (Method: AD-H_5_5_40_2.35 ML). LC-MS MS (ESI) m/z 481.1[M+H]⁺, 503.1[M+Na]⁺. ¹H NMR (CDCl₃ 400 MHz): δ 8.75-8.73 (m, 1H), 7.95 (s, 1H), 7.73-7.70 (m, 1H), 7.65 (d, J₁=8.4 Hz, J₂=1.6 Hz, 1H), 6.51-6.48 (m, 1H), 5.94-5.92 (m, 1H), 5.25-5.22 (m, 1H), 4.41-4.36 (m, 1H), 4.15-4.10 (m, 1H), 3.88-3.84 (m, 1H), 3.08 (s, 3H), 2.54 (s, 3H), 2.34-2.27 (m, 1H), 1.21-1.18 (m, 3H), 1.07-1.05 (m, 3H).

### Example 23

### (R)-2-(2-(1-isopropyl-7-(methylsulfonyl)-3,4-dihydropyrazino[1,2-a]indol-2 (1H)— yl)-4-(trifluoromethyl)pyrimidin-5-yl)propan-2-ol and

### (S)-2-(2-(1-isopropyl-7-(methylsulfonyl)-3,4-dihydropyrazino[1,2-a]indol-2(1H)— yl)-4-(trifluoromethyl)pyrimidin-5-yl)propan-2-ol

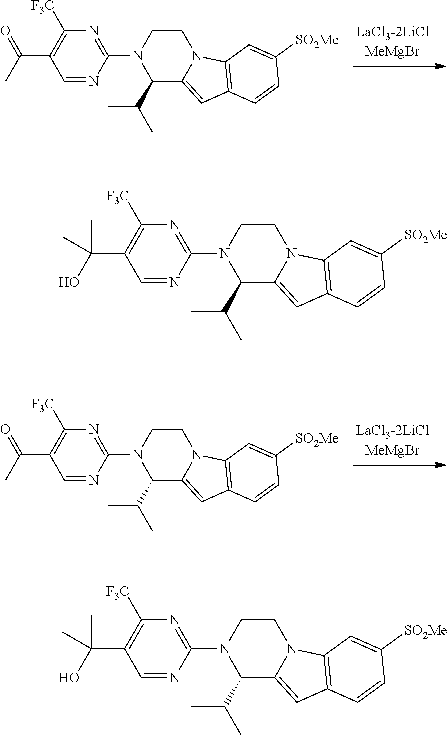 [SMILES: CC(=O)c1cnc(N2CCn3c(cc4ccc(S(C)(=O)=O)cc43)[C@H]2C(C)C)nc1C(F)(F)F ; CC(C)[C@@H]1c2cc3ccc(S(C)(=O)=O)cc3n2CCN1c1ncc(C(C)(C)O)c(C(F)(F)F)n1 ; CC(=O)c1cnc(N2CCn3c(cc4ccc(S(C)(=O)=O)cc43)[C@@H]2C(C)C)nc1C(F)(F)F ; CC(C)[C@H]1c2cc3ccc(S(C)(=O)=O)cc3n2CCN1c1ncc(C(C)(C)O)c(C(F)(F)F)n1]

To a solution of (R)-1-(2-(1-isopropyl-7-(methylsulfonyl)-3,4-dihydropyrazino[1,2-a]indol-2(1H)-yl)-4-(trifluoromethyl)pyrimidin-5-yl)ethanone (5 mg, 0.01 mmol) in THF (1 mL) was added LaCl₃-2LiCl (0.2 mL, 0.12 mmol, 0.6M) at 0° C. under nitrogen. The reaction mixture was stirred at 0° C. for 1 h. MeMgCl (0.3 mL, 0.9 mmol, 3M) was added to the formed mixture and then the reaction mixture was stirred at 0° C. for 1 h. The reaction was quenched with sat. NH₄Cl (5 mL) at 0° C. and then extracted with EtOAc (30 mL×3). The combined organic layers were dried over anhydrous Na2SO4, filtered and concentrated, purified by preparative TLC eluting with PE/EtOAc 1:1 and then by SFC separation to afford (R)-2-(2-(1-isopropyl-7-(methylsulfonyl)-3,4-dihydropyrazino[1,2-a]indol-2(1H)-yl)-4-(trifluoromethyl)pyrimidin-5-yl)propan-2-ol (1.20 mg, 24.68% yield, Isomer 1) as a colorless oil. Analytical chiral HPLC: t_{R}=6.394 min in 15 min chromatography (Method: OJ-H_3_5_40_2.35 ML). LC-MS m/z 497.3 [M+H]⁺. ¹H NMR (CDCl₃ 400 MHz): δ 8.75 (s, 1H), 7.93 (s, 1H), 7.70 (d, J=8.4 Hz, 1H), 7.62 (d, J₁=8.4 Hz, J₂=1.6 Hz, 1H), 6.48 (s, 1H), 5.89 (d, J=8.8 Hz, 1H), 5.16-5.12 (m, 1H), 4.32-4.28 (m, 1H), 4.14-4.07 (m, 1H), 3.86-3.77 (m, 1H), 3.07 (s, 3H), 2.34-2.25 (m, 1H), 1.92 (s, 1H), 1.68 (s, 6H), 1.18 (d, J=7.2 Hz, 3H), 1.06 (d, J=7.2 Hz, 3H)

The (S)-2-(2-(1-isopropyl-7-(methylsulfonyl)-3,4-dihydropyrazino[1,2-a]indol-2(1H)-yl)-4-(trifluoromethyl)pyrimidin-5-yl)propan-2-ol (Isomer 2) was prepared in similar manner from (S)-1-(2-(1-isopropyl-7-(methylsulfonyl)-3,4-dihydropyrazino[1,2-a]indol-2(1H)-yl)-4-(trifluoromethyl)pyrimidin-5-yl)ethanone. Analytical chiral HPLC: t_{R}=7.631 min in 15 min chromatography (Method: AS-H_3_5_40_2.35 ML). LC-MS m/z 497.2[M+H]⁺. ¹H NMR (CDCl₃ 400 MHz): δ 8.75 (s, 1H), 7.93 (s, 1H), 7.70 (d, J=8.4 Hz, 1H), 7.62 (d, J₁=8.4 Hz, J₂=1.6 Hz, 1H), 6.48 (s, 1H), 5.89 (d, J=8.8 Hz, 1H), 5.16-5.12 (m, 1H), 4.32-4.28 (m, 1H), 4.14-4.07 (m, 1H), 3.86-3.77 (m, 1H), 3.07 (s, 3H), 2.34-2.25 (m, 1H), 1.92 (s, 1H), 1.68 (s, 6H), 1.18 (d, J=7.2 Hz, 3H), 1.06 (d, J=7.2 Hz, 3H).

### Example 24

### 1-(2-(1-isopropyl-7-(methylsulfonyl)-3,4-dihydropyrazino[1,2-a]indol-2(1H)-yl)-4-(trifluoromethyl)pyrimidin-5-yl)ethanol (4 isomers)

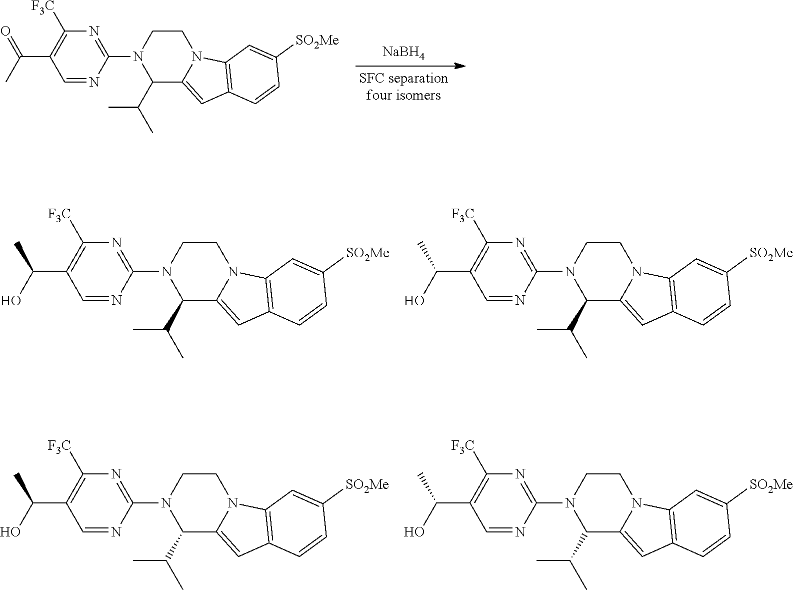

To a solution of 1-(2-(1-isopropyl-7-(methylsulfonyl)-3,4-dihydropyrazino[1,2-a]indol-2(1H)-yl)-4-(trifluoromethyl)pyrimidin-5-yl)ethanone (10 mg, 21 μmol) in methanol (2 mL) was added NaBH₄ (7.8 mg, 0.21 mmol) at 0° C. The reaction mixture was stirred at reflux for 2 h. The mixture was quenched with water (5 mL) and concentrated to remove methanol to give crude product which was extracted with EtOAc (30 mL×3). The combined organic layers were dried over anhydrous Na2SO4, filtered, concentrated and purified by preparative TLC on silica gel eluting with PE/EtOAc 1:1 and then by SFC separation to afford Isomer 1 (0.80 mg, 7.97% yield) as a colorless oil, Isomer 2 (0.90 mg, 8.96% yield) as a colorless oil, Isomer 3 (1.20 mg, 11.95% yield) as a colorless oil and Isomer 4 (1.30 mg, 12.94% yield) as a colorless oil.

Isomer 1: Analytical chiral HPLC: t_{R}=3.16 min in 15 min chromatography (Method: AD-H_3_30%_2.35 ML). LC-MS m/z 483.0[M+H]⁺. ¹H NMR (CDCl₃ 400 MHz): δ 8.83 (s, 1H), 7.93 (s, 1H), 7.68 (d, J=7.2 Hz, 1H), 7.52 (d, J=7.2 Hz, 1H), 6.47 (s, 1H), 5.89 (d, J=8.8 Hz, 1H), 5.24-5.14 (m, 2H), 4.33-4.31 (m, 1H), 4.16-4.09 (m, 1H), 3.85-3.81 (m, 1H), 3.08 (s, 3H), 2.36-2.29 (m, 1H), 1.53-1.51 (m, 3H), 1.19 (d, J=6.4 Hz, 3H), 1.07 (d, J=6.8 Hz, 3H).

Isomer 2: Analytical chiral HPLC: t_{R}=4.04 min in 15 min chromatography (Method: AD-H_3_30%_2.35 ML). LC-MS m/z 483.0 [M+H]⁺. ¹H NMR (CDCl₃ 400 MHz): δ 8.83 (s, 1H), 7.93 (s, 1H), 7.68 (d, J=7.2 Hz, 1H), 7.52 (d, J=7.2 Hz, 1H), 6.47 (s, 1H), 5.89 (d, J=8.8 Hz, 1H), 5.24-5.14 (m, 2H), 4.33-4.31 (m, 1H), 4.16-4.09 (m, 1H), 3.85-3.81 (m, 1H), 3.08 (s, 3H), 2.36-2.29 (m, 1H), 1.53-1.51 (m, 3H), 1.19 (d, J=6.4 Hz, 3H), 1.07 (d, J=6.8 Hz, 3H).

Isomer 3: Analytical chiral HPLC: t_{R}=6.08 min in 15 min chromatography (Method: AD-H_3_30%_2.35 ML). LC-MS m/z 483.0 [M+H]⁺. ¹H NMR (CDCl₃ 400 MHz): δ 8.83 (s, 1H), 7.93 (s, 1H), 7.68 (d, J=7.2 Hz, 1H), 7.61 (d, J=7.2 Hz, 1H), 6.47 (s, 1H), 5.89 (d, J=8.8 Hz, 1H), 5.24-5.14 (m, 2H), 4.33-4.31 (m, 1H), 4.16-4.09 (m, 1H), 3.85-3.81 (m, 1H), 3.08 (s, 3H), 2.36-2.29 (m, 1H), 1.53-1.51 (m, 3H), 1.19 (d, J=6.4 Hz, 3H), 1.07 (d, J=6.8 Hz, 3H).

Isomer 4: Analytical chiral HPLC: t_{R}=10.21 min in 15 min chromatography (Method: AD-H_3_30%_2.35 ML). LC-MS m/z 483.0 [M+H]⁺. ¹H NMR (CDCl₃ 400 MHz): δ 8.84 (s, 1H), 7.93 (s, 1H), 7.68 (d, J=7.2 Hz, 1H), 7.61 (d, J=7.2 Hz, 1H), 6.47 (s, 1H), 5.89 (d, J=8.8 Hz, 1H), 5.24-5.14 (m, 2H), 4.33-4.31 (m, 1H), 4.16-4.09 (m, 1H), 3.85-3.81 (m, 1H), 3.08 (s, 3H), 2.36-2.29 (m, 1H), 1.53-1.51 (m, 3H), 1.19 (d, J=6.4 Hz, 3H), 1.07 (d, J=6.8 Hz, 3H).

### Example 25

### (R)-(2-(1-isopropyl-7-(methylsulfonyl)-3,4-dihydropyrazino[1,2-a]indol-2(1H)-yl)-4-(trifluoromethyl)pyrimidin-5-yl)methanol and

### (S)-(2-(1-isopropyl-7-(methylsulfonyl)-3,4-dihydropyrazino[1,2-a]indol-2(1H)-yl)-4-(trifluoromethyl)pyrimidin-5-yl)methanol

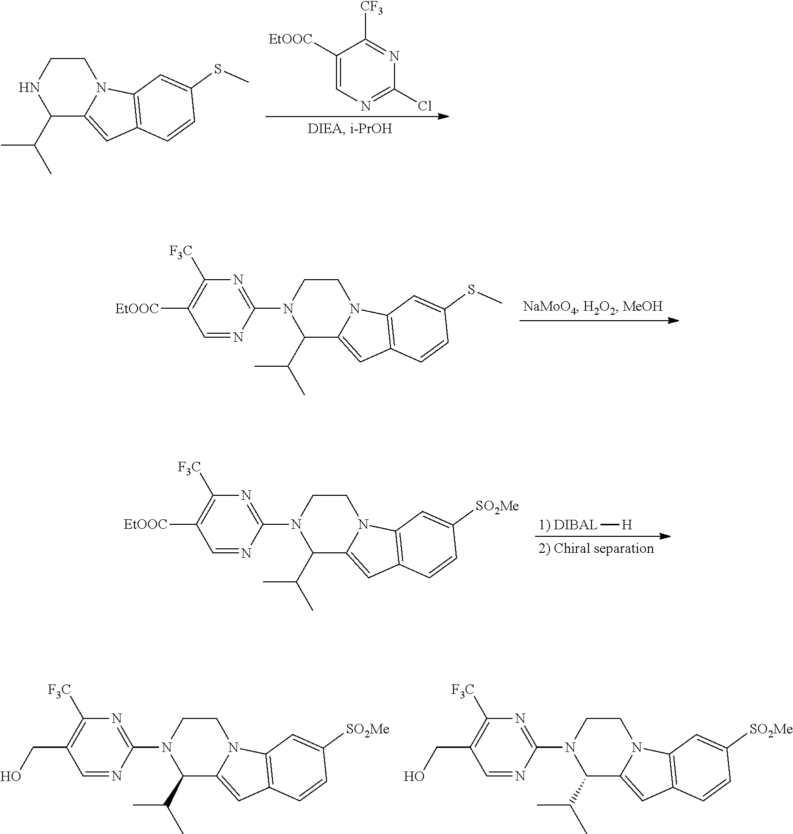

To a solution of 1-isopropyl-7-(methylthio)-1,2,3,4-tetrahydropyrazino[1,2-a]indole (100 mg, 0.38 mmol) and DIPEA (248.3 mg, 1.921 mmol) in i-PrOH (3 mL) was added ethyl 2-chloro-4-(trifluoromethyl)pyrimidine-5-carboxylate (196 mg, 0.77 mmol). The reaction mixture was stirred at rt overnight. The mixture was concentrated and purified by preparative TLC on silica gel eluting with PE/EtOAc 1:1 to afford ethyl 2-(1-isopropyl-7-(methylthio)-3,4-dihydropyrazino[1,2-a]indol-2(1H)-yl)-4-(trifluoromethyl)pyrimidine-5-carboxylate (60 mg, 32.6% yield) as a colorless oil. LC-MS MS (ESI) m/z 478.7 [M+H]⁺.

To a solution of ethyl 2-(1-isopropyl-7-(methylthio)-3,4-dihydropyrazino[1,2-a]indol-2(1H)-yl)-4-(trifluoromethyl)pyrimidine-5-carboxylate (54 mg, 0.11 mmol) in methanol (2 mL) at 0° C. was added NaMoO₄-2H₂O (54 mg, 0.24 mmol). The reaction mixture was stirred at 0° C. for 10 min H₂O₂ (2 mL, 30% wt) was added to the formed mixture. The mixture was stirred at rt for 1 h. The mixture was extracted with a mixture solvent of dichloromethane (30 mL)/i-PrOH (10 mL) three times. The combined organic layers were concentrated, purified by preparative TLC eluting with PE/EtOAc 1:1 to afford ethyl 2-(1-isopropyl-7-(methylsulfonyl)-3,4-dihydropyrazino[1,2-a]indol-2(1H)-yl)-4-(trifluoromethyl)pyrimidine-5-carboxylate (25 mg, 43.39% yield) as a white solid. LC-MS MS (ESI) m/z 511.1 [M+H]⁺.

To a solution of 2-(1-isopropyl-7-(methylsulfonyl)-3,4-dihydropyrazino[1,2-a]indol-2(1H)-yl)-4-(trifluoromethyl)pyrimidine-5-carboxylate (25 mg, 0.05 mmol) in dichloromethane (1 mL) was added DIBAL-H (0.25 mg, 0.25 mmol, 1M in toluene) at −78° C. under nitrogen. The reaction mixture was stirred at −78° C. for 1 h. The reaction was quenched with sat. NH₄Cl (5 mL) at −78° C. and then extracted with dichloromethane (30 mL×3). The combined organic layers were concentrated, purified by preparative TLC on silica gel eluting with PE/EtOAc 1:1 and then by SFC separation to afford (R)-(2-(1-isopropyl-7-(methylsulfonyl)-3,4-dihydropyrazino[1,2-a]indol-2(1H)-yl)-4-(trifluoromethyl)pyrimidin-5-yl)methanol (1.80 mg, 7.84% yield, isomer 1) as a colorless oil and (S)-(2-(1-isopropyl-7-(methylsulfonyl)-3,4-dihydropyrazino[1,2-a]indol-2(1H)-yl)-4-(trifluoromethyl)pyrimidin-5-yl)methanol (1.60 mg, 6.97% yield, isomer 2) as a colorless oil.

Isomer 1: (R)-(2-(1-isopropyl-7-(methylsulfonyl)-3,4-dihydropyrazino[1,2-a]indol-2(1H)-yl)-4-(trifluoromethyl)pyrimidin-5-yl)methanol. Analytical chiral HPLC: t_{R}=8.1 min in 15 min chromatography (Method: OD-3_5_5_40_2.5 ML). LC-MS m/z 469.0 [M+H]⁺. ¹H NMR (CDCl₃ 400 MHz): δ 8.63 (s, 1H), 7.94 (s, 1H), 7.70 (d, J=8.4 Hz, 1H), 7.63 (d, J₁=8.4 Hz, J₂=1.6 Hz, 1H), 6.48 (s, 1H), 5.89 (d, J=8.8 Hz, 1H), 5.19-5.14 (m, 1H), 4.71 (d, J=4.8 Hz, 2H), 3.86-3.82 (m, 1H), 4.14-4.07 (m, 1H), 3.86-3.77 (m, 1H), 3.07 (s, 3H), 2.34-2.25 (m, 1H), 1.82-1.78 (m, 1H), 1.18 (d, J=7.2 Hz, 3H), 1.04 (d, J=7.2 Hz, 3H).

Isomer 2: (S)-(2-(1-isopropyl-7-(methylsulfonyl)-3,4-dihydropyrazino[1,2-a]indol-2(1H)-yl)-4-(trifluoromethyl)pyrimidin-5-yl)methanol. Analytical chiral HPLC: t_{R}=11.33 min in 15 min chromatography (Method: OD-3_5_5_40_2.5 ML). LC-MS m/z 469.1 [M+H]⁺. ¹H NMR (CDCl₃ 400 MHz): δ 8.63 (s, 1H), 7.94 (s, 1H), 7.70 (d, J=8.4 Hz, 1H), 7.63 (d, J₁=8.4 Hz, J₂=1.6 Hz, 1H), 6.48 (s, 1H), 5.89 (d, J=8.8 Hz, 1H), 5.19-5.14 (m, 1H), 4.71 (s, 2H), 3.86-3.82 (m, 1H), 4.14-4.07 (m, 1H), 3.86-3.77 (m, 1H), 3.07 (s, 3H), 2.34-2.25 (m, 1H), 1.82-1.78 (m, 1H), 1.18 (d, J=7.2 Hz, 3H), 1.04 (d, J=7.2 Hz, 3H).

### Example 26

### (±)-1-(2-(8-(hydroxymethyl)-1-isopropyl-7-(methylsulfonyl)-3,4-dihydropyrazino[1,2-a]indol-2(1H)-yl)-4-(trifluoromethyl)pyrimidin-5-yl)ethanone

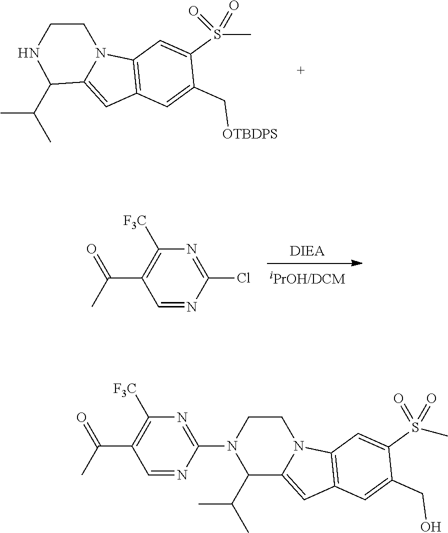

The intermediate 8-(((tert-butyldiphenylsilyl)oxy)methyl)-1-isopropyl-7-(methylsulfonyl)-1,2,3,4-tetrahydropyrazino[1,2-a]indole was prepared following a procedure analogous to that described in Preparation 4. The mixture of compound 8-(((tert-butyldiphenylsilyl)oxy)methyl)-1-isopropyl-7-(methylsulfonyl)-1,2,3,4-tetrahydropyrazino[1,2-a]indole (0.22 mmol), 1-(2-chloro-4-(trifluoromethyl)pyrimidin-5-yl)ethanone (100 mg, 0.44 mmol) and DIEA (115 μL, 0.66 mmol) in i-PrOH/CH₂Cl₂ (2 mL/1 mL) was stirred at 60° C. for 15 h. The solvent was removed under reduced pressure and the residue was purified by column chromatography on silica gel eluting with EtOAc/hexanes (1/1) to give racemic 1-(2-(8-(hydroxymethyl)-1-isopropyl-7-(methylsulfonyl)-3,4-dihydropyrazino[1,2-a]indol-2(1H)-yl)-4-(trifluoromethyl)pyrimidin-5-yl)ethanone. LC-MS m/z 510 [M+H]⁺. ¹H NMR (400 MHz, CD₃OD): δ 8.97 (s, 1H), 8.11 (s, 1H), 7.80 (s, 1H), 6.54 (s, 1H), 6.01-5.90 (m, 1H), 5.25-5.15 (m, 1H), 5.07 (s, 2H), 4.52-4.47 (m, 1H), 4.15-4.04 (m, 1H), 4.00-3.93 (m, 1H), 3.26 (s, 3H), 2.55 (s, 3H), 2.41-2.32 (m, 1H), 1.18 (d, J=6.8 Hz, 3H), 1.03 (d, J=6.8 Hz, 3H).

### Example 27

### (R)-2-(2-(8-(hydroxymethyl)-1-isopropyl-7-(methyl sulfonyl)-3,4-dihydropyrazino[1,2-a]indol-2(1H)-yl)-4-(trifluoromethyl)pyrimidin-5-yl)propan-2-ol and

### (S)-2-(2-(8-(hydroxymethyl)-1-isopropyl-7-(methylsulfonyl)-3,4-dihydropyrazino[1,2-a]-indol-2 (1H)— yl)-4-(trifluoromethyl)pyrimidin-5-yl)propan-2-ol

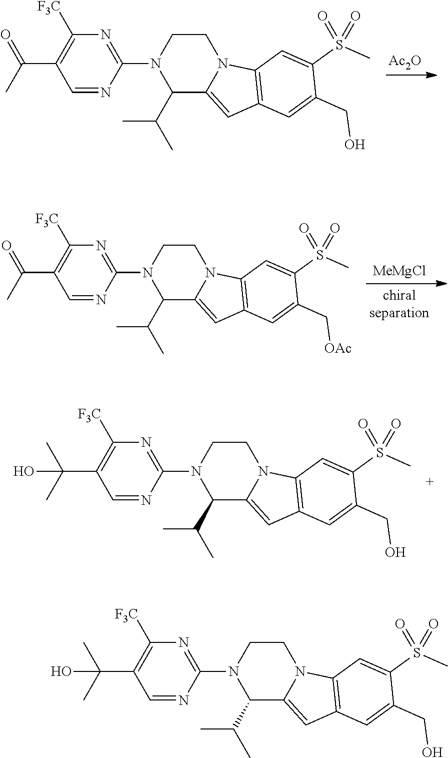

To a solution of 1-(2-(8-(hydroxymethyl)-1-isopropyl-7-(methylsulfonyl)-3,4-dihydropyrazino[1,2-a]indol-2(1H)-yl)-4-(trifluoromethyl)pyrimidin-5-yl)ethanone (132 mg, 0.26 mmol) in CH₂Cl₂ (5 mL) was added pyridine (1 mL) and AcCl (130 μL, 1.3 mmol). The mixture was stirred at rt for 10 h. The reaction was quenched with water (5 mL). The aqueous layer was extracted with CH₂Cl₂ (3×10 mL). The combined organic layers were washed with brine, and then dried over anhydrous Na₂SO₄. The mixture was filtered and concentrated under reduced pressure. The residue was purified by column chromatography on silica gel eluting with hexanes/EtOAc (1/1) to give (2-(5-acetyl-4-(trifluoromethyl)pyrimidin-2-yl)-1-isopropyl-7-(methylsulfonyl)-1,2,3,4-tetrahydropyrazino[1,2-a]indol-8-yl)methyl acetate. LC-MS m/z 553 [M+H]⁺.

To a solution of (2-(5-acetyl-4-(trifluoromethyl)pyrimidin-2-yl)-1-isopropyl-7-(methylsulfonyl)-1,2,3,4-tetrahydropyrazino[1,2-a]indol-8-yl)methyl acetate (37 mg, 67 μmol) in dry THF (2 mL) was added LaCl3.2 LiCl THF solution (0.12 mL, 70 μmol). The resulting mixture was stirred for 20 min at rt. The reaction mixture was cooled down to 0° C., MeMgCl in THF solution (3.0 M, 0.15 mL) was added slowly and the reaction mixture was allowed to stir at the same temperature for 0.5 h. Sat. aq. NH₄Cl (1 mL) and water (2 mL) were added. The aqueous layer was extracted with EtOAc (4×10 mL). Combined organic phases were dried (Na₂SO₄) and concentrated. The crude residue was purified by silica chromatography and SFC separation to give isomers of 2-(2-(8-(hydroxymethyl)-1-isopropyl-7-(methylsulfonyl)-3,4-dihydropyrazino[1,2-a]indol-2(1H)-yl)-4-(trifluoromethyl)pyrimidin-5-yl)propan-2-ol.

Isomer 1: Analytical chiral HPLC: t_{R}=12.31 min in 15 min chromatography (Method: OD-H_5_5_40_2.35 ML). LC-MS m/z 527 [M+H]⁺. ¹H NMR (400 MHz, CD₃OD): δ 8.83 (s, 1H), 8.08 (s, 1H), 7.77 (s, 1H), 6.50 (s, 1H), 5.87 (d, J=8.4 Hz, 1H), 5.10 (m, 1H), 5.06 (s, 2H), 4.44-4.40 (m, 1H), 4.09-4.02 (m, 1H), 3.91-3.84 (m, 1H), 3.26 (s, 3H), 2.36-2.29 (m, 1H), 1.59 (s, 6H), 1.16 (d, J=6.8 Hz, 3H), 1.02 (d, J=6.8 Hz, 3H).

Isomer 2: Analytical chiral HPLC: t_{R}=8.65 min in 15 min chromatography (Method: OD-H_5_5_40_2.35 ML). LC-MS m/z 527 [M+H]⁺. ¹H NMR (400 MHz, CD₃OD): δ 8.83 (s, 1H), 8.08 (s, 1H), 7.77 (s, 1H), 6.50 (s, 1H), 5.87 (d, J=8.4 Hz, 1H), 5.10 (m, 1H), 5.06 (s, 2H), 4.44-4.40 (m, 1H), 4.09-4.02 (m, 1H), 3.91-3.84 (m, 1H), 3.26 (s, 3H), 2.36-2.29 (m, 1H), 1.59 (s, 6H), 1.16 (d, J=6.8 Hz, 3H), 3 1.02 (d, J=6.8 Hz, 3H).

### Example 28

### (±)-Ethyl 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxylate

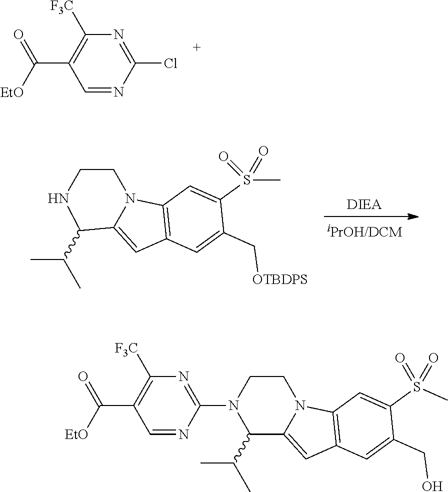

The intermediate 8-(((tert-butyldiphenylsilyl)oxy)methyl)-1-isopropyl-7-(methylsulfonyl)-1,2,3,4-tetrahydropyrazino[1,2-a]indole was prepared following a procedure analogous to that described in Preparation 4. The mixture of compound 8-(((tert-butyldiphenylsilyl)oxy)methyl)-1-isopropyl-7-(methylsulfonyl)-1,2,3,4-tetrahydropyrazino[1,2-a]indole (0.19 mmol), ethyl 2-chloro-4-(trifluoromethyl)pyrimidine-5-carboxylate (97 mg, 0.38 mmol) and DIEA (100 μL, 0.57 mmol) in i-PrOH/CH₂Cl₂ (1 mL/0.5 mL) was stirred at 50° C. for 8 h. The solvent was removed under reduced pressure and the residue was purified by column chromatography on silica gel eluting with EtOAc/hexanes (1/1) to give racemic ethyl 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxylate. LC-MS m/z 563 [M+Na]⁺. ¹H NMR (400 MHz, CD₃OD): δ 9.31 (s, 1H), 8.11 (s, 1H), 7.80 (s, 1H), 6.55 (s, 1H), 6.02-5.92 (m, 1H), 5.23-5.17 (m, 1H), 5.07 (s, 2H), 4.52-4.47 (m, 1H), 4.34 (q, J=7.2 Hz, 2H), 4.19-4.06 (m, 1H), 4.00-3.93 (m, 1H), 3.27 (s, 3H), 2.42-2.32 (m, 1H), 1.36 (t, J=7.2 Hz, 3H), 1.18 (d, J=6.8 Hz, 3H), 1.03 (d, J=6.8 Hz, 3H).

### Example 29

### (R)-2-(8-(hydroxymethyl)-1-isopropyl-7-(methylsulfonyl)-3,4-dihydropyrazino[1,2-a]indol-2(1H)— yl)-4-(trifluoromethyl)pyrimidine-5-carboxamide and

### (S)-2-(8-(hydroxymethyl)-1-isopropyl-7-(methylsulfonyl)-3,4-dihydropyrazino[1,2-a]indol-2(1H)— yl)-4-(trifluoromethyl)pyrimidine-5-carboxamide

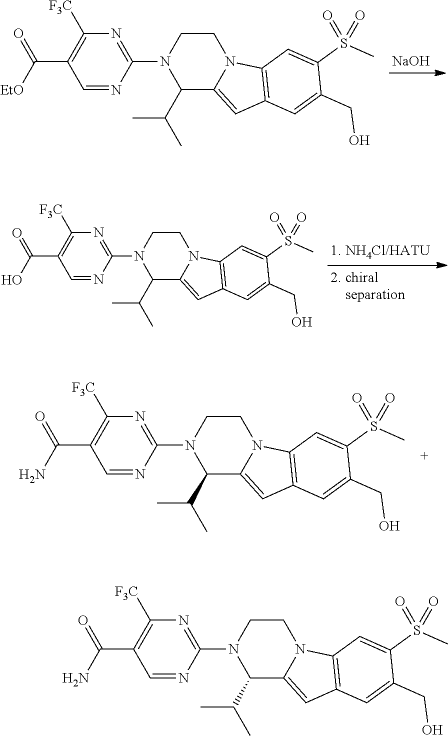

To a solution of ethyl 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxylate (30 mg, 55 mol) in THF (1 mL) was added 1 N NaOH aqueous solution (1 mL). The resulting mixture was stirred at rt for 3 h. The reaction mixture was acidified with 1N HCl solution (1.5 mL). The mixture was extracted with CH₂Cl₂ (4×5 mL). The combined organic solution was washed with brine, dried over anhydrous Na₂SO₄, filtered and concentrated under reduced pressure to provide 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxylic acid. It was used directly without further purification.

To a stirred solution of 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxylic acid (55 mol) in anhydrous DMF (1 mL) was added HATU (42 mg, 0.11 mmol), NH₄Cl (30 mg, 0.55 mmol) and N,N-diisopropylethylamine (100 μL, 0.55 mmol). The mixture was stirred at rt for 20 h. It was diluted with CH₂Cl₂ (10 mL) and washed with H₂O. The organic layer was separated, and the aqueous layer was extracted with CH₂Cl₂ (3×10 mL). The combined organic solution was washed with brine, dried over anhydrous Na₂SO₄, filtered and concentrated under reduced pressure. The crude residue was purified by silica chromatography and SFC separation to give isomers of 2-(8-(hydroxymethyl)-1-isopropyl-7-(methylsulfonyl)-3,4-dihydropyrazino[1,2-a]indol-2(1H)-yl)-4-(trifluoromethyl)pyrimidine-5-carboxamide.

Isomer 1: Analytical chiral HPLC: t_{R}=2.92 min in 8 min chromatography (Method: AS-H_S_3_40_3 ML). LC-MS m/z 494 [M+H−18]⁺, 512 [M+H]⁺. ¹H NMR (400 MHz, CD₃OD): δ 8.68 (s, 1H), 8.12 (s, 1H), 7.80 (s, 1H), 6.54 (s, 1H), 6.00-5.87 (m, 1H), 5.20-5.08 (m, 1H), 5.07 (s, 2H), 4.50-4.46 (m, 1H), 4.14-4.08 (m, 1H), 3.98-3.90 (m, 1H), 3.27 (s, 3H), 2.39-2.32 (m, 1H), 1.29 (s, 2H), 1.18 (d, J=6.8 Hz, 3H), 1.04 (d, J=6.8 Hz, 3H).

Isomer 2: Analytical chiral HPLC: t_{R}=4.91 min in 8 min chromatography (Method: AS-H_S_3_40_3 ML). LC-MS m/z 494 [M+H−18]⁺, 512 [M+H]⁺. ¹H NMR (400 MHz, CD₃OD): δ 8.68 (s, 1H), 8.12 (s, 1H), 7.80 (s, 1H), 6.54 (s, 1H), 6.00-5.87 (m, 1H), 5.20-5.08 (m, 1H), 5.07 (s, 2H), 4.50-4.46 (m, 1H), 4.14-4.08 (m, 1H), 3.98-3.90 (m, 1H), 3.27 (s, 3H), 2.39-2.32 (m, 1H), 1.29 (s, 2H), 1.18 (d, J=6.8 Hz, 3H), 1.04 (d, J=6.8 Hz, 3H).

### Example 30

### (R)-methyl 2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidine-5-carboxylate

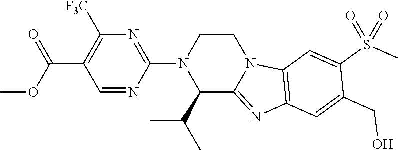

The title compound was prepared by a procedure analogous to those described in Example 12 by using methyl 2-chloro-4-(trifluoromethyl)pyrimidine-5-carboxylate instead of ethyl 2-chloro-4-(trifluoromethyl)pyrimidine-5-carboxylate as a reagent. LC-MS m/z 528 [M+H]⁺. ¹H NMR (400 MHz, CD₃OD): δ 9.04 (s, 1H), 8.25 (s, 1H), 7.96 (s, 1H), 6.12-6.06 (m, 1H), 5.46-5.34 (m, 1H), 5.11 (s, 2H), 4.54 (dd, J₁=12.4 Hz, J₂=3.2 Hz, 1H), 4.24 (td, =12.0 Hz, J₂=5.2 Hz, 1H), 3.95 (dddd, J₁=14.4 Hz, J₂=12.0 Hz, J₃=4.4 Hz, 1H), 3.89 (s, 3H), 3.26 (s, 3H), 2.64-2.54 (m, 1H), 1.29 (d, J=6.8 Hz, 3H), 1.07 (d, J=6.8 Hz, 3H).

### Example 31

### 1-isopropyl-7-(methylsulfonyl)-2-(4-(trifluoromethyl)pyrimidin-2-yl)-1,2,3,4-tetrahydrobenzo[4,5]imidazo[1,2-a]pyrazine

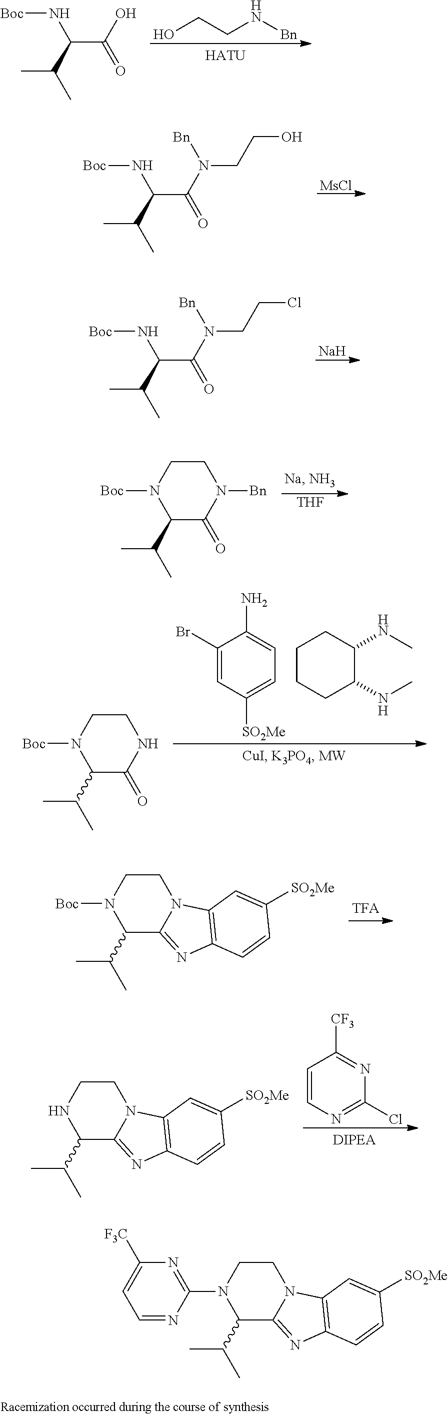

### Step 1:

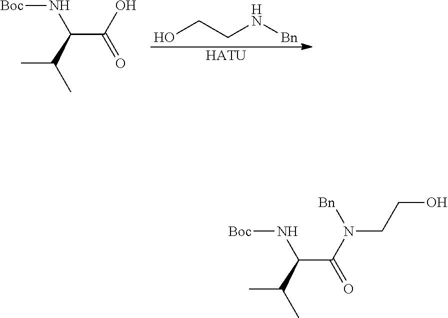

To a solution of (R)-2-((tert-butoxycarbonyl)amino)-3-methylbutanoic acid (2.0 g, 9.20 mmol) in CH₂Cl₂ (40 mL) were added 2-(benzylamino)ethanol (1.3 g, 8.80 mmol), HATU (5.30 g, 13.8 mmol) and Et₃N (2.80 g, 27.6 mmol) under N₂. The mixture was stirred at rt overnight. The mixture was added water (20 mL) and extracted with EtOAc (3×30 mL). The combined organic layers were washed with brine (20 mL), dried over anhydrous Na₂SO₄, filtered, concentrated and then purified by column chromatography on silica gel to afford (R)-tert-butyl (1-(benzyl(2-hydroxyethyl)amino)-3-methyl-1-oxobutan-2-yl)carbamate (2.80 g, 88% yield) as a white solid. LC-MS m/z 351.2 [M+H]⁺.

### Step 2:

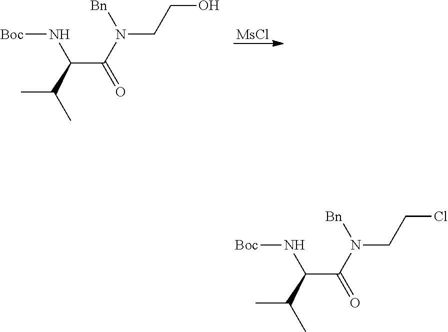 [SMILES: "MsCl" ; CC(C)[C@@H](NC(=O)OC(C)(C)C)C(=O)N(CCO)Cc1ccccc1 ; CC(C)[C@@H](NC(=O)OC(C)(C)C)C(=O)N(CCCl)Cc1ccccc1]

To a solution of (R)-tert-butyl (1-(benzyl(2-hydroxyethyl)amino)-3-methyl-1-oxobutan-2-yl)carbamate (2.80 g, 8.0 mmol) in CH₂Cl₂ (20 mL) was added Et₃N (1.60 g, 16 mmol) and MsCl (1.40 g, 12.0 mmol) dropwise at −10° C. under N₂. The mixture was stirred at rt overnight. The mixture was quenched with water (20 mL) and extracted with CH₂Cl₂ (3×20 mL). The combined organic layers were washed with brine (20 mL) and dried over anhydrous Na₂SO₄, filtered, concentrated to afford (R)-tert-butyl (1-(benzyl(2-chloroethyl)amino)-3-methyl-1-oxobutan-2-yl)carbamate (3.0 g, 100% yield) as a yellow solid, which was used for the next step without further purification. LC-MS m/z 369.2 [M+H]⁺. ¹H NMR (CDCl₃ 400 MHz): δ 7.37-7.28 (m, 3H), 7.22-7.20 (m, 2H), 5.27-5.18 (m, 1H), 4.93-4.86 (m, 1H), 4.64-4.39 (m, 2H), 3.85-3.66 (m, 2H), 3.61-3.39 (m, 2H), 2.03-1.97 (m, 1H), 1.45 (s, 9H), 0.98 (d, J=6.8 Hz, 3H), 0.93 (d, J=6.8 Hz, 3H).

### Step 3:

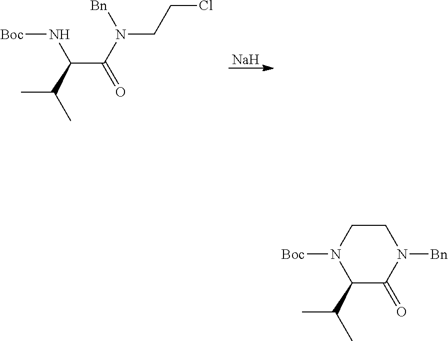

To a solution of (R)-tert-butyl (1-(benzyl(2-chloroethyl)amino)-3-methyl-1-oxobutan-2-yl)carbamate (2.0 g, 5.40 mmol) in DMF (30 mL) was added NaH (1.0 g, 27.0 mmol, 60% in mineral oil) at 0° C. under N₂. The mixture was stirred at rt for 2 h. The mixture was quenched with water (20 mL) and extracted with EtOAc (3×20 mL). The combined organic layers were washed with brine (20 mL) and dried over anhydrous Na₂SO₄, filtered, concentrated and purified by column chromatography to afford (R)-tert-butyl 4-benzyl-2-isopropyl-3-oxopiperazine-1-carboxylate (1.13 g, 63% yield) as a white solid. LC-MS m/z 277.1 [M−56+H]⁺. ¹H NMR (CDCl₃ 400 MHz): δ 7.38-7.29 (m, 3H), 7.29-7.22 (m, 2H), 5.02-4.86 (m, 1H), 4.49-4.39 (m, 1H), 4.31-4.06 (m, 2H), 3.41-3.18 (m, 3H), 2.42-2.31 (m, 1H), 1.46 (s, 9H), 1.12 (d, J=6.8 Hz, 3H), 1.00 (d, J=6.8 Hz, 3H).

### Step 4:

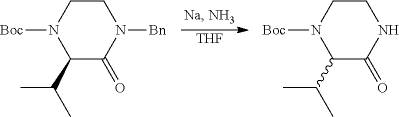 [SMILES: CC(C)[C@@H]1C(=O)N(Cc2ccccc2)CCN1C(=O)OC(C)(C)C ; CC(C)C1C(=O)NCCN1C(=O)OC(C)(C)C ; "THF"]

To a three-necked bottle containing THF (10 mL) was bubbled with NH₃ (gas) at −78° C. for 5 mins. Then Na (300 mg, 13.0 mmol) was added to the mixture slowly at −78° C. After stirring for 30 min, (R)-tert-butyl 4-benzyl-2-isopropyl-3-oxopiperazine-1-carboxylate (700 mg, 2.11 mmol) was added dropwise at −78° C. The mixture was stirred at −78° C. for 30 min. The mixture was quenched with sat. aq. NH₄Cl (10 mL) and extracted with EtOAc (3×10 mL). The combined organic layers were washed with brine (10 mL), dried over anhydrous Na₂SO₄, filtered, concentrated and purified by preparative TLC with petroleum ether/EtOAc 1/1 to afford tert-butyl 2-isopropyl-3-oxopiperazine-1-carboxylate (300 mg, 59% yield) as a white solid. The product was found to be a racemic mixture. The cause of racemization was not investigated. LC-MS m/z 187.1 [M−56+H]⁺, 265.1 [M+Na]⁺. ¹H NMR (CDCl₃ 400 MHz): δ 6.29 (s, 1H), 4.55-3.99 (m, 2H), 3.51-3.36 (m, 1H), 3.32-3.12 (m, 2H), 2.34-2.29 (m, 1H), 1.46 (s, 9H), 1.09 (d, J=6.8 Hz, 3H), 0.99 (d, J=7.2 Hz, 3H). Step 5:

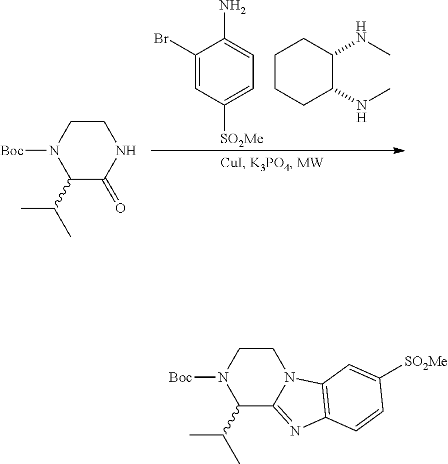

To a solution of tert-butyl 2-isopropyl-3-oxopiperazine-1-carboxylate (200 mg, 0.83 mmol) in NMP (3 mL) were added 2-bromo-4-(methylsulfonyl)aniline (207 mg, 0.83 mmol), (1R,2S)—N1,N2-dimethylcyclohexane-1,2-diamine (12.0 mg, 0.08 mmol), K₃PO₄.3H₂O (660 mg, 2.48 mmol) and Cul (16 mg, 0.08 mmol). The mixture was stirred at 150° C. for 1 h in a microwave oven. The mixture was diluted with water (10 mL) and extracted with EtOAc (3×10 mL). The combined organic layers were washed with brine (10 mL), dried over anhydrous Na₂SO₄, filtered, concentrated and purified by preparative TLC with CH₂Cl₂/MeOH 35/1 to afford tert-butyl 1-isopropyl-7-(methylsulfonyl)-3,4-dihydrobenzo[4,5]imidazo[1,2-a]pyrazine-2(1H)-carboxylate (110 mg, 34% yield) as a white solid. LC-MS m/z 394.1 [M+H]⁺. ¹H NMR (CDCl₃ 400 MHz): δ 7.94 (s, 1H), 7.83-7.76 (m, 2H), 5.35-5.17 (m, 1H), 4.73-4.42 (m, 1H), 4.22-4.12 (m, 1H), 4.11-3.99 (m, 1H), 3.53-3.37 (m, 1H), 3.03 (s, 3H), 2.38-2.27 (m, 1H), 1.42 (s, 9H), 1.19 (d, J=6.8 Hz, 3H), 0.97 (d, J=6.8 Hz, 3H).

### Step 6:

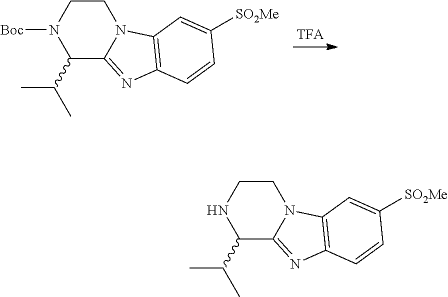 [SMILES: CC(C)C1c2nc3ccc(S(C)(=O)=O)cc3n2CCN1C(=O)OC(C)(C)C ; CC(C)C1NCCn2c1nc1ccc(S(C)(=O)=O)cc12]

To a solution of tert-butyl 1-isopropyl-7-(methylsulfonyl)-3,4-dihydrobenzo[4,5]imidazo[1,2-a]pyrazine-2(1H)-carboxylate (20. mg, 0.05 mmol) in CH₂Cl₂ (1 mL) was added TFA (0.3 mL) under N₂. The mixture was stirred at rt for 1 h. The mixture was concentrated to afford 1-isopropyl-7-(methylsulfonyl)-1,2,3,4-tetrahydrobenzo[4,5]imidazo[1,2-a]pyrazine (20 mg, TFA salt, 100% yield) as a yellow solid, which was used for the next step without further purification. LC-MS m/z 352.1 [M+H]⁺. Step 7:

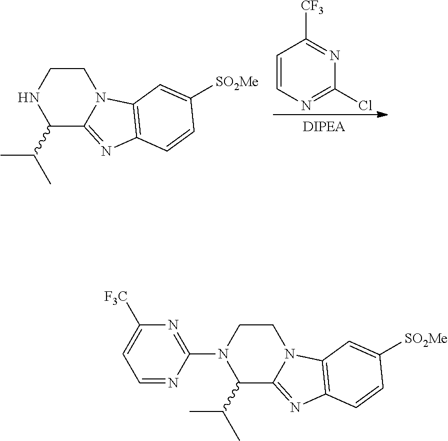 [SMILES: CC(C)C1NCCn2c1nc1ccc(S(C)(=O)=O)cc12 ; FC(F)(F)c1ccnc(Cl)n1 ; CC(C)C1c2nc3ccc(S(C)(=O)=O)cc3n2CCN1c1nccc(C(F)(F)F)n1]

To a solution of 1-isopropyl-7-(methylsulfonyl)-1,2,3,4-tetrahydrobenzo[4,5]imidazo[1,2-a]pyrazine (15 mg, 0.05 mmol) in DMSO (3 mL) was added 2-chloro-4-(trifluoromethyl)pyrimidine (19 mg, 0.10 mmol) and DIEA (20 mg, 0.15 mmol) under N₂. The mixture was stirred at 100° C. for 2 h. Water (10 mL) was added and the mixture was extracted with EtOAc (3×10 mL). The combined organic layers were washed with brine (10 mL), dried over anhydrous Na₂SO₄, filtered, concentrated and then purified by preparative TLC to afford 1-isopropyl-7-(methylsulfonyl)-2-(4-(trifluoromethyl)pyrimidin-2-yl)-1,2,3,4-tetrahydrobenzo[4,5]imidazo[1,2-a]pyrazine (5.10 mg, 23% yield) as a white solid. LC-MS m/z 440.2 (MH⁺). ¹H NMR (CDCl₃ 400 MHz): δ 8.58 (d, J=4.8 Hz, 1H), 8.01 (d, J=0.8 Hz, 1H), 7.90-7.81 (m, 2H), 6.89 (d, J=4.8 Hz, 1H), 6.12 (d, J=8.0 Hz, 1H), 5.39 (dd, J=4.0 and 14.0 Hz, 1H), 4.34-4.30 (m, 1H), 4.23-4.16 (m, 1H), 3.83-3.75 (m, 1H), 3.09 (s, 3H), 2.55-2.49 (m, 1H), 1.33 (d, J=6.8 Hz, 3H), 1.09 (d, J=6.8 Hz, 3H).

### Example 32

(R)-(1-isopropyl-7-(methyl sulfonyl)-2-(4-(trifluoromethyl)pyrimidin-2-yl)-1,2,3,4-tetrahydrobenzo[4,5]imidazo[1,2-a]pyrazin-8-yl)methanol and
(S)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol

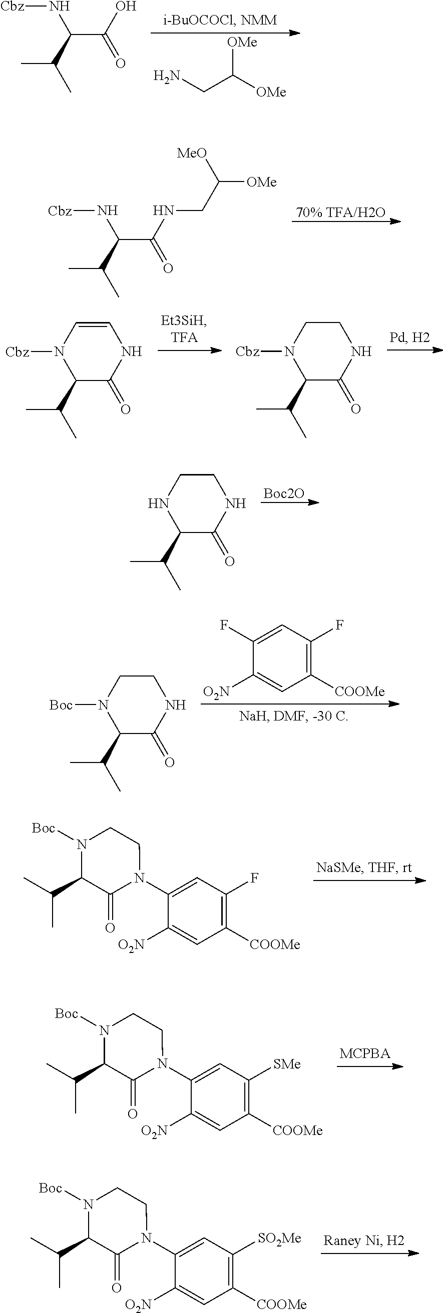

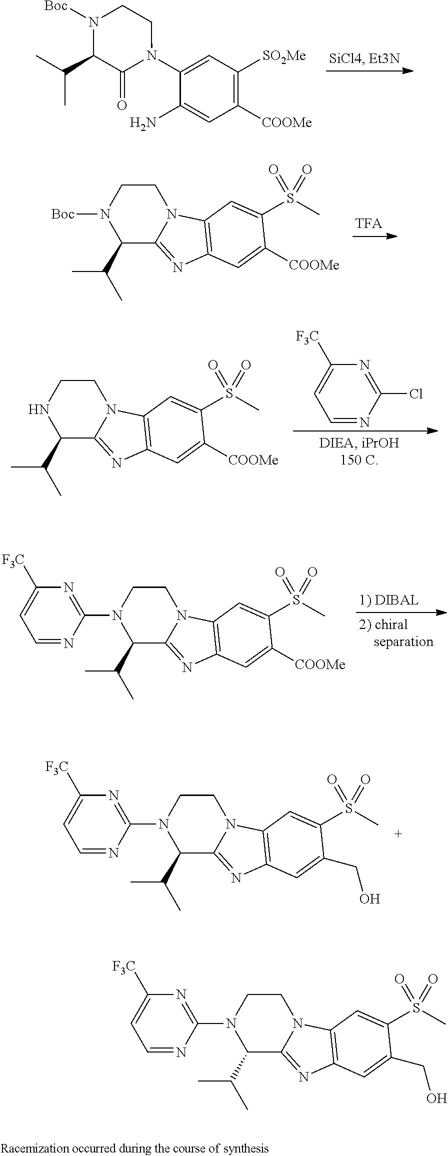

### Step 1:

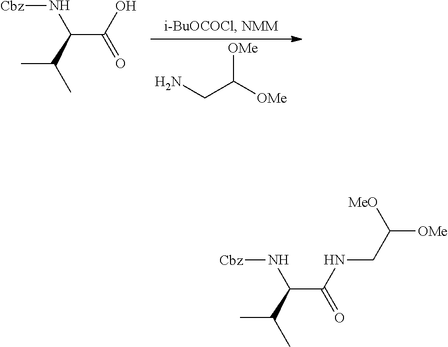 [SMILES: CC(C)[C@@H](NC(=O)OCc1ccccc1)C(=O)O ; COC(CN)OC ; COC(CNC(=O)[C@H](NC(=O)OCc1ccccc1)C(C)C)OC]

A solution of Cbz-D-Valine (500 g, 1.99 mol) and N-methylmorpholine (201.8 g, 1.99 mol) in anhydrous THF (8 L) was cooled to −15° C. and i-butyl chlorofomate (299 g, 2.19 mol) was added dropwise under stirring. After 30 min, a solution of 1-amino-2,2-dimethyoxyethane (209.5 g, 1.99 mol) in THF (1 L) was added slowly and the temperature was maintained at −15° C. for 2 h. The reaction mixture was washed with brine (2 L) and the organic phase was concentrated to remove the THF. The residue was diluted with EtOAc (4 L), washed with 1N aq HCl (2×2 L), sat. aq. NaHCO₃ (2 L) and sat. aq. Na₂CO₃ (2 L) and brine (1.5 L). After drying over Na₂SO₄, the organic solvent was removed under reduce pressure to afford (R)-benzyl (1-((2,2-dimethoxyethyl)amino)-3-methyl-1-oxobutan-2-yl)carbamate as a white solid (670 g, yield 99.5%), which was used for next step without further purification. LC-MS m/z 360.9 [M+Na]⁺¹H NMR (CD₃OD 300 MHz): δ 7.35-7.30 (m, 5H), 5.08 (s, 2H), 4.45-4.35 (m, 1H), 3.95-3.85 (m, 1H), 3.34-3.25 (m, 8H), 2.10-1.90 (m, 1H), 0.94-0.91 (m, 6H).

### Step 2:

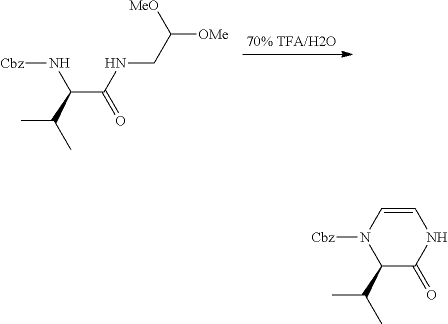 [SMILES: COC(CNC(=O)[C@H](NC(=O)OCc1ccccc1)C(C)C)OC ; CC(C)[C@@H]1C(=O)NC=CN1C(=O)OCc1ccccc1]

(R)-benzyl (1-((2,2-dimethoxyethyl)amino)-3-methyl-1-oxobutan-2-yl)carbamate (335 g, 0.99 mol) was added in portions to a cooled TFA-H₂O (temperature <5° C., V_{TFA}/V_{H20}=7/3, 2 L), and the solution was stirred at rt for 12 h. The solution was added slowly into stirring cooled sat. aq. Na₂CO₃ (2.5 L) to keep the pH >8. The mixture was extracted with EtOAc (5×2 L). The combined organic layers were washed with brine (2 L), dried over anhydrous Na₂SO₄, filtered and evaporated in vacuo to give (R)-benzyl 2-isopropyl-3-oxo-3,4-dihydropyrazine-1(2H)-carboxylate as a white solid (259 g, 95.4%), which was used for next step without further purification. LC-MS m/z 274.9 [M+H]⁺. ¹H NMR (CD₃OD 300 MHz): δ7.36-7.34 (m, 5H), 6.33-6.30 (m, 1H), 5.79-5.68 (m, 1H), 5.26-5.13 (m, 2H), 4.38-4.29 (m, 1H), 2.01-1.96 (m, 1H), 1.00-0.84 (m, 6H).

### Step 3:

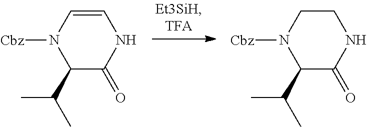 [SMILES: CC(C)[C@@H]1C(=O)NC=CN1C(=O)OCc1ccccc1 ; CC(C)[C@@H]1C(=O)NCCN1C(=O)OCc1ccccc1]

To a stirring solution of (R)-benzyl 2-isopropyl-3-oxo-3,4-dihydropyrazine-1(2H)-carboxylate (400 g, 1.46 mol) in DCE (2 L) was added Et₃SiH (424 g, 3.65 mol) and TFA (665 g, 5.8 mol) at rt. The reaction was stirred under reflux for 36 h. After cooling to rt, the solution was concentrated to remove the solvent. The residue was diluted with EtOAc (2 L), and added slowly into stirring cooled sat. aq. NaHCO₃ (2 L) to make sure that the pH >8. The mixture was extracted with EtOAc (2×2.5 L). The combined organic layers were washed with brine, dried over anhydrous Na₂SO₄, filtered and concentrated to give (R)-benzyl 2-isopropyl-3-oxopiperazine-1-carboxylate (402 g, yield 99.75%), which was used for next step without further purification. LC-MS m/z 276.9 [M+H]⁺. ¹H NMR (DMSO-d₆ 400 MHz): δ 7.93 (s, 1H), 7.39-7.31 (m, 5H), 5.09 (s, 2H), 4.06-4.01 (m, 1H), 3.99-3.92 (m, 1H), 3.23-3.14 (m, 3H), 2.20-2.12 (m, 1H), 0.96-0.94 (m, 3H), 0.85 (d, J=6.0 Hz, 3H).

### Step 4:

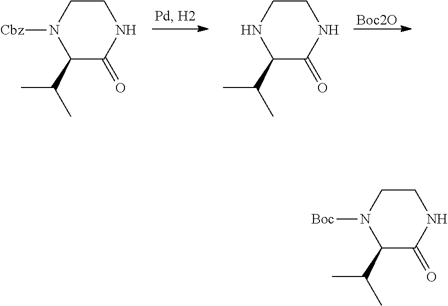

To a 1 L round-bottom flask containing (R)-benzyl 2-isopropyl-3-oxopiperazine-1-carboxylate (50 g, 0.181 mol) in MeOH (800 mL) was added Pd/C (dry, w/w 15%, 5 g). The mixture was stirred at rt under H₂ (1 atm) overnight. When TLC and LC-MS showed that the starting material was consumed, (Boc)₂O (76.74 g, 0.352 mol) was added to the reaction mixture, and the mixture was stirred at rt overnight until the intermediate (R)-3-isopropylpiperazin-2-one was consumed. The mixture was filtered and concentrated under vacuum. The residue was purified by column chromatography on silica gel (eluting with petroleum:EtOAc=3:1) to give (R)-tert-butyl 2-isopropyl-3-oxopiperazine-1-carboxylate as a white solid (26 g, yield 61%).

For (R)-3-isopropyl-piperazin-2-one:

LC-MS m/z 143.2 [M+H]⁺. ¹H NMR (HCl salt, CD₃OD 400 MHz): δ 3.95 (d, J=3.6 Hz, 1H), 3.65-3.39 (m, 4H), 2.63-2.54 (m, 1H), 1.15 (d, J=6.8 Hz, 3H), 1.09 (d, J=7.2 Hz, 3H).

For (R)-tert-butyl 2-isopropyl-3-oxopiperazine-1-carboxylate: LC-MS m/z 186.9 [M−56+H]⁺. ¹H NMR (DMSO-d₆ 400 MHz): δ 7.93 (s, 1H), 4.02-3.82 (m, 2H), 3.17-3.15 (m, 3H), 2.16 (s, 1H), 1.41 (s, 9H), 0.98 (d, J=6.8 Hz, 3H), 0.89 (d, J=6.4 Hz, 3H).

### Step 5:

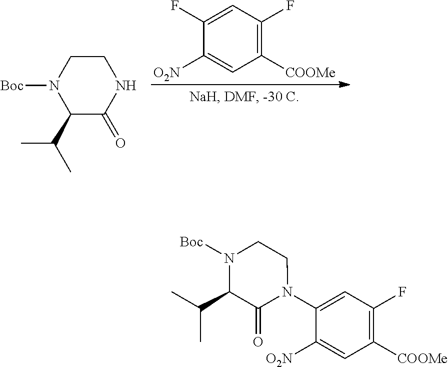 [SMILES: CC(C)[C@@H]1C(=O)NCCN1C(=O)OC(C)(C)C ; COC(=O)c1cc([N+](=O)[O-])c(F)cc1F ; COC(=O)c1cc([N+](=O)[O-])c(N2CCN(C(=O)OC(C)(C)C)[C@H](C(C)C)C2=O)cc1F]

Under N₂ atmosphere, NaH (8.8 g, 0.22 mol, 60% in mineral oil, 1.1 eq.) was added in portions at −10° C. to a 1 L three-neck flask containing (R)-tert-butyl 2-isopropyl-3-oxopiperazine-1-carboxylate (26.66 g, 0.11 mol) in DMF (300 mL). The mixture was stirred at −10° C. for 30 min. Then the mixture was added dropwise to a 1 L three-neck flask containing methyl 2,4-difluoro-5-nitrobenzoate (26.3 g, 0.121 mol, 1.1 eq.) in DMF (200 mL) at −20° C. over 10 min After addition, the resulting mixture was stirred between −20° C. and 30° C. for another 10 min. The reaction was quenched with sat. aq. NH₄Cl (200 mL) and then water (800 mL). The aqueous layer was extracted with EtOAc (3×1 L). The combined organic layers were washed with water (3×1 L) and brine, and dried over anhydrous Na₂SO₄. The mixture was filtered and the filtrate was evaporated under vacuum. The residue was purified by column chromatography on silica gel eluting with petroleum ether: EtOAc 8:1˜4:1 to give (R)-tert-butyl 4-(5-fluoro-4-(methoxycarbonyl)-2-nitrophenyl)-2-isopropyl-3-oxopiperazine-1-carboxylate (32 g, 66.3% yield) as a yellow solid. LC-MS MS (ESI) m/z 384.1 [M−56+H]⁺, 462.1[M+Na]⁺. ¹H NMR (CDCl₃ 300 MHz): δ 8.63 (d, J=6.9 Hz, 1H), 7.16 (d, J=10.2 Hz, 1H), 4.61-4.30 (m, 2H), 3.97-3.89 (m, 4H), 3.62-3.48 (m, 2H), 2.40-2.34 (m, 1H), 1.49 (s, 9H), 1.08 (d, J=6.9 Hz, 3H), 1.01 (d, J=6.9 Hz, 3H).

### Step 6.

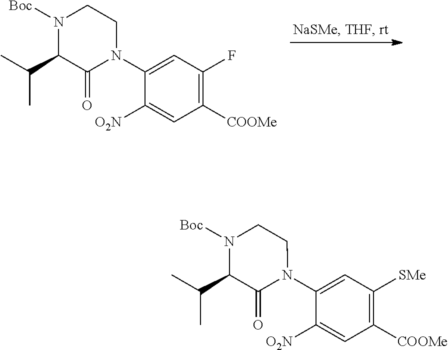 [SMILES: COC(=O)c1cc([N+](=O)[O-])c(N2CCN(C(=O)OC(C)(C)C)[C@H](C(C)C)C2=O)cc1F ; COC(=O)c1cc([N+](=O)[O-])c(N2CCN(C(=O)OC(C)(C)C)[C@H](C(C)C)C2=O)cc1SC]

To a 1 L round-bottom flask containing (R)-tert-butyl 4-(5-fluoro-4-(methoxycarbonyl)-2-nitrophenyl)-2-isopropyl-3-oxopiperazine-1-carboxylate was added NaSMe (14.3 g, 0.204 mmol, 3 eq.). The mixture was stirred at rt for 1 h. Water (500 mL) was added and the mixture was concentrated under vacuum to remove THF. The aqueous layer was extracted with EtOAc (3×800 mL). The combined organic layers were washed with brine, dried over anhydrous Na₂SO₄, filtered and concentrated under vacuum to give (R)-tert-butyl 2-isopropyl-4-(4-(methoxycarbonyl)-5-(methylthio)-2-nitrophenyl)-3-oxopiperazine-1-carboxylate (31.9 g, 100% yield) as a yellow solid. The residue was used directly for the next step without further purification. LC-MS MS (ESI) m/z 412.1 [M−56+H]⁺, 490.2[M+Na]⁺.

### Step 7:

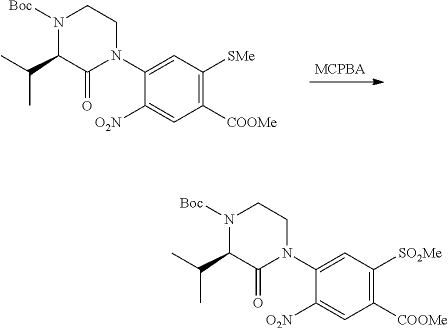 [SMILES: COC(=O)c1cc([N+](=O)[O-])c(N2CCN(C(=O)OC(C)(C)C)[C@H](C(C)C)C2=O)cc1SC ; COC(=O)c1cc([N+](=O)[O-])c(N2CCN(C(=O)OC(C)(C)C)[C@H](C(C)C)C2=O)cc1S(C)(=O)=O]

To a 2 L round-bottom flask containing (R)-tert-butyl 2-isopropyl-4-(4-(methoxycarbonyl)-5-(methylthio)-2-nitrophenyl)-3-oxopiperazine-1-carboxylate (crude 91.7 g, 0.196 mol) in CH₂Cl₂ (1 L) was added m-CPBA (84.6 g, 0.49 mmol, 2.5 eq). The mixture was stirred at rt overnight. Sat. Na₂S₂O₃ solution was added slowly to quench the reaction. The mixture was extracted with CH₂Cl₂ (4×3 L). The combined organic layers were washed successively with Na₂S₂O₃ solution (500 mL), NaHCO₃ solution (500 mL) and brine, dried over anhydrous Na₂SO₄, filtered and concentrated under vacuum. The residue was purified by column chromatography on silica gel eluting with dichloromethane to give (R)-tert-butyl 2-isopropyl-4-(4-(methoxycarbonyl)-5-(methylsulfonyl)-2-nitrophenyl)-3-oxopiperazine-1-carboxylate (83.7 g, 85.4% yield) as a yellow solid. LC-MS MS (ESI) m/z 444.0 [M−56+H]⁺, 522.1 [M+Na]⁺. ¹H NMR (CDCl₃ 300 MHz): δ 8.29 (s, 1H), 8.12 (s, 1H), 4.61-4.17 (m, 2H), 4.00-3.94 (m, 4H), 3.70-3.60 (m, 1H), 3.51-3.43 (m, 4H), 2.39-2.32 (m, 1H), 1.50 (s, 9H), 1.07 (d, J=6.9 Hz, 3H), 1.01 (d, J=6.9 Hz, 3H).

### Step 8:

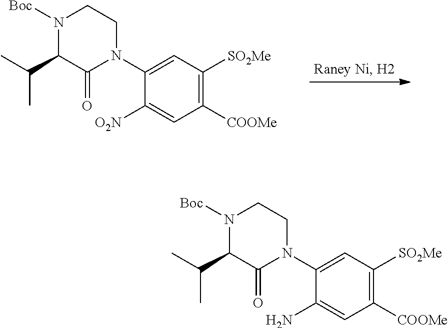 [SMILES: COC(=O)c1cc([N+](=O)[O-])c(N2CCN(C(=O)OC(C)(C)C)[C@H](C(C)C)C2=O)cc1S(C)(=O)=O ; COC(=O)c1cc(N)c(N2CCN(C(=O)OC(C)(C)C)[C@H](C(C)C)C2=O)cc1S(C)(=O)=O]

To a 1 L round-bottom flask containing (R)-tert-butyl 2-isopropyl-4-(4-(methoxycarbonyl)-5-(methylsulfonyl)-2-nitrophenyl)-3-oxopiperazine-1-carboxylate (26.3 g, 0.0526 mol) in THF (200 mL) and methanol (200 mL) was added Raney Nickel (in H₂O, 4 g). The mixture was stirred under H₂ (30 psi) at rt overnight. The mixture was filtered and concentrated under vacuum to give (R)-tert-butyl 4-(2-amino-4-(methoxycarbonyl)-5-(methylsulfonyephenyl)-2-isopropyl-3-oxopiperazine-1-carboxylate (24.7 g, 100% yield) as a yellow solid. The residue was used directly for the next step without further purification. LC-MS MS (ESI) m/z 414.0 [M−56+H]⁺, 492.0 [M+Na]⁺. ¹H NMR (CDCl₃ 300 MHz): δ 7.77 (brs, 1H), 7.04 (s, 1H), 4.68-4.45 (m, 1H), 4.45-4.38 (m, 2H), 3.92 (s, 3H), 3.70-3.58 (m, 1H), 3.58-3.41 (m, 1H), 3.30 (s, 3H), 2.49-2.25 (m, 1H), 1.50 (s, 9H), 1.12 (d, J=6.9 Hz, 3H), 1.05 (d, J=6.9 Hz, 3H).

### Step 9:

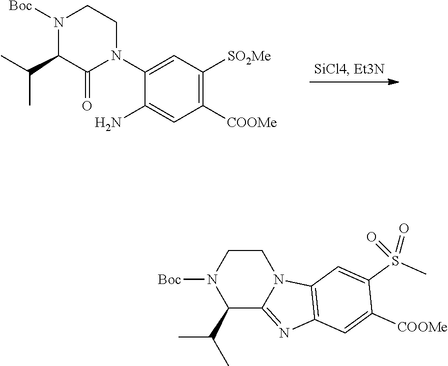 [SMILES: COC(=O)c1cc(N)c(N2CCN(C(=O)OC(C)(C)C)[C@H](C(C)C)C2=O)cc1S(C)(=O)=O ; COC(=O)c1cc2nc3n(c2cc1S(C)(=O)=O)CCN(C(=O)OC(C)(C)C)[C@@H]3C(C)C]

To a 1 L round-bottom flask containing (R)-tert-butyl 4-(2-amino-4-(methoxycarbonyl)-5-(methylsulfonyl)phenyl)-2-isopropyl-3-oxopiperazine-1-carboxylate (25 g, 0.0532 mol) in dichloromethane (500 mL) were added Et₃N (64.5 g, 0.638 mol, 12 eq.) and SiCl₄ (27.1 g, 0.160 mol, 3 eq.). The mixture was stirred at rt overnight. The mixture was added dropwise to aq. NaHCO₃ solution (54.1 g in 1 L of water, 0.644 mol, 12.1 eq.) at 0° C. slowly and adjusted to pH=8. The mixture was filtered and the aqueous layer was extracted with dichloromethane (3×600 mL). The combined organic layers were washed with brine, and then dried over anhydrous Na₂SO₄. The mixture was filtered and concentrated under vacuum to give the residue. The residue was purified by column chromatography on silica gel eluting with petroleum ether: EtOAc 2:1 to give (R)-2-tert-butyl 8-methyl 1-isopropyl-7-(methylsulfonyl)-3,4-dihydrobenzo[4,5]imidazo[1,2-a]pyrazine-2,8(1H)-dicarboxylate (13.2 g, 55% yield) as a pale yellow solid. Analytical chiral HPLC: t_{R}=9.03 min in 15 min chromatography (Method: OD-3_3_5_40_2.5 ML). LC-MS MS (ESI) m/z 452.2 [M+H]⁺. ¹H NMR (CD₃OD 400 MHz): δ 8.31 (s, 1H), 8.01 (s, 1H), 5.30-5.18 (m, 1H), 4.70-4.52 (m, 1H), 4.47 (dd, J=3.2 and 12.4 Hz, 1H), 4.18 (dt, J=5.2 and 11.6 Hz, 1H), 3.98 (s, 3H), 3.70-3.52 (m, 1H), 3.44 (s, 3H), 2.50-2.38 (m, 1H), 1.53 (s, 9H), 1.25 (d, J=6.8 Hz, 3H), 1.06 (d, J=6.8 Hz, 3H).

### Step 10:

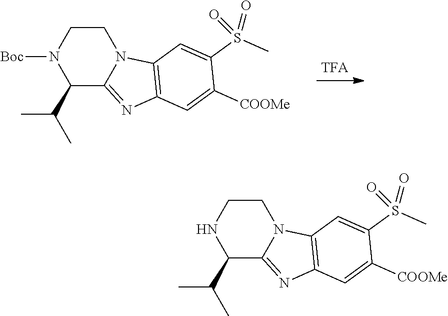

TFA (4 mL) was added dropwise to a solution of containing (R)-2-tert-butyl 8-methyl 1-isopropyl-7-(methylsulfonyl)-3,4-dihydrobenzo[4,5]imidazo[1,2-a]pyrazine-2,8(1H)-dicarboxylate (2.0 g, 4.4 mmol) in DCM (20 mL) at rt over 2 min. The mixture was stirred at rt for 3 h. TLC showed the starting material was consumed completely. The solvent was removed in vacuo at 30° C., and then DCM (10 mL) was added. The mixture was neutralized with sat. aq. NaHCO₃ to pH=7. The mixture was extracted with DCM (3×20 mL) and the combined organic layers were dried over anhydrous Na₂SO₄, filtered and concentrated under vacuum to afford (R)-methyl 1-isopropyl-7-(methylsulfonyl)-1,2,3,4-tetrahydrobenzo[4,5]imidazo[1,2-a]pyrazine-8-carboxylate (1.5 g, 96.4% yield) as a white solid. LC-MS m/z 351.9 [M+H]⁺, 374.0 [M+Na]⁺. ¹H NMR (CDCl₃ 300 MHz): δ 8.15 (s, 1H), 8.07 (s, 1H), 4.26-4.05 (m, 3H), 3.97 (s, 3H), 3.63-3.50 (m, 1H), 3.44 (s, 3H), 3.32-3.16 (m, 1H), 2.85-2.66 (m, 1H), 1.16 (d, J=6.9 Hz, 3H), 0.88 (d, J=6.6 Hz, 3H).

### Step 11:

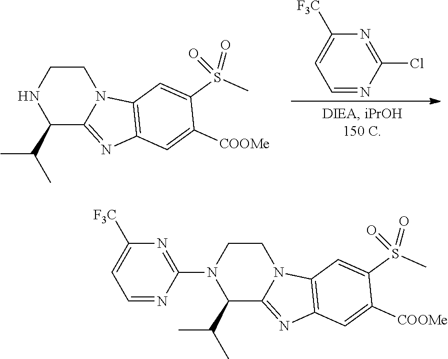 [SMILES: COC(=O)c1cc2nc3n(c2cc1S(C)(=O)=O)CCN[C@@H]3C(C)C ; FC(F)(F)c1ccnc(Cl)n1 ; COC(=O)c1cc2nc3n(c2cc1S(C)(=O)=O)CCN(c1nccc(C(F)(F)F)n1)[C@@H]3C(C)C]

A mixture of (R)-methyl 1-isopropyl-7-(methylsulfonyl)-1,2,3,4-tetrahydrobenzo[4,5]imidazo[1,2-a]pyrazine-8-carboxylate (0.9 g, 2.56 mmol), 2-chloro-4-(trifluoromethyl)pyrimidine (1.0 g, 5.1 mmol, 2 eq.) and DIEA (1.0 g, 7.7 mmol, 3 eq.) in i-PrOH (6 mL) was stirred in a microwave oven at 150° C. for 2 h. TLC showed the starting material was consumed completely (PE:EtOAc=3:1). The solvent was removed in vacuo at 40° C., and the residue was purified by column chromatography on silica gel eluting with PE/EtOAc=6/1 to give (R)-methyl 1-isopropyl-7-(methylsulfonyl)-2-(4-(trifluoromethyl)pyrimidin-2-yl)-1,2,3,4-tetrahydrobenzo[4,5]imidazo[1,2-a]pyrazine-8-carboxylate (1.0 g, 78% yield) as a white solid. LC-MS m/z 498.1 [M+H]⁺. ¹H NMR (CDCl₃ 300 MHz): δ 8.52 (d, J=4.8 Hz, 1H), 8.13 (s, 1H), 8.06 (s, 1H), 6.83 (d, J=4.8 Hz, 1H), 6.06 (d, J=7.8 Hz, 1H), 5.39-5.28 (m, 1H), 4.33-4.24 (m, 1H), 4.20-4.12 (m, 1H), 3.93 (s, 3H), 3.77-3.65 (m, 1H), 3.39 (s, 3H), 2.52-2.38 (m, 1H), 1.25 (d, J=6.9 Hz, 3H), 1.02 (d, J=6.6 Hz, 3H).

### Step 12:

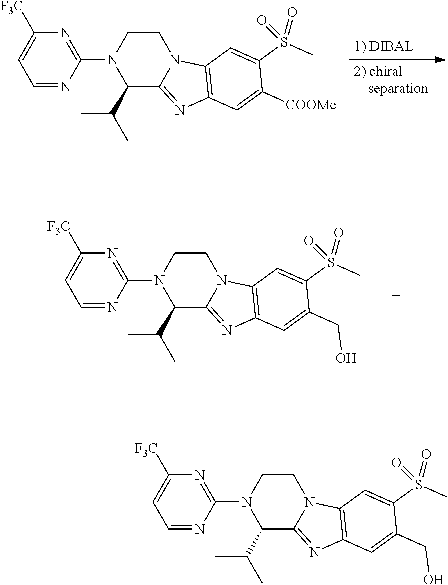 [SMILES: COC(=O)c1cc2nc3n(c2cc1S(C)(=O)=O)CCN(c1nccc(C(F)(F)F)n1)[C@@H]3C(C)C ; CC(C)[C@@H]1c2nc3cc(CO)c(S(C)(=O)=O)cc3n2CCN1c1nccc(C(F)(F)F)n1 ; CC(C)[C@H]1c2nc3cc(CO)c(S(C)(=O)=O)cc3n2CCN1c1nccc(C(F)(F)F)n1]

To a solution of (R)-methyl 1-isopropyl-7-(methylsulfonyl)-2-(4-(trifluoromethyl)pyrimidin-2-yl)-1,2,3,4-tetrahydrobenzo[4,5]imidazo[1,2-a]pyrazine-8-carboxylate (1.3 g, 2.6 mmol) in DCM (15 mL) was added DIBAL-H (1M in toluene, 10.4 mL, 10.4 mmol, 4 eq.) at −78° C. The mixture was stirred at −78° C. for 2 h. Sat. aq NH₄Cl (25 mL) was added and the mixture was filtered. The aqueous layer was extracted with DCM (3×20 mL). The combined organic layers were washed with brine, dried over anhydrous Na₂SO₄, filtered and concentrated under vacuum. The residue was purified by column chromatography on silica gel eluting with DCM/MeOH=30/1 to give a partially racemized mixture (1.1 g, 91.6% yield) as a white solid. The racemized mixture was purified by SFC separation on a chiral column to give (R)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol (Isomer 1) (0.65 g, 54.1% yield) as a white solid and (S)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol (Isomer 2) (0.15 g, 12.5% yield) as a white solid.

Isomer 1: (R)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol: Analytical chiral HPLC: t_{R}=8.768 min in 15 min chromatography (Method: AD-H_5_5_40_2.35 ML). LC-MS m/z 470.1 [M+H]⁺. ¹H NMR (CDCl₃ 400 MHz): δ 8.58 (d, J=4.8 Hz, 1H), 8.13 (s, 1H), 7.89 (s, 1H), 6.89 (d, J=4.8 Hz, 1H), 6.12 (d, J=8.0 Hz, 1H), 5.40-5.36 (m, 1H), 5.06-5.03 (m, 2H), 4.35-4.31 (m, 1H), 4.21-4.16 (m, 1H), 3.82-3.76 (m, 1H), 3.23 (s, 3H), 3.09 (t, J=6.8 Hz, 1H), 2.52-2.50 (m, 1H), 1.32 (d, J=6.8 Hz, 3H), 1.08 (d, J=6.8 Hz, 3H). ¹H NMR (CD₃OD 400 MHz): δ 8.69 (d, J=4.8 Hz, 1H), 8.22 (s, 1H), 7.94 (s, 1H), 7.02 (d, J=4.8 Hz, 1H), 6.05 (d, J=8.0 Hz, 1H), 5.34 (d, J=10.0 Hz, 1H), 5.10 (s, 2H), 4.50 (dd, J₁=12.0 Hz, J₂=3.6 Hz, 1H), 4.22 (td, J₁=12.0 Hz, J₂=5.2 Hz, 1H), 3.88 (dddd, J₁=14.4 Hz, J₂=10.0 Hz, J₃=4.4 Hz, 1H), 3.26 (s, 3H), 2.60-2.52 (m, 1H), 1.28 (d, J=6.8 Hz, 3H), 1.06 (d, J=6.8 Hz, 3H).

Isomer 1 was recrystalized as a crystalline solid by following procedure:

(R)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol (470 mg) was dissolved into EtOAc (3.0 mL) followed by slow addition of hexanes (ca. 5 mL) until the solution turns cloudy. Several drops of EtOAc were added to cause the cloudiness to disappear. The solution was allowed to stand at rt until crystals formed. The crystalline solid was collected by filtration. m.p. 188-189° C.

Isomer 2: (S)-(1-isopropyl-7-(methylsulfonyl)-2-(4-(trifluoromethyl)pyrimidin-2-yl)-1,2,3,4-tetrahydrobenzo[4,5]imidazo[1,2-a]pyrazin-8-yl)methanol:

Analytical chiral HPLC: t_{R}=7.780 min in 15 min chromatography (Method: AD-H_5_5_40_2.35 ML). LC-MS m/z 470.1 [M+H]⁺. ¹H NMR (CDCl₃ 400 MHz): δ 8.58 (d, J=5.2 Hz, 1H), 8.12 (s, 1H), 7.88 (s, 1H), 6.89 (d, J=4.8 Hz, 1H), 6.11 (d, J=8.0 Hz, 1H), 5.40-5.35 (m, 1H), 5.04-5.00 (m, 2H), 4.34-4.31 (m, 1H), 4.21-4.16 (m, 1H), 3.82-3.75 (m, 1H), 3.22 (s, 3H), 2.52-2.50 (m, 1H), 1.31 (d, J=6.8 Hz, 3H), 1.08 (d, J=6.8 Hz, 3H).

Alternatively, a racemic mixture of methyl 1-isopropyl-7-(methylsulfonyl)-2-(4-(trifluoromethyl)pyrimidin-2-yl)-1,2,3,4-tetrahydrobenzo[4,5]imidazo[1,2-a]pyrazine-8-carboxylate was prepared by the following method.

### (rac)-methyl 1-isopropyl-7-(methylsulfonyl)-2-(4-(trifluoromethyl)pyrimidin-2-yl)-1,2,3,4-tetrahydrobenzo[4,5]imidazo[1,2-a]pyrazine-8-carboxylate

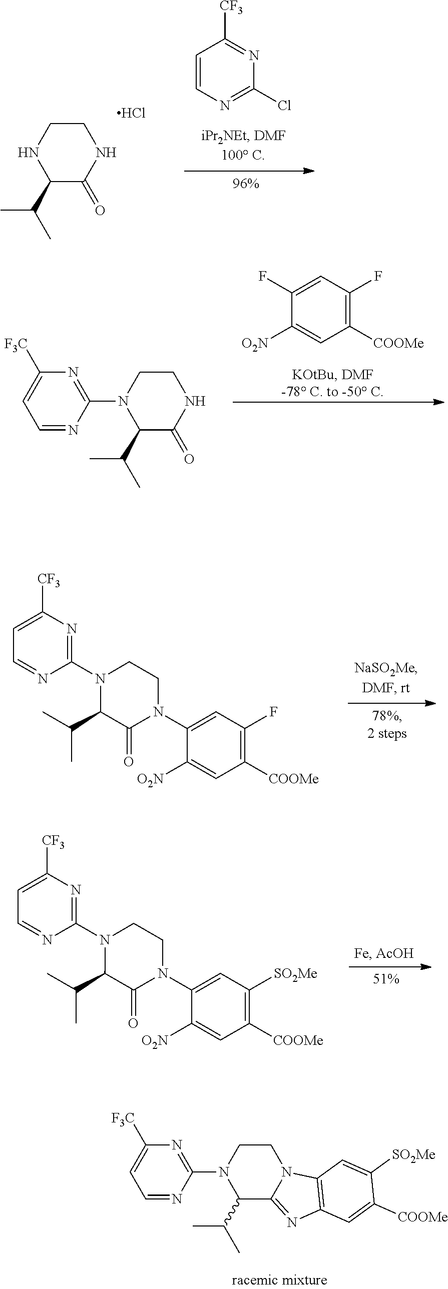

### Step 1:

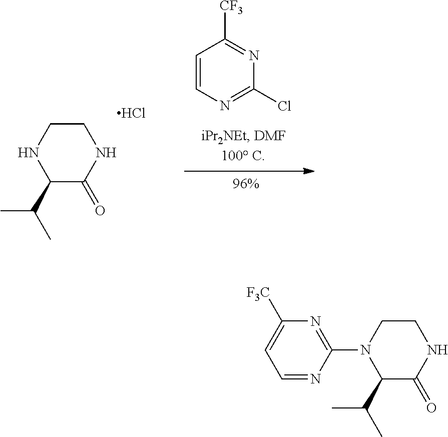

To a solution of (R)-3-isopropylpiperazin-2-one hydrochloride (2.61 g, 14.62 mmol) and iPr₂NEt (7.60 mL, 43.86 mmol) in DMF (20 mL) was added a solution of 2-chloro-4-(trifluoromethyl)pyrimidine (3.47 g, 19.00 mmol) in DMF (2 mL). The resulting solution was stirred at 100° C. under N₂ for 3 h at which point the reaction was deemed complete by LC-MS. Sat. aq. NH₄Cl (30 mL) was added to quench the reaction, followed by addition of EtOAc (30 mL). The EtOAc layer was separated and the aqueous layer was extracted with EtOAc (3×20 mL). The EtOAc layers were combined, dried using Na₂SO₄ and evaporated to give nearly pure crude product. Purification on a silica cartridge using ISCO FCC eluting with 100% EtOAc gave 4.03 grams of (R)-3-isopropyl-4-(4-(trifluoromethyl)pyrimidin-2-yl)piperazin-2-one (96%) as a slightly orange thick oil. LC-MS m/z 289.17 [M+H]⁺. ¹H NMR (CDCl₃, 400 MHz): δ 8.52 (d, J=4.8 Hz, 1H), 6.82 (d, J=4.4 Hz, 1H), 6.56 (br, 1H), 5.20 (d, J=6.8 Hz, 1H), 4.83-4.77 (m, 1H), 3.55-3.37 (m, 3H), 2.49-2.41 (m, 1H), 1.15 (d, J=6.8 Hz, 3H), 1.04 (d, J=6.8 Hz, 3H).

### Steps 2 and 3:

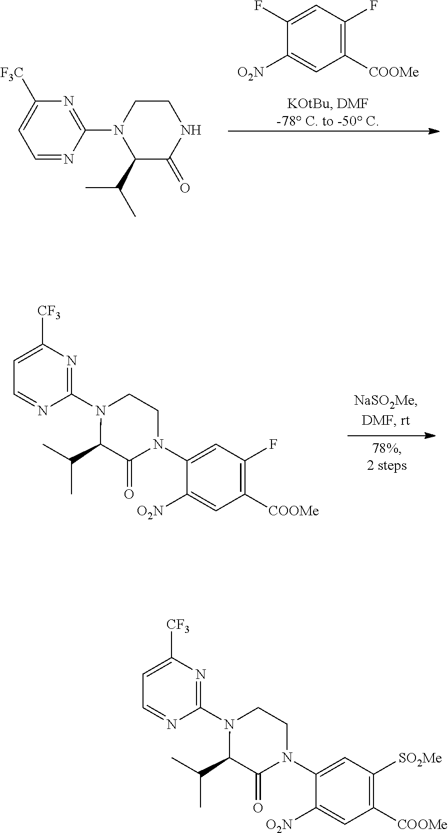 [SMILES: COC(=O)c1cc([N+](=O)[O-])c(F)cc1F ; CC(C)[C@@H]1C(=O)NCCN1c1nccc(C(F)(F)F)n1 ; COC(=O)c1cc([N+](=O)[O-])c(N2CCN(c3nccc(C(F)(F)F)n3)[C@H](C(C)C)C2=O)cc1F ; COC(=O)c1cc([N+](=O)[O-])c(N2CCN(c3nccc(C(F)(F)F)n3)[C@H](C(C)C)C2=O)cc1S(C)(=O)=O]

To a solution of (R)-3-isopropyl-4-(4-(trifluoromethyl)pyrimidin-2-yl)piperazin-2-one (986 mg, 3.42 mmol) in DMF (5 mL) was added a 2 M solution of KOtBu in THF (2.14 mL, 4.28 mmol) dropwise at 0° C. The reaction stirred for 1 h at 0° C. and was then cooled to −78° C. In a separate flask, a solution of methyl 2,4-difluoro-5-nitrobenzoate (928 mg, 4.28 mmol) in DMF (15 mL) was cooled to −78° C. To this solution was added a solution of the above anion via cannula at −78° C. over a 5 min period. The reaction was allowed to warm to −50° C. and stirred at this temperature for 2 h. Sat. aq. NH₄Cl (20 mL) was added to quench the reaction, followed by EtOAc (30 mL). The EtOAc layer was separated and the aqueous layer was extracted with EtOAc (3×15 mL). The EtOAc layers were combined, dried and evaporated to give crude (R)-methyl-2-fluoro-4-(3-isopropyl-2-oxo-4-(4-(trifluoromethyl)pyrimidin-2-yl)piperazin-1-yl)-5-nitrobenzoate which was taken on directly for the next step without further purification.

LC-MS m/z 486.20 [M+H]⁺.

To a solution of the above crude (R)-methyl 2-fluoro-4-(3-isopropyl-2-oxo-4-(4-(trifluoromethyl)pyrimidin-2-yl)piperazin-1-yl)-5-nitrobenzoate in DMF (15 mL) was added NaSO₂Me (1.05 g, 10.30 mmol) in one portion at rt. After stirring for 3 h, the reaction was deemed complete by LC-MS analysis. Water (100 mL) was added and the mixture stirred vigorously for 20 minutes before filtering off the solid material. To this solid material was added 20% EtOAc in Hexanes and the mixture stirred vigorously for 10 minutes. The EtOAc/Hexanes filtrate was collected and evaporated to give 1.45 g of (R)-methyl 4-(3-isopropyl-2-oxo-4-(4-(trifluoromethyl)pyrimidin-2-yl)piperazin-1-yl)-2-(methylsulfonyl)-5-nitrobenzoate as an off-white solid (78%, 2 steps). LC-MS m/z 546.27[M+H]⁺. ¹H NMR (CDCl₃, 400 MHz): δ 8.59 (d, J=4.4 Hz, 1H), 8.32 (s, 1H), 8.14 (s, 1H), 6.92 (d, J=5.2 Hz, 1H), 5.32 (d, J=6.8 Hz, 1H), 5.04-5.00 (m, 1H), 4.12-4.02 (m, 1H), 4.03 (s, 3H), 3.88-3.80 (m, 2H), 3.44 (s, 3H), 2.55-2.50 (m, 1H), 1.15 (d, J=6.8 Hz, 3H), 1.06 (d, J=6.8 Hz, 3H).

### Step 4:

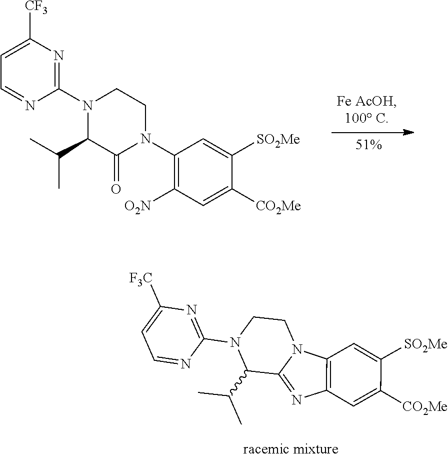 [SMILES: COC(=O)c1cc([N+](=O)[O-])c(N2CCN(c3nccc(C(F)(F)F)n3)[C@H](C(C)C)C2=O)cc1S(C)(=O)=O ; COC(=O)c1cc2nc3n(c2cc1S(C)(=O)=O)CCN(c1nccc(C(F)(F)F)n1)C3C(C)C]

To a solution of (R)-methyl-4-(3-isopropyl-2-oxo-4-(4-(trifluoromethyl)pyrimidin-2-yl)piperazin-1-yl)-2-(methylsulfonyl)-5-nitrobenzoate (1.45 g, 2.66 mmol) in glacial acetic acid (17 mL) was added iron powder (445 mg, 7.97 mmol). The mixture was heated to 100° C. After 5 min, the suspended iron dissolved into solution. The mixture was stirred at 100° C. for 48 h, at which point the flask was cooled to rt and the contents were poured into ice. The mixture was extracted with EtOAc (2×75 mL), then the combined organic layers were washed with water (2×50 mL) and brine (50 mL). The solution was dried over MgSO₄, filtered through cotton, and concentrated in vacuo. The residue was purified on a silica cartridge (0% EtOAc in hexanes, then 50%) to yield 680 mg of methyl-1-isopropyl-7-(methylsulfonyl)-2-(4-(trifluoromethyl)pyrimidin-2-yl)-1,2,3,4-tetrahydrobenzo[4,5]imidazo[1,2-a]pyrazine-8-carboxylate as a racemic mixture (51%). LC-MS: m/z 498.32 (M+H]⁺. ¹H NMR (CDCl₃, 400 MHz): δ 8.58 (d, J=4.8 Hz, 1H), 8.20 (s, 1H), 8.12 (s, 1H), 6.90 (d, J=4.8 Hz, 1H), 6.13 (d, J=8.4 Hz, 1H), 5.39 (dd, J=4.8 Hz, 14.4 Hz, 1H), 4.35 (ddd, J=1.2 Hz, 4.4 Hz, 12.0 Hz, 1H), 4.21 (dt, J=4.8 Hz, 12.0 Hz, 1H), 4.00 (s, 3H), 3.78 (ddd, J=4.4 Hz, 11.6 Hz, 14.4 Hz, 1H), 3.45 (s, 3H), 2.48 (sept, J=7.2 Hz, 1H), 1.32 (d, J=6.4 Hz, 3H), 1.08 (d, J=6.8 Hz, 3H).

### Example 33

### (R)-1-isopropyl-7-(methylsulfonyl)-2-(4-(trifluoromethyl)pyrimidin-2-yl)-1,2,3,4-tetrahydropyrazino[1,2-a]indole and

### (S)-1-isopropyl-7-(methylsulfonyl)-2-(4-(trifluoromethyl)pyrimidin-2-yl)-1,2,3,4-tetrahydropyrazino[1,2-a]indole

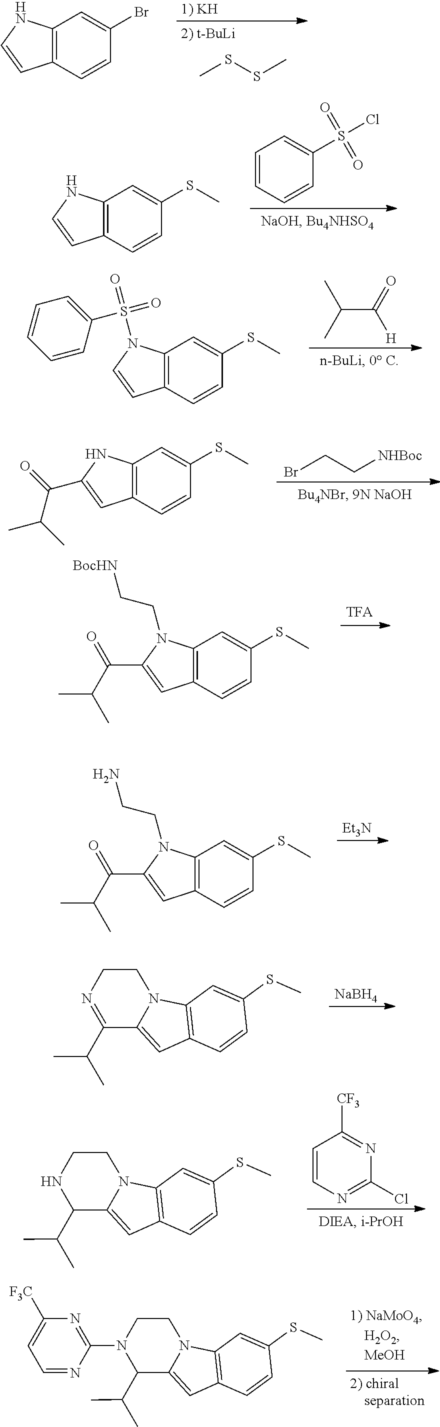

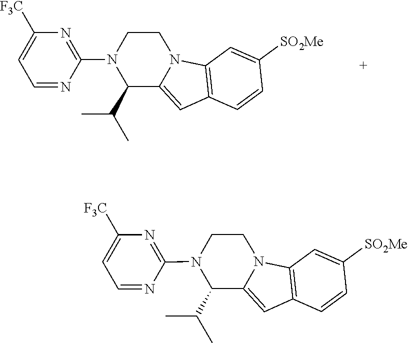 [SMILES: CC(C)[C@@H]1c2cc3ccc(S(C)(=O)=O)cc3n2CCN1c1nccc(C(F)(F)F)n1 ; CC(C)[C@H]1c2cc3ccc(S(C)(=O)=O)cc3n2CCN1c1nccc(C(F)(F)F)n1]

### Step 1:

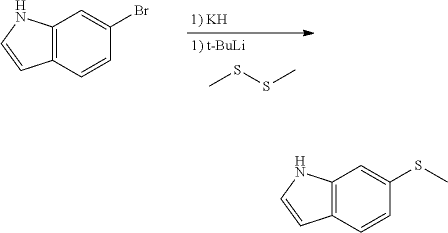

To a solution of 6-bromo-1H-indole (5 g, 25.50 mmol) in anhydrous THF (60 mL) at 0° C. was added KH (6.80 g, 51.00 mmol, 30% wt in mineral oil). After stirring for 30 min, the mixture was cooled to −78° C. and t-BuLi (39.23 mL, 51.0 mmol, 1.3 M) was added under nitrogen. After 30 min, 1,2-dimethyldisulfane (4.80 g, 51.0 mmol) was added to the mixture. The reaction mixture was stirred at −78° C. for 1 h and quenched with sat. aq NH₄Cl (30 mL) at −78° C. slowly (Caution: flame), adjusted pH=7 with 1 N aqueous phosphoric acid and extracted with EtOAc (50 mL×3). The combined organic layers were dried over anhydrous Na₂SO₄, filtered, concentrated and purified by column chromatography on silica gel eluted with (petroleum ether/EtOAc 10:1) to give 6-(methylthio)-1H-indole (3.9 g, 93.67% yield) as a grey solid. LC-MS MS (ESI) m/z 164.1 [M+H]⁺. ¹H NMR (CDCl₃ 400 MHz): δ 8.14 (brs, 1H), 7.56 (d, J=8.0 Hz, 1H), 7.37 (s, 1H), 7.18-7.11 (m, 1H), 6.56-6.51 (m, 1H), 2.52 (s, 3H).

### Step 2:

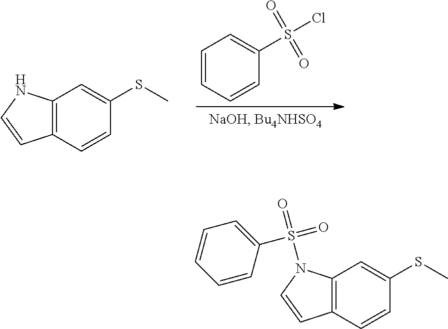

To a solution of 6-(methylthio)-1H-indole (1 g, 6.13 mmol), NaOH (4.90 g, 122.6 mmol) and Bu₄NHSO₄ (207.8 mg, 0.613 mmol) in dichloromethane (20 mL) was added benzenesulfonyl chloride (1.29 g, 7.36 mmol). The reaction mixture was stirred at rt overnight. The mixture was quenched with water (30 mL) and extracted with CH₂Cl₂ (30 mL×3). The combined organic layers were dried over anhydrous Na₂SO₄, filtered, concentrated and purified by column chromatography on silica gel eluted with (petroleum ether/EtOAc 10:1) to afford 6-(methylthio)-1-(phenylsulfonyl)-1H-indole (1.1 g, 59.18% yield) as a white solid. LC-MS MS (ESI) m/z 304.0 [M+H]⁺. ¹H NMR (CDCl₃ 400 MHz): δ 7.93-7.75 (m, 3H), 7.58-7.41 (m, 5H), 7.17 (dd, J₁=8.0 Hz, J₂=1.6 Hz, 1H), 6.63-6.60 (m, 1H), 2.53 (s, 3H).

### Step 3:

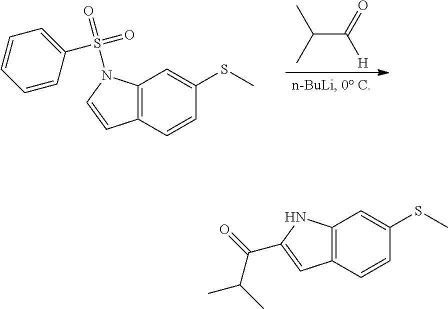

To a solution of 6-(methylthio)-1-(phenylsulfonyl)-1H-indole (890 mg, 2.93 mmol) in anhydrous THF (10 mL) at 0° C. under nitrogen was added n-BuLi (5.86 mL, 14.65 mmol, 2.5 M). After stirring for 30 min, isobutyraldehyde (1.05 g, 14.65 mmol) was added. The reaction mixture was stirred at 0° C. for 1 h and quenched with sat. aq NH₄Cl (10 mL) at 0° C. and extracted with EtOAc (20 mL×3). The combined organic layers were dried over anhydrous sodium sulfate, filtered, concentrated and purified by column chromatography on silica gel eluted with (petroleum ether/EtOAc 20:1) to give 2-methyl-1-(6-(methylthio)-1H-indol-2-yl)propan-1-one (440 mg, 64.28% yield) as a colorless oil.

LC-MS MS (ESI) m/z 234.1 [M+H]⁺. ¹H NMR (CDCl₃ 400 MHz): δ 8.86 (brs, 1H), 7.52 (d, J=8.4 Hz, 1H), 7.19 (s, 1H), 7.14-7.11 (m, 1H), 7.01 (dd, J₁=8.4 Hz, J₂=1.6, 1H), 3.42-3.38 (m, 1H), 2.47 (s, 3H), 1.20 (d, J=6.8 Hz, 6H).

### Step 4:

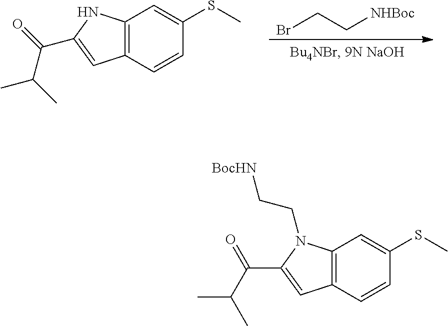 [SMILES: CSc1ccc2cc(C(=O)C(C)C)[nH]c2c1 ; CC(C)(C)OC(=O)NCCBr ; CSc1ccc2cc(C(=O)C(C)C)n(CCNC(=O)OC(C)(C)C)c2c1]

To a solution of 2-methyl-1-(6-(methylthio)-1H-indol-2-yl)propan-1-one (600 mg, 2.57 mmol) and Bu₄NBr (4.12 g, 12.85 mmol) in 9 N NaOH (10 mL, cooled) was added tert-butyl (2-bromoethyl)carbamate (2.87 g, 12.85 mmol). The reaction mixture was stirred at rt for 72 h. The mixture was diluted with water (20 mL) at 0° C. and extracted with EtOAc (20 mL×3). The combined organic layers were dried over anhydrous Na₂SO₄, filtered, concentrated and purified by column chromatography on silica gel eluting with (petroleum ether/EtOAc 10:1) to afford tert-butyl (2-(2-isobutyryl-6-(methylthio)-1H-indol-1-yl)ethyl)carbamate (200 mg, 20.66% yield) as a colorless oil. LC-MS MS (ESI) m/z 321.1 [M−56+H]⁺, 277.1 [M−100+H]⁺. ¹H NMR (CDCl₃ 400 MHz): δ 7.57 (d, J=8.4 Hz, 1H), 7.38 (s, 1H), 7.29 (s, 1H), 7.10 (d, J=8.4 Hz, 1H), 4.80 (brs, 1H), 4.62 (t, J=6.4 Hz, 2H), 3.58-3.42 (m, 3H), 2.58 (s, 3H), 1.38 (s, 9H), 1.24 (d, J=6.8 Hz, 6H).

### Step 5:

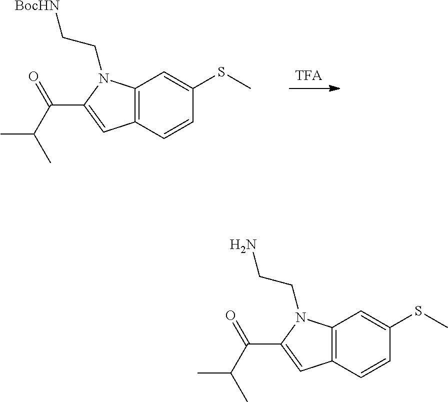 [SMILES: CSc1ccc2cc(C(=O)C(C)C)n(CCNC(=O)OC(C)(C)C)c2c1 ; CSc1ccc2cc(C(=O)C(C)C)n(CCN)c2c1]

To a solution of tert-butyl (2-(2-isobutyryl-6-(methylthio)-1H-indol-1-yl)ethyl)carbamate (200 mg, 0.53 mmol) in CH₂Cl₂ (9 mL) at 0° C. was added TFA (1 mL). The reaction mixture was stirred at rt for 1 h. The mixture was concentrated (T<25° C.), treated with water (5 mL), adjusted to pH=11 with sat. NaHCO₃ and extracted with EtOAc (20 mL×3). The combined organic layers were dried over anhydrous Na₂SO₄, filtered, concentrated to afford 1-(1-(2-aminoethyl)-6-(methylthio)-1H-indol-2-yl)-2-methylpropan-1-one (210 mg, 100% yield) as a colorless oil. LC-MS MS (ESI) m/z 258.8 [M−18+H]⁺.

### Step 6:

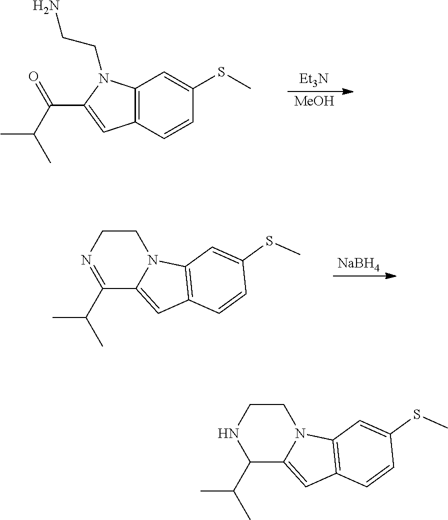

To a solution of 1-(1-(2-aminoethyl)-6-(methylthio)-1H-indol-2-yl)-2-methylpropan-1-one (200 mg, 0.724 mmol) in MeOH (5 mL) was added Et₃N (219.3 mg, 2.172 mmol). The reaction mixture was stirred at 60° C. for 1 h. NaBH₄ (82.53 mg, 2.172 mmol) was added. The mixture was stirred at 60° C. for 1 h. The mixture was concentrated, treated with water (10 mL) and extracted with EtOAc (20 mL×3). The combined organic layers were dried over anhydrous Na₂SO₄, filtered, concentrated and purified by preparative TLC on silica gel eluted with (petroleum ether/EtOAc 1:1) to afford 1-isopropyl-7-(methylthio)-1,2,3,4-tetrahydropyrazino[1,2-a]indole (80 mg, 42.46% yield, store at 0° C.) as a colorless oil.

LC-MS of 1-Isopropyl-7-methylsulfanyl-3,4-dihydro-pyrazino[1,2-a]indole MS (ESI) m/z 259.1 [M+H]⁺. LC-MS of 1-isopropyl-7-(methylthio)-1,2,3,4-tetrahydropyrazino[1,2-a]indole MS (ESI) m/z 261.2 [M+H]⁺. ¹H NMR (CDCl₃ 400 MHz): δ 7.41 (d, J=8.4 Hz, 1H), 7.20 (s, 1H), 7.05 (dd, J₁=8.4 Hz, J₂=1.6 Hz, 1H), 6.12 (s, 1H), 4.02-3.97 (m, 2H), 3.86-3.80 (m, 1H), 3.46-3.42 (m, 1H), 3.16-3.10 (m, 1H), 2.48 (s, 3H), 2.32-2.27 (m, 1H), 1.09 (d, J=6.8 Hz, 3H), 0.86 (d, J=6.8 Hz, 3H).

### Step 7:

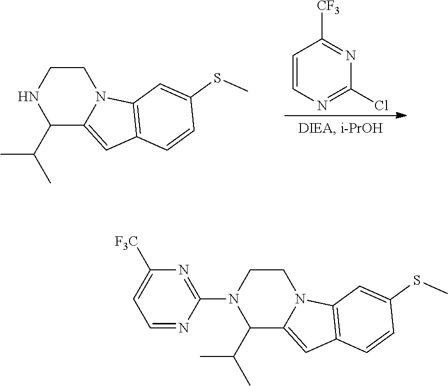

To a solution of 1-isopropyl-7-(methylthio)-1,2,3,4-tetrahydropyrazino[1,2-a]indole (50 mg, 0.19 mmol) in ⁱPrOH (2 mL) was added 2-chloro-4-(trifluoromethyl)pyrimidine (105 mg, 0.58 mmol) and DIEA (185 mg, 0.96 mmol). The mixture was stirred at 100° C. for 4 h. The mixture was concentrated under vacuum and the residue was purified by preparative TLC to afford 1-isopropyl-7-(methylthio)-2-(4-(trifluoromethyl)pyrimidin-2-yl)-1,2,3,4-tetrahydropyrazino[1,2-a]indole (45 mg, 57.7% yield) as a yellow oil. LC-MS MS (ESI) m/z 407.1[M+H]⁺.

### Step 8:

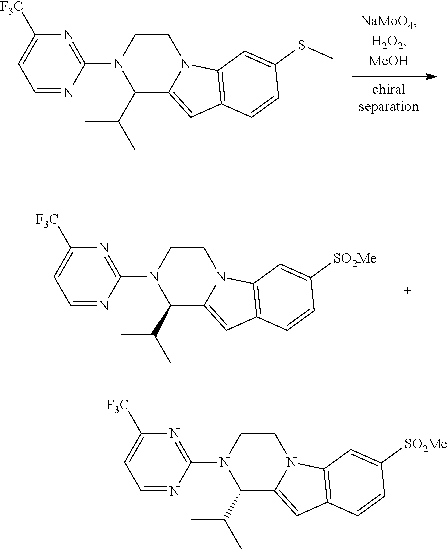 [SMILES: CSc1ccc2cc3n(c2c1)CCN(c1nccc(C(F)(F)F)n1)C3C(C)C ; CC(C)[C@@H]1c2cc3ccc(S(C)(=O)=O)cc3n2CCN1c1nccc(C(F)(F)F)n1 ; CC(C)[C@H]1c2cc3ccc(S(C)(=O)=O)cc3n2CCN1c1nccc(C(F)(F)F)n1]

To a solution of 1-isopropyl-7-(methylthio)-1,2,3,4-tetrahydropyrazino[1,2-a]indole (45 mg, 0.11 mmol) in MeOH (1 mL). was added NaMoO₄-2H₂O (61 mg, 0.33 mmol) and 30% H₂O₂ (67 mg, 0.55 mmol) at 0° C. The mixture was stirred at rt for 2 h. Sat. Na₂S₂O₃ (5 mL) was added and the mixture was concentrated under vacuum. The aqueous layer was extracted with EtOAc (3×10 mL). The combined organic layers were washed with brine, dried over anhydrous Na₂SO₄, filtered and concentrated under vacuum. The residue was purified by preparative TLC and SFC separation on a chiral column to afford isomer 1 (20.10 mg, 46.6% yield) as a white solid and isomer 2 (20.30 mg, 47.1% yield) as a white solid.

Isomer 1: Analytical chiral HPLC: t_{R}=6.64 min in 15 min chromatography (Method: AD-H_5_5_40_2.35 ML). LC-MS MS (ESI) m/z 439.0 [M+H]⁺. ¹H NMR (CD₃OD 300 MHz): δ 8.63 (d, J=4.8 Hz, 1H), 7.98 (s, 1H), 7.69 (d, J=8.4 Hz, 1H), 7.57 (dd, J=1.5 and 8.4 Hz, 1H), 6.94 (d, J=4.8 Hz, 1H), 6.50 (s, 1H), 5.88 (d, J=8.4 Hz, 1H), 5.11-5.07 (m, 1H), 4.45-4.38 (m, 1H), 4.05 (dt, J=4.8 and 11.4 Hz, 1H), 3.91-3.83 (m, 1H), 3.11 (s, 3H), 2.35-2.27 (m, 1H), 1.14 (d, J=6.6 Hz, 3H), 1.01 (d, J=6.6 Hz, 3H).

Isomer 2: Analytical chiral HPLC: t_{R}=7.37 min in 15 min chromatography (Method: AD-H_5_5_40_2.35 ML). LC-MS MS (ESI) m/z 439.0 [M+H]⁺, 461.0 [M+Na]⁺. ¹H NMR (CD₃OD 300 MHz): δ 8.63 (d, J=4.5 Hz, 1H), 7.98 (s, 1H), 7.68 (d, J=8.4 Hz, 1H), 7.57 (dd, J=1.5 and 8.4 Hz, 1H), 6.94 (d, J=4.8 Hz, 1H), 6.49 (s, 1H), 5.87 (d, J=8.1 Hz, 1H), 5.10-5.06 (m, 1H), 4.44-4.37 (m, 1H), 4.03 (dt, J=4.8 and 11.4 Hz, 1H), 3.90-3.80 (m, 1H), 3.11 (s, 3H), 2.34-2.26 (m, 1H), 1.14 (d, J=6.6 Hz, 3H), 1.00 (d, J=6.6 Hz, 3H).

### Example 34

### (R)-(1-isopropyl-7-(methyl sulfonyl)-2-(4-(trifluoromethyl)pyrimidin-2-yl)-1,2,3,4-tetrahydropyrazino[1,2-a]indol-8-yl)methanol and

### (S)-(1-isopropyl-7-(methylsulfonyl)-2-(4-(trifluoromethyl)pyrimidin-2-yl)-1,2,3,4-tetrahydropyrazino[1,2-a]indol-8-yl)methanol

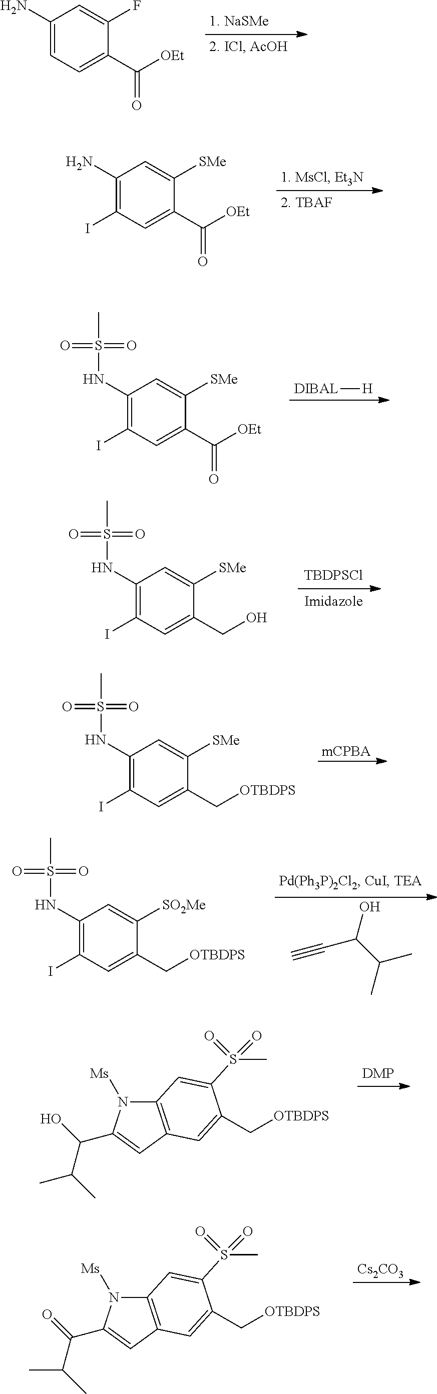

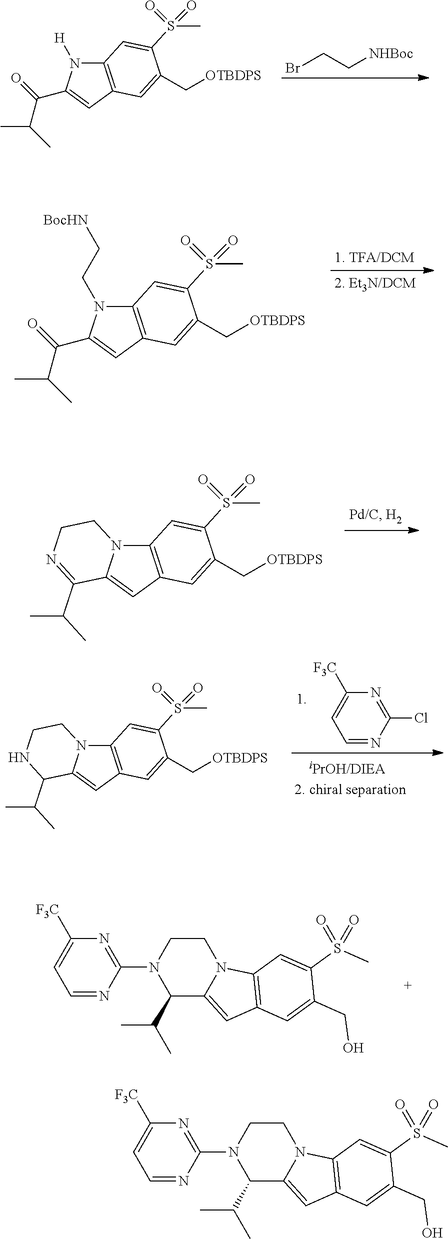

### Step 1:

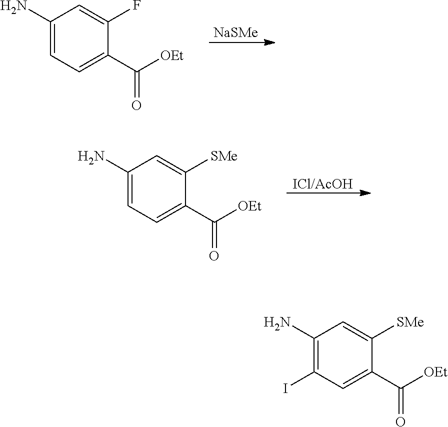

To a solution of ethyl 4-amino-2-fluorobenzoate (12 g, 65.5 mmol) in DMF (100 mL) was added NaSMe (9.17 g, 131 mmol) and the mixture was stirred at 60° C. for 20 h. After cooling to rt the reaction was diluted with H₂O and extracted with EtOAc (3×100 mL). The combined organic phases were washed with brine, dried over anhydrous Na₂SO₄, filtered and concentrated in vacuo to afford ethyl 4-amino-2-(methylthio)benzoate.

To a pre-heated, 60° C. solution of ethyl 4-amino-2-(methylthio)benzoate (65 mmol) in acetic acid (150 mL) was added ICl/AcOH solution (1M, 72 mL, 72 mmol) dropwise during 40 min and the temperature was maintained at 60° C. for 3 h. After cooling to rt the reaction was diluted with EtOAc (500 mL), washed with 5% Na₂S₂O₃ solution (3×100 mL) and brine (200 mL), dried over anhydrous sodium sulfate, filtered and concentrated in vacuo. The crude product was purified by silica gel chromatography (0-20% EtOAc/Hexanes) afford ethyl 4-amino-5-iodo-2-(methylthio)benzoate (13.67 g, 53% yield). For ethyl 4-amino-2-(methylthio)benzoate: LC-MS m/z 212 [M+H]⁺. For ethyl 4-amino-5-iodo-2-(methylthio)benzoate: LC-MS m/z 338 [M+H]⁺. ¹H NMR (400 MHz, CDCl₃): δ 8.29 (s, 1H), 6.47 (s, 1H), 4.49 (br s, 2H), 4.31 (q, J=7.2 Hz, 2H), 2.38 (s, 3H), 1.37 (t, J=7.2 Hz, 3H).

### Step 2:

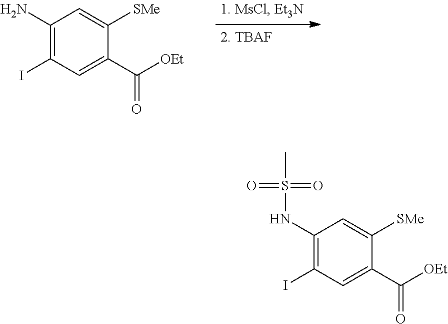 [SMILES: CCOC(=O)c1cc(I)c(N)cc1SC ; CCOC(=O)c1cc(I)c(NS(C)(=O)=O)cc1SC]

To a solution of ethyl 4-amino-5-iodo-2-(methylthio)benzoate (13.6 g, 40 mmol) in DCM (100 mL) was added Et₃N (13.8 mL, 100 mmol), followed by MsCl (7.7 mL, 100 mmol) at 0° C. After addition the mixture was stirred at rt for 2 h. 1N HCl solution (50 mL) was added to the mixture and the aqueous phase was extracted with DCM (1×100 mL). The organic solution was washed with brine, dried over anhydrous Na₂SO₄, filtered and concentrated in vacuo to give ethyl 5-iodo-4-(N-(methylsulfonyl)methylsulfonamido)-2-(methylthio)benzoate.

The crude reaction mixture above was dissolved into 100 mL THF. To this solution was added TBAF solution in THF (1 M, 100 mL) and the mixture was stirred at rt for 2 h. H₂O was added to the mixture and the aqueous phase was extracted with EtOAc (3×100 mL). The combined organic solution was washed with brine, dried over anhydrous Na₂SO₄, filtered and concentrated in vacuo to afford ethyl 5-iodo-4-(methylsulfonamido)-2-(methylthio)benzoate. It was used for next step without further purification. For ethyl 5-iodo-4-(N-(methylsulfonyl)methylsulfonamido)-2-(methylthio)benzoate: LC-MS m/z 494 [M+H]⁺. For ethyl 5-iodo-4-(methylsulfonamido)-2-(methylthio)benzoate: LC-MS m/z 415 [M+H]⁺.

### Step 3:

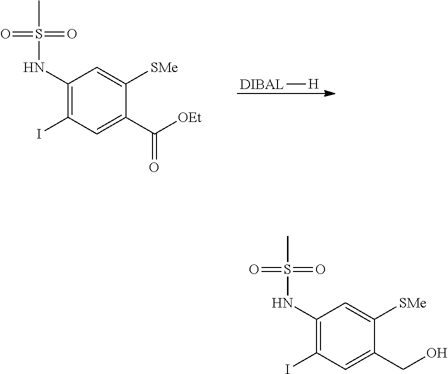

To a solution of ethyl 5-iodo-4-(methylsulfonamido)-2-(methylthio)benzoate (crude, from step 2) in dry toluene (200 mL) at 0° C. was added diisobutylaluminium hydride (1.0 M in toluene, 100 mL, 100 mmol) slowly. After addition, the mixture was stirred at 0° C. for 3 h and quenched with methanol/H₂O (1/1). The reaction mixture was poured into a vigorously stirred solution of potassium sodium tartrate (1M, 300 mL) and stirred vigorously for 2 h, after which time it settled to two clear phases. The organic layer was separated, and the aq layer was extracted with EtOAc (3×200 mL). The combined organic solution was washed with brine, dried over anhydrous sodium sulfate, filtered and concentrated in vacuo. The crude product was purified by silica gel chromatography (0-40% EtOAc/Hexanes) afford N-(4-(hydroxymethyl)-2-iodo-5-(methylthio)phenyl)methanesulfonamide (11.9 g, 80% yield for two steps). LC-MS m/z 356 [M+H]⁺. ¹H NMR (400 MHz, CDCl₃): δ 7.82 (s, 1H), 7.49 (s, 1H), 4.67 (s, 2H), 2.99 (s, 3H), 2.50 (s, 3H).

### Step 4:

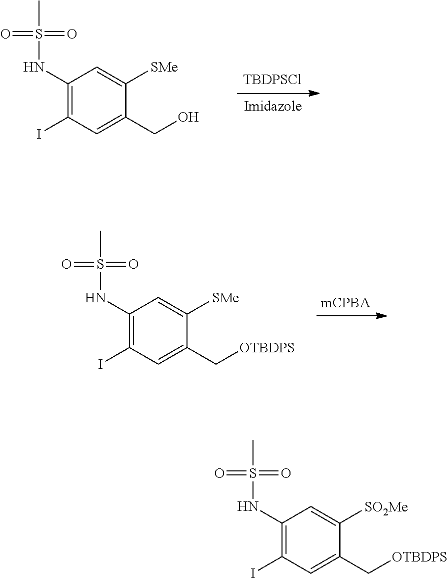

To a stirred solution of N-(4-(hydroxymethyl)-2-iodo-5-(methylthio)phenyl)methanesulfonamide (6.4 g, 17.2 mmol) and imidazole (1.76 g, 25.8 mmol) in CH₂Cl₂ (100 mL) and DMF (50 mL) at 0° C. was added tert-butyldiphenylsilyl chloride (5.8 mL, 22.4 mmol). The mixture was allowed to stir at rt overnight. The mixture was diluted with CH₂Cl₂ (100 mL), washed with 1N HCl solution, sat. aq. NaHCO₃ and brine, dried over anhydrous Na₂SO₄, filtered and concentrated in vacuo to afford N-(4-(((tert-butyldiphenylsilyl)oxy)methyl)-2-iodo-5-(methylthio)phenyl)methanesulfonamide. It was used for next step without further purification.

A suspension of crude N-(4-(((tert-butyldiphenylsilyl)oxy)methyl)-2-iodo-5-(methylthio)phenyl)methanesulfonamide and mCPBA (8.9 g, 51.6 mmol) in CH₂Cl₂ (100 mL) was stirred for 2 h at rt. Sat. aq. NaHCO₃ (50 mL) and Na₂S₂O₃ (50 mL) were added and the layers separated. The aqueous layer was extracted with CH₂Cl₂ (2×100 mL). The combined organic layers were dried over anhydrous Na₂SO₄, filtered and concentrated under reduced pressure. The residue was purified by flash chromatography over silica gel eluting with EtOAc/hexanes (3/7) to provide N-(4-(((tert-butyldiphenylsilyl)oxy)methyl)-2-iodo-5-(methylsulfonyl)phenyl)methanesulfonamide (8.8 g, 80% yield for two steps). For N-(4-(((tert-butyldiphenylsilyl)oxy)methyl)-2-iodo-5-(methylthio)phenyl)methanesulfonamide LC-MS m/z 612 [M+H]⁺. For N-(4-(((tert-butyldiphenylsilyl)oxy)methyl)-2-iodo-5-(methylsulfonyl)phenyl)methanesulfonamide: LC-MS m/z 644 [M+H]⁺. ¹H NMR (400 MHz, CDCl₃): δ 8.25 (s, 1H), 8.08 (s, 1H), 7.67-7.65 (m, 4H), 7.46-7.37 (m, 6H), 6.77 (s, 1H), 5.05 (s, 2H), 3.11 (s, 3H), 2.83 (s, 3H), 1.12 (s, 9H).

### Step 5:

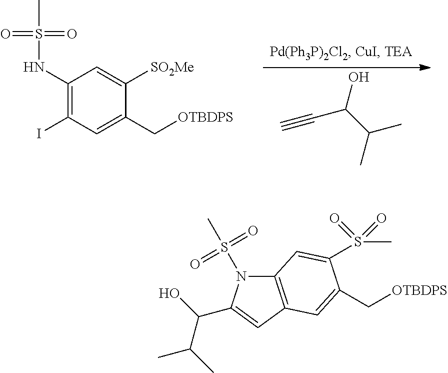

PdCl₂(PPh₃)₂ (277 mg, 0.38 mmol) and CuI (73 mg, 0.38 mmol) were added to a solution of N-(4-(((tert-butyldiphenylsilyl)oxy)methyl)-2-iodo-5-(methylsulfonyl)phenyl)methanesulfonamide (2.45 g, 3.8 mmol) in THF (20 mL) and Et₃N (10 mL). The mixture was purged with nitrogen for 10 min followed by addition of 4-methylpent-1-yn-3-ol (745 mg, 7.6 mmol) and stirred at 65° C. for 8 h. The reaction mixture was diluted with EtOAc (50 mL) and washed with 1N HCl (50 mL). The organic layer was separated, and the aq layer was extracted with EtOAc (3×50 mL). The combined organic solution was washed with brine, dried over anhydrous sodium sulfate, filtered and concentrated under reduced pressure. The residue was purified by flash chromatography over silica gel eluting with EtOAc/hexanes (3/7) to provide 1-(5-(((tert-butyldiphenylsilyl)oxy)methyl)-1,6-bis(methylsulfonyl)-1H-indol-2-yl)-2-methylpropan-1-ol (2.1 g, 90% yield). LC-MS m/z 614 [M+H]⁺. ¹H NMR (400 MHz, CDCl₃): δ 8.68 (s, 1H), 7.90 (s, 1H), 7.71-7.67 (s, 4H), 7.46-7.35 (m, 6H), 6.77 (s, 1H), 5.21 (d, J=3.2 Hz, 2H), 6.94 (t, J=6.8 Hz, 1H), 3.22 (s, 3H), 2.90 (s, 3H), 2.61 (d, J=6.8 Hz, 1H), 2.37-2.32 (m, 1H), 1.12 (s, 9H), 1.05 (d, J=6.8 Hz, 3H), 1.03 (d, J=6.8 Hz, 3H). ¹³C NMR (100 MHz, CDCl₃): δ 147.25, 135.54, 135.28, 135.00, 133.66, 133.00, 132.89, 129.96, 127.85, 121.68, 115.96, 108.69, 72.30, 62.98, 44.33, 41.59, 32.88, 26.89, 20.23, 19.30, 17.61.

### Step 6:

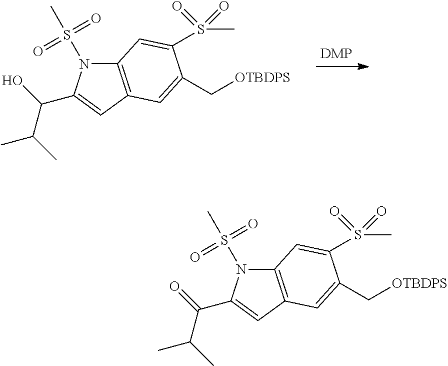

To a stirred solution of 1-(5-(((tert-butyldiphenylsilyl)oxy)methyl)-1,6-bis(methylsulfonyl)-1H-indol-2-yl)-2-methylpropan-1-ol (2.3 g, 3.8 mmol) in dry CH₂Cl₂ (25 mL) was added Dess-Martin periodiane (1.94 g, 4.56 mmol) in one portion. The mixture was allowed to stir at rt for 2 h. The reaction was quenched with a solution of Na₂S₂O₃ (5 g in 30 mL H₂O) and sat. aq. NaHCO₃ (40 mL). The mixture was extracted with EtOAc (3×80 mL). The combined organic solution was washed with brine, dried over anhydrous Na₂SO₄, filtered and concentrated under reduced pressure. The residue was purified by flash chromatography over silica gel eluting with EtOAc/hexanes (2/8) to provide 1-(5-(((tert-butyldiphenylsilyl)oxy)methyl)-1,6-bis(methylsulfonyl)-1H-indol-2-yl)-2-methylpropan-1-one (2.0 g, 86% yield). LC-MS m/z 612 [M+H]⁺. ¹H NMR (400 MHz, CDCl₃): δ 8.69 (s, 1H), 8.03 (s, 1H), 7.70-7.68 (m, 4H), 7.46-7.36 (m, 6H), 7.22 (s, 1H), 5.20 (s, 2H), 3.80 (s, 3H), 3.36 (m, 1H), 2.89 (s, 3H), 1.29 (d, J=6.8 Hz, 6H), 1.13 (s, 9H). ¹³C NMR (100 MHz, CDCl₃): δ 197.83, 141.53, 136.69, 136.05, 135.50, 135.04, 132.81, 131.14, 129.99, 127.88, 123.17, 117.12, 114.21, 62.87, 44.19, 44.03, 39.09, 26.88, 19.30, 18.41.

### Step 7:

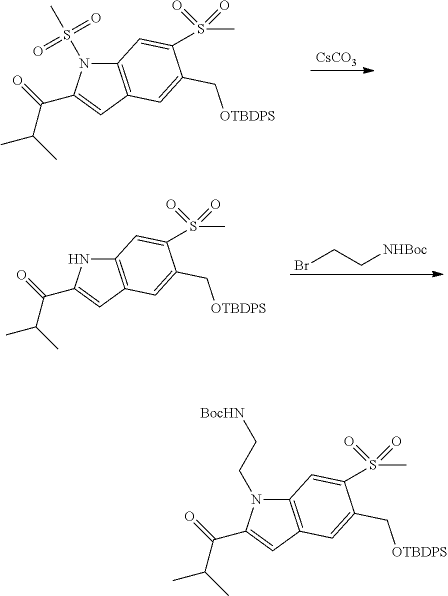

To a stirred solution of 1-(5-(((tert-butyldiphenylsilyl)oxy)methyl)-1,6-bis(methylsulfonyl)-1H-indol-2-yl)-2-methylpropan-1-one (780 mg, 1.27 mmol) in THF/methanol (15 mL/15 mL) was added Cs₂CO₃ (1.25 g, 3.83 mmol) in one portion. The mixture was allowed to stir at rt for 4 h and concentrated in vacuo to afford the crude product 1-(5-(((tert-butyldiphenylsilyl)oxy)methyl)-6-(methylsulfonyl)-1H-indol-2-yl)-2-methylpropan-1-one. It was used for the next step reaction without further purification.

To a solution of crude 1-(5-(((tert-butyldiphenylsilyl)oxy)methyl)-6-(methylsulfonyl)-1H-indol-2-yl)-2-methylpropan-1-one, 2-(Boc-amino)ethyl bromide (2.8 g, 12 mmol), tetrabutylammonium iodide (235 mg, 0.63 mmol) in CH₂Cl₂/toluene (2 mL/4 mL) was added 40% NaOH aqueous solution (20 mL). The mixture was allowed to stir at rt for 20 h. The reaction mixture was diluted with CH₂Cl₂ (40 mL) and washed with H₂O (50 mL). The organic layer was separated, and the aqueous layer was extracted with CH₂Cl₂ (4×50 mL). The combined organic solution was washed with brine, dried over anhydrous sodium sulfate, filtered and concentrated under reduced pressure. The residue was purified by flash chromatography over silica gel eluting with CH₂Cl₂/Methanol (95/5) to provide tert-butyl (2-(5-(((tert-butyldiphenylsilyl)oxy)methyl)-2-isobutyryl-6-(methylsulfonyl)-1H-indol-1-yl)ethyl)carbamate (300 mg, 35% yield for two steps).

For 1-(5-(((tert-butyldiphenylsilyl)oxy)methyl)-6-(methylsulfonyl)-1H-indol-2-yl)-2-methylpropan-1-one: LC-MS m/z 556 [M+Na]⁺.

For tert-butyl (2-(5-(((tert-butyldiphenylsilyl)oxy)methyl)-2-isobutyryl-6-(methylsulfonyl)-1H-indol-1-yl)ethyl)carbamate: LC-MS m/z 699 [M+Na]⁺. ¹H NMR (400 MHz, CDCl₃): δ 8.20 (s, 1H), 7.93 (s, 1H), 7.72 (dd, J₁=8.0 Hz, J₂=1.6 Hz, 4H), 7.47-7.35 (m, 7H), 5.21 (s, 2H), 4.72 (d, J=6.8 Hz, 2H), 3.55 (d, J=6.8 Hz, 2H), 3.33-3.26 (m, 1H), 3.00 (s, 3H), 1.46 (s, 9H), 1.30 (d, J=6.4 Hz, 3H), 1.28 (d, J=6.4 Hz, 3H), 1.11 (s, 9H).

### Step 8:

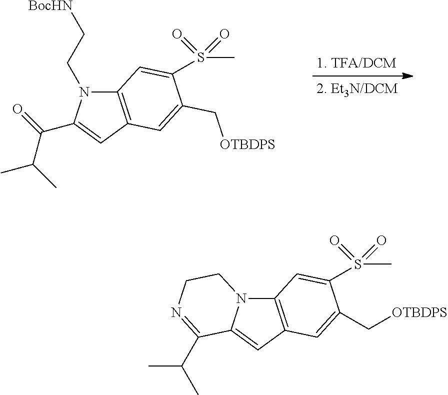

To a solution of tert-butyl (2-(5-(((tert-butyldiphenylsilyl)oxy)methyl)-2-isobutyryl-6-(methylsulfonyl)-1H-indol-1-yl)ethyl)carbamate (250 mg, 0.37 mmol) in CH₂Cl₂ (5.0 mL) was added trifluoroacetic acid (1.0 mL) and the mixture was allowed to stir at rt for 1 h. The excess amount of TFA was removed by azeotropic evaporation with toluene under reduced pressure. The residue was redissolved in CH₂Cl₂ (5 mL) and Et₃N (0.5 mL) was added. The reaction mixture was stirred at rt for 45 min and concentrated in vacuo. The residue was purified by flash chromatography over silica gel eluting with CH₂Cl₂/methanol (98/2) to provide 8-(((tert-butyldiphenylsilyl)oxy)methyl)-1-isopropyl-7-(methylsulfonyl)-3,4-dihydropyrazino[1,2-a]indole (135 mg, 65% yield). LC-MS m/z 559 [M+H]⁺.

### Step 9:

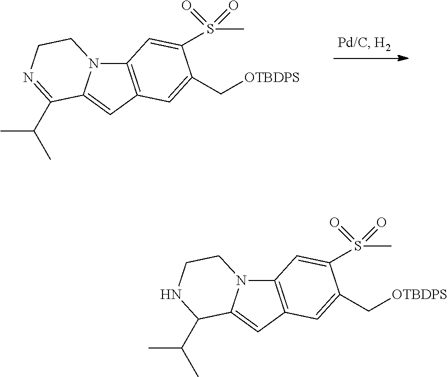

A solution of 8-(((tert-butyldiphenylsilyl)oxy)methyl)-1-isopropyl-7-(methylsulfonyl)-3,4-dihydropyrazino[1,2-a]indole (140 mg, 0.25 mmol), 10% palladium on charcoal (37 mg, 0.025 mmol), and methanol (5 mL) was stirred at rt under 1 atmosphere of hydrogen for 3 h. The mixture was filtered through Celite® and the Celite® was washed thoroughly with methanol. Combined solvent was removed under reduced pressure to afford 8-(((tert-butyldiphenylsilyl)oxy)methyl)-1-isopropyl-7-(methylsulfonyl)-1,2,3,4-tetrahydropyrazino[1,2-a]indole. It was used directly without further purification. A small portion of product was purified by chromatography for characterization. LC-MS m/z 561 [M+H]⁺. ¹H NMR (400 MHz, CD₃OD): δ 8.00 (s, 1H), 7.73-7.70 (m, 5H), 7.47-7.40 (m, 6H), 6.36 (s, 1H), 5.20 (d, J=2.0 Hz, 2H), 4.24-4.19 (m, 1H), 4.11-4.00 (m, 2H), 3.52-3.47 (m, 1H), 3.20-3.13 (m, 1H), 3.03 (s, 3H), 2.47-2.39 (m, 1H), 1.18 (d, J=6.8 Hz, 3H), 1.09 (s, 9H), 0.96 (d, J=6.8 Hz, 3H). ¹³C NMR (100 MHz, CDCl₃): δ 143.06, 135.68, 134.04, 133.31, 131.52, 130.26, 129.79, 129.51, 127.77, 121.39, 111.22, 97.14, 63.76, 59.28, 45.00, 42.94, 42.47, 31.55, 26.94, 19.72, 19.31, 16.49.

### Step 10:

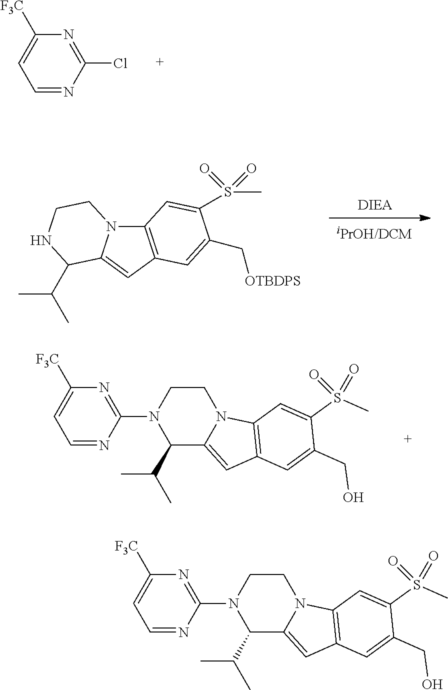

A mixture of 2-chloro-4-(trifluoromethyl)pyrimidine (80 mg, 0.44 mmol), 8-(((tert-butyldiphenylsilyl)oxy)methyl)-1-isopropyl-7-(methylsulfonyl)-1,2,3,4-tetrahydropyrazino[1,2-a]indole (crude, from step 9), and DIEA (115 μL, 0.66 mmol) in i-PrOH/CH₂Cl₂ (2 mL/1 mL) was stirred at 110° C. for 30 h. The solvent was removed under reduced pressure and the crude residue was purified by silica chromatography and SFC separation on a chiral column to give isomers of (1-isopropyl-7-(methylsulfonyl)-2-(4-(trifluoromethyl)pyrimidin-2-yl)-1,2,3,4-tetrahydropyrazino[1,2-a]indol-8-yl)methanol (75 mg, 72% yield for two steps).

Isomer 1: Analytical chiral HPLC: t_{R}=11.8 min in 15 min chromatography (Method: AD-H_5_5_40_2.35 ML). LC-MS m/z 469 [M+H]⁺. ¹H NMR (400 MHz, CD₃OD): δ 8.65 (d, J=4.8 Hz, 1H), 8.10 (s, 1H), 7.78 (s, 1H), 6.95 (d, J=4.8 Hz, 1H), 6.52 (s, 1H), 5.91-5.89 (m, 1H), 5.14-5.09 (m, 1H), 5.06 (s, 2H), 4.47-4.42 (m, 1H), 4.12-4.05 (m, 1H), 3.94-3.86 (m, 1H), 3.26 (s, 3H), 2.37-2.29 (m, 1H), 1.17 (d, J=6.8 Hz, 3H), 1.03 (d, J=6.8 Hz, 3H).

Isomer 2: Analytical chiral HPLC: t_{R}=9.7 min in 15 min chromatography (Method: AD-H_5_5_40_2.35 ML). LC-MS m/z 469 [M+H]⁺. ¹H NMR (400 MHz, CD₃OD): δ 8.65 (d, J=4.8 Hz, 1H), 8.10 (s, 1H), 7.78 (s, 1H), 6.95 (d, J=4.8 Hz, 1H), 6.52 (s, 1H), 5.91-5.89 (m, 1H), 5.14-5.09 (m, 1H), 5.06 (s, 2H), 4.47-4.42 (m, 1H), 4.12-4.05 (m, 1H), 3.94-3.86 (m, 1H), 3.26 (s, 3H), 2.37-2.29 (m, 1H), 1.17 (d, J=6.8 Hz, 3H), 1.03 (d, J=6.8 Hz, 3H).

### Example 35

Compound No. E7a was tested for its ability to prevent cholesterol accumulation and decrease inflammation in a lipid rich plaque in an experimental mouse model of accelerated atherosclerosis, correlative of ACS in humans. Such models are well known in the art (see, for example, Daugherty, 2002, Am. J. Med. Sci., 323:3-9 and Kuo et al., 2008, J. Lipid Res., 49:1353-1363). Apolipoprotein E knockout (ApoE−/−) mice were put on a high fat diet for two weeks. The left common carotid artery was surgically ligated and the mice continued the high fat diet for two additional weeks. Under these conditions, atherosclerotic lesions form within the carotid artery, characterized by an increase in cholesterol esters as well as an increase in vascular inflammation. Cholesterol esters were analyzed by LC/MS/MS in accordance with Kuo et al. In order to assess vascular inflammation, the mice were injected with non-radioactive fluorodeoxyglucose (FCG) 30 minutes prior to euthanasia, and a prominent daughter ion of the glucose metabolite FDG-6-phosphate (daughter ion 139.2 (mass to charge ratio)) was quantified in arterial extracts by LC/MS/MS. See e.g., Conway et al., 2012, PLoS One, Vol 7, Issue 11, 2012: e50349.

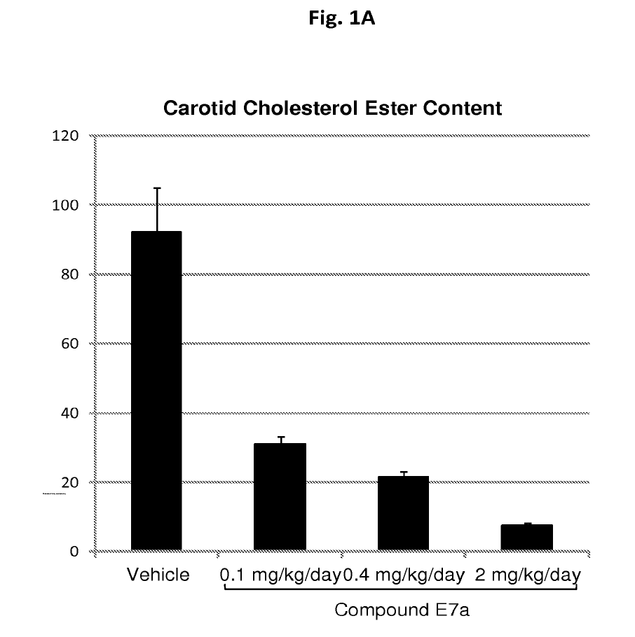

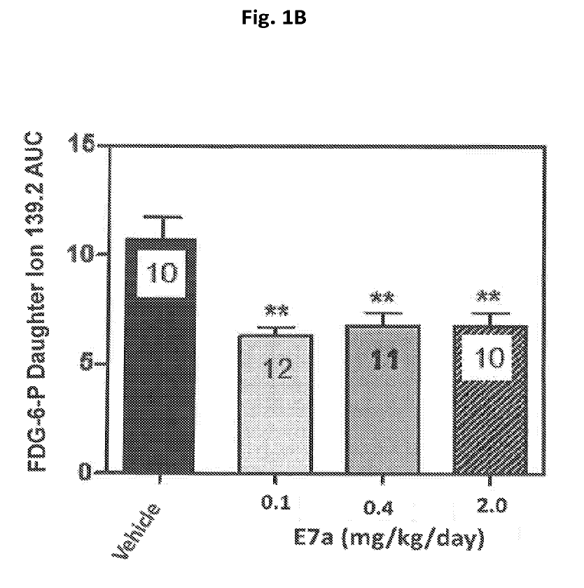

Compound No. E7a was orally administered to these mice twice a day for two weeks, beginning at the time of ligation surgery, and it produced a significant, dose-dependent decrease in cholesterol esters at all doses (see FIG. 1A). In addition, vascular inflammation was diminished at all doses of E7a, as evidenced by a decrease in the 1-DG-6-phosphate, an indirect marker of vascular inflammation (FIG. 1B; one-way ANOVA p<0.0001; Dunnett's 2-tailed t-test, vehicle versus all 3 concentrations of E7a **p<0.01), demonstrating the significant and potent anti-atherosclerotic effect of Compound No. E7a in this experimental model of atherosclerotic plaque inflammation.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a condition selected from acute coronary syndrome, heart attack, myocardial infarction, acute myocardial infarction, non-ST-segment elevation myocardial infarction, ST-segment elevation myocardial infarction, unstable angina, stable angina, angina pectoris, exercise induced angina, coronary artery disease, coronary heart disease, acute myocardial ischemia, ischaemic heart disease, ischemia, recurrent ischemia, congestive heart disease, congestive heart failure, cardiomyopathy, hypertensive heart disease, heart failure, diastolic heart failure, systolic heart failure, cor pulmonale, cardiac dysrhythmias, abnormalities of heart rhythm, inflammatory heart disease, endocarditis, inflammatory cardiomegaly, myocarditis, cerebrovascular disease, peripheral arterial disease, reperfusion injury, restenosis, atherosclerotic lesions, and chronic atherosclerotic inflammation in a subject, comprising administering to the subject an effective amount of a compound represented by structural formula I: 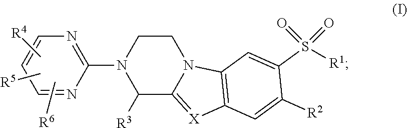
or a pharmaceutically acceptable salt thereof, wherein:
X is N or CR^{c};
R¹ is alkyl or —NR^{a}R^{b};
R² is H; halogen; —CN; —NRC(O)R; —C(O)OR; —C(O)NR^{a}R^{b}; monocyclic heteroaromatic optionally substituted with one or more groups selected from alkyl, —CN, —NRC(O)R, —C(O)OR, —C(O)NR^{a}R^{b} and halogen; monocyclic non-aromatic heterocycle optionally substituted with one or more groups selected from alkyl, halogen, —CN and ═O; or alkyl optionally substituted by one or more groups selected from halogen, hydroxy, alkoxy, —NR^{a}R^{b}, —NRC(O)R, —NRC(O)O(alkyl), —NRC(O)N(R)₂, —C(O)OR, thiol, alkylthiol, nitro, —CN, ═O, —OC(O)H, —OC(O)(alkyl), —OC(O)O(alkyl), —OC(O)N(R)₂ and —C(O)NR^{a}R^{b};
R³ is alkyl, haloalkyl, hydroxyalkyl, alkoxyalkyl, cycloalkyl, monocyclic non-aromatic heterocycle, monocyclic heteroaromatic or phenyl, wherein the phenyl, monocyclic non-aromatic heterocycle and monocyclic heteroaromatic group represented by R³ are optionally substituted with one or more groups selected from alkyl, halogen, haloalkyl, alkoxy, haloalkoxy, nitro and —CN;
R⁴ is halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic or alkyl, wherein the alkyl, monocyclic non-aromatic heterocycle and monocyclic heteroaromatic group represented by R⁴ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;
R⁵ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl, monocyclic non-aromatic heterocycle, monocyclic heteroaromatic or alkyl, wherein the alkyl, monocyclic non-aromatic heterocycle and monocyclic heteroaromatic group represented by R⁵ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;
R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂; or
R⁵ and R⁶, taken together with the carbon atoms to which they are bonded, form a monocyclic non-aromatic heterocycle optionally substituted with one or more groups selected from alkyl, halogen, hydroxyalkyl, alkoxyalkyl, haloalkyl and ═O;
each R independently is H or alkyl;
R^{a} and R^{b} are independently H, alkyl or R^{a} and R^{b} can be taken together with the nitrogen to which they are attached to form a monocyclic non-aromatic heterocycle; and
R^{c} is H, alkyl, or halogen.

2. The method of claim 1, wherein:
R³ is alkyl, haloalkyl, hydroxyalkyl, alkoxyalkyl, cycloalkyl, or phenyl, wherein the phenyl group represented by R³ is optionally substituted with one or more groups selected from alkyl, halogen, haloalkyl, alkoxy, haloalkoxy, nitro and —CN;
R⁴ is halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl represented by R⁴ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂;
R⁵ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl represented by R⁵ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂; and
R⁶ is H, halogen, —CN, —OR, —SR, —N(R)₂, —C(O)R, —C(O)OR, —OC(O)O(alkyl), —C(O)O(haloalkyl), —OC(O)R, —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂, —NRSO₂N(R)₂, haloalkyl, haloalkoxy, cycloalkoxy, cycloalkyl or alkyl, wherein the alkyl group represented by R⁶ is optionally substituted with one or more groups selected from —CN, —OR, —SR, —N(R)₂, ═O, —C(O)R, —C(O)OR, —C(O)O(haloalkyl), —OC(O)R, —OC(O)O(alkyl), —C(O)N(R)₂, —OC(O)N(R)₂, —NRC(O)R, —NRC(O)O(alkyl), —S(O)R, —SO₂R, —SO₂N(R)₂, —NRS(O)R, —NRSO₂R, —NRC(O)N(R)₂ and —NRSO₂N(R)₂.

3. The method of claim 2, wherein the compound is represented by the following structural formula II: 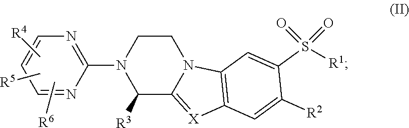
or a pharmaceutically acceptable salt thereof.

4. The method of claim 3, wherein the compound is represented by the following structural formula III: 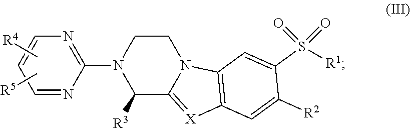
or a pharmaceutically acceptable salt thereof.

5. The method of claim 4, wherein the compound is represented by the following structural formula (IV): 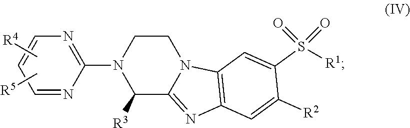
or a pharmaceutically acceptable salt thereof.

6. The method of claim 5, wherein the compound is represented by the following structural formula (V): 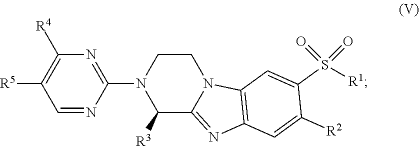
or a pharmaceutically acceptable salt thereof.

7. The method of claim 1, wherein the compound is represented by the following structural formula (VI): 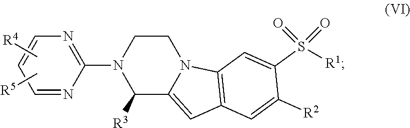
or a pharmaceutically acceptable salt thereof.

8. The method of claim 7, wherein the compound is represented by the following structural formula (VII): 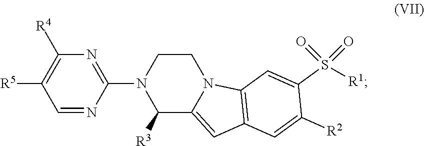
or a pharmaceutically acceptable salt thereof.

9. The method of claim 8, wherein:
R¹ is methyl or —NH₂;
R² is H or methyl, wherein the methyl group represented by R² is optionally substituted with one or more groups selected from halogen, hydroxy, alkoxy, —NR^{a}R^{b}, —NRC(O)R, —NRC(O)O(alkyl), —NRC(O)N(R)₂, —C(O)OR, thiol, alkylthiol, nitro, —CN, ═O, —OC(O)H, —OC(O)(alkyl), —OC(O)O(alkyl), —C(O)NR^{a}R^{b} and —OC(O)N(R)₂;
R³ is methyl, ethyl, propyl, isopropyl, tert-butyl, sec-butyl, iso-butyl, —CH₂CF₃, —CH(CH₂F)₂, —CH(CHF₂)₂, —CH(CF₃)₂, —CF(CH₃)₂, —CF₃, cyclopropyl, cyclobutyl, cyclopentyl, cyclohexyl, —C(OH)(CH₃)₂, —CH(OH)(CH₃), or phenyl, wherein the phenyl group represented by R³ is optionally substituted with one or more groups selected from alkyl, halogen, haloalkyl, alkoxy, haloalkoxy, nitro and —CN; and
R^{c}, where present, is H.

10. The method of claim 9, wherein R² is H or —CH₂OH.

11. The method of claim 10, wherein R¹ is methyl; R² is —CH₂OH; and R³ is isopropyl.

12. The method of claim 11, wherein
R⁴ is halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl or dihydroxyalkyl; and
R⁵ is H, halogen, hydroxy, alkyl, cycloalkyl, cycloalkoxy, alkoxy, haloalkoxy, haloalkyl, —N(R)₂, —C(O)OH, —C(O)O(alkyl), —C(O)O(haloalkyl), —C(O)(alkyl), —C(O)N(R)₂, —NRC(O)R, —SO₂N(R)₂, —OC(O)N(R)₂, —CN, hydroxyalkyl or dihydroxyalkyl.

13. The method of claim 12, wherein
R⁴ is methyl, ethyl, hydroxy, CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂ or —OCH₃; and
R⁵ is H, methyl, ethyl, hydroxy, CF₃, isopropyl, cyclopropyl, —CH₂OH, —CH(OH)(CH₂)(OH), —C(OH)(CH₃)₂, —CH(OH)(CH₃), —CH(OH)(CH₂)(CH₃), —CH(OH)(CH₂)₂(CH₃), —C(O)NH₂, —C(O)N(CH₃)₂, —C(O)OH, —C(O)NH(CH₃), —C(O)CH₃, —C(O)CH₂CH₃, —C(O)O(CH₂)(CH₃), —C(O)O(tert-butyl), —C(O)O(C)(CH₃)₂(CF₃), —NHC(O)CH₃, —OCHF₂, —OCF₃, —OCH₂CH₃, —OCH(CH₃)₂ or —OCH₃.

14. The method of claim 12, wherein R⁴ is alkyl, haloalkyl, cycloalkyl, alkoxy, or haloalkoxy.

15. The method claim 14, wherein R⁴ is methyl, halogenated methyl, cyclopropyl, —OCHF₂ or —OCH₃.

16. The method of claim 15, wherein R⁴ is CF₃.

17. The method of claim 16, wherein R⁵ is H or C(OH)(CH₃)₂.

18. (canceled)

19. (canceled)

20. The method of claim 1, wherein the compound is 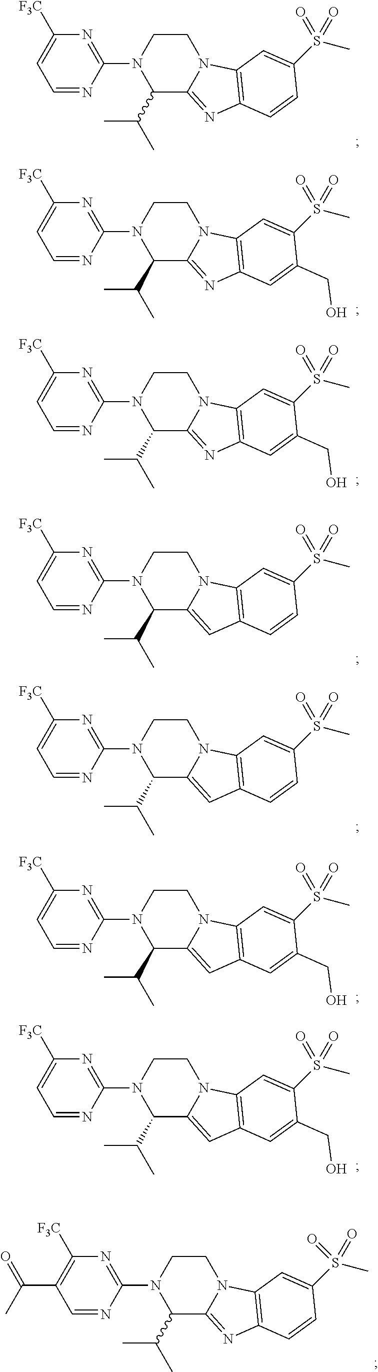 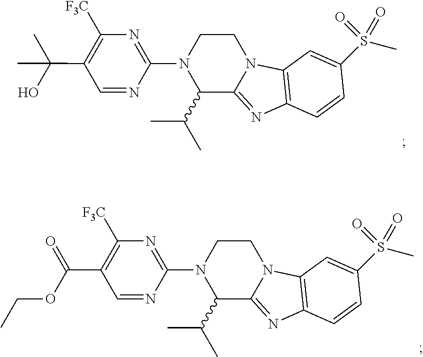 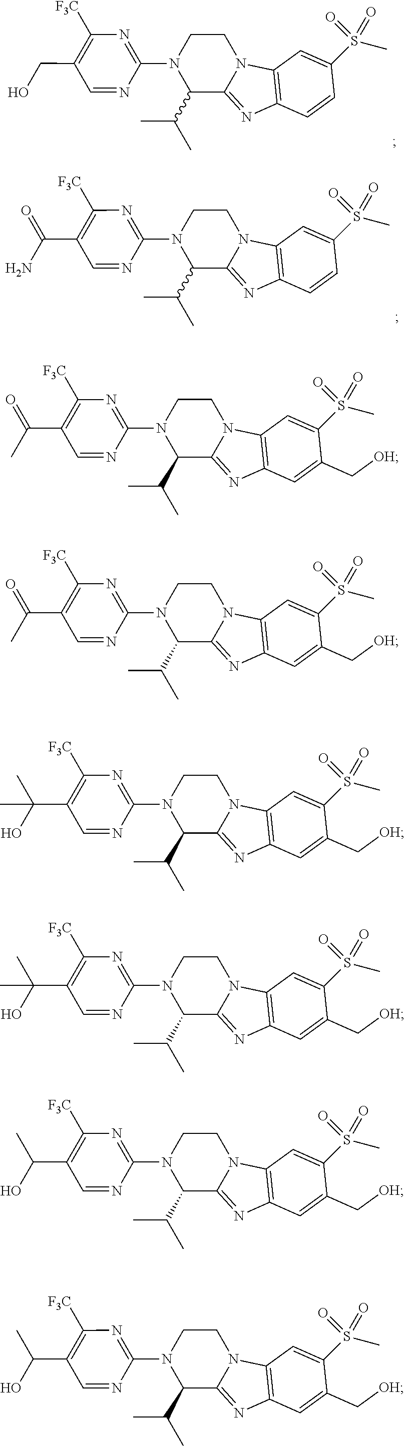 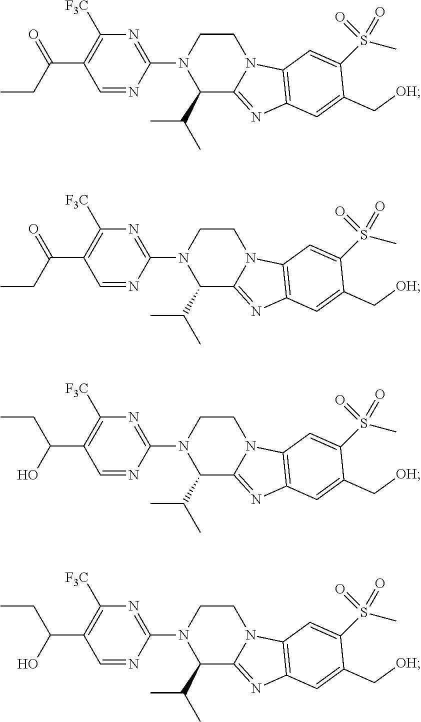 [SMILES: CCC(=O)c1cnc(N2CCn3c(nc4cc(CO)c(S(C)(=O)=O)cc43)[C@H]2C(C)C)nc1C(F)(F)F ; CCC(=O)c1cnc(N2CCn3c(nc4cc(CO)c(S(C)(=O)=O)cc43)[C@@H]2C(C)C)nc1C(F)(F)F ; CCC(O)c1cnc(N2CCn3c(nc4cc(CO)c(S(C)(=O)=O)cc43)[C@@H]2C(C)C)nc1C(F)(F)F ; CCC(O)c1cnc(N2CCn3c(nc4cc(CO)c(S(C)(=O)=O)cc43)[C@H]2C(C)C)nc1C(F)(F)F] 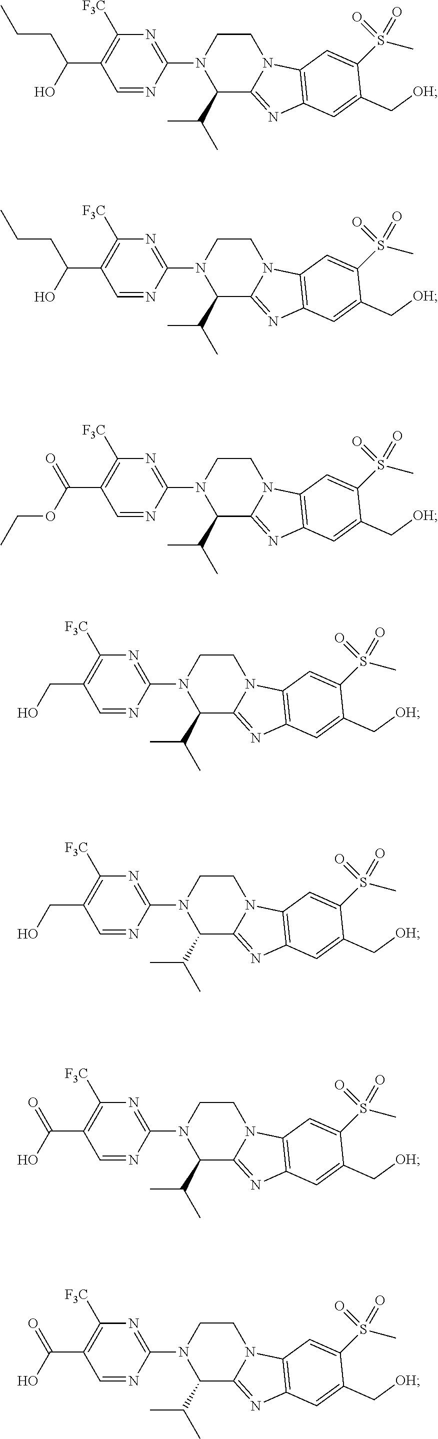 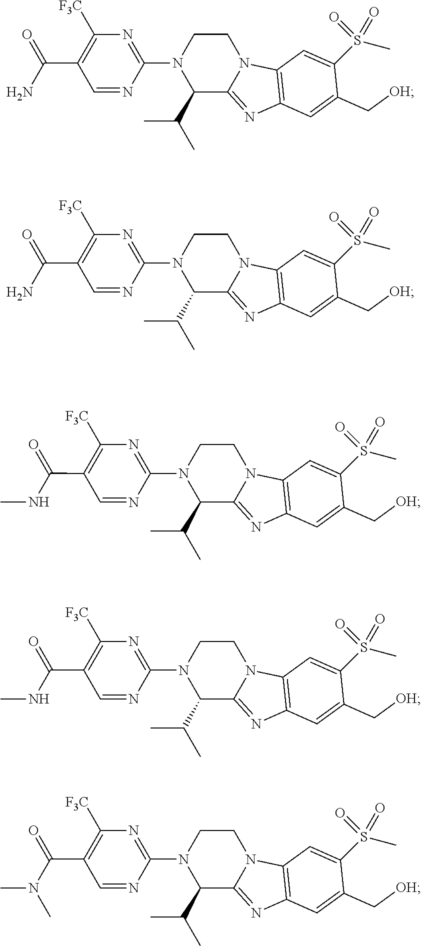 [SMILES: CC(C)[C@@H]1c2nc3cc(CO)c(S(C)(=O)=O)cc3n2CCN1c1ncc(C(N)=O)c(C(F)(F)F)n1 ; CC(C)[C@H]1c2nc3cc(CO)c(S(C)(=O)=O)cc3n2CCN1c1ncc(C(N)=O)c(C(F)(F)F)n1 ; CNC(=O)c1cnc(N2CCn3c(nc4cc(CO)c(S(C)(=O)=O)cc43)[C@H]2C(C)C)nc1C(F)(F)F ; CNC(=O)c1cnc(N2CCn3c(nc4cc(CO)c(S(C)(=O)=O)cc43)[C@@H]2C(C)C)nc1C(F)(F)F ; CC(C)[C@@H]1c2nc3cc(CO)c(S(C)(=O)=O)cc3n2CCN1c1ncc(C(=O)N(C)C)c(C(F)(F)F)n1] 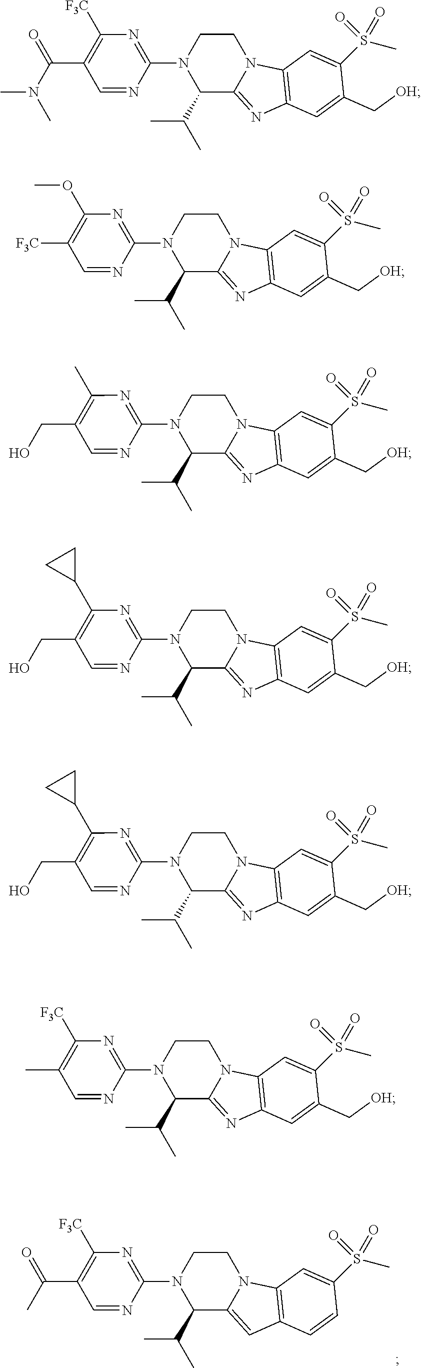 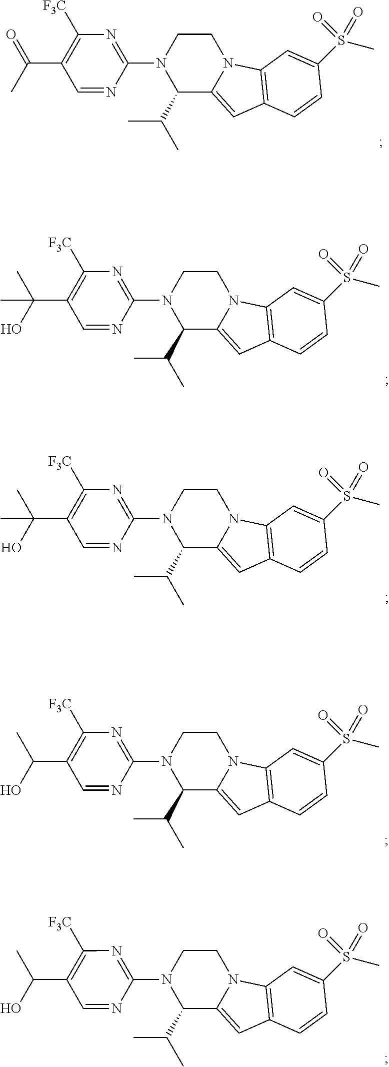 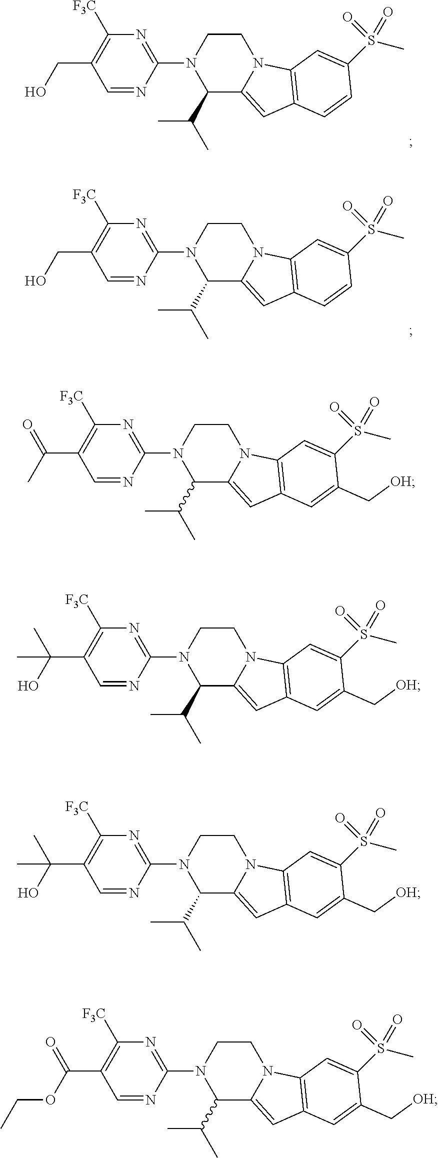 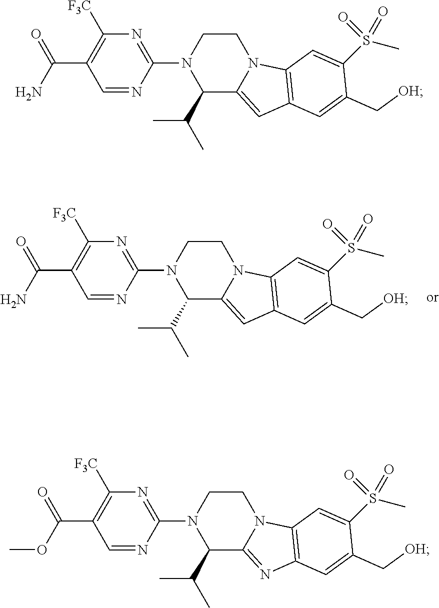 [SMILES: CC(C)[C@@H]1c2cc3cc(CO)c(S(C)(=O)=O)cc3n2CCN1c1ncc(C(N)=O)c(C(F)(F)F)n1 ; CC(C)[C@H]1c2cc3cc(CO)c(S(C)(=O)=O)cc3n2CCN1c1ncc(C(N)=O)c(C(F)(F)F)n1 ; "or" ; COC(=O)c1cnc(N2CCn3c(nc4cc(CO)c(S(C)(=O)=O)cc43)[C@H]2C(C)C)nc1C(F)(F)F]
or a pharmaceutically acceptable salt thereof.

21. The method of claim 1, wherein the condition is acute coronary syndrome.

22. The method of claim 21, wherein the compound is of the formula: 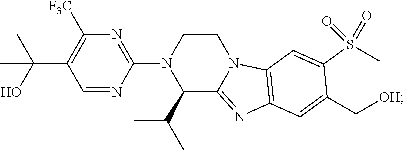 [SMILES: CC(C)[C@@H]1c2nc3cc(CO)c(S(C)(=O)=O)cc3n2CCN1c1ncc(C(C)(C)O)c(C(F)(F)F)n1]
or a pharmaceutically acceptable salt thereof.
